(19) United States
(12) Patent Application Publication
(10) Pub. No.: US 2026/0317399 A1
(43) Pub. Date: Oct. 15, 2026

(54) FCgammaRIIB-SPECIFIC FC REGION VARIANT

(71) Applicant: Chugai Seiyaku Kabushiki Kaisha, Tokyo (JP)

(72) Inventors: Hitoshi Katada, Shizuoka (JP); Shojiro Kadono, Kanagawa (JP); Futa Mimoto, Shizuoka (JP); Tomoyuki Igawa, Shizuoka (JP)

(73) Assignee: Chugai Seiyaku Kabushiki Kaisha, Tokyo (JP)

(21) Appl. No.: 19/354,304

(22) Filed: Oct. 9, 2025

Related U.S. Application Data
(62) Division of application No. 17/028,210, filed on Sep. 22, 2020, now abandoned.
Application No. 17/028,210 is a division of application No. 14/423,269, filed on Aug. 23, 2013, now Pat. No. 10,919,953.

(30) Foreign Application Priority Data
Aug. 24, 2012 (JP) 2012-185868

(51) Int. Cl. C07K 16/00; A61K 9/00; A61K 39/00; C07K 16/28; C07K 16/30

(52) U.S. Cl. CPC C07K 16/00; C07K 16/28; C07K 16/2866; C07K 16/303; A61K 9/0019; A61K 2039/505; C07K 2317/52; C07K 2317/524; C07K 2317/53; C07K 2317/71; C07K 2317/72; C07K 2317/92; C07K 2317/94; C07K 2319/30; Y02A 50/30

(57) ABSTRACT

An objective of the present invention is to provide an Fc region variant with enhanced FcγRIIb-binding activity, and/or enhanced binding selectivity to FcγRIIb compared to FcγRIIa (type R), as compared to those of a polypeptide containing an Fc region to which an amino acid alteration(s) has not been introduced; a polypeptide which includes the Fc region variant; a pharmaceutical composition containing the polypeptide; preventing therapeutic or preventive agent for immunological inflammatory diseases that includes the pharmaceutical composition; a production method thereof; and a method of enhancing FcγRIIb-binding activity and also enhancing binding selectivity to FcγRIIb compared to FcγRIIa (type R).

It was found that a polypeptide containing an antibody Fc region variant that contains an amino acid sequence in which an amino-acid alteration at position 238 (EU numbering) is combined with other specific amino-acid alterations enhances FcγRIIb-binding activity, and/or enhances binding selectivity to FcγRIIb compared to FcγRIIa (type R).

### CROSS-REFERENCE TO RELATED APPLICATIONS

This application is a divisional application of U.S. application Ser. No. 17/028,210, filed on Sep. 22, 2020, which is a divisional application of U.S. application Ser. No. 14/423,269, filed on Feb. 23, 2015 (now U.S. Pat. No. 10,919,953), which is the National Stage of International Application No. PCT/JP2013/072507, filed on Aug. 23, 2013, which claims the benefit of Japanese Application No. 2012-185868, filed on Aug. 24, 2012. The entire content of parent application Ser. No. 17/028,210 is hereby incorporated by reference.

### SEQUENCE LISTING

This application contains a Sequence Listing that has been submitted electronically as an XML file named 38856-0245003_SL_ST26.xml. The XML file, created on Oct. 7, 2025, is 101,908 bytes in size. The material in the XML file is hereby incorporated by reference in its entirety.

### TECHNICAL FIELD

The present invention relates to Fc region variants introduced with amino acid alteration(s) into an antibody Fc region, which have enhanced FcγRIIb-binding activity, and/or enhanced binding selectivity to FcγRIIb compared to FcγRIIa (type R), as compared to an Fc region to which an amino acid alteration(s) has not been introduced; polypeptides comprising the Fc region variants; and pharmaceutical compositions comprising the polypeptides.

### BACKGROUND ART

Antibodies are drawing attention as pharmaceuticals since they are highly stable in blood and have few side effects (Non-patent Documents 1 and 2). Almost all therapeutic antibodies currently on the market are antibodies of the human IgG1 subclass. One of the known functions of IgG class antibodies is antibody-dependent cell-mediated cytotoxicity (hereinafter denoted as ADCC activity) (Non-patent Document 3). For an antibody to exhibit ADCC activity, the antibody Fc region must bind to an Fcγ receptor (hereinafter denoted as FcγR) which is an antibody-binding receptor present on the surface of effector cells such as killer cells, natural killer cells, and activated macrophages.

In humans, the FcγRIa (CD64A), FcγRIIa (CD32A), FcγRIIb (CD32B), FcγRIIIa (CD16A), and FcγRIIIb (CD16B) isoforms have been reported as the FcγR protein family, and the respective allotypes have also been reported (Non-patent Document 7). FcγRIa, FcγRIIa, and FcγRIIIa are called activating FcγR since they have immunologically active functions, and FcγRIIb is called inhibitory FcγR since it has immunosuppressive functions (Non-patent Document 8).

In the binding between the Fc region and FcγR, several amino acid residues in the antibody hinge region and CH2 domain, and a sugar chain attached to Asn at position 297 (EU numbering) bound to the CH2 domain have been shown to be important (Non-patent Documents 4, 5, and 6). Various variants having FcγR-binding properties, mainly antibodies with mutations introduced into these sites, have been studied so far; and Fc region variants having higher binding activities towards activating FcγR have been obtained (Patent Documents 1, 2, 3, and 4).

When activating FcγR is cross-linked with an immune complex, it phosphorylates immunoreceptor tyrosine-based activating motifs (ITAMs) contained in the intracellular domain or FcR common γ-chain (an interaction partner), activates a signal transducer SYK, and triggers inflammatory immune response by initiating an activation signal cascade (Non-patent Document 9).

FcγRIIb is the only FcγR expressed on B cells (Non-patent Document 10). Interaction of the antibody Fc region with FcγRIIb has been reported to suppress the primary immune response of B cells (Non-patent Document 11). Furthermore, it is reported that when FcγRIIb on B cells and a B cell receptor (BCR) are cross-linked via an immune complex in blood, B cell activation is suppressed, and antibody production by B cells is suppressed (Non-patent Document 12). In this immunosuppressive signal transduction mediated by BCR and FcγRIIb, the immunoreceptor tyrosine-based inhibitory motif (ITIM) contained in the intracellular domain of FcγRIIb is necessary (Non-patent Documents 13 and 14). When ITIM is phosphorylated upon signaling, SH2-containing inositol polyphosphate 5-phosphatase (SHIP) is recruited, transduction of other activating FcγR signal cascades is inhibited, and inflammatory immune response is suppressed (Non-patent Document 15). Furthermore, aggregation of FcγRIIb alone has been reported to transiently suppress calcium influx due to BCR cross-linking and B cell proliferation in a BCR-independent manner without inducing apoptosis of IgM-producing B cells (Non-patent Document 16).

Furthermore, FcγRIIb is also expressed on dendritic cells, macrophages, activated neutrophils, mast cells, and basophils. FcγRIIb inhibits the functions of activating FcγR such as phagocytosis and release of inflammatory cytokines in these cells, and suppresses inflammatory immune responses (Non-patent Document 8).

The importance of immunosuppressive functions of FcγRIIb has been elucidated so far through studies using FcγRIIb knockout mice. There are reports that in FcγRIIb knockout mice, humoral immunity is not appropriately regulated (Non-Patent Document 17), sensitivity towards collagen-induced arthritis (CIA) is increased (Non-patent Document 18), lupus-like symptoms are presented, and Goodpasture's syndrome-like symptoms are presented (Non-patent Document 19).

Furthermore, regulatory inadequacy of FcγRIIb has been reported to be related to human autoimmune diseases. For example, the relationship between genetic polymorphism in the transmembrane region and promoter region of FcγRIIb, and the frequency of development of systemic lupus erythematosus (SLE) (Non-patent Documents 20, 21, 22, 23, and 24), and decrease of FcγRIIb expression on the surface of B cells in SLE patients (Non-patent Document 25 and 26) have been reported.

From mouse models and clinical findings as such, FcγRIIb is considered to play the role of controlling autoimmune diseases and inflammatory diseases through involvement in particular with B cells, and it is a promising target molecule for controlling autoimmune diseases and inflammatory diseases.

IgG1, mainly used as a commercially available therapeutic antibody, is known to bind not only to FcγRIIb, but also strongly to activating FcγR (Non-patent Document 27). It may be possible to develop therapeutic antibodies having greater immunosuppressive properties compared with those of IgG1, by utilizing an Fc region with enhanced FcγRIIb binding, or improved FcγRIIb-binding selectivity compared with activating FcγR. For example, it has been suggested that the use of an antibody having a variable region that binds to BCR and an Fc with enhanced FcγRIIb binding may inhibit B cell activation (Non-patent Document 28). It has been reported that crosslinking FcγRIIb on B cells and IgE bound to a B-cell receptor suppresses differentiation of B cells into plasma cells, which as a result causes suppression of IgE production; and in human PBMC-transplanted mice, human IgG and IgM concentrations are maintained whereas the human IgE concentration is decreased (Non-patent Document 29). Besides IgE, it has been reported that when FcγRIIB and CD79b which is a constituent molecule of a B-cell receptor complex are cross-linked by an antibody, B cell proliferation is suppressed in vitro, and arthritis symptoms are alleviated in the collagen arthritis model (Non-patent Document 30).

Besides B cells, it has been reported that crosslinking of FcεRI and FcγRIIb on mast cells using molecules, in which the Fc portion of an IgG with enhanced FcγRIIb binding is fused to the Fc portion of IgE that binds to an IgE receptor FcεRI, causes FcγRIIb phosphorylation of FcγRIIb, thereby suppressing FcεRI-dependent calcium influx. This suggests that inhibition of degranulation via FcγRIIb stimulation is possible by enhancing FcγRIIb binding (Non-patent Document 31).

Accordingly, an antibody having an Fc with improved FcγRIIb-binding activity is suggested to be promising as a therapeutic agent for inflammatory diseases such as autoimmune diseases.

Furthermore, it has been reported that activation of macrophages and dendritic cells via Toll-like receptor 4 due to LPS stimulation is suppressed in the presence of an antibody-antigen immune complex, and this effect is also suggested to be actions of the immune complex via FcγRIIb (Non-patent Documents 32 and 33). Therefore, use of antibodies with enhanced FcγRIIb binding is expected to enable enhancement of TLR-mediated activation signal-suppressing actions; thus such antibodies have been suggested as being promising as therapeutic agents for inflammatory diseases such as autoimmune diseases.

Furthermore, mutants with enhanced FcγRIIb binding have been suggested to be promising therapeutic agents for cancer, as well as therapeutic agents for inflammatory diseases such as autoimmune diseases. So far, FcγRIIb has been found to play an important role in the agonistic activity of agonist antibodies against the anti-TNF receptor superfamily. Specifically, it has been suggested that interaction with FcγRIIb is required for the agonistic activity of antibodies against CD40, DR4, DR5, CD30, and CD137, which are included in the TNF receptor family (Non-patent Documents 34, 35, 36, 37, 38, 39 and 40). Non-patent Document 34 shows that the use of antibodies with enhanced FcγRIIb binding enhances the anti-tumor effect of anti-CD40 antibodies. Accordingly, antibodies with enhanced FcγRIIb are expected to have an effect of enhancing agonistic activity of agonist antibodies including antibodies against the anti-TNF receptor superfamily.

In addition, it has been shown that cell proliferation is suppressed when using an antibody that recognizes Kit, a type of receptor tyrosine kinase (RTK), to crosslink FcγRIIb and Kit on Kit-expressing cells. Similar effects have been reported even in cases where this Kit is constitutively activated and has mutations that cause oncogenesis (Non-patent Document 41). Therefore, it is expected that use of antibodies with enhanced FcγRIIb binding may enhance inhibitory effects on cells expressing RTK having constitutively activated mutations.

Antibodies having an Fc with improved FcγRIIb-binding activity have been reported (Non-patent Document 28). In this Document, FcγRIIb-binding activity was improved by adding alterations such as S267E/L328F, G236D/S267E, and S239D/S267E to an antibody Fc region. Among them, the antibody introduced with the S267E/L328F mutation most strongly binds to FcγRIIb, and maintains the same level of binding to FcγRIa and FcγRIIa type H in which a residue at position 131 of FcγRIIa is His as that of a naturally-occurring IgG1. However, another report shows that this alteration enhances the binding to FcγRIIa type R in which a residue at position 131 of FcγRIIa is Arg several hundred times to the same level of FcγRIIb binding, which means the FcγRIIb-binding selectivity is not improved in comparison with type-R FcγRIIa (Patent Document 5).

Only the effect of enhancing FcγRIIa binding and not the enhancement of FcγRIIb binding is considered to have influence on cells such as platelets which express FcγRIIa but do not express FcγRIIb (Non-patent Document 8). For example, the group of patients who were administered bevacizumab, an antibody against VEGF, is known to have an increased risk for thromboembolism (Non-patent Document 42). Furthermore, thromboembolism has been observed in a similar manner in clinical development tests of antibodies against the CD40 ligand, and the clinical study was discontinued (Non-patent Document 43). In both cases of these antibodies, later studies using animal models and such have suggested that the administered antibodies aggregate platelets via FcγRIIa binding on the platelets, and form blood clots (Non-patent Documents 44 and 45). In systemic lupus erythematosus which is an autoimmune disease, platelets are activated via an FcγRIIa-dependent mechanism, and platelet activation has been reported to correlate with the severity of symptoms (Non-patent Document 46). Administering an antibody with enhanced FcγRIIa binding to such patients who already have a high risk for developing thromboembolism will increase the risk for developing thromboembolism, thus is extremely dangerous.

Furthermore, antibodies with enhanced FcγRIIa binding have been reported to enhance macrophage-mediated antibody dependent cellular phagocytosis (ADCP) (Non-patent Document 47). When antigens to be bound by the antibodies are phagocytized by macrophages, antibodies themselves are considered to be also phagocytized at the same time. When antibodies are administered as pharmaceuticals, it is supposed that peptide fragments derived from the administered antibodies are likely to be also presented as an antigen, thereby increasing the risk of production of antibodies against therapeutic antibodies (anti-therapeutic antibodies). More specifically, enhancing FcγRIIa binding will increase the risk of production of antibodies against the therapeutic antibodies, and this will remarkably decrease their value as pharmaceuticals. Furthermore, FcγRIIb on dendritic cells have been suggested to contribute to peripheral tolerance by inhibiting dendritic cell activation caused by immune complexes formed between antigens and antibodies, or by suppressing antigen presentation to T cells via activating Fcγ receptors (Non-patent Document 48). Since FcγRIIa is also expressed on dendritic cells, when antibodies having an Fc with enhanced selective binding to FcγRIIb are used as pharmaceuticals, antigens are not readily presented by dendritic cells and such due to enhanced selective binding to FcγRIIb, and risk of anti-drug antibody production can be relatively decreased. Such antibodies may be useful in that regard as well.

More specifically, the value as pharmaceuticals will be considerably reduced when FcγRIIa binding is enhanced, which leads to increased risk of thrombus formation via platelet aggregation and increased risk of anti-therapeutic antibody production due to an increased immunogenicity.

From such a viewpoint, the aforementioned Fc variant with enhanced FcγRIIb binding shows remarkably enhanced type-R FcγRIIa binding compared with that of a naturally-occurring IgG1. Therefore, its value as a pharmaceutical for patients carrying type-R FcγRIIa is considerably reduced. Types H and R of FcγRIIa are observed in Caucasians and African-Americans with approximately the same frequency (Non-patent Documents 49 and 50). Therefore, when this Fc variant was used for treatment of autoimmune diseases, the number of patients who can safely use it while enjoying its effects as a pharmaceutical will be limited.

Furthermore, in dendritic cells deficient in FcγRIIb or dendritic cells in which the interaction between FcγRIIb and the antibody Fc portion is inhibited by an anti-FcγRIIb antibody, dendritic cells have been reported to mature (Non-patent Documents 51 and 52). This report suggests that FcγRIIb is actively suppressing maturation of dendritic cells in a steady state where inflammation and such are not taking place and activation does not take place. FcγRIIa is expressed on the dendritic cell surface in addition to FcγRIIb; therefore, even if binding to inhibitory FcγRIIb is enhanced and if binding to activating FcγR such as FcγRIIa is also enhanced, maturation of dendritic cells may be promoted as a result. More specifically, improving not only the FcγRIIb-binding activity but also the ratio of FcγRIIb-binding activity relative to FcγRIIa-binding activity is considered to be important in providing antibodies with an immunosuppressive action.

Therefore, when considering generation of pharmaceuticals that utilize the FcγRIIb binding-mediated immunosuppressive action, there is a need for an Fc variant that not only has enhanced FcγRIIb-binding activity, but also has binding to both FcγRIIa, types H and R allotypes, which is maintained at a similar level or is weakened to a lower level than that of a naturally-occurring IgG1.

Meanwhile, cases where amino acid alterations were introduced into the Fc region to increase the FcγRIIb-binding selectivity have been reported so far (Non-patent Document 53). However, all variants said to have improved FcγRIIb selectivity as reported in this document showed decreased FcγRIIb binding compared with that of a naturally-occurring IgG1. Therefore, it is considered to be difficult for these variants to actually induce an FcγRIIb-mediated immunosuppressive reaction more strongly than IgG1.

Furthermore, since FcγRIIb plays an important role in the agonist antibodies mentioned above, enhancing their binding activity is expected to enhance the agonistic activity. However, when FcγRIIa binding is similarly enhanced, unintended activities such as ADCC activity and ADCP activity will be exhibited, and this may cause side effects. Also from such viewpoint, it is preferable to be able to selectively enhance FcγRIIb-binding activity.

From these results, in producing therapeutic antibodies to be used for treating autoimmune diseases and cancer utilizing FcγRIIb, it is important that compared with those of a naturally-occurring IgG, the activities of binding to both FcγRIIa allotypes are maintained or decreased, and FcγRIIb binding is enhanced. However, FcγRIIb shares 93% sequence identity in the extracellular region with that of FcγRIIa which is one of the activating FcγRs, and they are very similar structurally. There are allotypes of FcγRIIa, H type and R type, in which the amino acid at position 131 is His (type H) or Arg (type R), and yet each of them reacts differently with the antibodies (Non-patent Document 54). Therefore, the difficult problem may be producing an Fc region variant with enhanced selective FcγRIIb binding as compared to each allotype of FcγRIIa, which involves distinguishing highly homologous sequences between FcγRIIa and FcγRIIb. In fact, variants having sufficient binding activity and selectivity to FcγRIIb have not been obtained so far. Patent Document 5 reports variants with enhanced FcγRIIb-binding activity; however, the degree of enhancement is low, and there is a demand for development of variants having properties similar to those described above.

### PRIOR ART DOCUMENTS

### Patent Documents

[Patent Document 1] WO 2000/42072
[Patent Document 2] WO 2006/019447
[Patent Document 3] WO 2004/99249
[Patent Document 4] WO 2004/29207
[Patent Document 5] US2009/0136485

### Non-Patent Documents

[Non-patent Document 1] Nat Biotechnol, 23, 1073-1078, 2005
[Non-patent Document 2] Eur J Pharm Biopharm, 59(3), 389-96. 2005
[Non-patent Document 3] Chem Immunol, 65, 88-110, 1997
[Non-patent Document 4] J Biol Chem, 276, 16478-16483, 2001
[Non-patent Document 5] Eur J Immunol 23, 1098-1104, 1993
[Non-patent Document 6] Immunology, 86, 319-324, 1995
[Non-patent Document 7] Immunol Lett, 82, 57-65, 2002
[Non-patent Document 8] Nat Rev Immunol, 10, 328-343, 2010
[Non-patent Document 9] Nat Rev Immunol, 8, 34-47, 2008
[Non-patent Document 10] Eur J Immunol 19, 1379-1385, 1989
[Non-patent Document 11] J Exp Med 129, 1183-1201, 1969
[Non-patent Document 12] Immunol Lett 88, 157-161, 2003
[Non-patent Document 13] Science, 256, 1808-1812, 1992
[Non-patent Document 14] Nature, 368, 70-73, 1994
[Non-patent Document 15] Science, 290, 84-89, 2000
[Non-patent Document 16] J Imunol, 181, 5350-5359, 2008
[Non-patent Document 17] J Immunol, 163, 618-622, 1999
[Non-patent Document 18] J Exp Med, 189, 187-194, 1999
[Non-patent Document 19] J Exp Med, 191, 899-906, 2000
[Non-patent Document 20] Hum, Genet, 117, 220-227, 2005
[Non-patent Document 21] J Biol Chem, 282, 1738-1746, 2007
[Non-patent Document 22] Arthritis Rheum, 54, 3908-3917, 2006
[Non-patent Document 23] Nat Med, 11, 1056-1058, 2005
[Non-patent Document 24] J Immunol, 176, 5321-5328, 2006
[Non-patent Document 25] J Exp Med, 203, 2157-2164, 2006
[Non-patent Document 26] J Immunol. 178, 3272-3280, 2007
[Non-patent Document 27] Blood, 113, 3716-3725, 2009
[Non-patent Document 28] Mol Immunol, 45, 3926-3933, 2008
[Non-patent Document 29] J Allergy Clin Immunol, 2012, 129:1102-1115
[Non-patent Document 30] Arthritis Rheum, 62, 1933-1943, 2010
[Non-patent Document 31] Immunol let, doi: 10.1016/j.imlet.2012.01.008)
[Non-patent Document 32] J. Immunol, 2009, 183:4509-4520
[Non-patent Document 33] J. Immunol, 2009, 182:554-562
[Non-patent Document 34] Science, 333, 1030-1034, 2011
[Non-patent Document 35] Cancer Cell 19, 101-113, 2011
[Non-patent Document 36] J Clin Invest, 122 (3), 1066-1075, 2012
[Non-patent Document 37] J Immunol 171, 562-, 2003
[Non-patent Document 38] Blood, 108, 705-, 2006
[Non-patent Document 39] J Immunol 166, 4891, 2001
[Non-patent Document 40] doi: 10.1073/pnas.1208698109
[Non-patent Document 41] Immunol let, 2002, 143:28-33
[Non-patent Document 42] J Natl Cancer Inst, 99, 1232-1239, 2007
[Non-patent Document 43] Arthritis Rheum, 48, 719-727, 2003
[Non-patent Document 44] J Thromb Haemost, 7, 171-181, 2008
[Non-patent Document 45] J Immunol, 185, 1577-1583, 2010
[Non-patent Document 46] Sci Transl Med, vol 2, issue 47, 47-63, 2010
[Non-patent Document 47] Mol Cancer Ther 7, 2517-2527, 2008
[Non-patent Document 48] J. Immunol, 2007, 178:6217-6226
[Non-patent Document 49] J Clin Invest, 97, 1348-1354, 1996
[Non-patent Document 50] Arthritis Rheum, 41, 1181-1189, 1998
[Non-patent Document 51] J Clin Invest 115, 2914-2923, 2005
[Non-patent Document 52] Proc Natl Acad Sci USA, 102, 2910-2915, 2005
[Non-patent Document 53] Mol Immunol, 40, 585-593, 2003
[Non-patent Document 54] J Exp Med, 172, 19-25, 1990

### SUMMARY OF THE INVENTION

### Problems to be Solved by the Invention

The present invention was achieved in view of the above circumstances. An objective of the present invention is to provide an Fc region variant by introducing an amino acid alteration(s) into an antibody Fc region, which variant has enhanced FcγRIIb-binding activity, and/or enhanced binding selectivity to FcγRIIb compared to FcγRIIa (type R), as compared to when no amino acid alteration has been introduced into the Fc region; a polypeptide comprising the Fc region variant; and a pharmaceutical composition containing the polypeptide.

### Means for Solving the Problems

The present inventors carried out dedicated research on: an Fc region variant with enhanced FcγRIIb-binding and enhanced binding selectivity to FcγRIIb compared to FcγRIIa (type R), as compared to when the Fc region is unaltered, by introducing amino acid alteration(s) into the Fc region; and a polypeptide comprising the Fc region variant. As a result, the present inventors found that FcγRIIb-binding activity is enhanced and/or binding selectivity to FcγRIIb compared to FcγRIIa (type R) is enhanced by combining an Fc region variant in which the amino acid at position 238 (EU numbering) in the Fc Region has been altered with other amino acid alteration(s).

More specifically, the present invention relates to the following:
[1] an Fc region variant in which amino acid at position 238 according to EU numbering and at least one amino acid selected from those at positions 233, 234, 235, 237, 264, 265, 266, 267, 268, 269, 271, 272, 274, 296, 326, 327, 330, 331, 332, 333, 334, 355, 356, 358, 396, 409, and 419 according to EU numbering are altered to other amino acids, wherein binding activity of the variant to Fcγ receptors [KD value for FcγRIIb of a polypeptide comprising an Fc region to which an amino acid alteration(s) has not been introduced]/[KD value for FcγRIIb of a polypeptide comprising the Fc region variant] has a value which is 15.0 or greater;
[2] the Fc region variant of [1], wherein the amino acids at positions 238, 268, and 271 according to EU numbering are altered to other amino acids, and in addition, at least one amino acid selected from amino acids at positions 233, 237, 264, 267, 272, 296, 327, 330, 332, and 396 according to EU numbering are altered to other amino acids;
[3] an Fc region variant whose amino acid at position 238 according to EU numbering is Asp, and which comprises at least one amino acid selected from the amino acid group consisting of Asp at amino acid position 233, Tyr at amino acid position 234, Phe at amino acid position 235, Asp at amino acid position 237, Ile at amino acid position 264, Glu at amino acid position 265, Phe, Leu, or Met at amino acid position 266, Ala, Glu, Gly, or Gln at amino acid position 267, Asp, Gln, or Glu at amino acid position 268, Asp at amino acid position 269, Gly at amino acid position 271, Asp, Phe, Ile, Met, Asn, Pro, or Gln at amino acid position 272, Gln at amino acid position 274, Asp or Phe at amino acid position 296, Ala or Asp at amino acid position 326, Gly at amino acid position 327, Lys, Arg, or Ser at amino acid position 330, Ser at amino acid position 331, Lys, Arg, Ser, or Thr at amino acid position 332, Lys, Arg, Ser, or Thr at amino acid position 333, Arg, Ser, or Thr at amino acid position 334, Ala or Gln at amino acid position 355, Glu at amino acid position 356, Met at amino acid position 358, Ala, Asp, Glu, Phe, Gly, His, Ile, Lys, Leu, Met, Asn, Gln, Arg, Ser, Thr, Val, Trp, or Tyr at amino acid position 396, Arg at amino acid position 409, and Glu at amino acid position 419, wherein binding activity of the variant to Fcγ receptors [KD value for FcγRIIb of a polypeptide comprising an Fc region to which an amino acid alteration(s) has not been introduced]/[KD value for FcγRIIb of a polypeptide comprising the Fc region variant] has a value which is 15.0 or greater;
[4] the Fc region variant of [3], wherein the amino acid at position 238 is Asp, the amino acid at position 268 is Asp or Glu, and the amino acid at position 271 is Gly, according to EU numbering, and wherein the Fc region variant further comprises at least one amino acid selected from the amino acid group consisting of Asp at amino acid position 233, Asp at amino acid position 237, Ile at amino acid position 264, Ala or Gly at amino acid position 267, Asp or Pro at amino acid position 272, Asp at amino acid position 296, Gly at amino acid position 327, Arg at amino acid position 330, Thr at amino acid position 332, and Leu or Met at amino acid position 396;
[5] the Fc region variant of any one of [1] to [4], wherein the value for [KD value for FcγRIIb of a polypeptide comprising an Fc region to which an amino acid alteration(s) has not been introduced]/[KD value for FcγRIIb of a polypeptide comprising an Fc region variant] is 50.0 or more;
[6] the Fc region variant of any one of [1] to [4], wherein the value for [KD value for FcγRIIb of a polypeptide comprising an Fc region to which an amino acid alteration(s) has not been introduced]/[KD value for FcγRIIb of a polypeptide comprising an Fc region variant] is 100.0 or more;
[7] the Fc region variant of any one of [1] to [6], wherein the value for [KD value for FcγRIIa (type R) of a polypeptide comprising an Fc region variant]/[KD value for FcγRIIb of a polypeptide comprising an Fc region variant] is 10.0 or greater;
[8] the Fc region variant of any one of [1] to [6], wherein the value for [KD value for FcγRIIa (type R) of a polypeptide comprising an Fc region variant]/[KD value for FcγRIIb of a polypeptide comprising an Fc region variant] is 20.0 or greater;
[9] The Fc region variant of any one of [1] to [8], wherein the Fc region variant comprises any one of the following set of amino acid alterations of (a) to (x):
(a) amino acid alterations at positions 238, 233, 237, 268, 271, 296, and 330 (EU numbering) of an Fc region;
(b) amino acid alterations at positions 238, 237, 268, 271, 296, and 330 (EU numbering) of an Fc region;
(c) amino acid alterations at positions 238, 233, 237, 268, 271, 296, 330, and 332 (EU numbering) of an Fc region;
(d) amino acid alterations at positions 238, 233, 237, 264, 267, 268, 271, and 330 (EU numbering) of an Fc region;
(e) amino acid alterations at positions 238, 233, 237, 267, 268, 271, 296, 330, and 332 (EU numbering) of an Fc region;
(f) amino acid alterations at positions 238, 237, 267, 268, 271, 296, 330, and 332 (EU numbering) of an Fc region;
(g) amino acid alterations at positions 238, 233, 237, 268, 271, 296, 327, and 330 (EU numbering) of an Fc region;
(h) amino acid alterations at positions 238, 233, 237, 264, 267, 268, and 271 (EU numbering) of an Fc region;
(i) amino acid alterations at positions 238, 233, 237, 264, 267, 268, 271, 296, and 330 (EU numbering) of an Fc region;
(j) amino acid alterations at positions 238, 233, 237, 264, 267, 268, 271, 296, 330, and 396 (EU numbering) of an Fc region;
(k) amino acid alterations at positions 238, 237, 264, 267, 268, 271, and 330 (EU numbering) of an Fc region;
(l) amino acid alterations at positions 238, 237, 264, 267, 268, 271, 296, and 330 (EU numbering) of an Fc region;
(m) amino acid alterations at positions 238, 264, 267, 268, and 271 (EU numbering) of an Fc region;
(n) amino acid alterations at positions 238, 264, 267, 268, 271, and 296 (EU numbering) of an Fc region;
(o) amino acid alterations at positions 238, 237, 267, 268, 271, 296, and 330 (EU numbering) of an Fc region;
(p) amino acid alterations at positions 238, 233, 237, 264, 267, 268, 271, 330, and 396 (EU numbering) of the Fc region;
(q) amino acid alterations at positions 238, 233, 237, 264, 267, 268, 271, 296, 327, 330, and 396 (EU numbering) of an Fc region;
(r) amino acid alterations at positions 238, 233, 237, 264, 267, 268, 271, 272, and 296 (EU numbering) of an Fc region;
(s) amino acid alterations at positions 238, 237, 264, 267, 268, 271, 272, and 330 (EU numbering) of an Fc region;
(t) amino acid alterations at positions 238, 237, 264, 267, 268, 271, 272, 296, and 330 (EU numbering) of an Fc region;
(u) amino acid alterations at positions 238, 233, 264, 267, 268, and 271 (EU numbering) of an Fc region;
(v) amino acid alterations at positions 238, 237, 267, 268, 271, 296, and 330 (EU numbering) of an Fc region;
(w) amino acid alterations at positions 238, 264, 267, 268, 271, 272, and 296 (EU numbering) of an Fc region; and
(x) amino acid alterations at positions 238, 233, 264, 267, 268, 271, and 296 (EU numbering) of an Fc region;
[10] the Fc region variant of any one of [1] to [8], wherein the Fc region variant comprises any one of the following amino acid sequences of (a) to (x):
(a) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, and the amino acid at position 330 is Arg, according to EU numbering, in an Fc region;
(b) an amino acid sequence in which the amino acid at position 238 is Asp, the amino acid at position 237 is Asp, the amino acid at position 268 is Asp or Glu, the amino acid at position 271 is Gly, the amino acid at position 296 is Asp, and the amino acid at position 330 is Arg, according to EU numbering, in an Fc region;
(c) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 330 is Arg, and the amino acid at position 332 is Thr, according to EU numbering, in an Fc region;
(d) an amino acid sequence in which the amino acid at position 238 is Asp, the amino acid at position 233 is Asp, the amino acid at position 237 is Asp, the amino acid at position 264 is Ile, the amino acid at position 267 is Gly or Ala, the amino acid at position 268 is Glu, the amino acid at position 271 is Gly, and the amino acid at position 330 is Arg, according to EU numbering, in an Fc region;
(e) an amino acid sequence in which the amino acid at position 238 is Asp, the amino acid at position 233 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(f) an amino acid sequence in which the amino acid at position 238 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(g) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 327 is Gly, and the amino acid at position 330 is Arg, according to EU numbering, in an Fc region;
(h) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, and the amino acid at position 271 is Gly, according to EU numbering, in an Fc region;
(i) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(j) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 330 is Arg, and the amino acid at position 396 is Met or Leu, according to EU numbering, in an Fc region;
(k) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, and the amino acid at position 330 is Arg, according to EU numbering, in an Fc region;
(l) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(m) an amino acid sequence in which the amino acid at position 238 is Asp, the amino acid at position 264 is Ile, the amino acid at position 267 is Ala, the amino acid at position 268 is Glu, and the amino acid at position 271 is Gly, according to EU numbering, in an Fc region;
(n) an amino acid sequence in which the amino acid at position 238 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region;
(o) an amino acid sequence in which the amino acid at position 238 is Asp, the amino acid at position 237 is Asp, the amino acid at position 267 is Ala or Gly, the amino acid at position 268 is Glu, the amino acid at position 271 is Gly, the amino acid at position 296 is Asp, and the amino acid at position 330 is Arg, according to EU numbering, in an Fc region;
(p) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 330 is Arg, and the amino acid at position 396 is Met or Leu, according to EU numbering, in an Fc region;
(q) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 327 is Gly, the amino acid at position 330 is Arg, and the amino acid at position 396 is Met, according to EU numbering, in an Fc region;
(r) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region;
(s) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, and the amino acid at position 330 is Arg, according to EU numbering, in an Fc region;
(t) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, the amino acid at position 296 is Asp, and the amino acid at position 330 is Arg, according to EU numbering, in an Fc region;
(u) an amino acid sequence in which the amino acid at position 238 is Asp, the amino acid at position 233 is Asp, the amino acid at position 264 is Ile, the amino acid at position 267 is Ala, the amino acid at position 268 is Glu, and the amino acid at position 271 is Gly, according to EU numbering, in an Fc region;
(v) an amino acid sequence in which the amino acid at position 238 is Asp, the amino acid at position 237 is Asp, the amino acid at position 267 is Gly, the amino acid at position 268 is Asp, the amino acid at position 271 is Gly, the amino acid at position 296 is Asp, and the amino acid at position 330 is Arg, according to EU numbering, in an Fc region;
(w) an amino acid sequence in which the amino acid at position 238 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region; and
(x) an amino acid sequence in which the amino acid at position 238 is Asp, the amino acid at position 233 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region;
[11] an Fc region variant consisting of any one amino acid sequence selected from among SEQ ID NOs: 43 to 68, SEQ ID NO: 70, SEQ ID NO: 71, and SEQ ID NOs: 75 to 77;
[12] a polypeptide comprising at least two Fc region variants of any one of [1] to [11], wherein the two Fc region variants are associated;
[13] the polypeptide of [12], wherein the amino acid sequences of the two associated Fc region variants in the polypeptide are the same;
[14] the polypeptide of [12], wherein the amino acid sequences of the two associated Fc region variants in the polypeptide are different;
[15] the polypeptide of [14], wherein the amino acid sequences of the two associated Fc region variants have different amino acid(s) at at least one amino acid position selected from positions 235, 236, 237, 238, and 239 according to EU numbering in the Fc region variant;
[16] the polypeptide of [15], wherein one of the amino acid sequences of the two associated Fc region variants is an amino acid sequence comprising at least one amino acid selected from Asp, Gln, Glu, or Thr at amino acid position 235, Asn at amino acid position 236, Phe or Trp at amino acid position 237, Glu, Gly, or Asn at amino acid position 238, and Asp or Glu at amino acid position 239 according to EU numbering;
[17] the polypeptide of any one of [12] to [16], wherein the polypeptide comprising the Fc region variant is an IgG antibody;
[18] the polypeptide of any one of [12] to [16], wherein the polypeptide comprising the Fc region variant is an Fc fusion protein molecule; and
[19] a pharmaceutical composition comprising the polypeptide of any one of [12] to [18].

Furthermore, the present invention relates to a method of enhancing FcγRIIb-binding activity of an Fc region and enhancing binding selectivity to FcγRIIb compared to FcγRIIa (type R), by introducing an amino acid alteration(s) into the Fc region of the present invention. The present invention also relates to a method of suppressing production of antibodies against a polypeptide containing an Fc region, by introducing an amino acid alteration(s) of the present invention into the Fc region.

The present invention also relates to a therapeutic or preventive agent for immune inflammatory diseases that comprises a polypeptide of the present invention. Furthermore, the present invention relates to a method for treating or preventing immune inflammatory diseases, which comprises the step of administering a polypeptide of the present invention to a subject. In addition, the present invention relates to a kit for use in the method of the present invention for treating or preventing immune inflammatory diseases, which comprises a polypeptide of the present invention. The present invention also relates to use of a polypeptide of the present invention in the production of a therapeutic or preventive agent for immune inflammatory diseases. Furthermore, the present invention relates to a polypeptide of the present invention for use in the method for treating or preventing immune inflammatory diseases of the present invention.

The present invention relates to an activation inhibitor for B cells, mast cells, dendritic cells, and/or basophils, which comprises a polypeptide of the present invention. Furthermore, the present invention relates to a method of inhibiting activation of B cells, mast cells, dendritic cells, and/or basophils, which comprises administering a polypeptide of the present invention to a subject. The present invention also relates to a kit for use in the method of inhibiting activation of B cells, mast cells, dendritic cells, and/or basophils, which comprises a polypeptide of the present invention. The present invention relates to a use of a polypeptide of the present invention in producing activation inhibitors for B cells, mast cells, dendritic cells, and/or basophils. The present invention also relates to a polypeptide of the present invention for use in the method of the present invention of inhibiting activation of B cells, mast cells, dendritic cells, and/or basophils.

Furthermore, the present invention relates to a therapeutic agent for diseases in which a protein necessary for an organism is deficient, wherein the agent comprises a polypeptide of the present invention. The present invention also relates to a method for treating diseases in which a protein necessary for an organism is deficient, which comprises administering a polypeptide of the present invention to a subject. Furthermore, the present invention relates to a kit for use in the method of the present invention for treating diseases in which a protein necessary for an organism is deficient, wherein the kit comprises a polypeptide of the present invention. The present invention relates to use of a polypeptide of the present invention in producing a therapeutic agent for diseases in which a protein necessary for an organism is deficient. The present invention also relates to a polypeptide of the present invention for use in the method of the present invention for treating diseases in which a protein necessary for an organism is deficient.

In addition, the present invention relates to an agent for suppressing virus proliferation, which comprises a polypeptide of the present invention. The present invention also relates to a method of suppressing virus proliferation, which comprises administering a polypeptide of the present invention to a subject. Furthermore, the present invention relates to a kit of the present invention for use in the method of suppressing virus proliferation, wherein the kit comprises a polypeptide of the present invention. The present invention relates to use of a polypeptide of the present invention in producing an agent for suppressing virus proliferation. The present invention also relates to a polypeptide of the present invention for use in the method of the present invention of suppressing virus proliferation.

### Effects of the Invention

Fc region variants with enhanced FcγRIIb-binding activity and/or enhanced binding selectivity to FcγRIIb compared to FcγRIIa (type R), as compared to when the Fc region is unaltered, are provided by the present invention. By using the polypeptides containing the Fc region variants, it is possible to enhance inhibitory signals of inflammatory immune responses mediated by phosphorylation of ITIM of FcγRIIb. Also, by conferring an Fc region with the property of selective FcγRIIb binding, it may be possible to suppress anti-drug antibody production. Also, by using an Fc region variant of the present invention as a polypeptide having human FcRn-binding activity under an acidic pH range condition and comprising an antigen-binding domain in which an antigen-binding activity of an antigen-binding molecule changes depending on the ion concentration conditions, it is possible to promote elimination of antigens that bind to the polypeptide, which are present in plasma.

### BRIEF DESCRIPTION OF THE DRAWINGS

FIG. 1 is a graph showing the results of evaluating platelet aggregation due to the omalizumab-G1d-3/IgE immune complex in a platelet aggregation assay using platelets derived from a donor having FcγRIIa (R/H) polymorphism.

FIG. 2 is a graph showing the results of evaluating platelet aggregation due to the omalizumab-G1d-3/IgE immune complex in a platelet aggregation assay using platelets derived from a donor having FcγRIIa (H/H) polymorphism.

FIG. 3 is a graph showing the results of evaluating CD62p expression on a washed platelet membrane surface. The black-filled curve indicates the result obtained when reaction with PBS was followed by stimulation by ADP addition, and the unfilled curve indicates the result obtained when reaction with an immune complex was followed by stimulation with ADP.

FIG. 4 is a graph showing the results of evaluating active integrin expression on a washed platelet membrane surface. The black-filled curve indicates the result obtained when reaction with PBS was followed by stimulation by ADP addition, and the unfilled curve indicates the result obtained when reaction with an immune complex was followed by stimulation with ADP.

FIG. 5 shows the Fc(P208)/FcγRIIb extracellular region complex as determined by X-ray crystal structure analysis. For each of the Fc-region CH2 domain and CH3 domain, the portion shown on the left was defined as domain A and the portion shown on the right was defined as domain B.

FIG. 6 shows a comparison made by superimposing the X-ray crystal structure of the Fc(P208)/FcγRIIb extracellular region complex and the structure of the Fc(WT)/FcγRIIa extracellular region complex (PDB code: 3RY6), with respect to Fc CH2 domain A by least square fitting based on Cα atom pair distances. In the figure, the Fc(P208)/FcγRIIb extracellular region complex structure is depicted using thick lines and the Fc(WT)/FcγRIIa extracellular region complex structure is depicted using thin lines. Regarding the structure of the Fc(WT)/FcγRIIa extracellular region complex, only the Fc portion CH2 domain A is depicted.

FIG. 7 shows the detailed structure around Asp at position 237 (EU numbering) in Fc portion CH2 domain A whose main chain forms a hydrogen bond with Tyr at position 160 of FcγRIIb in the X-ray crystal structure of the Fc(P208)/FcγRIIb extracellular region complex.

FIG. 8 shows the structure of amino acid residues around the side chain of Asp at position 237 (EU numbering) in Fc portion CH2 domain A whose main chain forms a hydrogen bond with Tyr at position 160 of FcγRIIb in the X-ray crystal structure of the Fc(208)/FcγRIIb extracellular region complex.

FIG. 9 shows an image of superimposing the X-ray crystal structure of the Fc(P238D)/FcγRIIb extracellular region complex shown in Reference Example 7 and the X-ray crystal structure of the Fc(P208)/FcγRIIb extracellular region complex, with respect to Fc portion CH2 domain B by least square fitting based on Cα atom pair distances, to compare the region around the loop of positions 266 to 271 according to EU numbering. In the loop, Fc(P208) has an H268D alteration at position 268 (EU numbering) and a P271G alteration at position 271 (EU numbering) when compared to Fc(P238D).

FIG. 10 shows the structure around Ser239 of the Fc portion CH2 domain B along with electron density obtained by X-ray crystal structure analysis which uses 2Fo-Fc as the coefficient in the X-ray crystal structure of the Fc(P208)/FcγRIIb extracellular region complex.

FIG. 11 shows a comparison made by superimposing the three-dimensional structure of the Fc(P208)/FcγRIIa type R extracellular region complex and three-dimensional structure of the Fc(P208)/FcγRIIb extracellular region complex, which are determined by X-ray crystal structure analysis, by least square fitting based on Cα atom pair distances.

FIG. 12 shows a comparison of the X-ray crystal structure of the Fc(P208)/FcγRIIa type R extracellular region complex and the X-ray crystal structure of the Fc(P208)/FcγRIIb extracellular region complex around Asp at position 237 (EU numbering) in Fc portion CH2 domain A, along with electron density obtained by X-ray crystal structure analysis which uses 2Fo-Fc as the coefficient.

FIG. 13 shows a comparison of the X-ray crystal structure of the Fc(P208)/FcγRIIa type R extracellular region complex and the X-ray crystal structure of the Fc(P208)/FcγRIIb extracellular region complex around Asp at position 237 (EU numbering) in Fc portion CH2 domain B, along with electron density obtained by X-ray crystal structure analysis which uses 2Fo-Fc as the coefficient.

FIG. 14 shows a comparison of the constant region sequences of G1d and G4d. In the figure, the boxed amino acids show portions where the amino acid residues are different between G1d and G4d.

FIG. 15 shows the values of the binding amount of each variant to FcgRIIb on the horizontal axis, and the binding amount of each variant to FcgRIIaR on the vertical axis. The alterations indicated in the figure, G237W, G237F, G236N, P238G, P238N, P238E, and P238D, refer to alterations introduced into GpH7-B3. A5/B3 refers to GpH7-A5/GpH7-B3/GpL16-k0 without any introduction of alterations to both chains, and a variant containing P238D in only one of the chains refers to GpH7-A5/GpH7-BF648/GpL16-k0.

FIG. 16 shows the KD values of each variant for FcgRIIb on the horizontal axis, and the KD values of each variant for FcgRIIaR on the vertical axis. IL6R-B3/IL6R-L and IL6R-G1d/IL6R-L in the figure refer to antibodies having native human IgG sequences which serve as a comparison control when evaluating each of the variants. IL6R-BP264/IL6R-L is an original variant when producing each of the variants. IL6R-BP404/IL6R-L is a variant introduced with L234Y into both chains of IL6R-BP264/IL6R-L, which has improved FcgRIIb binding compared to that of the IL6R-BP264/IL6R-L before introducing alteration.

FIG. 17 shows comparison of FcγRIa binding and FcγRIIb binding. Binding of the antibody with substitution of Pro at position 238 (EU numbering) with Asp, and binding of the antibody with substitution of Leu at position 328 (EU numbering) with Glu have been labeled. “Mutation A” refers to an alteration produced by substituting Pro at position 238 (EU numbering) with Asp and “mutation B” refers to an alteration produced by substituting Leu at position 328 (EU numbering) with Glu.

FIG. 18 shows comparison of FcγRIIa type H binding and FcγRIIb binding. Binding of the antibody with substitution of Pro at position 238 (EU numbering) with Asp, and binding of the antibody with substitution of Leu at position 328 (EU numbering) with Glu have been labeled. “Mutation A” refers to an alteration produced by substituting Pro at position 238 (EU numbering) with Asp, and “mutation B” refers to an alteration produced by substituting Leu at position 328 (EU numbering) with Glu.

FIG. 19 shows comparison of FcγRIIa type R binding and FcγRIIb binding. Binding of the antibody with substitution of Pro at position 238 (EU numbering) with Asp, and binding of the antibody with substitution of Leu at position 328 (EU numbering) with Glu have been labeled. “Mutation A” refers to an alteration produced by substituting Pro at position 238 (EU numbering) with Asp, and “mutation B” refers to an alteration produced by substituting Leu at position 328 (EU numbering) with Glu.

FIG. 20 shows comparison of FcγRIIIa binding and FcγRIIb binding. Binding of the antibody with substitution of Pro at position 238 (EU numbering) with Asp, and binding of the antibody with substitution of Leu at position 328 (EU numbering) with Glu have been labeled. “Mutation A” refers to an alteration produced by substituting Pro at position 238 (EU numbering) with Asp, and “mutation B” refers to an alteration produced by substituting Leu at position 328 (EU numbering) with Glu.

FIG. 21 shows the relationship between the amino acid residues constituting the constant regions of IgG1, IgG2, IgG3, and IgG4, and EU numbering (herein, also referred to as EU INDEX).

FIG. 22 shows a graph in which the horizontal axis shows the relative value of FcγRIIb-binding activity of each PD variant, and the vertical axis shows the relative value of FcγRIIa type R-binding activity of each PD variant. The value for the amount of binding of each PD variant to each FcγR was divided by the value for the amount of binding of IL6R-F652/IL6R-L, which is a control antibody prior to introduction of the alteration (altered Fc with substitution of Pro at position 238 (EU numbering) with Asp), to each FcγR; and then the obtained value was multiplied by 100, and used as the relative binding activity value for each PD variant to each FcγR. The F652 plot in the figure shows the value for IL6R-F652/IL6R-L.

FIG. 23 shows a graph in which the vertical axis shows the relative value of FcγRIIb-binding activity of variants produced by introducing each alteration into GpH7-B3 which does not have the P238D alteration, and the horizontal axis shows the relative value of FcγRIIb-binding activity of variants produced by introducing each alteration into IL6R-F652 which has the P238D alteration. The value for the amount of FcγRIIb binding of each variant was divided by the value for the amount of FcγRIIb binding of the pre-altered antibody; and then the obtained value was multiplied by 100, and used as the value of relative binding activity. Here, region A contains alterations that exhibit the effect of enhancing FcγRIIb binding in both cases where an alteration is introduced into GpH7-B3 which does not have P238D and where an alteration is introduced into IL6R-F652 which has P238D. Region B contains alterations that exhibit the effect of enhancing FcγRIIb binding when introduced into GpH7-B3 which does not have P238D, but do not exhibit the effect of enhancing FcγRIIb binding when introduced into IL6R-F652 which has P238D.

FIG. 24 shows a crystal structure of the Fc(P238D)/FcγRIIb extracellular region complex.

FIG. 25 shows an image of superimposing the crystal structure of the Fc(P238D)/FcγRIIb extracellular region complex and the model structure of the Fc(WT)/FcγRIIb extracellular region complex, with respect to the FcγRIIb extracellular region and the Fc CH2 domain A by least square fitting based on Cα atom pair distances.

FIG. 26 shows comparison of the detailed structure around P238D after superimposing the crystal structure of the Fc(P238D)/FcγRIIb extracellular region complex and the model structure of the Fc(WT)/FcγRIIb extracellular region complex with respect to the only Fc CH2 domain A or the only Fc CH2 domain B by least square fitting based on Cα atom pair distances.

FIG. 27 shows that a hydrogen bond is found between the main chain of Gly at position 237 (EU numbering) in Fc CH2 domain A, and Tyr at position 160 in FcγRIIb in the crystal structure of the Fc(P238D)/FcγRIIb extracellular region complex.

FIG. 28 shows that an electrostatic interaction is found between Asp at position 270 (EU numbering) in Fc CH2 domain B, and Arg at position 131 in FcγRIIb in the crystal structure of the Fc(P238D)/FcγRIIb extracellular region complex.

FIG. 29 shows a graph in which the horizontal axis shows the relative value of FcγRIIb-binding activity of each 2B variant, and the vertical axis shows the relative value of FcγRIIa type R-binding activity of each 2B variant. The value for the amount of binding of each 2B variant to each FcγR was divided by the value for the amount of binding of a control antibody prior to alteration (altered Fc with substitution of Pro at position 238 (EU numbering) with Asp) to each FcγR; and then the obtained value was multiplied by 100, and used as the value of relative binding activity of each 2B variant towards each FcγR.

FIG. 30 shows Glu at position 233 (EU numbering) in Fc Chain A and the surrounding residues in the extracellular region of FcγRIIb in the crystal structure of the Fc(P238D)/FcγRIIb extracellular region complex.

FIG. 31 shows Ala at position 330 (EU numbering) in Fc Chain A and the surrounding residues in the extracellular region of FcγRIIb in the crystal structure of the Fc(P238D)/FcγRIIb extracellular region complex.

FIG. 32 shows the structures of Pro at position 271 (EU numbering) of Fc Chain B after superimposing the crystal structures of the Fc(P238D)/FcγRIIb extracellular region complex and the Fc(WT)/FcγRIIIa extracellular region complex by least square fitting based on Cα atom pair distances with respect to Fc Chain B.

FIG. 33 shows the change in plasma concentrations of the administered antigen-binding molecules of human FcgRIIb transgenic mice when Fv4-IgG1 or Fv4-P587 was administered to the mice.

FIG. 34 shows the change in plasma concentrations of the administered human IL-6R of human FcgRIIb transgenic mice when Fv4-IgG1 or Fv4-P587 was administered to the mice.

FIG. 35 shows a non-limiting action mechanism for the elimination of soluble antigens from plasma by administration of antibodies that bind to antigens in an ion-concentration-dependent manner, which are antibodies with enhanced FcγR-binding at neutral pH as compared to that of existing neutralizing antibodies.

FIG. 36 shows the change in plasma concentrations of the administered antigen-binding molecules of human FcgRIIb and human FcRn transgenic mice when Fv4-IgG1, Fv4-P587, or Fv4-P587_LS was administered to the mice.

FIG. 37 shows the change in plasma concentrations of the administered human IL-6R of human FcgRIIb and human FcRn transgenic mice when Fv4-IgG1, Fv4-P587, or Fv4-P587_LS was administered to the mice.

### MODE FOR CARRYING OUT THE INVENTION

The present invention provides an Fc region variant with enhanced FcγRIIb-binding activity, and/or enhanced binding selectivity to FcγRIIb compared to FcγRIIa (type R), as compared to an Fc region to which an amino acid alteration(s) has not been introduced; and a polypeptide comprising the Fc region variant.

More specifically, the invention provides an Fc region variant containing an amino acid sequence that has a combination of an amino acid alteration at position 238 (EU numbering) and other specific amino acid alteration(s); and a polypeptide comprising the Fc region variant. Furthermore, the present invention provides a method of enhancing FcγRIIb-binding activity, and/or enhancing binding selectivity to FcγRIIb, compared to FcγRIIa (type R) as compared to those of an Fc region to which an amino acid alteration(s) has not been introduced, by introducing amino acid alteration(s) into the Fc region. The present invention also provides a method of suppressing production of antibodies against an Fc region by introducing an amino acid alteration(s) into the Fc region when the Fc region variant is administered to an organism, as compared to when the Fc region without introduction of amino acid alteration(s) is administered. “Polypeptides” of the present invention generally refers to peptides or proteins approximately ten amino acids or more in length. Furthermore, they are generally polypeptides derived from organisms, but are not particularly limited, and for example, they may be polypeptides comprising an artificially designed sequence. Furthermore, they may be any of naturally-occurring polypeptides, synthetic polypeptides, recombinant polypeptides, or such.

Preferred examples of the polypeptides of the present invention include antibodies. More preferred examples include naturally-occurring IgGs, particularly naturally-occurring human IgGs. “Naturally-occurring (native) IgGs” refers to polypeptides belonging to a class of antibodies practically encoded by immunoglobulin gamma genes and comprising an amino acid sequence identical to those of IgGs found in nature. For example, a naturally-occurring human IgG means a naturally-occurring human IgG1, naturally-occurring human IgG2, naturally-occurring human IgG3, naturally-occurring human IgG4, or such. Naturally-occurring IgGs also include mutants spontaneously produced from them.

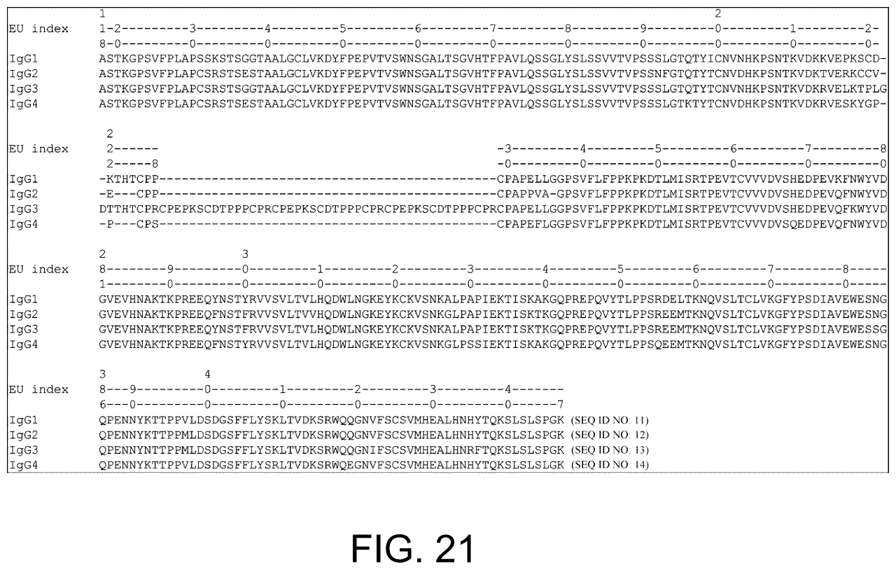

While an IgK (Kappa, κ chain), IgL1, IgL2, IgL3, IgL6, and IgL7 (Lambda, λ chain)-type constant region is present in the antibody light chain constant region, it may be any light chain constant region. For the human IgK (Kappa) constant region and human IgL7 (Lambda) constant region, a plurality of allotype sequences due to genetic polymorphism are described in “Sequences of proteins of immunological interest”, NIH Publication No. 91-3242, and any of them may be used in the present invention. Furthermore, in the present invention, a light chain constant region may be a light chain constant region that has been altered with amino acid substitutions, additions, deletions, insertions, and/or modifications or such. For the antibody Fc region, for example, Fc regions of the IgA1, IgA2, IgD, IgE, IgG1, IgG2, IgG3, IgG4, and IgM types exist. For example, a human IgG antibody Fc region can be used as the antibody Fc region of the present invention, and human IgG1 antibody Fc regions are preferred. Fc regions that can be used as an Fc region of the present invention are, for example, those derived from naturally-occurring IgG constant regions, or specifically, a constant region derived from naturally-occurring human IgG1 (SEQ ID NO: 11), a constant region derived from naturally-occurring human IgG2 (SEQ ID NO: 12), a constant region derived from naturally-occurring human IgG3 (SEQ ID NO: 13), and a constant region derived from naturally-occurring human IgG4 (SEQ ID NO: 14). FIG. 21 shows the constant region sequences of the naturally-occurring IgG1, IgG2, IgG3, and IgG4. Constant regions of naturally-occurring IgGs also include mutants spontaneously produced from them. For the constant regions of human IgG1, human IgG2, human IgG3, and human IgG4 antibodies, a plurality of allotype sequences due to genetic polymorphism are described in “Sequences of proteins of immunological interest”, NIH Publication No. 91-3242, and any of them may be used in the present invention. In particular, for the human IgG1 sequence, the amino acid sequence at positions 356 to 358 (EU numbering) may be either DEL or EEM.

“Fcγ receptors” (herein, referred to as Fcγ receptors, FcγR or FcgR) refers to receptors that may bind to the Fc region of IgG1, IgG2, IgG3, and IgG4 monoclonal antibodies, and practically means any member of the family of proteins encoded by the Fcγ receptor genes. In humans, this family includes FcγRI (CD64) including isoforms FcγRIa, FcγRIb, and FcγRIc; FcγRII (CD32) including isoforms FcγRIIa (including allotypes H131 (type H) and R131 (type R)), FcγRIIb (including FcγRIIb-1 and FcγRIIb-2), and FcγRIIc; and FcγRIII (CD16) including isoforms FcγRIIIa (including allotypes V158 and F158), and FcγRIIIb (including allotypes FcγRIIIb-NA1 and FcγRIIIb-NA2), and any human FcγRs, FcγR isoforms or allotypes yet to be discovered, but is not limited thereto. FcγRIIb1 and FcγRIIb2 have been reported as splicing variants of human FcγRIIb. In addition, a splicing variant named FcγRIIb3 has been reported (J. Exp. Med, 1989, 170:1369). In addition to these splicing variants, human FcγRIIb includes all splicing variants registered in NCBI, which are NP_001002273.1, NP_001002274.1, NP_001002275.1, NP_001177757.1, and NP_003992.3. Furthermore, human FcγRIIb includes every previously-reported genetic polymorphism, as well as FcγRIIb (Arthritis Rheum, 2003, 48:3242-52; Hum Mol Genet, 2005, 14:2881-92; and Arthritis Rheum. 2002 May; 46(5): 1242-54), and every genetic polymorphism that will be reported in the future.

The FcγR includes human, mouse, rat, rabbit, and monkey-derived FcγRs but is not limited thereto, and may be derived from any organism. Mouse FcγRs include FcγRI (CD64), FcγRII (CD32), FcγRIII (CD16), and FcγRIII-2 (CD16-2), and any mouse FcγRs, or FcγR isoforms or allotypes yet to be discovered, but are not limited thereto. Favorable examples of such Fcγ receptors include human FcγRI (CD64), FcγRIIA (CD32), FcγRIIB (CD32), FcγRIIIA (CD16), and/or FcγRIIIB (CD16).

The polynucleotide sequence and amino acid sequence of FcγRI are set forth in SEQ ID NOs: 1 (NM_000566.3) and 2 (NP_000557.1), respectively;
the polynucleotide sequence and amino acid sequence of FcγRIIA are set forth in SEQ ID NOs: 3 (BC020823.1) and 4 (AAH20823.1), respectively;
the polynucleotide sequence and amino acid sequence of FcγRIIB are set forth in SEQ ID NOs: 5 (BC146678.1) and 6 (AAI46679.1), respectively;
the polynucleotide sequence and amino acid sequence of FcγRIIIA are set forth in SEQ ID NOs: 7 (BC033678.1) and 8 (AAH33678.1), respectively; and
the polynucleotide sequence and amino acid sequence of FcγRIIIB are set forth in SEQ ID NOs 9 (BC128562.1) and 10 (AAI28563.1), respectively (the RefSeq Registration number is indicated inside the parentheses).

In FcγRIIa, there are two allotypes, one where the amino acid at position 131 of FcγRIIa is histidine (type H) and the other where this amino acid is substituted with arginine (type R) (J. Exp. Med, 172:19-25, 1990).

Herein, an “Fc region to which an amino acid alteration(s) has not been introduced”, or a similar expression, refers to an Fc region prior to introduction of amino acid alteration(s) of the present invention. In the present invention, this may be, for example, a native IgG Fc region, or an IgG Fc region produced by adding an alteration other than the amino acid alteration(s) of the present invention to a native IgG. Furthermore, in the present invention, “Fc region variant” means an Fc region in which at least one amino acid has been altered to another amino acid of the present invention in the Fc region without introduction of amino acid alteration(s) of the present invention. Herein, Fc region with “at least one amino acid has been altered to another amino acid” includes an Fc region introduced with this amino acid alteration, and an Fc region consisting of the same amino acid sequence.

“Naturally-occurring IgGs” refers to polypeptides belonging to a class of antibodies practically encoded by immunoglobulin gamma genes and comprising an amino acid sequence identical to those of IgGs found in nature. For example, a naturally-occurring human IgG means a native human IgG1, native human IgG2, native human IgG3, naturally-occurring human IgG4, or such. Naturally-occurring IgGs also include mutants spontaneously produced from them.

The Fc region of a native IgG means an Fc region comprising an amino acid sequence identical to that of the Fc region derived from an IgG found in nature. The heavy-chain constant region of a native IgG is shown in FIG. 21 (SEQ ID NOs: 11-14), and for example, it refers to, in FIG. 21, Fc regions in heavy-chain constant region derived from native human IgG1, Fc regions in heavy-chain constant region derived from native human IgG2, Fc regions in heavy-chain constant region derived from native human IgG3, and Fc regions in heavy-chain constant region derived from native human IgG4. The Fc regions of native IgGs also include mutants spontaneously produced from them.

In the present invention, whether or not the binding activity towards each type of FcγR is enhanced, or maintained or decreased in a polypeptide comprising an Fc region variant or an Fc region variant of the present invention can be determined, for example, by observing whether there is a decrease or an increase in the dissociation constant (KD) value obtained from the results of sensorgram analysis, where various FcγRs are subjected to interaction as an analyte with antibodies immobilized onto the sensor chips or captured onto the sensor chips using Protein A, Protein L, Protein A/G, Protein G, anti-lambda chain antibodies, anti-kappa chain antibodies, antigenic peptides, antigenic proteins, or such using a BIACORE™ system that is an interaction analyzer that utilizes the surface plasmon resonance (SPR) phenomena, as shown in the Examples. Alternatively, it can also be determined by observing whether there is an increase or a decrease in the value obtained by dividing the amount of change in the resonance unit (RU) value on the sensorgram before and after various types of FcγRs are subjected to interaction as an analyte with antibodies immobilized onto the sensor chips or captured onto the sensor chips using Protein A, Protein L, Protein A/G, Protein G, anti-lambda chain antibodies, anti-kappa chain antibodies, antigenic peptides, antigenic proteins, or such, by the amount of change of resonance units (RU) before and after antibodies are immobilized or captured onto the sensor chip. Furthermore, it can be determined by observing an increase or a decrease in the dissociation constant (KD) values obtained from sensorgram analysis, where a sample such as an antibody to be evaluated is subjected to interaction as an analyte using a sensor chip onto which FcγR is immobilized directly or via an anti-tag antibody. Alternatively, it can be determined by observing whether the amount of change in sensorgram values increases or decreases before and after a sample such as an antibody to be evaluated is subjected to interaction as an analyte with the sensor chip onto which FcγR is immobilized directly or via an anti-tag antibody.

Specifically, the binding activity of an Fc region variant towards an Fcγ receptor can be measured by the Amplified Luminescent Proximity Homogeneous Assay (ALPHA) screening, the BIACORE™ method which utilizes the surface plasmon resonance (SPR) phenomena, or such, in addition to ELISA or fluorescence activated cell sorting (FACS) (Proc. Natl. Acad. Sci. USA (2006) 103 (11): 4005-4010).

ALPHA screening is performed by ALPHA technology which uses two beads, a donor and an acceptor, based on the following principles. Luminescent signals are detected only when molecules bound to donor beads physically interact with molecules bound to the acceptor beads, and the two beads are in close proximity to each other. Laser-excited photosensitizer in the donor beads converts ambient oxygen to excited-state singlet oxygen. Singlet oxygen is dispersed around the donor beads, and when it reaches the adjacent acceptor beads, chemiluminescent reaction is induced in the beads, and light is ultimately emitted. When the molecules bound to the donor beads do not interact with the molecules bound to the acceptor beads, the chemiluminescent reaction does not take place because singlet oxygen produced by the donor beads does not reach the acceptor beads.

For example, a biotinylated polypeptide complex is bound to the donor beads, and Fcγ receptor tagged with glutathione S transferase (GST) is linked to the acceptor beads. In the absence of a competing polypeptide complex comprising an Fc region variant, the polypeptide complex comprising a wild-type Fc region interacts with the Fcγ receptor and produces 520-620 nm signals. The polypeptide complex comprising an untagged mutant Fc region competes with the polypeptide complex comprising a wild-type Fc region for interaction with the Fcγ receptor. Relative binding activities can be determined by quantifying the decrease in fluorescence observed as a result of the competition. Biotinylation of polypeptide complexes such as antibodies using Sulfo-NHS-biotin and such is well known. The method of expressing the Fcγ receptor and GST in a cell carrying a fusion gene produced by fusing a polynucleotide encoding the Fcγ receptor in frame with a polynucleotide encoding GST in an expressible vector, and performing purification using a glutathione column is appropriately adopted as a method for tagging an Fcγ receptor with GST. The obtained signals are preferably analyzed, for example, by fitting them to a one-site competition model which uses a non-linear regression analysis using software such as GRAPHPAD PRISM (GraphPad, San Diego).

One of the substances (the ligand) in observation of an interaction is immobilized onto a gold thin film on a sensor chip, and by shining light from the reverse side of the sensor chip so that total reflection takes place at the interface between the gold thin film and glass, a portion of reduced reflection intensity is formed in part of the reflected light (SPR signal). When the other one of the substances (the analyte) in observation of an interaction is made to flow on the sensor chip surface and the ligand binds to the analyte, the mass of the immobilized ligand molecule increases and the refractive index of the solvent on the sensor chip surface changes. The position of the SPR signal shifts as a result of this change in refractive index (on the other hand, the signal position returns when this binding dissociates). The Biacore™ system indicates the amount of shift mentioned above, or more specifically the time variable of mass by plotting the change in mass on the sensor chip surface on the ordinate as the measurement data (sensorgram). The amount of analyte bound to the ligand trapped on the sensor chip surface is determined from the sensorgram. Kinetic parameters such as association rate constants (ka) and dissociation rate constants (kd) are determined from the curves of the sensorgram, and the dissociation constants (KD) are determined from the ratio of these constants. In the BIACORE™ method, a method for measuring inhibition is preferably used. An example of the method for measuring inhibition is described in Proc. Natl. Acad. Sci USA (2006) 103 (11): 4005-4010.

An Fc region with decreased FcγR-binding activity or a polypeptide comprising this Fc region refers to an Fc region variant or a polypeptide comprising the Fc region variant which binds to FcγR with essentially weaker binding activity than a polypeptide comprising the parent Fc region when assays are performed by using substantially the same amount of a polypeptide comprising an Fc region to which an amino acid alteration(s) has not been introduced (also referred to as polypeptides comprising parent Fc regions or parent polypeptides) and a polypeptide comprising at least one amino acid alteration in the Fc region (also referred to as a polypeptide comprising an Fc region variant or an altered polypeptide).

Furthermore, an Fc region with enhanced FcγR-binding activity or a polypeptide comprising the Fc region refers to an Fc region variant or a polypeptide comprising the Fc region variant which binds to FcγR with essentially stronger binding activity than a polypeptide containing the parent Fc region when assays are performed by using substantially the same amount of a polypeptide comprising a parent Fc region and a polypeptide comprising an Fc region variant.

A polypeptide with maintained FcγR-binding activity refers to a polypeptide that binds to FcγR with binding activity equivalent to or essentially not different from that of the parent polypeptide when assays are performed by using substantially the same amount of a polypeptide comprising a parent Fc region and a polypeptide comprising the Fc region variant.

In the present invention, enhanced FcγRIIb-binding activity preferably means, for example, that the KD value ratio for [KD value of a polypeptide comprising a parent Fc region for FcγRIIb]/[KD value of a polypeptide comprising an Fc region variant for FcγRIIb] in the KD values measured by the above-mentioned measurement method preferably becomes 15.0 or greater, 20.0 or greater, 25.0 or greater, 30.0 or greater, 35.0 or greater, 40.0 or greater, 45.0 or greater, or even 50.0 or greater, 55.0 or greater, 60.0 or greater, 65.0 or greater, 70.0 or greater, 75.0 or greater, 80.0 or greater, 85.0 or greater, 90.0 or greater, 95.0 or greater, or 100.0 or greater.

Furthermore, “an Fc region variant of the present invention shows enhanced binding selectivity to FcγRIIb compared to FcγRIIa” means that:
(i) FcγRIIb-binding activity is enhanced, and FcγRIIa-binding activity is maintained or decreased;
(ii) FcγRIIb-binding activity is enhanced and FcγRIIa-binding activity is also enhanced, but the degree of enhancement of FcγRIIa-binding activity is lower than the degree of enhancement of FcγRIIb-binding activity; or
(iii) FcγRIIb-binding activity is decreased, but the degree of decrease of the binding activity is less than the degree of decrease of FcγRIIa-binding activity. Whether or not an Fc region variant of the present invention is a variant with improved binding selectivity for FcγRIIb rather than for FcγRIIa can be determined, for example, by comparing the ratio of the KD value for FcγRIIa to the KD value for FcγRIIb of the polypeptide comprising an Fc region variant of the present invention (KD value for FcγRIIa/KD value for FcγRIIb) with the ratio of the KD value for FcγRIIa to the KD value for FcγRIIb of the polypeptide comprising the parent Fc region (KD value for FcγRIIa/KD value for FcγRIIb), which were determined according to the above-mentioned examples. Specifically, when the value of the KD ratio for the polypeptide comprising the Fc region variant of the present invention is greater than that of the polypeptide comprising the parent Fc region, the polypeptide comprising the Fc region variant of the present invention can be determined to have an improved binding selectivity for FcγRIIb rather than for FcγRIIa in comparison with the polypeptide comprising the parent Fc region variant. In particular, since FcγRIIa (type R)-binding activity is likely to correlate with binding activity to FcγRIIb than to FcγRIIa (type H), finding amino acid alteration(s) that can enhance binding selectivity to FcγRIIb compared to FcγRIIa (type R) is important for enhancing binding selectivity to FcγRIIb compared to other FcγRs other than FcγRIIb.

The binding selectivity between FcγRIIa (type R) and FcγRIIb is, for example, a KD value ratio [KD value of the polypeptide comprising the Fc region variant for FcγRIIa (type R)]/[KD value of the polypeptide comprising the Fc region variant for FcγRIIb] of preferably 10.0 or more for the KD values measured by the measurement method described above, and more preferably 20.0 or more.

The binding selectivity between FcγRIIa (type H) and FcγRIIb is, for example, a KD value ratio [KD value of the polypeptide comprising the Fc region variant for FcγRIIa (type H)]/[KD value of the polypeptide comprising the Fc region variant for FcγRIIb] of preferably 100.0 or more, 200 or more, 300 or more, 400 or more, or 500 or more for the KD values measured by the measurement method described above, and more preferably 600 or more, 700 or more, 800 or more, or 900 or more.

Furthermore, whether or not the binding activities of the polypeptides of the present invention towards various FcγRs were maintained, enhanced, or decreased can be determined from the increase or decrease in the amount of binding of the various FcγRs to the polypeptides of the present invention, which were determined according to the examples described above. Here, the amount of binding of the various FcγRs to the polypeptides refers to values obtained by determining the difference in the RU values of sensorgrams that changed before and after interaction of various FcγRs as the analyte with each polypeptide, and dividing them by differences in the RU values of sensorgrams that changed before and after capturing polypeptides to the sensor chips.

Fc region variants of the present invention are not particularly limited in terms of their KD values (mol/L) for FcγRIIb, and for example, the values may be 9×10⁻⁷ or less, preferably 5×10⁻⁷ or less, more preferably 3×10⁻⁷ or less, even more preferably 1×10⁻⁷ or less, and yet even more preferably 5×10⁻⁸ or less.

“Fc region” refers to the fragment consisting of a hinge portion or a part thereof, CH2 domain, and CH3 domain in an antibody molecule. According to EU numbering (herein, also called the EU INDEX) (see FIG. 21), an IgG-class Fc region refers to, for example, the region from cysteine at position 226 to the C terminus, or from proline at position 230 to the C terminus, but is not limited thereto.

The Fc region may be obtained preferably by re-eluting the fraction adsorbed onto protein A column after partially digesting IgG1, IgG2, IgG3, IgG4 monoclonal antibodies or such using a protease such as pepsin. The protease is not particularly limited as long as it can digest a full-length antibody so that Fab and F(ab′) 2 will be produced in a restrictive manner by appropriately setting the enzyme reaction conditions such as pH, and examples include pepsin and papain.

The present invention provides Fc region variants having a combination of alterations which includes alteration of amino acid at position 238 (EU numbering) to another amino acid and alteration of at least one amino acid selected from amino acids at positions 233, 234, 235, 237, 264, 265, 266, 267, 268, 269, 271, 272, 274, 296, 326, 327, 330, 331, 332, 333, 334, 355, 356, 358, 396, 409, and 419 (EU numbering) to another amino acid in the Fc region of a human IgG (IgG1, IgG2, IgG3, or IgG4). By combining alteration of amino acid at position 238 (EU numbering) to another amino acid with alteration of at least one amino acid selected from amino acids at positions 233, 234, 237, 264, 265, 266, 267, 268, 269, 271, 272, 274, 296, 326, 327, 330, 331, 332, 333, 334, 355, 356, 358, 396, 409, and 419 (EU numbering) to another amino acid in the human IgG Fc region, it is possible to provide a polypeptide comprising an Fc region variant with enhanced FcγRIIb-binding activity, and/or enhanced binding selectivity to FcγRIIb, compared to FcγRIIa, to FcγRIIa (type R) in particular, as compared to those of a polypeptide containing an Fc region to which an amino acid alteration(s) has not been introduced. Other amino acid alterations that are to be combined with the amino acid alteration at position 238 (EU numbering) are preferably those at positions 233, 237, 264, 267, 268, 271, 272, 296, 327, 330, 332, 333, and 396 (EU numbering), and particularly preferably those at positions 233, 237, 264, 267, 268, 271, 296, 330, and 396 (EU numbering). In particular, in terms of enhancement of FcγRIIb-binding activity, or enhancement of binding selectivity to FcγRIIb compared to FcγRIIa, an example of a preferred combination of amino acid alterations include combination of alterations at amino acid positions 238, 268, and 271 (EU numbering) with at least one amino acid position selected from 233, 237, 264, 267, 272, 296, 327, 330, 332, and 396 (EU numbering).

Amino acids to be altered are not particularly limited as long as they lead to enhancement of FcγRIIb-binding activity or enhancement of binding selectivity to FcγRIIb compared to FcγRIIa as compared to before the alteration, but it is preferred that the amino acid at position 238 is Asp, the amino acid at position 233 is Asp, the amino acid at position 234 is Tyr, the amino acid at position 235 is Phe, the amino acid at position 237 is Asp, the amino acid at position 264 is Ile, the amino acid at position 265 is Glu, the amino acid at position 266 is Phe, Leu, or Met, the amino acid at position 267 is Ala, Glu, Gly, or Gln, the amino acid at position 268 is Asp, Gln, or Glu, the amino acid at position 269 is Asp, the amino acid at position 271 is Gly, the amino acid at position 272 is Asp, Phe, Ile, Met, Asn, Pro, or Gln, the amino acid at position 274 is Gln, the amino acid at position 296 is Asp or Phe, the amino acid at position 326 is Ala or Asp, the amino acid at position 327 is Gly, the amino acid at position 330 is Lys, Arg, or Ser, the amino acid at position 331 is Ser, the amino acid at position 332 is Lys, Arg, Ser, or Thr, the amino acid at position 333 is Lys, Arg, Ser, or Thr, the amino acid at position 334 is Arg, Ser, or Thr, the amino acid at position 355 is Ala or Gln, the amino acid at position 356 is Glu, the amino acid at position 358 is Met, the amino acid at position 396 is Ala, Asp, Glu, Phe, Gly, His, Ile, Lys, Leu, Met, Asn, Gln, Arg, Ser, Thr, Val, Trp or Tyr, the amino acid at position 409 is Arg, and the amino acid at position 419 is Glu, according to EU numbering. In particular, when combining alterations at amino acid positions 238, 268, and 271 (EU numbering) with at least one amino acid position selected from 233, 264, 267, 272, 296, 327, 330, 332, and 396 (EU numbering), it is preferred that the amino acid at position 238 is Asp, the amino acid at position 268 is Asp or Glu, the amino acid at position 271 is Gly, the amino acid at position 233 is Asp, the amino acid at position 237 is Asp, the amino acid at position 264 is Ile, the amino acid at position 267 is Ala or Gly, the amino acid at position 272 is Asp or Pro, the amino acid at position 296 is Asp, the amino acid at position 327 is Gly, the amino acid at position 330 is Arg, the amino acid at position 332 is Thr, and the amino acid at position 396 is Leu or Met, according to EU numbering.

In addition to these alterations, at least one different Fc region alteration may be added in the present invention. The added alteration is not particularly limited as long as FcγRIIb-binding activity is enhanced and/or binding selectivity to FcγRIIb compared to FcγRIIa is enhanced. Furthermore, alterations can be made by combining an alteration where a portion of an Fc region is substituted with a corresponding portion of Fc region of a different isotype. For example, it is possible to enhance FcγRIIb-binding activity and/or binding selectivity to FcγRIIb by combining the above-mentioned amino acid alterations with substitution of the amino acid sequence from Ala at position 118 to Thr at position 225 (EU numbering) in the IgG1-derived Fc region with the amino acid sequence from Ala at position 118 to Pro at position 222 (EU numbering) in the IgG4-derived Fc region. A specific example includes a combination of amino acid alterations introduced into IL6R-BP230, and the alteration of substituting the amino acid sequence from Ala at position 118 to Thr at position 225 (EU numbering) in G1d with the amino acid sequence from Ala at position 118 to Pro at position 222 (EU numbering) in G4d, as of IL6R-BP478/IL6R-L described in Example 7.

Among them, alterations that lead to greater enhancement of FcγRIIb-binding activity, or lead to greater enhancement of binding selectivity to FcγRIIb compared to FcγRIIa (type R) are preferred. Examples of such a preferred combination of amino acid alterations include the following (a) to (x):
(a) amino acid alterations at positions 238, 233, 237, 268, 271, 296, and 330 (EU numbering) of an Fc region;
(b) amino acid alterations at positions 238, 237, 268, 271, 296, and 330 (EU numbering) of an Fc region;
(c) amino acid alterations at positions 238, 233, 237, 268, 271, 296, 330, and 332 (EU numbering) of an Fc region;
(d) amino acid alterations at positions 238, 233, 237, 264, 267, 268, 271, and 330 (EU numbering) of an Fc region;
(e) amino acid alterations at positions 238, 233, 237, 267, 268, 271, 296, 330, and 332 (EU numbering) of an Fc region;
(f) amino acid alterations at positions 238, 237, 267, 268, 271, 296, 330, and 332 (EU numbering) of an Fc region;
(g) amino acid alterations at positions 238, 233, 237, 268, 271, 296, 327, and 330 (EU numbering) of an Fc region;
(h) amino acid alterations at positions 238, 233, 237, 264, 267, 268, and 271 (EU numbering) of an Fc region;
(i) amino acid alterations at positions 238, 233, 237, 264, 267, 268, 271, 296, and 330 (EU numbering) of an Fc region;
(j) amino acid alterations at positions 238, 233, 237, 264, 267, 268, 271, 296, 330, and 396 (EU numbering) of an Fc region;
(k) amino acid alterations at positions 238, 237, 264, 267, 268, 271, and 330 (EU numbering) of an Fc region;
(l) amino acid alterations at positions 238, 237, 264, 267, 268, 271, 296, and 330 (EU numbering) of an Fc region;
(m) amino acid alterations at positions 238, 264, 267, 268, and 271 (EU numbering) of an Fc region;
(n) amino acid alterations at positions 238, 264, 267, 268, 271, and 296 (EU numbering) of an Fc region;
(o) amino acid alterations at positions 238, 237, 267, 268, 271, 296, and 330 (EU numbering) of an Fc region;
(p) amino acid alterations at positions 238, 233, 237, 264, 267, 268, 271, 330, and 396 (EU numbering) of the Fc region;
(q) amino acid alterations at positions 238, 233, 237, 264, 267, 268, 271, 296, 327, 330, and 396 (EU numbering) of an Fc region;
(r) amino acid alterations at positions 238, 233, 237, 264, 267, 268, 271, 272, and 296 (EU numbering) of an Fc region;
(s) amino acid alterations at positions 238, 237, 264, 267, 268, 271, 272, and 330 (EU numbering) of an Fc region;
(t) amino acid alterations at positions 238, 237, 264, 267, 268, 271, 272, 296, and 330 (EU numbering) of an Fc region;
(u) amino acid alterations at positions 238, 233, 264, 267, 268, and 271 (EU numbering) of an Fc region;
(v) amino acid alterations at positions 238, 237, 267, 268, 271, 296, and 330 (EU numbering) of an Fc region;
(w) amino acid alterations at positions 238, 264, 267, 268, 271, 272, and 296 (EU numbering) of an Fc region; and
(x) amino acid alterations at positions 238, 233, 264, 267, 268, 271, and 296 (EU numbering) of an Fc region.

In addition, among these combinations, the following combinations of amino acid alterations (a) to (x) below are more preferred amino acid combinations:
(a) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, and the amino acid at position 330 is Arg, according to EU numbering, in an Fc region;
(b) an amino acid sequence in which the amino acid at position 238 is Asp, the amino acid at position 237 is Asp, the amino acid at position 268 is Asp or Glu, the amino acid at position 271 is Gly, the amino acid at position 296 is Asp, and the amino acid at position 330 is Arg, according to EU numbering, in an Fc region;
(c) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 330 is Arg, and the amino acid at position 332 is Thr, according to EU numbering, in an Fc region;
(d) an amino acid sequence in which the amino acid at position 238 is Asp, the amino acid at position 233 is Asp, the amino acid at position 237 is Asp, the amino acid at position 264 is Ile, the amino acid at position 267 is Gly or Ala, the amino acid at position 268 is Glu, the amino acid at position 271 is Gly, and the amino acid at position 330 is Arg, according to EU numbering, in an Fc region;
(e) an amino acid sequence in which the amino acid at position 238 is Asp, the amino acid at position 233 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(f) an amino acid sequence in which the amino acid at position 238 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(g) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 327 is Gly, and the amino acid at position 330 is Arg, according to EU numbering, in an Fc region;
(h) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, and the amino acid at position 271 is Gly, according to EU numbering, in an Fc region;
(i) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(j) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 330 is Arg, and the amino acid at position 396 is Met or Leu, according to EU numbering, in the Fc region;
(k) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, and the amino acid at position 330 is Arg, according to EU numbering, in an Fc region;
(l) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(m) an amino acid sequence in which the amino acid at position 238 is Asp, the amino acid at position 264 is Ile, the amino acid at position 267 is Ala, the amino acid at position 268 is Glu, and the amino acid at position 271 is Gly, according to EU numbering, in an Fc region;
(n) an amino acid sequence in which the amino acid at position 238 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region;
(o) an amino acid sequence in which the amino acid at position 238 is Asp, the amino acid at position 237 is Asp, the amino acid at position 267 is Ala or Gly, the amino acid at position 268 is Glu, the amino acid at position 271 is Gly, the amino acid at position 296 is Asp, and the amino acid at position 330 is Arg, according to EU numbering, in an Fc region;
(p) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 330 is Arg, and the amino acid at position 396 is Met or Leu, according to EU numbering, in an Fc region;
(q) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 327 is Gly, the amino acid at position 330 is Arg, and the amino acid at position 396 is Met, according to EU numbering, in an Fc region;
(r) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region;
(s) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, and the amino acid at position 330 is Arg, according to EU numbering, in an Fc region;
(t) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, the amino acid at position 296 is Asp, and the amino acid at position 330 is Arg, according to EU numbering, in an Fc region;
(u) an amino acid sequence in which the amino acid at position 238 is Asp, the amino acid at position 233 is Asp, the amino acid at position 264 is Ile, the amino acid at position 267 is Ala, the amino acid at position 268 is Glu, and the amino acid at position 271 is Gly, according to EU numbering, in an Fc region;
(v) an amino acid sequence in which the amino acid at position 238 is Asp, the amino acid at position 237 is Asp, the amino acid at position 267 is Gly, the amino acid at position 268 is Asp, the amino acid at position 271 is Gly, the amino acid at position 296 is Asp, and the amino acid at position 330 is Arg, according to EU numbering, in an Fc region;
(w) an amino acid sequence in which the amino acid at position 238 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region; and
(x) an amino acid sequence in which the amino acid at position 238 is Asp, the amino acid at position 233 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region.

In addition, amino acid alterations performed for other purpose(s) can be combined in polypeptides comprising an Fc region variant of the present invention. For example, amino acid substitutions that improve FcRn-binding activity (J. Immunol. 2006 Jan. 1; 176(1): 346-56; J Biol Chem. 2006 Aug. 18; 281(33): 23514-24; Int. Immunol. 2006 December; 18(12): 1759-69; Nat Biotechnol. 2010 February; 28(2): 157-9; WO/2006/019447; WO/2006/053301; and WO/2009/086320), and amino acid substitutions for improving antibody heterogeneity or stability (WO/2009/041613) may be added. Alternatively, polypeptides produced by conferring polypeptides comprising an Fc region variant of the present invention with the property of promoting disappearance of antigens, which are described in WO 2011/122011 or PCT/JP2011/072550, and polypeptides conferring the property for repeated binding to a plurality of antigen molecules, which are described in WO 2009/125825, WO 2012/073992 or WO 2013/047752, are also included in the present invention. Alternatively, with the objective of increasing plasma retention, amino acid alterations that decrease the pI of the constant region (WO/2012/016227) may be combined in a polypeptide comprising an Fc region variant of the present invention. Alternatively, with the objective of conferring binding ability to other antigens, the amino acid alterations disclosed in EP1752471 and EP1772465 may be combined in CH3 of a polypeptide comprising an Fc region variant of the present invention.

When a polypeptide comprising an Fc region variant of the present invention is an antigen-binding molecule such as an antibody, amino acid alterations of enhancing human FcRn-binding activity under an acidic pH range condition can be combined to enhance the effect of the antigen-binding molecule to eliminate antigens from plasma. More specifically, alterations used to enhance human FcRn-binding activity under an acidic pH range condition may be carried out on an IgG antibody, for example, by a method of substituting Leu for Met at position 428, and substituting Ser for Asn at position 434, according to EU numbering (Nat Biotechnol, 2010 28:157-159); a method of substituting Ala for Asn at position 434 (Drug Metab Dispos. 2010 April; 38(4): 600-5); a method of substituting Tyr for Met at position 252, substituting Thr for Ser at position 254, and substituting Glu for Thr at position 256 (J Biol Chem, 2006, 281:23514-23524); a method for substituting Gln for Thr at position 250, and substituting Leu for Met at position 428 (J Immunol. 2006, 176(1): 346-56); method of substituting His for Asn at position 434 (Clinical Pharmacology & Therapeutics (2011) 89(2): 283-290), or by using alterations such as those described in WO2010106180, WO2010045193, WO2009058492, WO2008022152, WO2006050166, WO2006053301, WO2006031370, WO2005123780, WO2005047327, WO2005037867, WO2004035752, WO2002060919, or such.

Furthermore, an antibody molecule produced by substituting His for Asn at position 434 (EU numbering) in humanized anti-CD4 antibody to enhance human FcRn-binding activity under an acidic pH range condition and to improve plasma retention properties was recently reported to bind to rheumatoid factors (RF) (Clin Pharmacol Ther. 2011 February; 89(2): 283-90). This antibody has a human IgG1 Fc region, but by substituting His for Asn at position 434 which is positioned at the FcRn-binding site, it has been shown to bind to rheumatoid factors that recognize this substituted site.

As described above, various alterations have been reported as alterations for enhancing human FcRn-binding activity under an acidic pH range condition; however, by introducing these alterations into the FcRn-binding site in an Fc region, affinity to rheumatoid factors which recognize this site may become enhanced.

However, by introducing alterations which do not reduce FcRn-binding activity and reduce only binding activity to rheumatoid factors into the site in the Fc region, antigen-binding molecules with enhanced human FcRn-binding activity under an acidic pH range condition and without affinity to rheumatoid factors can be produced.

For alterations that reduce binding activity to rheumatoid factors, alterations to positions 248-257, 305-314, 342-352, 380-386, 388, 414-421, 423, 425-437, 439, and 441-444 according to EU numbering are used. Preferably, alterations to positions 387, 422, 424, 426, 433, 436, 438, and 440 are used. Particularly preferably, alteration of substituting Glu or Ser for Val at position 422, alteration of substituting Arg for Ser at position 424, alteration of substituting Asp for His at position 433, alteration of substituting Thr for Tyr at position 436, alteration of substituting Arg or Lys for Gln at position 438, and alteration of substituting Glu or Asp for Ser at position 440 are used. These alterations may be used alone or by combining alterations at multiple positions.

Alternatively, to decrease binding activity to rheumatoid factors, an N-type glycosylation sequence may be introduced into this site. Specifically, Asn-Xxx-Ser/Thr (Xxx is any amino acid other than Pro) is known as an N-type glycosylation sequence. Adding an N-type sugar chain by introducing this sequence into the site in the Fc region enables inhibition of binding to RF by steric hindrance due to the N-type sugar chain. Alterations used to add an N-type sugar chain are preferably alteration which substitutes Asn for Lys at position 248, alteration which substitutes Asn for Ser at position 424, alteration which substitutes Asn for Tyr at position 436 and substitutes Thr for Gln at position 438, and alteration which substitutes Asn for Gln at position 438. Particularly preferably, the alteration which substitutes Asn for Ser at position 424 is used.

Preferred example of a polypeptide comprising an Fc region variant of the present invention includes a polypeptide comprising at least two Fc region variants wherein the two Fc region variants are associated, much like in an IgG antibody. When an IgG antibody is used as a polypeptide of the present invention, the type of constant region is not limited, and an IgG isotypes (subclasses) such as IgG1, IgG2, IgG3, and IgG4 can be used. IgG antibodies of the present invention are preferably human IgG, and more preferably human IgG1 and human IgG4. The amino acid sequences of the heavy-chain constant regions of human IgG1 and human IgG4 are known. A plurality of allotype sequences due to genetic polymorphisms have been described in Sequences of Proteins of Immunological Interest, NIH Publication No. 91-3242 for the human IgG1 constant region, and any of the sequences may be used in the present invention.

The two associated Fc region variants included in the aforementioned polypeptide may be Fc region variants introduced with the same amino acid alteration(s) (hereinafter, referred to as a polypeptide containing homologous Fc region variants) or Fc region variants comprising different amino acid sequences where each have been introduced with different amino acid alteration(s), or alternatively Fc region variants comprising different amino acid sequences where only one of the Fc regions has been introduced with amino acid alteration(s) (hereinafter, referred as a polypeptide containing heterologous Fc region variants). As the amino acid alteration to be introduced into only one of the Fc regions, alteration in the loop structure portion from positions 233 to 239 (EU numbering) in the Fc region CH2 domain involved in binding with FcγRIIb and FcγRIIa is preferred; and preferably, an alteration that enhances FcγRIIb-binding activity and/or enhances binding selectivity to FcγRIIb compared to FcγRIIa (type R) of the loop structure of the CH2 region of one of the Fc regions is introduced and an amino acid alteration that destabilizes the loop structure of the CH2 region of the other Fc region is introduced. Examples of amino acid alterations that can destabilize the loop structure of the CH2 region may be substitution of at least one amino acid selected from amino acids at positions 235, 236, 237, 238, and 239 to another amino acid. Specifically, the CH2 region loop structure can be destabilized, for example, by altering the amino acid at position 235 to Asp, Gln, Glu, or Thr, altering the amino acid at position 236 to Asn, altering the amino acid at position 237 to Phe or Trp, altering the amino acid at position 238 to Glu, Gly, or Asn, and altering the amino acid at position 239 to Asp or Glu, according to EU numbering.

To produce a polypeptide comprising heterologous Fc region variants of the present invention, it is required that Fc region variants having amino acids that differ from each other are associated, or a polypeptide comprising heterologous Fc region variants of interest is separated from other polypeptides comprising homologous Fc region variants.

For association of polypeptides having different amino acids from each other, a technique of suppressing unintended association between H chains by introducing electrostatic repulsion into the interface of the second constant region of the antibody H chain (CH2) or the third constant region of the H chain (CH3) (WO 2006/106905) can be applied.

In the technology of suppressing unintended association between H chains by introducing electrostatic repulsion into the interface of CH2 or CH3, examples of amino acid residues in contact at the interface of other constant regions of the H chain include the residue at position 356 (EU numbering), the residue at position 439 (EU numbering), the region facing the residue at position 357 (EU numbering), the residue at position 370 (EU numbering), the residue at position 399 (EU numbering), and the residue at position 409 (EU numbering) in the CH3 domain.

More specifically, for example, in an antibody containing two types of H chain CH3 domains, the antibody in which one to three pairs of amino acid residues selected from the amino acid residues shown below in (1) to (3) in the first H chain CH3 domain have the same type of charge can be produced:
(1) amino acid residues at positions 356 and 439 (EU numbering) which are amino acid residues contained in the H chain CH3 domain;
(2) amino acid residues at positions 357 and 370 (EU numbering) which are amino acid residues contained in the H chain CH3 domain; and
(3) amino acid residues at positions 399 and 409 (EU numbering) which are amino acid residues contained in the H chain CH3 domain.

Furthermore, an antibody can be produced in which one to three pairs of amino acid residues corresponding to the amino acid residue pairs indicated above in (1) to (3) having the same type of charge in the first H chain CH3 domain have charges opposite to the corresponding amino acid residues in the aforementioned first H chain CH3 domain, wherein the amino acid residue pairs are selected from the amino acid residue pairs indicated above in (1) to (3) in the second H chain CH3 domain which differs from the first H chain CH3 domain.

The respective amino acid residues of (1) to (3) mentioned above are positioned close to each other when associated. Those skilled in the art can find sites that correspond to the above-mentioned amino acid residues of (1) to (3) by homology modeling and such using commercially available software for the desired H chain CH3 domain or H chain constant region, and amino acid residues of these sites can be altered when appropriate.

In the above-mentioned antibodies, for example, “charged amino acid residues” are preferably selected from amino acid residues included in either of groups (X) or (Y) below:
(X) glutamic acid (E) and aspartic acid (D); and
(Y) lysine (K), arginine (R), and histidine (H).

In the above-mentioned antibodies, the phrase “having the same type of charge” means that, for example, all of the two or more amino acid residues are amino acid residues included in either of the above-mentioned groups (X) and (Y). The phrase “having the opposite charge” means that, for example, when at least one of the two or more amino acid residues is an amino acid residue included in either one of the above-mentioned groups (X) and (Y), the remaining amino acid residues are amino acid residues included in the other group.

In a preferred embodiment of the above-mentioned antibody, the first H chain CH3 domain and the second H chain CH3 domain may be cross-linked by disulfide bonds.

In the present invention, the amino acid residues to be altered are not limited to amino acid residues of the antibody constant region or antibody variable region described above. Those skilled in the art can find amino acid residues that form the interface in polypeptide mutants or heteromultimers through homology modeling and such using commercially available software, and can alter the amino acid residues at those sites to regulate association.

Other known techniques can be used additionally for association of heterologous Fc region variants. Specifically, such a technique is conducted by substituting an amino acid side chain present in a variable region of one of the H chains in an antibody with a larger side chain (knob; which means “bulge”), and substituting an amino acid side chain present in a variable region of the other H chain with a smaller side chain (hole; which means “void”), to place the knob within the hole. This can promote efficient association between Fc-region-containing polypeptides having different amino acids (WO 1996/027011; Ridgway J B et al., Protein Engineering (1996) 9, 617-621; Merchant A M et al., Nature Biotechnology (1998) 16, 677-681).

In addition, other known techniques can also be used for heterologous association of Fc region variants. Association of polypeptides having different sequences can be induced efficiently by complementary association of CH3 by using a strand-exchange engineered domain CH3 produced by changing a portion of one of the H chain CH3 of an antibody to an IgA-derived sequence corresponding to that portion and introducing to the complementary portion of the other H chain CH3, an IgA-derived sequence corresponding to that portion (Protein Engineering Design & Selection, 23; 195-202, 2010). This known technique can also be used to efficiently induce association between Fc region-containing polypeptides having different amino acids from each other.

In addition, heterodimerized antibody production techniques that use association of antibody CH1 and CL, and association of VH and VL, which are described in WO2011/028952, can also be used.

As with the method described in WO2008/119353 and WO2011/131746, it is also possible to use the technique of producing heterodimerized antibodies by producing two types of homodimerized antibodies in advance, incubating them under reducing conditions to dissociate them, and allowing them to associate again.

As with the method described in J. Mol. (2012) 420, 204-219, it is also possible to use the technique of producing heterodimerized antibodies by introducing charged residues such as Lys, Arg, Glu, and Asp so that electrostatic repulsion is introduced into CH3 of IgG1 and IgG2.

Furthermore, as with the method described in WO2012/058768, it is also possible to use the technique of producing heterodimerized antibodies by adding alterations to the CH2 and CH3 regions.

Furthermore, even in cases where polypeptides comprising heterologous Fc region variants cannot be formed efficiently, polypeptides comprising heterologous Fc region variants can be obtained by separating and purifying them from polypeptides comprising homologous Fc region variants. When producing a polypeptide comprising heterologous Fc region variants consisting of a first polypeptide and a second polypeptide which have different sequences from each other, polypeptides comprising homologous Fc region consisting of only two first polypeptides, and polypeptide comprising homologous Fc region consisting of only two second polypeptide are mixed in as impurities. Known technologies can be used as a method for efficiently removing these two types of polypeptides comprising homologous Fc region. A method has been reported to be able to purify two types of homodimers and the heterodimerized antibody of interest by ion exchange chromatography, by creating a difference in isoelectric points by introducing amino acid substitutions into the variable regions of the two types of H chains (WO 2007114325). To date, as a method for purifying heterodimerized antibodies, a method using Protein A has been reported to purify a heterodimerized antibody comprising a mouse IgG2a H chain that binds to Protein A and a rat IgG2b H chain that does not bind to Protein A (WO 98050431 and WO 95033844).

Furthermore, a heterodimerized antibody alone can be efficiently purified by using H chains in which amino acid residues at the IgG-Protein A binding site, positions 435 and 436 (EU numbering), are substituted with amino acids yielding different Protein A affinities such as Tyr or His to change interaction of each of the H chains with Protein A, and using a Protein A column.

A plurality of these substitutions and technologies, for example, two or more of them can be used in combination. Furthermore, these alterations can be made separately to the first polypeptide and the second polypeptide when necessary. Polypeptides of the present invention may also be those produced based on the products of the above-mentioned alterations.

In the present invention, amino acid alteration means any of substitution, deletion, addition, insertion, and modification, or a combination thereof. In the present invention, amino acid alteration may be rephrased as amino acid mutation, and they are used synonymously.

When substituting amino acid residues, substitution to a different amino acid residue is carried out with the objective of altering aspects such as (a)-(c) described below:
(a) polypeptide backbone structure in the sheet-structure or helical-structure region;
(b) electric charge or hydrophobicity at the target site; or
(c) size of the side chain.

Amino acid residues are classified into the following groups based on their general side chain properties:
(1) hydrophobic: norleucine, met, ala, val, leu, and ile;
(2) neutral hydrophilic: cys, ser, thr, asn, and gln;
(3) acidic: asp and glu;
(4) basic: his, lys, and arg;
(5) residues that affect the chain orientation: gly and pro; and
(6) aromatic: trp, tyr, and phe.

Substitution between amino acid residues within each of these amino acid groups is referred to as conservative substitution, and amino acid residue substitution between different groups is referred to as non-conservative substitution. Substitutions in the present invention may be conservative substitutions or non-conservative substitutions, or a combination of conservative substitutions and non-conservative substitutions.

Amino acid sequence alterations are produced by various methods known to those skilled in the art. Such methods include the site-directed mutagenesis method (Hashimoto-Gotoh, T, Mizuno, T, Ogasahara, Y, and Nakagawa, M. (1995) An oligodeoxyribonucleotide-directed dual amber method for site-directed mutagenesis. Gene 152:271-275; Zoller, M J, and Smith, M. (1983) Oligonucleotide-directed mutagenesis of DNA fragments cloned into M13 vectors. Methods Enzymol. 100:468-500; Kramer, W, Drutsa, V, Jansen, H W, Kramer, B, Pflugfelder, M, and Fritz, H J (1984) The gapped duplex DNA approach to oligonucleotide-directed mutation construction. Nucleic Acids Res. 12:9441-9456; Kramer W, and Fritz H J (1987) Oligonucleotide-directed construction of mutations via gapped duplex DNA Methods. Enzymol. 154, 350-367; and Kunkel, TA (1985) Rapid and efficient site-specific mutagenesis without phenotypic selection. Proc Natl Acad Sci USA. 82:488-492), the PCR mutation method, and the cassette mutation method, but are not limited thereto.

Amino acid modification of the present invention includes post-translational modification. A specific post-translational modification may be addition or deletion of a sugar chain. For example, in the IgG1 constant region consisting of the amino acid sequence of SEQ ID NO: 11, the amino acid residue at position 297 (EU numbering) may be sugar chain-modified. The sugar-chain structure for the modification is not limited. Generally, antibodies expressed in eukaryotic cells comprise glycosylation in the constant region. Therefore, antibodies expressed in cells such as those below are normally modified by some type of sugar chain:
antibody-producing cells of mammals
eukaryotic cells transformed with an expression vector comprising a DNA encoding an antibody

Eukaryotic cells shown here include yeast and animal cells. For example, CHO cells and HEK293H cells are representative animal cells used in transformation with an expression vector comprising an antibody-encoding DNA. On the other hand, those without glycosylation at this site are also included in the constant region of the present invention. Antibodies whose constant region is not glycosylated can be obtained by expressing an antibody-encoding gene in prokaryotic cells such as Escherichia coli.

Specifically, for example, sialic acid may be added to the sugar chain of an Fc region (MAbs. 2010 September-October; 2(5): 519-27).

Furthermore, the present invention provides antibodies comprising any of Fc region variant described above.

The term “antibody/antibodies” in the present invention is used in the broadest sense, and as long as the desired biological activity is shown, it comprises any antibody such as monoclonal antibodies (including full-length monoclonal antibodies), polyclonal antibodies, antibody variants, antibody fragments, polyspecific antibodies (multi-specific antibodies) (for example, bispecific antibodies (diabodies)), chimeric antibodies, and humanized antibodies.

Regarding the antibodies of the present invention, the antigen type and antibody origin are not limited, and they may be any type of antibodies. The origin of the antibodies is not particularly limited, but examples include human antibodies, mouse antibodies, rat antibodies, and rabbit antibodies.

Methods for producing the antibodies are well known to those skilled in the art, and for example, monoclonal antibodies may be produced by the hybridoma method (Kohler and Milstein, Nature 256:495 (1975)), or the recombination method (U.S. Pat. No. 4,816,567). Alternatively, they may be isolated from a phage antibody library (Clackson et al., Nature 352:624-628 (1991); Marks et al., J. Mol. Biol. 222:581-597 (1991)).

A humanized antibody is also called a reshaped human antibody. Specifically, humanized antibodies prepared by grafting the CDRs of a non-human animal antibody such as a mouse antibody to a human antibody and such are known. Common genetic engineering techniques for obtaining humanized antibodies are also known. Specifically, for example, overlap extension PCR is known as a method for grafting mouse antibody CDRs to human FRs.

A vector for expressing a humanized antibody can be produced by inserting a DNA encoding an antibody variable region in which three CDRs and four FRs are ligated and a DNA encoding a human antibody constant region into an expression vector so that these DNAs are fused in frame. After this integration vector is transfected into a host to establish recombinant cells, these cells are cultured, and the DNA encoding the humanized antibody is expressed to produce the humanized antibody in the culture of the cells (see, European Patent Publication No. EP 239,400, and International Patent Publication No. WO 1996/002576).

As necessary, an amino acid residue in an FR may be substituted so that the CDRs of a reshaped human antibody form an appropriate antigen-binding site. For example, a mutation can be introduced into the amino acid sequence of an FR by applying the PCR method used for grafting mouse CDRs to human FRs.

A desired human antibody can be obtained by DNA immunization using a transgenic animal having the complete repertoire of human antibody genes (see International Publication Nos. WO 1993/012227, WO 1992/003918, WO 1994/002602, WO 1994/025585, WO 1996/034096, and WO 1996/033735) as an animal for immunization.

Furthermore, technologies for obtaining a human antibody by panning using a human antibody library are known. For example, a human antibody V region is expressed on the surface of a phage as a single-chain antibody (scFv) by the phage display method. The scFv-expressing phage that binds to the antigen can be selected. The DNA sequence that encodes the V region of the antigen-bound human antibody can be determined by analyzing the genes of the selected phage. After determining the DNA sequence of the scFv that binds to the antigen, an expression vector can be prepared by fusing the V-region sequence in-frame with the sequence of a desired human antibody C region, and then inserting this into a suitable expression vector. The expression vector is introduced into suitable expression cells such as those described above, and the human antibody can be obtained by expressing the human antibody-encoding gene. These methods are already known (see, International Publication Nos. WO 1992/001047, WO 1992/020791, WO 1993/006213, WO 1993/011236, WO 1993/019172, WO 1995/001438, and WO 1995/15388).

Variable regions constituting the antibodies of the present invention can be variable regions that recognize any antigen.

Herein, there is no particular limitation on the antigen, and it may be any antigens. Examples of such antigens preferably include ligands (cytokines, chemokines, and such), receptors, cancer antigens, MHC antigens, differentiation antigens, immunoglobulins, and immune complexes partly containing immunoglobulins.

Examples of cytokines include interleukins 1 to 18, colony stimulating factors (G-CSF, M-CSF, GM-CSF, etc.), interferons (IFN-α, IFN-β, IFN-γ, etc.), growth factors (EGF, FGF, IGF, NGF, PDGF, TGF, HGF, etc.), tumor necrosis factors (TNF-α and TNF-β), lymphotoxin, erythropoietin, leptin, SCF, TPO, MCAF, and BMP.

Examples of chemokines include CC chemokines such as CCL1 to CCL28, CXC chemokines such as CXCL1 to CXCL17, C chemokines such as XCL1 to XCL2, and CX3C chemokines such as CX3CL1.

Examples of receptors include receptors belonging to receptor families such as the hematopoietic growth factor receptor family, cytokine receptor family, tyrosine kinase-type receptor family, serine/threonine kinase-type receptor family, TNF receptor family, G protein-coupled receptor family, GPI anchor-type receptor family, tyrosine phosphatase-type receptor family, adhesion factor family, and hormone receptor family. The receptors belonging to these receptor families and their characteristics have been described in many documents such as Cooke B A., King R J B., van der Molen H J. ed. New Comprehesive Biochemistry Vol. 18B “Hormones and their Actions Part II” pp. 1-46 (1988) Elsevier Science Publishers BV; Patthy (Cell (1990) 61 (1): 13-14); Ullrich et al. (Cell (1990) 61 (2): 203-212); Massagué (Cell (1992) 69 (6): 1067-1070); Miyajima et al. (Annu. Rev. Immunol. (1992) 10:295-331); Taga et al. (FASEB J. (1992) 6, 3387-3396); Fantl et al. (Annu. Rev. Biochem. (1993), 62:453-481); Smith et al. (Cell (1994) 76 (6): 959-962); and Flower D R. Flower (Biochim. Biophys. Acta (1999) 1422 (3): 207-234).

Examples of specific receptors belonging to the above-mentioned receptor families preferably include human or mouse erythropoietin (EPO) receptors (Blood (1990) 76 (1): 31-35; and Cell (1989) 57 (2): 277-285), human or mouse granulocyte-colony stimulating factor (G-CSF) receptors (Proc. Natl. Acad. Sci. USA. (1990) 87 (22): 8702-8706, mG-CSFR; Cell (1990) 61 (2): 341-350), human or mouse thrombopoietin (TPO) receptors (Proc Natl Acad Sci USA. (1992) 89 (12): 5640-5644; EMBO J. (1993) 12 (7): 2645-53), human or mouse insulin receptors (Nature (1985) 313 (6005): 756-761), human or mouse Flt-3 ligand receptors (Proc. Natl. Acad. Sci. USA. (1994) 91 (2): 459-463), human or mouse platelet-derived growth factor (PDGF) receptors (Proc. Natl. Acad. Sci. USA. (1988) 85 (10): 3435-3439), human or mouse interferon (IFN)-α and β receptors (Cell (1990) 60 (2): 225-234; and Cell (1994) 77 (3): 391-400), human or mouse leptin receptors, human or mouse growth hormone (GH) receptors, human or mouse interleukin (IL)-10 receptors, human or mouse insulin-like growth factor (IGF)-I receptors, human or mouse leukemia inhibitory factor (LIF) receptors, and human or mouse ciliary neurotrophic factor (CNTF) receptors.

Cancer antigens are antigens that are expressed as cells become malignant, and they are also called tumor-specific antigens. Abnormal sugar chains that appear on cell surfaces or protein molecules when cells become cancerous are also cancer antigens, and they are also called sugar-chain cancer antigens. Examples of cancer antigens preferably include GPC3 which is a receptor belonging to the GPI anchor-type receptor family mentioned above, and is also expressed in several cancers including liver cancer (Int J Cancer. (2003) 103 (4): 455-65), as well as EpCAM which is expressed in several cancers including lung cancer (Proc Natl Acad Sci USA. (1989) 86 (1): 27-31), CA19-9, CA15-3, and sialyl SSEA-1 (SLX).

MHC antigens are roughly classified into MHC class I antigens and MHC class II antigens. MHC class I antigens include HLA-A, -B, -C, -E, -F, -G, and -H, and MHC class II antigens include HLA-DR, -DQ, and -DP.

Differentiation antigens may include CD1, CD2, CD4, CD5, CD6, CD7, CD8, CD10, CD11a, CD11b, CD11c, CD13, CD14, CD15s, CD16, CD18, CD19, CD20, CD21, CD23, CD25, CD28, CD29, CD30, CD32, CD33, CD34, CD35, CD38, CD40, CD41a, CD41b, CD42a, CD42b, CD43, CD44, CD45, CD45RO, CD48, CD49a, CD49b, CD49c, CD49d, CD49e, CD49f, CD51, CD54, CD55, CD56, CD57, CD58, CD61, CD62E, CD62L, CD62P, CD64, CD69, CD71, CD73, CD95, CD102, CD106, CD122, CD126, and CDw130.

Immunoglobulins include IgA, IgM, IgD, IgG, and IgE. Immune complexes include a component of at least any of the immunoglobulins.

Other antigens include, for example, the molecules below: 17-IA, 4-1BB, 4Dc, 6-keto-PGF1a, 8-iso-PGF2a, 8-oxo-dG, A1 adenosine receptor, A33, ACE, ACE-2, activin, activin A, activin AB, activin B, activin C, activin RIA, activin RIA ALK-2, activin RIB ALK-4, activin RIIA, activin RIIB, ADAM, ADAM10, ADAM12, ADAM15, ADAM17/TACE, ADAM8, ADAM9, ADAMTS, ADAMTS4, ADAMTS5, addressin, aFGF, ALCAM, ALK, ALK-1, ALK-7,alpha-1-antitrypsin, alpha-V/beta-1 antagonist, ANG, Ang, APAF-1, APE, APJ, APP, APRIL, AR, ARC, ART, artemin, anti-Id, ASPARTIC, atrial natriuretic peptide, av/b3 integrin, Axl, b2M, B7-1, B7-2, B7-H, B-lymphocyte stimulating factor (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMP, b-NGF, BOK, bombesin, bone-derived neurotrophic factor, BPDE, BPDE-DNA, BTC, complement factor 3 (C3), C3a, C4, C5, C5a, C10, CA125, CAD-8, calcitonin, cAMP, carcinoembryonic antigen (CEA), cancer associated antigen, cathepsin A, cathepsin B, cathepsin C/DPPI, cathepsin D, cathepsin E, cathepsin H, cathepsin L, cathepsin O, cathepsin S, cathepsin V, cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 protein), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6,cytokeratin tumor associated antigen, DAN, DCC, DcR3, DC-SIGN, complement regulatory factor (Decay accelerating factor), des (1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, enkephalinase, eNOS, Eot, eotaxin 1, EpCAM, ephrin B2/EphB4, EPO, ERCC, E-selectin, ET-1, factor IIa, factor VII, factor VIIIc, factor IX, fibroblast activation protein (FAP), Fas, FcR1, FEN-1, ferritin, FGF, FGF-19, FGF-2, FGF3, FGF-8, FGFR, FGFR-3, fibrin, FL, FLIP, Flt-3, Flt-4, follicle stimulating hormone, fractalkine, FZD1, FZD2, FZD3, FZD4, FZD5, FZD6, FZD7, FZD8, FZD9, FZD10, G250, Gas6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (myostatin), GDF-9, GDF-15 (MIC-1), GDNF, GDNF, GFAP, GFRa-1, GFR-alpha1, GFR-alpha2, GFR-alpha3, GITR, glucagon, Glut4, glycoprotein IIb/IIIa (GPIIb/IIIa), GM-CSF, gp130, gp72, GRO, growth hormone releasing hormone, hapten (NP-cap or NIP-cap), HB-EGF, HCC, HCMV gB envelope glycoprotein, HCMV gH envelope glycoprotein, HCMV UL, hematopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, high molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF binding protein, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, inhibin, iNOS, insulin A chain, insulin B chain, insulin-like growth factor1, integrin alpha2, integrin alpha3, integrin alpha4, integrin alpha4/beta1, integrin alpha4/beta7, integrin alpha5 (alpha V), integrin alpha5/beta1, integrin alpha5/beta3, integrin alpha6, integrin beta1, integrin beta2, interferon gamma, IP-10, I-TAC, JE, kallikrein 2, kallikrein 5, kallikrein 6, kallikrein 11, kallikrein 12, kallikrein 14, kallikrein 15, kallikrein L1, kallikrein L2, kallikrein L3, kallikrein LA, KC, KDR, keratinocyte growth factor (KGF), laminin 5, LAMP, LAP, LAP (TGF-1), latent TGF-1, latent TGF-1 bp1, LBP, LDGF, LECT2, lefty, Lewis-Y antigen, Lewis-Y associated antigen, LFA-1, LFA-3, Lfo, LIF, LIGHT, lipoprotein, LIX, LKN, Lptn, L-selectin, LT-a, LT-b, LTB4, LTBP-1, lung surface, luteinizing hormone, lymphotoxin beta receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Mullerian-inhibiting substance, Mug, MuSK, NAIP, NAP, NCAD, N-C adherin, NCA 90, NCAM, NCAM, neprilysin, neurotrophin-3, -4, or -6, neurturin, nerve growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, parathyroid hormone, PARC, PARP, PBR, PBSF, PCAD, P-cadherin, PCNA, PDGF, PDGF, PDK-1, PECAM, PEM, PF4, PGE, PGF, PGI2, PGJ2, PIN, PLA2, placental alkaline phosphatase (PLAP), PlGF, PLP, PP14, proinsulin, prorelaxin, protein C, PS, PSA, PSCA, prostate-specific membrane antigen (PSMA), PTEN, PTHrp, Ptk, PTN, R51, RANK, RANKL, RANTES, RANTES, relaxin A chain, relaxin B chain, renin, respiratory syncytial virus (RSV) F, RSV Fgp, Ret, Rheumatoid factor, RLIP76, RPA2, RSK, S100, SCF/KL, SDF-1, SERINE, serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptor (for example, T-cell receptor alpha/beta), TdT, TECK, TEM1, TEM5, TEM7, TEM8, TERT, testis PLAP-like alkaline phosphatase, TfR, TGF, TGF-alpha, TGF-beta, TGF-beta Pan Specific, TGF-betaRI (ALK-5), TGF-betaRII, TGF-betaRIIb, TGF-betaRIII, TGF-beta1, TGF-beta2, TGF-beta3, TGF-beta4, TGF-beta5, thrombin, thymus Ck-1, thyroid-stimulating hormone, Tie, TIMP, TIQ, tissue factor, TMEFF2, Tmpo, TMPRSS2, TNF, TNF-alpha, TNF-alphabeta, TNF-beta2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 ligand, TL2), TNFSF11 (TRANCE/RANK ligand ODF, OPG ligand), TNFSF12 (TWEAK Apo-3 ligand, DR3 ligand), TNFSF13 (APRIL TALL2), TNFSF13B (BAFF BLYS, TALL1, THANK, TNFSF20), TNFSF14 (LIGHT HVEM ligand, LTg), TNFSF15 (TL1A/VEGI), TNFSF18 (GITR ligand AITR ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 ligand gp34, TXGP1), TNFSF5 (CD40 ligand CD154, gp39, HIGM1, IMD3, TRAP), TNFSF6 (Fas ligand Apo-1 ligand, APT1 ligand), TNFSF7 (CD27 ligand CD70), TNFSF8 (CD30 ligand CD153), TNFSF9 (4-1BB ligand CD137 ligand), TP-1, t-PA, Tpo, TRAIL, TRAIL R, TRAIL-R1, TRAIL-R2, TRANCE, transferrin receptor, TRF, Trk, TROP-2, TSG, TSLP, tumor associated antigen CA125, tumor associated antigen expressing Lewis-Y associated carbohydrates, TWEAK, TXB2, Ung, uPAR, uPAR-1, urokinase, VCAM, VCAM-1, VECAD, VE-Cadherin, VE-cadherin-2, VEFGR-1 (flt-1), VEGF, VEGFR, VEGFR-3 (flt-4), VEGI, VIM, virus antigen, VLA, VLA-1, VLA-4, VNR integrin, von Willebrand factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, HMGB1, IgA, AB, CD81, CD97, CD98, DDR1, DKK1, EREG, Hsp90, IL-17/IL-17R, IL-20/IL-20R, oxidized LDL, PCSK9, prekallikrein, RON, TMEM16F, SOD1, Chromogranin A, Chromogranin B, tau, VAP1, high molecular weight kininogen, IL-31, IL-31R, Nav1.1, Nav1.2, Nav1.3, Nav1.4, Nav1.5, Nav1.6, Nav1.7, Nav1.8, Nav1.9, EPCR, C1, Clq, Clr, Cls, C2, C2a, C2b, C3, C3a, C3b, C4, C4a, C4b, C5, C5a, C5b, C6, C7, C8, C9, factor B, factor D, factor H, properdin, sclerostin, fibrinogen, fibrin, prothrombin, thrombin, tissue factor, factor V, factor Va, factor VII, factor VIIa, factor VIII, factor VIIIa, factor IX, factor IXa, factor X, factor Xa, factor XI, factor XIa, factor XII, factor XIIa, factor XIII, factor XIIIa, TFPI, antithrombin III, EPCR, thrombomodulin, TAPI, tPA, plasminogen, plasmin, PAI-1, PAI-2, GPC3, Syndecan-1, Syndecan-2, Syndecan-3, Syndecan-4, LPA, and S1P; and receptors for hormone and growth factors.

One or more amino acid residue alterations are allowed in the amino acid sequences constituting the variable regions as long as their antigen-binding activities are maintained. When altering a variable region amino acid sequence, there is no particularly limitation on the site of alteration and number of amino acids altered. For example, amino acids present in CDR and/or FR can be altered appropriately. When altering amino acids in a variable region, the binding activity is preferably maintained without particular limitation; and for example, as compared to before alteration, the binding activity is 50% or more, preferably 80% or more, and more preferably 100% or more. Furthermore, the binding activity may be increased by amino acid alterations. For example, the binding activity may be 2-, 5-, 10-times higher or such than that before alteration. In the antibodies of the present invention, alteration of amino acid sequence may be at least one of amino acid residue substitution, addition, deletion, and modification.

For example, the modification of the N-terminal glutamine of a variable region into pyroglutamic acid by pyroglutamylation is a modification well known to those skilled in the art. Thus, when the heavy-chain N terminus is glutamine, the antibodies of the present invention comprise the variable regions in which the glutamine is modified to pyroglutamic acid.

Antibody variable regions of the present invention may have any sequences, and they may be antibody variable regions of any origin, such as mouse antibodies, rat antibodies, rabbit antibodies, goat antibodies, camel antibodies, humanized antibodies produced by humanizing these non-human antibodies, and human antibodies. “Humanized antibodies”, also referred to as “reshaped human antibodies”, are antibodies in which the complementarity determining regions (CDRs) of an antibody derived from a non-human mammal, for example, a mouse antibody, are transplanted into the CDRs of a human antibody. Methods for identifying CDRs are known (Kabat et al., Sequence of Proteins of Immunological Interest (1987), National Institute of Health, Bethesda, Md.; Chothia et al., Nature (1989) 342:877). Their common genetic recombination techniques are also known (see, European Patent Application Publication No. EP 125023 and WO 96/02576). Furthermore, these antibodies may have various amino acid substitutions introduced into their variable regions to improve their antigen binding, pharmacokinetics, stability, and immunogenicity. Variable regions of the antibodies of the present invention may be able to bind antigens repeatedly due to their pH dependability in antigen binding (WO 2009/125825).

κ chain and λ chain-type constant regions are present in antibody light-chain constant regions, but either one of the light chain constant regions is acceptable. Furthermore, light-chain constant regions of the present invention may be light-chain constant regions with amino acid alterations such as substitutions, deletions, additions, and/or insertions.

For example, for the heavy chain constant regions of an antibody of the present invention, heavy chain constant regions of human IgG antibodies may be used and heavy chain constant regions of human IgG1 antibodies and those of human IgG4 antibodies are preferred. Furthermore, Fc region variants of the present invention may be made into Fc fusion protein molecules by linking to other proteins, physiologically active peptides, and such. Herein, fusion protein refers to a chimeric polypeptide comprising at least two different polypeptides, which do not spontaneously link with each other in natural. Examples of the other proteins and biologically active peptides include receptors, adhesion molecules, ligands, and enzymes, but are not limited thereto.

Preferred examples of Fc fusion protein molecules of the present invention include proteins with Fc region fused to a receptor protein that binds to a target, and such examples include TNFR-Fc fusion protein, ILIR-Fc fusion protein, VEGFR-Fc fusion protein, and CTLA4-Fc fusion protein (Nat Med. 2003 January; 9(1): 47-52; BioDrugs. 2006; 20(3): 151-60). Furthermore, a protein to be fused to a polypeptide of the present invention may be any molecule as long as it binds to a target molecule, and examples include scFv molecules (WO 2005/037989), single-domain antibody molecules (WO 2004/058821; WO 2003/002609), antibody-like molecules (Current Opinion in Biotechnology 2006, 17:653-658; Current Opinion in Biotechnology 2007, 18:1-10; Current Opinion in Structural Biology 1997, 7:463-469; and Protein Science 2006, 15:14-27) such as DARPins (WO 2002/020565), Affibody (WO 1995/001937), Avimer (WO 2004/044011; WO 2005/040229), and Adnectin (WO 2002/032925). Furthermore, antibodies and Fc fusion protein molecules may be multispecific antibodies that bind to multiple types of target molecules or epitopes.

Furthermore, the antibodies of the present invention include antibody modification products. Such antibody modification products include, for example, antibodies linked with various molecules such as polyethylene glycol (PEG) and cytotoxic substances. Such antibody modification products can be obtained by chemically modifying antibodies of the present invention. Methods for modifying antibodies are already established in this field.

The antibodies of the present invention may also be bispecific antibodies. “Bispecific antibody” refers to an antibody that has in a single molecule variable regions that recognize different epitopes. The epitopes may be present in a single molecule or in different molecules.

The polypeptides of the present invention can be prepared by the methods known to those skilled in the art. For example, the antibodies can be prepared by the methods described below, but the methods are not limited thereto.

A DNA encoding an antibody heavy chain in which one or more amino acid residues in the Fc region have been substituted with other amino acids of interest and DNA encoding an antibody light chain, are expressed. A DNA encoding a heavy chain in which one or more amino acid residues in the Fc region are substituted with other amino acids of interest can be prepared, for example, by obtaining a DNA encoding the Fc region of a natural heavy chain, and introducing an appropriate substitution so that a codon encoding a particular amino acid in the Fc region encodes another amino acid of interest.

Alternatively, a DNA encoding a heavy chain in which one or more amino acid residues in the Fc region are substituted with other amino acids of interest can also be prepared by designing and then chemically synthesizing a DNA encoding a protein in which one or more amino acid residues in the Fc region of the natural heavy chain are substituted with other amino acids of interest. The position and type of amino acid substitution are not particularly limited. Furthermore, alteration is not limited to substitution, and alteration may be any of deletion, addition, or insertion, or combination thereof.

Alternatively, a DNA encoding a heavy chain in which one or more amino acid residues in the Fc region are substituted with other amino acids of interest can be prepared as a combination of partial DNAs. Such combinations of partial DNAs include, for example, the combination of a DNA encoding a variable region and a DNA encoding a constant region, and the combination of a DNA encoding an Fab region and a DNA encoding an Fc region, but are not limited thereto. Furthermore, a DNA encoding a light chain can similarly be prepared as a combination of partial DNAs.

Methods for expressing the above-described DNAs include the methods described below. For example, a heavy chain expression vector is constructed by inserting a DNA encoding a heavy chain variable region into an expression vector along with a DNA encoding a heavy chain constant region. Likewise, a light chain expression vector is constructed by inserting a DNA encoding a light chain variable region into an expression vector along with a DNA encoding a light chain constant region. Alternatively, these heavy and light chain genes may be inserted into a single vector.

When inserting a DNA encoding the antibody of interest into an expression vector, the DNA is inserted so that the antibody is expressed under the control of an expression-regulating region such as an enhancer or promoter. Next, host cells are transformed with this expression vector to express the antibody. In such cases, an appropriate combination of host and expression vector may be used.

Examples of the vectors include M13 vectors, pUC vectors, pBR322, pBluescript, and pPCR-Script. Alternatively, when aiming to subclone and excise cDNA, in addition to the vectors described above, pGEM-T, pDIRECT, pT7, and such can be used.

Expression vectors are particularly useful when using vectors for producing the polypeptides of the present invention. For example, when a host cell is E. coli such as JM109, DH5α, HB101, and XL1-Blue, the expression vectors must carry a promoter that allows efficient expression in E. coli, for example, lacZ promoter (Ward et al., Nature (1989) 341:544-546; FASEB J. (1992) 6:2422-2427; its entirety are incorporated herein by reference), araB promoter (Better et al., Science (1988) 240:1041-1043; its entirety are incorporated herein by reference), T7 promoter, or such. Such vectors include pGEX-5X-1 (Pharmacia), “QIAexpress system” (QIAGEN), pEGFP, or pET (in this case, the host is preferably BL21 that expresses T7 RNA polymerase) in addition to the vectors described above.

The vectors may contain signal sequences for polypeptide secretion. As a signal sequence for polypeptide secretion, a pelB signal sequence (Lei, S. P. et al J. Bacteriol. (1987) 169:4379; its entirety are incorporated herein by reference) may be used when a polypeptide is secreted into the E. coli periplasm. The vector can be introduced into host cells by lipofectin method, calcium phosphate method, and DEAE-Dextran method, for example.

In addition to E. coli expression vectors, the vectors for producing the polypeptides of the present invention include mammalian expression vectors (for example, pcDNA3 (Invitrogen), pEGF-BOS (Nucleic Acids. Res. 1990, 18(17): p5322; its entirety are incorporated herein by reference), pEF, and pCDM8), insect cell-derived expression vectors (for example, the “Bac-to-BAC baculovirus expression system” (GIBCO-BRL) and pBacPAK8), plant-derived expression vectors (for example, pMH1 and pMH2), animal virus-derived expression vectors (for example, pHSV, pMV, and pAdexLcw), retroviral expression vectors (for example, pZIPneo), yeast expression vectors (for example, “Pichia Expression Kit” (Invitrogen), pNV11, and SP-Q01), and Bacillus subtilis expression vectors (for example, pPL608 and pKTH50), for example.

When aiming for expression in animal cells such as CHO, COS, and NIH3T3 cells, the vectors must have a promoter essential for expression in cells, for example, SV40 promoter (Mulligan et al., Nature (1979) 277:108; its entirety are incorporated herein by reference), MMTV-LTR promoter, EF1α promoter (Mizushima et al., Nucleic Acids Res. (1990) 18:5322; its entirety are incorporated herein by reference), CAG promoter (Gene. (1990) 18:5322; its entirety are incorporated herein by reference), and CMV promoter, and more preferably they have a gene for selecting transformed cells (for example, a drug resistance gene that allows evaluation using an agent (neomycin, G418, or such)). Vectors with such characteristics include pMAM, pDR2, pBK-RSV, pBK-CMV, pOPRSV, and pOP13, for example.

In addition, the following method can be used for stable gene expression and gene copy number amplification in cells: CHO cells deficient in a nucleic acid synthesis pathway are introduced with a vector that carries a DHFR gene which compensates for the deficiency (for example, pCHOI), and the vector is amplified using methotrexate (MTX). Alternatively, the following method can be used for transient gene expression: COS cells with a gene expressing SV40 T antigen on their chromosome are transformed with a vector with an SV40 replication origin (pcD and such). Replication origins derived from polyoma virus, adenovirus, bovine papilloma virus (BPV), and such can also be used. To amplify gene copy number in host cells, the expression vectors may further carry selection markers such as aminoglycoside transferase (APH) gene, thymidine kinase (TK) gene, E. coli xanthine-guanine phosphoribosyltransferase (Ecogpt) gene, and dihydrofolate reductase (dhfr) gene.

Antibodies can be collected, for example, by culturing transformed cells, and then separating the antibodies from the inside of the transformed cells or from the culture media. Antibodies can be separated and purified using an appropriate combination of methods such as centrifugation, ammonium sulfate fractionation, salting out, ultrafiltration, 1q, FcRn, protein A, protein G column, affinity chromatography, ion exchange chromatography, and gel filtration chromatography.

Furthermore, the present invention provides methods for producing a polypeptide comprising an antibody Fc region variant having enhanced FcγRIIb-binding activity in comparison with a polypeptide comprising a parent Fc region, which comprises adding at least one amino acid alteration to the Fc region variant.

Examples include production methods comprising the following steps:
(a) adding at least one amino acid alteration to an Fc region of polypeptides comprising the Fc region;
(b) measuring the FcγRIIb-binding activity of the polypeptides altered in step (a); and
(c) selecting polypeptides comprising an Fc region variant having enhanced FcγRIIb-binding activity in comparison with a polypeptide comprising a parent Fc region.

A preferred embodiment is a method for producing a polypeptide comprising an Fc region variant, which comprises the steps of:
(a) altering a nucleic acid encoding the polypeptide so that the FcγRIIb-binding activity is enhanced in comparison with the polypeptide comprising a parent Fc region;
(b) introducing the nucleic acid into host cells and culturing them to induce expression; and
(c) collecting the polypeptide from the host cell culture.

Furthermore, antibodies and Fc fusion protein molecules produced by this production method are also included in the present invention.

The present invention also provides a method of producing a polypeptide which comprises an Fc region variant with enhanced binding selectivity to FcγRIIb compared to FcγRIIa (type R) as compared to that of a polypeptide which comprises a parent Fc region, wherein the method comprises adding at least one amino acid alteration to an antibody Fc region variant in a polypeptide comprising the Fc region variant.

An example is a production method comprising the steps of:
(a) adding at least one amino acid alteration to an Fc region in a polypeptide comprising the Fc region;
(b) determining the FcγRIIa-binding activity and FcγRIIb-binding activity of the polypeptide altered in step (a); and
(c) selecting a polypeptide comprising an Fc region variant with enhanced binding selectivity to FcγRIIb compared to FcγRIIa (type R) as compared to that of a polypeptide comprising a parent Fc region.

In a preferred embodiment, it is a method of producing polypeptides comprising an Fc region variant, wherein the method comprises the steps of:
(a) modifying a nucleic acid encoding a polypeptide comprising a parent Fc region to achieve enhancement of binding selectivity to FcγRIIb compared to FcγRIIa (type R) as compared to that of the polypeptide;
(b) transfecting the nucleic acid into a host cell and culturing the cell for expression of the nucleic acid; and
(c) collecting the polypeptide from the host cell culture.

Antibodies and Fc fusion protein molecules produced by the production method are also included in the present invention.

Furthermore, the present invention provides a method of producing a polypeptide comprising an Fc region variant with enhanced FcγRIIb-binding activity and enhanced binding selectivity to FcγRIIb compared to FcγRIIa (type R) as compared to those of a polypeptide comprising a parent Fc region, wherein the method comprises adding at least one amino acid alteration to an antibody Fc region variant in a polypeptide comprising the antibody Fc region variant.

An example is a production method comprising the steps of:
(a) adding at least one amino acid alteration to an Fc region in a polypeptide comprising the Fc region;
(b) determining the FcγRIIa-binding activity and FcγRIIb-binding activity of the polypeptide altered in step (a); and
(c) selecting a polypeptide comprising an Fc region variant with enhanced FcγRIIb-binding activity and enhanced binding selectivity to FcγRIIb compared to FcγRIIa (type R) as compared to those of a polypeptide comprising a parent Fc region.

In a preferred embodiment, it is a method of producing polypeptides comprising an Fc region variant, wherein the method comprises the steps of:
(a) modifying nucleic acid encoding a polypeptide comprising a parent Fc region to achieve enhancement of FcγRIIb-binding activity and enhancement of binding selectivity to FcγRIIb compared to FcγRIIa (type R) as compared to those of the polypeptide;
(b) transfecting the nucleic acid into a host cell and culturing the cell for expression of the nucleic acid; and
(c) collecting the polypeptide from the host cell culture.

Antibodies and Fc fusion protein molecules produced by the production method are also included in the present invention.

The present invention also provides methods for producing a polypeptide in which antibody production against the polypeptide is suppressed compared with a polypeptide comprising a parent Fc region when administered in vivo, which comprise adding at least one amino acid alteration in the Fc region of a polypeptide comprising an antibody Fc region.

Examples include a production method comprising the following steps:
(a) adding at least one amino acid alteration in the Fc region of a polypeptide comprising an Fc region; and
(b) confirming that antibody production is suppressed when the polypeptide comprising an Fc region altered in step (a) is administered in vivo in comparison with a polypeptide comprising a parent Fc region.

Whether or not production of antibodies against the polypeptide has been suppressed can be confirmed by methods of administering the polypeptide to an animal and such. Alternatively, suppression of antibody production can be determined by measuring the binding activities towards FcγRIIa and FcγRIIb, and observing an increase in the value obtained by dividing the KD value for FcγRIIa by the KD value for FcγRIIb. Such polypeptides are considered to be useful as pharmaceuticals since they can suppress antibody production without activating activating FcγR.

In the above-mentioned production methods, it is preferred that FcγRIIb-binding activity is enhanced and binding selectivity to FcγRIIb compared to FcγRIIa (type R) is enhanced.

An example of a preferred embodiment of the above-mentioned production method is altering an Fc region of human IgG so that alteration of the amino acid at position 238 (EU numbering) to another amino acid and alteration of at least one amino acid selected from amino acids at positions 233, 234, 235, 237, 264, 265, 266, 267, 268, 269, 271, 272, 274, 296, 326, 327, 330, 331, 332, 333, 334, 355, 356, 358, 396, 409, and 419 according to EU numbering to another amino acid are introduced into the Fc region. Other amino acid alterations that are combined with the amino acid alteration at position 238 (EU numbering) are preferably those at amino acid positions 233, 237, 264, 267, 268, 271, 272, 296, 327, 330, 332, 333, and 396 according to EU numbering, and in particular those at amino acid positions 233, 237, 264, 267, 268, 271, 296, 330, and 396. In particular, a preferred combination of amino acid alterations in terms of enhancement of FcγRIIb-binding activity or enhancement of binding selectivity to FcγRIIb compared to FcγRIIa includes, for example, the combination of amino acid alterations at amino acid positions 238, 268, and 271 (EU numbering) with at least one amino acid alteration selected from positions 233, 237, 264, 267, 272, 296, 327, 330, 332, and 396 (EU numbering).

The amino acids to be altered are not particularly limited as long as those enhance FcγRIIb-binding activity or enhance binding selectivity to FcγRIIb compared to FcγRIIa as compared to before the alteration, but preferably, the amino acid at position 238 is Asp, the amino acid at position 233 is Asp, the amino acid at position 234 is Tyr, the amino acid at position 237 is Asp, the amino acid at position 264 is Ile, the amino acid at position 265 is Glu, the amino acid at position 266 is Phe, Leu or Met, the amino acid at position 267 is Ala, Glu, Gly, or Gln, the amino acid at position 268 is Asp, Gln, or Glu, the amino acid at position 269 is Asp, the amino acid at position 271 is Gly, the amino acid at position 272 is Asp, Phe, Ile, Met, Asn, Pro, or Gln, the amino acid at position 274 is Gln, the amino acid at position 296 is Asp or Phe, the amino acid at position 326 is Ala or Asp, the amino acid at position 327 is Gly, the amino acid at position 330 is Lys, Arg, or Ser, the amino acid at position 331 is Ser, the amino acid at position 332 is Lys, Arg, Ser, or Thr, the amino acid at position 333 is Lys, Arg, Ser, or Thr, the amino acid at position 334 is Arg, Ser, or Thr, the amino acid at position 355 is Ala or Gln, the amino acid at position 356 is Glu, the amino acid at position 358 is Met, the amino acid at position 396 is Ala, Asp, Glu, Phe, Gly, His, Ile, Lys, Leu, Met, Asn, Gln, Arg, Ser, Thr, Val, Trp, or Tyr, the amino acid at position 409 is Arg, and the amino acid at position 419 is Glu. The amino acids to be altered are more preferably those that enhance FcγRIIb-binding activity and also enhance binding selectivity to FcγRIIb compared to FcγRIIa as compared to before the alteration. In particular, when combining alterations at amino acid positions 238, 268, and 271 (EU numbering) with at least one amino acid position selected from 233, 264, 267, 272, 296, 327, 330, 332, and 396 (EU numbering), it is preferred that the amino acid at position 238 is Asp, the amino acid at position 268 is Asp or Glu, the amino acid at position 271 is Gly, the amino acid at position 233 is Asp, the amino acid at position 237 is Asp, the amino acid at position 264 is Ile, the amino acid at position 267 is Ala or Gly, the amino acid at position 272 is Asp or Pro, the amino acid at position 296 is Asp, the amino acid at position 327 is Gly, the amino acid at position 330 is Arg, the amino acid at position 332 is Thr, and the amino acid at position 396 is Leu or Met, according to EU numbering.

Among these combinations, introducing alterations that lead to greater enhancement of FcγRIIb-binding activity, or lead to greater enhancement of binding selectivity to FcγRIIb compared to FcγRIIa (type R) are preferred. Examples of such a preferred combination of amino acid substitution as alterations include the following combinations (a) to (x):
(a) amino acid alterations at positions 238, 233, 237, 268, 271, 296, and 330 (EU numbering) of an Fc region;
(b) amino acid alterations at positions 238, 237, 268, 271, 296, and 330 (EU numbering) of an Fc region;
(c) amino acid alterations at positions 238, 233, 237, 268, 271, 296, 330, and 332 (EU numbering) of an Fc region;
(d) amino acid alterations at positions 238, 233, 237, 264, 267, 268, 271, and 330 (EU numbering) of an Fc region;
(e) amino acid alterations at positions 238, 233, 237, 267, 268, 271, 296, 330, and 332 (EU numbering) of an Fc region;
(f) amino acid alterations at positions 238, 237, 267, 268, 271, 296, 330, and 332 (EU numbering) of an Fc region;
(g) amino acid alterations at positions 238, 233, 237, 268, 271, 296, 327, and 330 (EU numbering) of an Fc region;
(h) amino acid alterations at positions 238, 233, 237, 264, 267, 268, and 271 (EU numbering) of an Fc region;
(i) amino acid alterations at positions 238, 233, 237, 264, 267, 268, 271, 296, and 330 (EU numbering) of an Fc region;
(j) amino acid alterations at positions 238, 233, 237, 264, 267, 268, 271, 296, 330, and 396 (EU numbering) of an Fc region;
(k) amino acid alterations at positions 238, 237, 264, 267, 268, 271, and 330 (EU numbering) of an Fc region;
(l) amino acid alterations at positions 238, 237, 264, 267, 268, 271, 296, and 330 (EU numbering) of an Fc region;
(m) amino acid alterations at positions 238, 264, 267, 268, and 271 (EU numbering) of an Fc region;
(n) amino acid alterations at positions 238, 264, 267, 268, 271, and 296 (EU numbering) of an Fc region;
(o) amino acid alterations at positions 238, 237, 267, 268, 271, 296, and 330 (EU numbering) of an Fc region;
(p) amino acid alterations at positions 238, 233, 237, 264, 267, 268, 271, 330, and 396 (EU numbering) of the Fc region;
(q) amino acid alterations at positions 238, 233, 237, 264, 267, 268, 271, 296, 327, 330, and 396 (EU numbering) of an Fc region;
(r) amino acid alterations at positions 238, 233, 237, 264, 267, 268, 271, 272, and 296 (EU numbering) of an Fc region;
(s) amino acid alterations at positions 238, 237, 264, 267, 268, 271, 272, and 330 (EU numbering) of an Fc region;
(t) amino acid alterations at positions 238, 237, 264, 267, 268, 271, 272, 296, and 330 (EU numbering) of an Fc region;
(u) amino acid alterations at positions 238, 233, 264, 267, 268, and 271 (EU numbering) of an Fc region;
(v) amino acid alterations at positions 238, 237, 267, 268, 271, 296, and 330 (EU numbering) of an Fc region;
(w) amino acid alterations at positions 238, 264, 267, 268, 271, 272, and 296 (EU numbering) of an Fc region; and
(x) amino acid alterations at positions 238, 233, 264, 267, 268, 271, and 296 (EU numbering) of an Fc region.

In addition, among these combinations, the following combinations of amino acid alterations (a) to (x) below are more preferred amino acid combinations:
(a) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, and the amino acid at position 330 is Arg, according to EU numbering, in an Fc region;
(b) an amino acid sequence in which the amino acid at position 238 is Asp, the amino acid at position 237 is Asp, the amino acid at position 268 is Asp or Glu, the amino acid at position 271 is Gly, the amino acid at position 296 is Asp, and the amino acid at position 330 is Arg, according to EU numbering, in an Fc region;
(c) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 330 is Arg, and the amino acid at position 332 is Thr, according to EU numbering, in an Fc region;
(d) an amino acid sequence in which the amino acid at position 238 is Asp, the amino acid at position 233 is Asp, the amino acid at position 237 is Asp, the amino acid at position 264 is Ile, the amino acid at position 267 is Gly or Ala, the amino acid at position 268 is Glu, the amino acid at position 271 is Gly, and the amino acid at position 330 is Arg, according to EU numbering, in an Fc region;
(e) an amino acid sequence in which the amino acid at position 238 is Asp, the amino acid at position 233 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(f) an amino acid sequence in which the amino acid at position 238 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(g) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 327 is Gly, and the amino acid at position 330 is Arg, according to EU numbering, in an Fc region;
(h) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, and the amino acid at position 271 is Gly, according to EU numbering, in an Fc region;
(i) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(j) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 330 is Arg, and the amino acid at position 396 is Met or Leu, according to EU numbering, in the Fc region;
(k) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, and the amino acid at position 330 is Arg, according to EU numbering, in an Fc region;
(l) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(m) an amino acid sequence in which the amino acid at position 238 is Asp, the amino acid at position 264 is Ile, the amino acid at position 267 is Ala, the amino acid at position 268 is Glu, and the amino acid at position 271 is Gly, according to EU numbering, in an Fc region;
(n) an amino acid sequence in which the amino acid at position 238 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region;
(o) an amino acid sequence in which the amino acid at position 238 is Asp, the amino acid at position 237 is Asp, the amino acid at position 267 is Ala or Gly, the amino acid at position 268 is Glu, the amino acid at position 271 is Gly, the amino acid at position 296 is Asp, and the amino acid at position 330 is Arg, according to EU numbering, in an Fc region;
(p) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 330 is Arg, and the amino acid at position 396 is Met or Leu, according to EU numbering, in an Fc region;
(q) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 327 is Gly, the amino acid at position 330 is Arg, and the amino acid at position 396 is Met, according to EU numbering, in an Fc region;
(r) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region;
(s) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, and the amino acid at position 330 is Arg, according to EU numbering, in an Fc region;
(t) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, the amino acid at position 296 is Asp, and the amino acid at position 330 is Arg, according to EU numbering, in an Fc region;
(u) an amino acid sequence in which the amino acid at position 238 is Asp, the amino acid at position 233 is Asp, the amino acid at position 264 is Ile, the amino acid at position 267 is Ala, the amino acid at position 268 is Glu, and the amino acid at position 271 is Gly, according to EU numbering, in an Fc region;
(v) an amino acid sequence in which the amino acid at position 238 is Asp, the amino acid at position 237 is Asp, the amino acid at position 267 is Gly, the amino acid at position 268 is Asp, the amino acid at position 271 is Gly, the amino acid at position 296 is Asp, and the amino acid at position 330 is Arg, according to EU numbering, in an Fc region;
(w) an amino acid sequence in which the amino acid at position 238 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region; and
(x) an amino acid sequence in which the amino acid at position 238 is Asp, the amino acid at position 233 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region.

The present invention further provides a method of altering a polypeptide to produce a polypeptide with enhanced FcγRIIb-binding activity or enhanced binding selectivity to FcγRIIb compared to FcγRIIa (type R) as compared to those of a polypeptide containing a parent Fc region. The present invention also provides a method of altering a polypeptide to produce a polypeptide with enhanced FcγRIIb-binding activity and enhanced binding selectivity to FcγRIIb compared to FcγRIIa (type R) as compared to those of a polypeptide containing a parent Fc region.

The present invention also provides methods for altering a polypeptide for the production of a polypeptide whose antibody production is suppressed compared with that of a polypeptide comprising a parent Fc region when it is administered in vivo.

An example of a preferred embodiment includes the combination of amino acid alterations described in the method of producing polypeptides comprising Fc region variants with enhanced FcγRIIb-binding activity or enhanced binding selectivity to FcγRIIb compared to FcγRIIa (type R). An example of a more preferred embodiment includes the above-described combination of amino acid alterations described in the method of producing polypeptides comprising Fc region variants with enhanced FcγRIIb-binding activity and enhanced binding selectivity to FcγRIIb compared to FcγRIIa (type R).

Furthermore, the present invention provides a nucleic acid encoding a polypeptide comprising an Fc region having at least one amino acid alteration, wherein the polypeptide comprises an Fc region variant with enhanced FcγRIIb-binding activity or enhanced binding selectivity to FcγRIIb compared to FcγRIIa (type R) as compared to those of a polypeptide comprising a parent Fc region. The present invention also provides a nucleic acid encoding a polypeptide comprising an Fc region having at least one amino acid alteration, wherein the polypeptide comprises an Fc region variant with enhanced FcγRIIb-binding activity and enhanced binding selectivity to FcγRIIb compared to FcγRIIa (type R)-binding activity, as compared to those of a polypeptide comprising a parent Fc region. The nucleic acid of the present invention may be in any form such as DNA or RNA.

The present invention also provides vectors carrying the above-described nucleic acids of the present invention. The type of vector can be appropriately selected by those skilled in the art depending on the host cells to be introduced with the vector. The vectors include, for example, those described above.

Furthermore, the present invention relates to host cells transformed with the above-described vectors of the present invention. Appropriate host cells can be selected by those skilled in the art. The host cells include, for example, those described above. Specific examples include the following host cells.

When eukaryotic cells are used as host cells, animal cells, plant cells, or fungal cells can be appropriately used. Specifically, the animal cells include, for example, the following cells.
(1) mammalian cells: CHO (Chinese hamster ovary cell line), COS (Monkey kidney cell line), myeloma (Sp2/O, NS0, and such), BHK (baby hamster kidney cell line), Hela, Vero, HEK293 (human embryonic kidney cell line with sheared adenovirus (Ad) 5 DNA), FreeStyle293™ cell line, PER.C6 cell (human embryonic retinal cell line transformed with the Adenovirus Type 5 (Ad5) E1A and E1B genes), and such (Current Protocols in Protein Science (May 2001, Unit 5.9, Table 5.9.1));
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells:
yeasts: the Saccharomyces genus such as Saccharomyces serevisiae, and the Pichia genus such as Pichia pastoris; and
filamentous fungi: the Aspergillus genus such as Aspergillus niger.

Furthermore, the present invention provides a method of enhancing FcγRIIb-binding activity and/or enhancing binding selectivity to FcγRIIb compared to FcγRIIa (type R)-binding activity, as compared to those of a polypeptide comprising a parent Fc region, wherein the method comprises adding at least one amino acid alteration to the Fc region in an Fc region-comprising polypeptide.

The present invention also provides methods for suppressing production of antibodies against a polypeptide comprising an Fc region, as compared with a polypeptide comprising a parent Fc region, when the polypeptide is administered in vivo, wherein the method comprises adding at least one amino acid alteration in the Fc region of the polypeptide.

An example of a preferred embodiment is the combination of amino acid alterations described in the method of producing polypeptides comprising Fc region variants with enhanced FcγRIIb-binding activity and/or enhanced binding selectivity to FcγRIIb compared to FcγRIIa (type R).

Polypeptides produced by any of the above-mentioned methods are also included in the present invention.

The present invention provides pharmaceutical compositions comprising the polypeptide comprising an Fc region variant of the present invention.

The pharmaceutical compositions of the present invention can be formulated, in addition to the antibody or Fc-fusion protein molecules of the present invention described above, with pharmaceutically acceptable carriers by known methods. For example, the compositions can be used parenterally, when the antibodies are formulated in a sterile solution or suspension for injection using water or any other pharmaceutically acceptable liquid. For example, the compositions can be formulated by appropriately combining the antibodies or Fc-fusion protein molecules with pharmaceutically acceptable carriers or media, specifically, sterile water or physiological saline, vegetable oils, emulsifiers, suspending agents, surfactants, stabilizers, flavoring agents, excipients, vehicles, preservatives, binding agents, and such, by mixing them at a unit dose and form required by generally accepted pharmaceutical implementations. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such. The content of the active ingredient in such a formulation is adjusted so that an appropriate dose within the required range can be obtained.

Sterile compositions for injection can be formulated using vehicles such as distilled water for injection, according to standard protocols.

Aqueous solutions used for injection include, for example, physiological saline and isotonic solutions containing glucose or other adjuvants such as D-sorbitol, D-mannose, D-mannitol, and sodium chloride. These can be used in conjunction with suitable solubilizers such as alcohol, specifically ethanol, polyalcohols such as propylene glycol and polyethylene glycol, and non-ionic surfactants such as Polysorbate 80™ and HCO-50.

Oils include sesame oils and soybean oils, and can be combined with solubilizers such as benzyl benzoate or benzyl alcohol. These may also be formulated with buffers, for example, phosphate buffers or sodium acetate buffers; analgesics, for example, procaine hydrochloride; stabilizers, for example, benzyl alcohol or phenol; or antioxidants. The prepared injections are typically aliquoted into appropriate ampules.

The administration is preferably carried out parenterally, and specifically includes injection, intranasal administration, intrapulmonary administration, and percutaneous administration. For example, injections can be administered systemically or locally by intravenous injection, intramuscular injection, intraperitoneal injection, or subcutaneous injection.

Furthermore, the method of administration can be appropriately selected according to the age and symptoms of the patient. A single dosage of the pharmaceutical composition containing an antibody or a polynucleotide encoding an antibody can be selected, for example, from the range of 0.0001 mg to 1000 mg per kg of body weight. Alternatively, the dosage may be, for example, in the range of 0.001 to 100000 mg/patient. However, the dosage is not limited to these values. The dosage and method of administration vary depending on the patient's body weight, age, and symptoms, and can be appropriately selected by those skilled in the art.

The above-mentioned polypeptides comprising an Fc region variant of the present invention are useful as active ingredients of pharmaceutical agents that suppress the activation of B cells, mast cells, dendritic cells, and/or basophils. Polypeptides comprising an Fc region variant of the present invention can suppress the activation of B cells, mast cells, dendritic cells, and/or basophils, by selectively working on FcγRIIb without activating activating FcγR. B cell activation includes proliferation, IgE production, IgM production, and IgA production. The above-mentioned polypeptides comprising an Fc region variant of the present invention cross-link FcγRIIb with IgE to suppress IgE production of B cells, with IgM to suppress IgM production of B cells, and with IgA to suppress IgA production. Other than the above, suppressive effects similar to those mentioned above are exhibited by directly or indirectly cross-linking FcγRIIb with molecules that are expressed on B cells and comprise the ITAM domain inside the cell or interact with the ITAM domain such as BCR, CD19, and CD79b. Furthermore, activation of mast cells includes proliferation, activation by IgE and such, and degranulation. In mast cells, the above-mentioned polypeptides comprising an Fc region variant of the present invention can suppress proliferation, activation by IgE and such, and degranulation by directly or indirectly cross-linking FcγRIIb with IgE receptor molecules that are expressed on mast cells and comprise the ITAM domain or interact with the ITAM domain such as FcεRI, DAP12, and CD200R3. Activation of basophils includes proliferation and degranulation of basophils. Also in basophils, the above-mentioned polypeptides comprising an Fc region variant of the present invention can suppress proliferation, activation, and degranulation by directly or indirectly cross-linking FcγRIIb with molecules on the cell membrane, which comprise the ITAM domain inside the cell or interact with the ITAM domain. Activation of dendritic cells includes proliferation and degranulation of dendritic cells. Also in dendritic cells, the above-mentioned polypeptides comprising an Fc region variant of the present invention can suppress activation, degranulation, and proliferation by directly or indirectly cross-linking FcγRIIb with molecules on the cell membrane, which comprise the ITAM domain inside the cell or interact with the ITAM domain.

In the present invention, the polypeptides comprising an Fc region variant of the present invention mentioned above are useful as an active ingredient of therapeutic agents or preventive agents for immunological inflammatory diseases. As described above, since polypeptides comprising an Fc region variant of the present invention can suppress activation of B cells, mast cells, dendritic cells and/or basophils, administration of the polypeptides comprising an Fc region variant of the present invention as a result can treat or prevent immunological inflammatory diseases. Without being limited thereto, the term “immunological inflammatory diseases” comprises, rheumatoid arthritis, autoimmune hepatitis, autoimmune thyroiditis, autoimmune blistering diseases, autoimmune adrenocortical disease, autoimmune hemolytic anemia, autoimmune thrombocytopenia purpura, megalocytic anemia, autoimmune atrophic gastritis, autoimmune neutropenia, autoimmune orchitis, autoimmune encephalomyelitis, autoimmune receptor disease, autoimmune infertility, chronic active hepatitis, glomerulonephritis, interstitial pulmonary fibrosis, multiple sclerosis, Paget's disease, osteoporosis, multiple myeloma, uveitis, acute and chronic spondylitis, gouty arthritis, inflammatory bowel disease, adult respiratory distress syndrome (ARDS), psoriasis, Crohn's disease, Basedow's disease, juvenile diabetes, Addison's disease, myasthenia gravis, lens-induced uveitis, systemic lupus erythematosus, allergic rhinitis, allergic dermatitis, ulcerative colitis, hypersensitivity, muscle degeneration, cachexia, systemic scleroderma, localized scleroderma, Sjogren's syndrome, Behchet's disease, Reiter's syndrome, type I and type II diabetes, bone resorption disorder, graft-versus-host reaction, ischemia-reperfusion injury, atherosclerosis, brain trauma, cerebral malaria, sepsis, septic shock, toxic shock syndrome, fever, malgias due to staining, aplastic anemia, hemolytic anemia, idiopathic thrombocytopenia, Goodpasture's syndrome, Guillain-Barre syndrome, Hashimoto's thyroiditis, pemphigus, IgA nephropathy, pollinosis, antiphospholipid antibody syndrome, polymyositis, Wegener's granulomatosis, arteritis nodosa, mixed connective tissue disease, fibromyalgia, asthma, atopic dermatitis, chronic atrophic gastritis, primary biliary cirrhosis, primary sclerosing cholangitis, autoimmune pancreatitis, aortitis syndrome, rapidly progressive glomerulonephritis, megaloblastic anemia, idiopathic thrombocytopenia purpura, primary hypothyroidism, idiopathic Addison's disease, insulin-dependent diabetes mellitus, chronic discoid lupus erythematosus, pemphigoid, herpes gestationis, linear IgA bullous dermatosis, epidermolysis bullosa acquisita, alopecia areata, vitiligo vulgaris, leukoderma acquisitum centrifugum of Sutton, Harada's disease, autoimmune optic neuropathy, idiopathic azoospermia, habitual abortion, hypoglycemia, chronic urticaria, ankylosing spondylitis, psoriatic arthritis, enteropathic arthritis, reactive arthritis, spondyloarthropathy, enthesopathy, irritable bowel syndrome, chronic fatigue syndrome, dermatomyositis, inclusion body myositis, Schmidt's syndrome, Graves' disease, pernicious anemia, lupoid hepatitis, presenile dementia, Alzheimer's disease, demyelinating disorder, amyotrophic lateral sclerosis, hypoparathyroidism, Dressler's syndrome, Eaton-Lambert syndrome, dermatitis herpetiformis, alopecia, progressive systemic sclerosis, CREST syndrome (calcinosis, Raynaud's phenomenon, esophageal dysmotility, sclerodactyly, and telangiectasia), sarcoidosis, rheumatic fever, erythema multiforme, Cushing's syndrome, transfusion reaction, Hansen's disease, Takayasu arteritis, polymyalgia rheumatica, temporal arteritis, giant cell arthritis, eczema, lymphomatoid granulomatosis, Kawasaki disease, endocarditis, endomyocardial fibrosis, endophthalmitis, fetal erythroblastosis, eosinophilic fasciitis, Felty syndrome, Henoch-Schonlein purpura, transplant rejection, mumps, cardiomyopathy, purulent arthritis, familial Mediterranean fever, Muckle-Wells syndrome, and hyper-IgD syndrome.

Furthermore, in autoimmune diseases which may be caused by production of antibodies against autoantigens (autoantibodies), the polypeptides comprising an Fc region variant of the present invention mentioned above are useful as an active ingredient of pharmaceutical agents for treating or preventing the autoimmune diseases by suppressing production of those autoantibodies. Use of a molecule produced by fusing an antibody Fc portion with AchR (an autoantigen of myasthenia gravis) has been reported to suppress proliferation of B cells which express AchR-recognizing BCR, and induce apoptosis (J. Neuroimmunol, 227:35-43, 2010). Use of a fusion protein formed between an antigen recognized by an autoantibody and an antibody Fc region of the present invention enables crosslinking of FcγRIIb with BCR of a B cell expressing BCR for that autoantigen, suppression of proliferation of B cells expressing BCR for the autoantigen, and induction of apoptosis. Such autoimmune diseases include Guillain-Barre syndrome, myasthenia gravis, chronic atrophic gastritis, autoimmune hepatitis, primary biliary cirrhosis, primary sclerosing cholangitis, autoimmune pancreatitis, aortitis syndrome, Goodpasture's syndrome, rapidly progressive glomerulonephritis, megaloblastic anemia, autoimmune hemolytic anemia, autoimmune neutropenia, idiopathic thrombocytopenia purpura, Basedow's disease, Hashimoto's thyroiditis, primary hypothyroidism, idiopathic Addison's disease, insulin-dependent diabetes mellitus, chronic discoid lupus erythematosus, localized scleroderma, pemphigus, pemphigoid, herpes gestationis, linear IgA bullous dermatosis, epidermolysis bullosa acquisita, alopecia areata, vitiligo vulgaris, leukoderma acquisitum centrifugum of Sutton, Harada's disease, autoimmune optic neuropathy, idiopathic azoospermia, habitual abortion, type II diabetes, hypoglycemia, and chronic urticaria; but are not limited thereto.

Furthermore, the above-mentioned polypeptides comprising an Fc Region variant of the present invention are useful as an active ingredient in therapeutic agents for diseases with deficiency of a biologically essential protein. For diseases with deficiency of a biologically essential protein, therapeutic methods that administer and supplement the protein as a pharmaceutical agent are used. However, since the patient lacks the protein from the beginning, the externally supplemented protein is recognized as a foreign substance and antibodies against that protein are produced. As a result, the protein becomes easily removed, and the effect as a pharmaceutical is reduced. Use of a fusion protein comprising such a protein and an antibody Fc region of the present invention enables crosslinking between FcγRIIb and BCR on B cells that recognize the protein, and enables suppression of antibody production against the protein. The proteins to be supplemented include Factor VIII, Factor IX, TPO, EPO, α-iduronidase, iduronate sulfatase, A-type heparan N-sulfatase, B type α-N-acetylglucosaminidase, C type acetyl CoA: α-glucosaminidase acetyltransferase, D type N-acetylglucosamine 6-sulfatase, galactose 6-sulfatase, N-acetylgalactosamine 4-sulfatase, β-glucuronidase, α-galactosidase, acidic α-galactosidase, and glucocerebrosidase. These proteins may be supplemented for diseases such as hemophilia, idiopathic thrombocytopenia purpura, renal anemia, and lysosomal disease (mucopolysaccharidosis, Fabry's disease, Pompe disease, and Gaucher's disease), without being limited thereto.

Furthermore, the above-mentioned polypeptides comprising an Fc region variant of the present invention are useful as an active ingredient for antiviral agents. Antibodies that comprise an Fc region of the present invention and are anti-virus antibodies can suppress antibody-dependent enhancement observed with anti-virus antibodies. Antibody-dependent enhancement is a phenomenon where a virus uses neutralizing antibodies against the virus to become phagocytosed via activating FcγRs, and infects FcγR-expressing cells so that the infection spreads. Binding of anti-dengue-virus neutralizing antibodies to FcγRIIb has been reported to play an important role in suppressing antibody-dependent enhancement (Proc. Natl. Acad. Sci. USA, 108:12479-12484, 2011). Crosslinking FcγRIIb with an immune complex with dengue virus, which is formed by the anti-dengue-virus neutralizing antibodies, inhibits FcγR-mediated phagocytosis, resulting in the suppression of antibody-dependent enhancement. Examples of such viruses include dengue virus (DENV1, DENV2, and DENV4) and HIV, but are not limited thereto.

Furthermore, polypeptides comprising an Fc region variant of the present invention described above are useful as an active ingredient in preventive agents or therapeutic agents for arteriosclerosis. Antibodies against oxidized LDL, i.e., a cause for arteriosclerosis, which are antibodies comprising an Fc region of the present invention, can prevent FcγRIIa-dependent adhesion of inflammatory cells. It has been reported that while anti-oxidized LDL antibodies inhibit the interaction between oxidized LDL and CD36, anti-oxidized LDL antibodies bind to endothelial cells, and monocytes recognize their Fc portion in an FcγRIIa-dependent or FcγRI-dependent manner; and this leads to adhesion (Immunol. Lett., 108:52-61, 2007). Using antibodies comprising an Fc region of the present invention for such antibodies may inhibit FcγRIIa-dependent binding and suppress monocyte adhesion by FcγRIIb-mediated inhibitory signals.

In the present invention, polypeptides comprising an Fc region variant of the present invention described above are useful as an active ingredient in therapeutic agents or preventive agents for cancer. As described above, it is known that enhancing the FcγRIIb binding enhances the agonistic activity of an agonist antibody, and enhances the antitumor effect of the antibody. Therefore, agonist antibodies using the Fc region variant of the present invention are useful for treatment or prevention of cancer. Specifically, the Fc region variant of the present invention enhances the agonistic activity of agonist antibodies against, for example, receptors of the TNF receptor family such as Aliases, CD120a, CD120b, Lymphotoxin β receptor, CD134, CD40, FAS, TNFRSF6B, CD27, CD30, CD137, TNFRSF10A, TNFRSF10B, TNFRSF10C, TNFRSF10D, RANK, Osteoprotegerin, TNFRSF12A, TNFRSF13B, TNFRSF13C, TNFRSF14, Nerve growth factor receptor, TNFRSF17, TNFRSF18, TNFRSF19, TNFRSF21, TNFRSF25, and Ectodysplasin A2 receptor and can be used for treating or preventing cancer. Furthermore, in addition to the above, agonistic activity is also enhanced for agonist antibodies against molecules which need to interact with FcγRIIb for exhibiting its agonistic activity. In addition, by incorporating the Fc region variant of the present invention into a polypeptide having binding activity to a molecule such as Kit, a type of receptor tyrosine kinase (RTK), which suppresses cell proliferation upon crosslinking with FcγRIIb, inhibitory effect against cells expressing such molecule may be enhanced. Without being limited thereto, cancer includes lung cancer (including small cell lung cancer, non-small cell lung cancer, pulmonary adenocarcinoma, and squamous cell carcinoma of the lung), large intestine cancer, rectal cancer, colon cancer, breast cancer, liver cancer, gastric cancer, pancreatic cancer, renal cancer, prostate cancer, ovarian cancer, thyroid cancer, cholangiocarcinoma, peritoneal cancer, mesothelioma, squamous cell carcinoma, cervical cancer, endometrial cancer, bladder cancer, esophageal cancer, head and neck cancer, nasopharyngeal cancer, salivary gland tumor, thymoma, skin cancer, basal cell tumor, malignant melanoma, anal cancer, penile cancer, testicular cancer, Wilms' tumor, acute myeloid leukemia (including acute myeloleukemia, acute myeloblastic leukemia, acute promyelocytic leukemia, acute myelomonocytic leukemia, and acute monocytic leukemia), chronic myelogenous leukemia, acute lymphoblastic leukemia, chronic lymphatic leukemia, Hodgkin's lymphoma, non-Hodgkin's lymphoma (Burkitt's lymphoma, chronic lymphocytic leukemia, mycosis fungoides, mantle cell lymphoma, follicular lymphoma, diffuse large-cell lymphoma, marginal zone lymphoma, pilocytic leukemia plasmacytoma, peripheral T-cell lymphoma, and adult T cell leukemia/lymphoma), Langerhans cell histiocytosis, multiple myeloma, myelodysplastic syndrome, brain tumor (including glioma, astroglioma, glioblastoma, meningioma, and ependymoma), neuroblastoma, retinoblastoma, osteosarcoma, Kaposi's sarcoma, Ewing's sarcoma, angiosarcoma, and hemangiopericytoma.

Furthermore, the present invention relates to methods for treating or preventing immunological inflammatory diseases, which comprise the step of administering to a subject (patient) a polypeptide comprising an Fc region variant of the present invention or a polypeptide comprising an Fc region variant produced by production methods of the present invention.

The present invention also provides kits for use in the therapeutic methods or preventive methods of the present invention, which comprises at least a polypeptide comprising an Fc region variant of the present invention or a polypeptide comprising an Fc region variant produced by production methods of the present invention, or a pharmaceutical composition of the present invention. In addition, pharmaceutically acceptable carriers, media, instructions on the method of use, and such may be included in the kit. Furthermore, the present invention relates to use of a polypeptide comprising an Fc region variant of the present invention or a polypeptide comprising an Fc region variant produced by production methods of the present invention in the production of agents for treating or preventing immunological inflammatory diseases. The present invention also relates to polypeptides comprising an Fc region variant of the present invention or polypeptides comprising an Fc region variant produced by production methods of the present invention for use in the therapeutic methods or preventive methods of the present invention.

As used herein, the three-letter and single-letter codes for respective amino acids are as follows:
Alanine: Ala (A)
Arginine: Arg (R)
Asparagine: Asn (N)
Aspartic acid: Asp (D)
Cysteine: Cys (C)
Glutamine: Gln (Q)
Glutamic acid: Glu (E)
Glycine: Gly (G)
Histidine: His (H)
Isoleucine: Ile (I)
Leucine: Leu (L)
Lysine: Lys (K)
Methionine: Met (M)
Phenylalanine: Phe (F)
Proline: Pro (P)
Serine: Ser(S)
Threonine: Thr (T)
Tryptophan: Trp (W)
Tyrosine: Tyr (Y)
Valine: Val (V)

All prior art documents cited herein are incorporated by reference in their entirety.

### EXAMPLES

Herein below, the present invention will be specifically described further with reference to the Examples, but it is not to be construed as being limited thereto.

### [Example 1] Assessment of Platelet Aggregation Ability of Existing Antibodies Comprising an Fc with Enhanced FcgRIIb-Binding

As shown in Table 16 in Reference Example 4, an existing FcgRIIb enhancement technique which introduces alterations involving substitution of Glu for Ser at position 267 and substitution of Phe for Leu at position 328 (EU numbering) into native human IgG1 (Non-patent Document 28) shows 408-fold enhanced binding to FcgRIIb and 0.51-fold decreased binding to FcgRIIaH, while showing 522-fold enhanced binding to FcgRIIaR, as compared to those of IgG1. As described in “Background Art”, even if FcgRIIb-binding is enhanced, when it comes to cells such as platelets which only express FcgRIIa, only enhancement effects on FcgRIIa may be affected. That is, existing techniques which enhance binding to FcgRIIaR have the danger of enhancing platelet-aggregating activity and increasing the risk for developing thrombosis. To confirm this, it was assessed whether platelet-aggregating activity is actually enhanced when FcgRIIa-binding of an antibody is enhanced.

Using the method of Reference Example 1, omalizumab_VH-G1d (SEQ ID NO: 25) was produced as the heavy chain and omalizumab_VL-CK (SEQ ID NO: 26) was produced as the light chain of a human IgG1 antibody that binds to IgE. Furthermore, to enhance human FcγRIIb-binding activity of omalizumab_VH-G1d, omalizumab_VH-G1d-v3 was produced by substituting Glu for Ser at position 267 and Phe for Leu at position 328 according to EU numbering in omalizumab_VH-G1d. Using the method of Reference Example 1, omalizumab-G1d-v3 which contains omalizumab_VH-G1d-v3 as the heavy chain and omalizumab_VL-CK as the light chain was produced. Platelet-aggregating ability was assessed using this antibody.

Platelet aggregation was assayed using the platelet aggregometer HEMA TRACER 712 (LMS Co.). First, about 50 ml of whole blood was collected at a fixed amount into 4.5-ml evacuated blood collection tubes containing 0.5 ml of 3.8% sodium citrate, and this was centrifuged at 200 g for 15 minutes. The resultant supernatant was collected and used as platelet-rich plasma (PRP). After PRP was washed with buffer 1 (137 mM NaCl, 2.7 mM KCl, 12 mM NaHCO₃, 0.42 mM NaH₂PO₄, 2 mM MgCl₂, 5 mM HEPES, 5.55 mM dextrose, 1.5 U/ml apyrase, 0.35% BSA), the buffer was replaced with buffer 2 (137 mM NaCl, 2.7 mM KCl, 12 mM NaHCO₃, 0.42 mM NaH₂PO₄, 2 mM MgCl₂, 5 mM HEPES, 5.55 mM dextrose, 2 mM CaCl₂), 0.35% BSA) to prepare about 300,000/μl washed platelets. 156 μl of the washed platelets was aliquoted into assay cuvettes containing a stir bar set in the platelet aggregometer. The platelets were stirred at 1000 rpm with the stir bar in the cuvettes maintained at 37.0° C. in the platelet aggregometer. 44 μl of the immune complex of omalizumab-G1d-v3 and IgE at a molar ratio of 1:1 (prepared at final concentrations of 600 μg/ml and 686 μg/ml, respectively) was added to the cuvettes. The platelets were reacted with the immune complex for five minutes. Then, at a concentration that does not allow secondary platelet aggregation, adenosine diphosphate (ADP, SIGMA) was added to the reaction mixture to test whether the aggregation is enhanced.

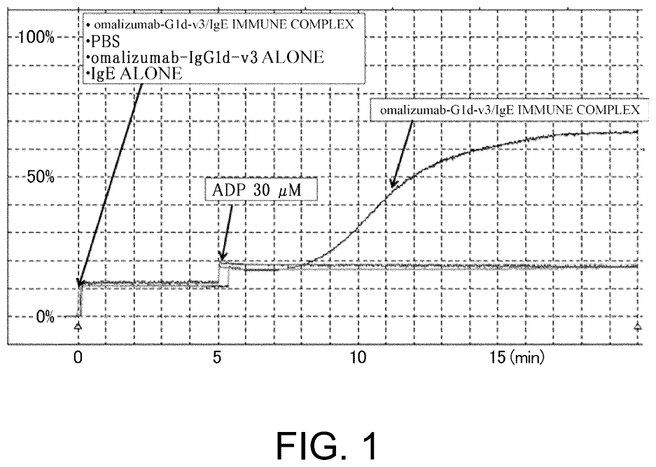

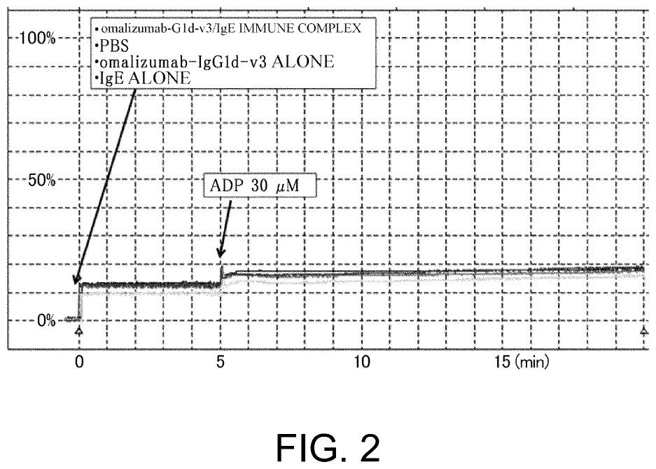

The result for each donor with an FcγRIIa polymorphic form (H/H or R/H) obtained from the above assay is shown in FIGS. 1 and 2. From the result in FIG. 1, it is shown that platelet aggregation was enhanced with the FcγRIIa polymorphic form (R/H) when the immune complex is added. Meanwhile, as shown in FIG. 2, platelet aggregation was not enhanced with the FcγRIIa polymorphic form (H/H).

Next, platelet activation was assessed using activation markers. Platelet activation can be measured based on the increased expression of an activation marker such as CD62p (p-selectin) or active integrin on the platelet membrane surface. 2.3 μl of the immune complex was added to 7.7 μl of the washed platelets prepared by the method described above. After five minutes of reaction at room temperature, activation was induced by adding ADP at a final concentration of 30 μM, and whether the immune complex enhances the ADP-dependent activation was assessed. A sample added with phosphate buffer (pH 7.4; Gibco), instead of the immune complex, was used as a negative control. Staining was performed by adding, to each post-reaction sample, PE-labeled anti-CD62 antibody (BECTON DICKINSON), PerCP-labeled anti-CD61 antibody, and FITC-labeled PAC-1 antibody (BD bioscience). Each of the fluorescence intensities was measured using a flow cytometer (FACS CantoII™ flow cytometer, BD bioscience).

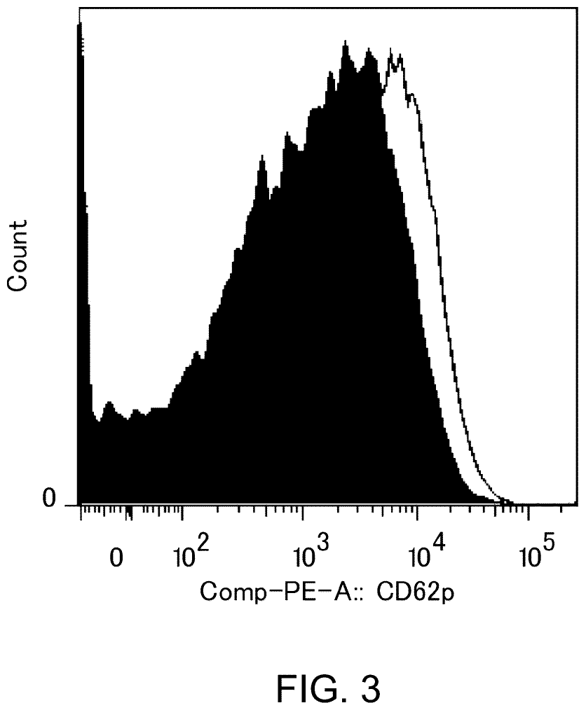

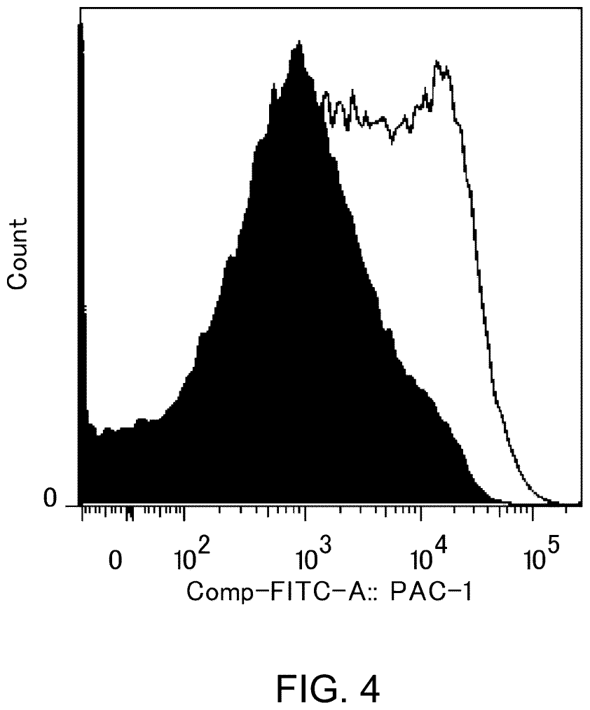

The result on CD62p expression, obtained by the above assay method, is shown in FIG. 3. The result on the activated integrin expression is shown in FIG. 4. The washed platelets used were obtained from a healthy person with the FcγRIIa polymorphic form R/H. Both CD62p and active integrin expressed on platelet membrane surface, which is induced by ADP stimulation, was enhanced in the presence of the immune complex.

From these results, in existing antibodies having an Fc with enhanced human FcγRIIb-binding, which have an Fc produced by introducing substitution of Ser at position 267 with Glu and Leu at position 328 with Phe (EU numbering) into an IgG1 Fc, the genetic polymorphs of FcγRIIa whose amino acid at position 131 is R showed enhanced platelet-aggregating activity compared to when the amino acid at position 131 is H. That is, existing antibodies having an Fc with enhanced human FcγRIIb binding was suggested to have the danger of increasing the risk for developing thrombosis due to platelet aggregation in humans carrying FcγRIIa type R, elucidating the value of an Fc with enhanced selective binding to FcγRIIb that overcomes this problem.

### [Example 2] Production of Variants with Enhanced Binding to FcgRIIb

As shown in Example 1, when enhancing binding to FcgRIIb, it is necessary to enhance FcgRIIb-binding while also suppressing binding to other activating FcgRs as much as possible. Therefore, production of variants with enhanced binding or selectivity to FcgRIIb was examined by combining alterations having the effect of enhancing binding or improving selectivity to FcgRIIb. Specifically, using as a base the P238D alteration, which shows excellent effects in both enhancement of binding and improvement of selectivity to FcgRIIb, alterations found to be effective upon combination with P238D in Reference Examples 6, 8, and 9 were further combined.

The variable region of IL6R-H (SEQ ID NO: 18), which is disclosed in WO2009/125825 and which is the variable region of an antibody against the human interleukin 6 receptor was produced as the antibody H chain variable region, and IL6R-G1d (SEQ ID NO: 19) which has G1d produced by removing the C-terminal Gly and Lys of human IgG1 was produced as the antibody H chain constant region. Furthermore, IL6R-B3 (SEQ ID NO: 23) was produced by introducing K439E into IL6R-G1d. Then, variants were produced from IL6R-B3 by combining E233D, L234Y, G237D, S267Q, H268D, P271G, Y296D, K326D, K326A, A330R, A330K, which are alterations found to be effective upon combination with P238D in Reference Examples 6, 8, and 9. IL6R-L (SEQ ID NO: 21) was commonly used as the antibody L chain. These variants were used for antibody expression and purification according to the method of Reference Example 1, and binding to each FcgR (FcgRIa, FcgRIIa type H, FcgRIIa type R, FcgRIIb, and FcgRIIIa type V) were assessed by the method of Reference Example 2.

The KD values of each variant for each FcgR are shown in Table 1. “Alteration” in the Table refers to alterations introduced into IL6R-B3 (SEQ ID NO: 23). Meanwhile, IL6R-B3/IL6R-L which is used as a template for producing each of the variants is indicated by an asterisk (*). “KD(IIaR)/KD(IIb)” in the Table shows the value obtained by dividing the KD of each variant for FcgRIIaR by the KD of each variant for FcgRIIb. The greater this value is, the higher the selectivity to FcgRIIb is. “Parent polypeptide KD(IIb)/altered polypeptide KD(IIb)” refers to a value obtained by dividing the KD value of IL6R-B3/IL6R-L for FcgRIIb by the KD value of each variant for FcgRIIb. Furthermore, “parent polypeptide KD(IIaR)/altered polypeptide KD(IIaR)” refers to a value obtained by dividing the KD value of IL6R-B3/IL6R-L for FcgR IIaR by the KD value of that variant for FcgRIIaR. In Table 1, values shown in bold italicized font were_calculated using the following equation

KD=C·Rₘₐₓ/(R_{eq}−RI)−C [Equation 2]

described in Reference Example 2 since the binding of FcgR to IgG was determined to be too weak to analyze accurately by kinetic analysis.

TABLE 1

| | | | | | | | | KD(IIb) OF | KD(IIaR) OF |
|---|---|---|---|---|---|---|---|---|---|
| | | | | | | | | PARENT | PARENT |
| | | | | | | | | POLYPEPTIDE/ | POLYPEPTIDE/ |
| | | KD FOR | KD FOR | KD FOR | KD FOR | KD FOR | | KD(IIb) OF | KD(IIaR) OF |
| VARIANT | | FcgRIa | FcgRIIaR | FcgRIIaH | FcgRIIb | FcgRIIIaV | KD(IIaR)/ | ALTERED | ALTERED |
| NAME | ALTERATION | (mol/L) | (mol/L) | (mol/L) | (mol/L) | (mol/L) | KD(IIb) | POLYPEPTIDE | POLYPEPTIDE |
| IL6R-G1d/ | | 3.2E−10 | 1.0E−06 | 6.7E−07 | 2.6E−06 | 3.5E−07 | 0.4 | 1.2 | 1.1 |
| IL6R-L | | | | | | | | | |
| IL6R-B3/ | * | 4.2E−10 | 1.1E−06 | 7.7E−07 | 3.1E−06 | 3.3E−07 | 0.3 | 1.0 | 1.0 |
| IL6R-L | | | | | | | | | |
| IL6R-BF648/ | P238D | 1.1E−08 | 1.5E−05 | [symbol] | 1.2E−06 | [symbol] | 12.5 | 2.6 | 0.1 |
| IL6R-L | | | | | | | | | |
| IL6R-BP215/ | G237D/P238D/H268D/ | 4.3E−08 | 1.3E−06 | [symbol] | 4.1E−08 | [symbol] | 31.7 | 75.6 | 0.8 |
| IL6R-L | P271G/Y296D/A330K | | | | | | | | |
| IL6R-BP216/ | G237D/P238D/S267Q/ | 6.2E−07 | 2.9E−06 | [symbol] | 1.4E−07 | [symbol] | 20.7 | 22.1 | 0.4 |
| IL6R-L | H268D/P271G/A330K | | | | | | | | |
| IL6R-BP217/ | G237D/P238D/S267Q/ | 2.8E−06 | 3.6E−06 | [symbol] | 1.5E−07 | [symbol] | 24.0 | 20.7 | 0.3 |
| IL6R-L | H268D/P271G/Y296D/ | | | | | | | | |
| | A330K | | | | | | | | |
| IL6R-BP218/ | G237D/P238D/H268D/ | 3.7E−08 | 1.5E−06 | [symbol] | 7.6E−08 | [symbol] | 19.7 | 40.8 | 0.7 |
| IL6R-L | P271G/K326D/A330K | | | | | | | | |
| IL6R-BP219/ | L234Y/G237D/P238D/ | 4.6E−08 | 6.1E−07 | [symbol] | 3.4E−08 | [symbol] | 17.9 | 91.2 | 1.8 |
| IL6R-L | H268D/P271G/A330K | | | | | | | | |
| IL6R-BP220/ | E233D/G237D/P238D/ | 2.0E−08 | 1.1E−06 | [symbol] | 3.6E−08 | [symbol] | 30.6 | 86.1 | 1.0 |
| IL6R-L | H268D/P271G/Y296D/ | | | | | | | | |
| | A330K | | | | | | | | |
| IL6R-BP221/ | L234Y/G237D/P238D/ | 1.3E−07 | 7.1E−07 | [symbol] | 2.8E−08 | [symbol] | 25.4 | 110.7 | 1.5 |
| IL6R-L | Y296D/K326A/A330R | | | | | | | | |
| IL6R-BP222/ | L234Y/G237D/P238D/ | 5.1E−08 | 7.1E−07 | [symbol] | 3.4E−08 | [symbol] | 20.9 | 91.2 | 1.5 |
| IL6R-L | P271G/K326A/A330R | | | | | | | | |
| IL6R-BP223/ | L234Y/G237D/P238D/ | 2.7E−08 | 6.0E−07 | [symbol] | 2.5E−08 | [symbol] | 24.0 | 124.0 | 1.8 |
| IL6R-L | H268D/P271G/K326A/ | | | | | | | | |
| | A330R | | | | | | | | |
| IL6R-BP224/ | L234Y/G237D/P238D/ | 6.2E−09 | 4.5E−07 | [symbol] | 3.5E−08 | [symbol] | 12.9 | 88.6 | 2.4 |
| IL6R-L | S267Q/H268D/P271G/ | | | | | | | | |
| | K326A/A330R | | | | | | | | |
| IL6R-BP225/ | L234Y/G237D/P238D/ | 6.5E−08 | 6.9E−07 | [symbol] | 3.5E−08 | [symbol] | 19.7 | 88.6 | 1.6 |
| IL6R-L | K326D/A330R | | | | | | | | |
| IL6R-BP226/ | L234Y/G237D/P238D/ | 5.2E−08 | 5.7E−07 | [symbol] | 3.3E−08 | [symbol] | 17.3 | 93.9 | 1.9 |
| IL6R-L | P271G/K326D/A330R | | | | | | | | |
| IL6R-BP227/ | L234Y/G237D/P238D/ | 2.7E−08 | 6.2E−07 | [symbol] | 3.2E−08 | [symbol] | 19.4 | 96.9 | 1.8 |
| IL6R-L | H268D/P271G/K326D/ | | | | | | | | |
| | A330R | | | | | | | | |
| IL6R-BP228/ | L234Y/G237D/P238D/ | 5.5E−09 | 4.2E−07 | [symbol] | 4.0E−08 | [symbol] | 10.5 | 77.5 | 2.6 |
| IL6R-L | S267Q/H268D/P271G/ | | | | | | | | |
| | K326D/A330R | | | | | | | | |
| IL6R-BP229/ | E233D/L234Y/G237D/ | 5.6E−08 | 8.1E−07 | [symbol] | 4.2E−08 | [symbol] | 19.3 | 73.8 | 1.4 |
| IL6R-L | P238D/P271G/K326A/ | | | | | | | | |
| | A330R | | | | | | | | |
| IL6R-BP230/ | E233D/G237D/P238D/ | 1.4E−08 | 5.7E−07 | [symbol] | 2.1E−08 | [symbol] | 27.1 | 147.6 | 1.9 |
| IL6R-L | H268D/P271G/Y296D/ | | | | | | | | |
| | A330R | | | | | | | | |
| IL6R-BP231/ | G237D/P238D/H268D/ | 9.4E−09 | 7.4E−07 | [symbol] | 2.3E−08 | [symbol] | 32.2 | 134.8 | 1.5 |
| IL6R-L | P271G/Y296D/A330R | | | | | | | | |
| IL6R-BP232/ | L234Y/G237D/P238D/ | 7.6E−08 | 8.4E−07 | [symbol] | 5.6E−08 | [symbol] | 15.0 | 55.4 | 1.3 |
| IL6R-L | P271G/K236A/A330K | | | | | | | | |
| IL6R-BP233/ | L234Y/G237D/P238D/ | 7.0E−08 | 6.9E−07 | [symbol] | 3.7E−08 | [symbol] | 18.6 | 83.8 | 1.6 |
| IL6R-L | P271G/A330K | | | | | | | | |
| IL6R-BP234/ | E233D/L234Y/G237D/ | 6.5E−09 | 1.2E−06 | [symbol] | 1.2E−07 | [symbol] | 10.0 | 25.8 | 0.9 |
| IL6R-L | P238D/S267Q/H268D/ | | | | | | | | |
| | P271G/Y296D/K326D/ | | | | | | | | |
| | A330K | | | | | | | | |
| IL6R-BP235/ | E233D/L234Y/G237D/ | 3.5E−09 | 6.8E−07 | [symbol] | 4.4E−08 | [symbol] | 15.5 | 70.5 | 1.6 |
| IL6R-L | P238D/H268D/P271G/ | | | | | | | | |
| | Y296D/K326D/A330R | | | | | | | | |
| IL6R-BP236/ | E233D/L237Y/G237D/ | 7.7E−09 | 8.4E−07 | [symbol] | 6.5E−08 | [symbol] | 12.9 | 47.7 | 1.3 |
| IL6R-L | P238D/S267Q/H268D/ | | | | | | | | |
| | P271G/Y296D/K326D/ | | | | | | | | |
| | A330R | | | | | | | | |
| IL6R-BP237/ | E233D/L237Y/G237D/ | 4.1E−09 | 1.1E−06 | [symbol] | 1.0E−07 | [symbol] | 11.0 | 31.0 | 1.0 |
| IL6R-L | P238D/S267Q/H268D/ | | | | | | | | |
| | P271G/Y296D/K326A/ | | | | | | | | |
| | A330K | | | | | | | | |
| IL6R-BP238/ | E233D/L237Y/G237D/ | 2.4E−09 | 6.4E−07 | [symbol] | 3.6E−08 | [symbol] | 17.8 | 86.1 | 1.7 |
| IL6R-L | P238D/H268D/P271G/ | | | | | | | | |
| | Y296D/K326A/A330R | | | | | | | | |
| IL6R-BP239/ | E233D/L237Y/G237D/ | 7.6E−09 | 8.1E−07 | [symbol] | 6.0E−08 | [symbol] | 13.5 | 51.7 | 1.4 |
| IL6R-L | P238D/S267Q/H268D/ | | | | | | | | |
| | P271G/Y296D/K326A/ | | | | | | | | |
| | A330R | | | | | | | | |
| IL6R-BP240/ | E233D/G237D/P238D/ | 7.6E−09 | 1.5E−06 | [symbol] | 9.5E−08 | [symbol] | 15.8 | 32.6 | 0.7 |
| IL6R-L | S267Q/H268D/P271G/ | | | | | | | | |
| | A330R | | | | | | | | |
| IL6R-BP241/ | E233D/G237D/P238D/ | 1.1E−09 | 6.8E−07 | [symbol] | 4.5E−08 | [symbol] | 15.1 | 68.9 | 1.6 |
| IL6R-L | H268D/P271G/K326D/ | | | | | | | | |
| | A330R | | | | | | | | |
| IL6R-BP242/ | E233D/G237D/P238D/ | 1.9E−09 | 7.5E−07 | [symbol] | 5.1E−08 | [symbol] | 14.7 | 60.8 | 1.5 |
| IL6R-L | H268D/P271G/K326A/ | | | | | | | | |
| | A330R | | | | | | | | |
| IL6R-BP243/ | E233D/L237Y/G237D/ | 3.0E−09 | 5.4E−07 | [symbol] | 3.6E−08 | [symbol] | 15.0 | 86.1 | 2.0 |
| IL6R-L | P238D/H268D/P271G/ | | | | | | | | |
| | A330R | | | | | | | | |
| IL6R-BP244/ | E233D/G237D/P238D/ | 7.8E−09 | 1.8E−06 | [symbol] | 1.1E−07 | [symbol] | 16.4 | 28.2 | 0.6 |
| IL6R-L | S267Q/H268D/P271G/ | | | | | | | | |
| | Y296D/A330R | | | | | | | | |
| IL6R-BP245/ | E233D/G237D/P238D/ | 6.3E−09 | 1.4E−06 | [symbol] | 8.3E−08 | [symbol] | 16.9 | 37.3 | 0.8 |
| IL6R-L | S267Q/H268D/P271G/ | | | | | | | | |
| | Y296D/K326D/A330R | | | | | | | | |
| IL6R-BP246/ | E233D/G237D/P238D/ | 8.0E−09 | 1.6E−06 | [symbol] | 9.2E−08 | [symbol] | 17.4 | 33.7 | 0.7 |
| IL6R-L | S267Q/H268D/P271G/ | | | | | | | | |
| | Y296D/K326A/A330R | | | | | | | | |
| IL6R-BP247/ | E233D/G237D/P238D/ | 7.5E−09 | 8.1E−07 | [symbol] | 3.7E−08 | [symbol] | 21.9 | 83.8 | 1.4 |
| IL6R-L | H268D/P271G/Y296D/ | | | | | | | | |
| | K326D/A330R | | | | | | | | |
| IL6R-BP248/ | E233D/G237D/P238D/ | 1.7E−09 | 8.2E−07 | [symbol] | 3.5E−08 | [symbol] | 23.4 | 88.6 | 1.3 |
| IL6R-L | H268D/P271G/Y296D/ | | | | | | | | |
| | K326A/A330R | | | | | | | | |
| IL6R-BP249/ | E233D/L234Y/G237D/ | 7.0E−09 | 6.2E−07 | [symbol] | 3.7E−08 | [symbol] | 16.8 | 83.8 | 1.8 |
| IL6R-L | P238D/H268D/P271G/ | | | | | | | | |
| | Y296D/A330R | | | | | | | | |

[symbol]

indicates data missing or illegible when filed

Binding of IL6R-G1d/IL6R-L which carries the native human IgG1 sequence was 1.3 fold for FcgRIa, 1.1 fold for FcgRIIaR, 1.1 fold for FcgRIIaH, 1.2 fold for FcgRIIb, and 0.9 fold for FcgRIIIaV, when binding of IL6R-B3/IL6R-L produced by introducing K439E into IL6R-G1d/IL6R-L to the respective FcgRs was defined as 1, and all of the binding to FcgRs were equivalent to that of IL6R-G1d/IL6R-L. Therefore, comparing binding of each variant with that of the IL6R-B3/IL6R-L prior to alteration can be considered to be equivalent to comparing each variant with IL6R-G1d/IL6R-L which carries the native human IgG1 sequence. Accordingly, in the Examples hereafter, binding activity of each variant is compared with that of the IL6R-B3/IL6R-L prior to alteration.

Table 1 shows that all variants showed improved affinity to FcgRIIb in comparison with the IL6R-B3 prior to alteration, and IL6R-BF648/IL6R-L showed 2.6 fold improved affinity which was the lowest and IL6R-BP230/IL6R-L showed 147.6 fold improved affinity which was the highest. Furthermore, the value of KD(IIaR)/KD(IIb), which shows the degree of selectivity, is 10.0 for IL6R-BP234/IL6R-L which showed the lowest value, and 32.2 for IL6R-BP231/IL6R-L which showed the highest value, and all variants improved their selectivity compared to the IL6R-B3/IL6R-L prior to alteration, which showed a value of 0.3. All variants showed lower binding to FcgRIa, FcgRIIaH, and FcgRIIIaV compared to the IL6R-B3/IL6R-L prior to alteration.

### [Example 3] X-Ray Structure Analysis of a Complex Formed Between an Fc with Enhanced FcγRIIb Binding and the Extracellular Region of FcγRIIb and a Complex Formed Between this Fc and the Extracellular Region of FcγRIIaR

The IL6R-BP230/IL6R-L variant showing the highest enhanced FcgRIIb binding in Example 2 showed approximately 150-fold enhanced binding to FcgRIIb and binding to FcgRIIa type R was suppressed to about 1.9-fold increase when compared to the IL6R-B3/IL6R-L prior to alteration. Therefore, IL6R-BP230/IL6R-L is an excellent variant in terms of both binding and selectivity to FcgRIIb; however, to produce more excellent variants, it is preferred that FcgRIIb binding is further enhanced as well as binding to FcgRIIaR is suppressed as much as possible.

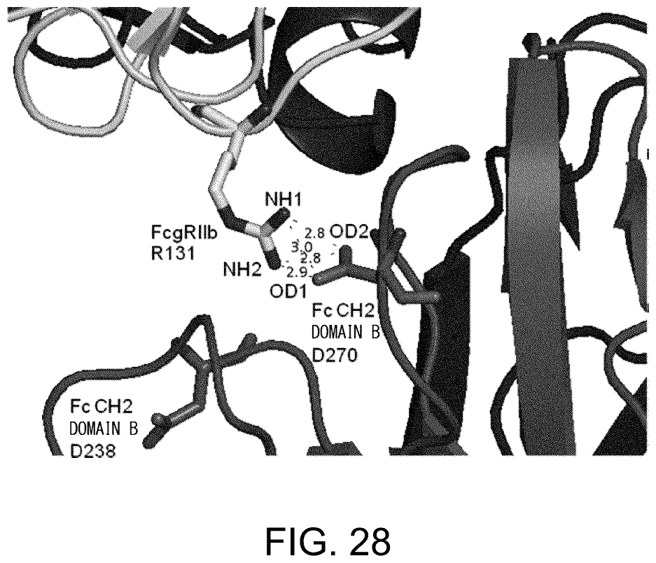

As shown in FIG. 28 of Reference Example 7, in Fc having the P238D alteration, formation of a strong electrostatic interaction is observed between Asp at position 270 (EU numbering) of the CH2 domain B and Arg at position 131 of FcγRIIb. While this residue at position 131 is His in FcγRIIIa and FcγRIIa type H, it is Arg in FcγRIIa type R as in the case with FcγRIIb. As a result, there are no differences in the interactions at this portion, and this is the reason that it is difficult to bring about selectivity for FcγRIIa type R.

Meanwhile, the extracellular regions of FcγRIIa and FcγRIIb match 93% in amino acid sequence, that is, have very high homology. When the crystal structure of the complex formed between the native IgG1 Fc(hereinafter “Fc(WT)”) and the extracellular region of FcγRIIa type R (J. Imunol. 2011, 187, 3208-3217) was analyzed, amino acid differences around an interacting interface were found to be only three (Gln127, Leu132, and Phe160) between FcγRIIa type R and FcγRIIb, and improvement of selectivity over FcγRIIa type R was expected to be very difficult.

Therefore, to achieve further improvement of selectivity and enhancement of FcγRIIb-binding activity, the present inventors considered that the amino acid mutations to be introduced must be examined in detail by conformationally elucidating the subtle differences in interactions caused by the difference in receptors by obtaining not only the three-dimensional structure of the complex formed between an Fc with enhanced FcγRIIb binding and the extracellular region of FcγRIIb, but also simultaneously, the three-dimensional structure of a complex formed with the extracellular region of FcγRIIa type R for which improvement of selectivity is considered most difficult. Then, X-ray structure analyses were performed on a complex formed between the extracellular region of FcγRIIb and Fc(P208), in which the K439E alteration is removed from the Fc of IL6R-BP208/IL6R-L (produced in Reference Example 9) which is a variant used as the basis for production of IL6R-BP230/IL6R-L, and a complex formed between the extracellular region of FcγRIIa type R and Fc(P208).

### 3-1. X-Ray Structure Analysis of a Complex Formed Between Fc(P208) and the Extracellular Region of FcγRIIb

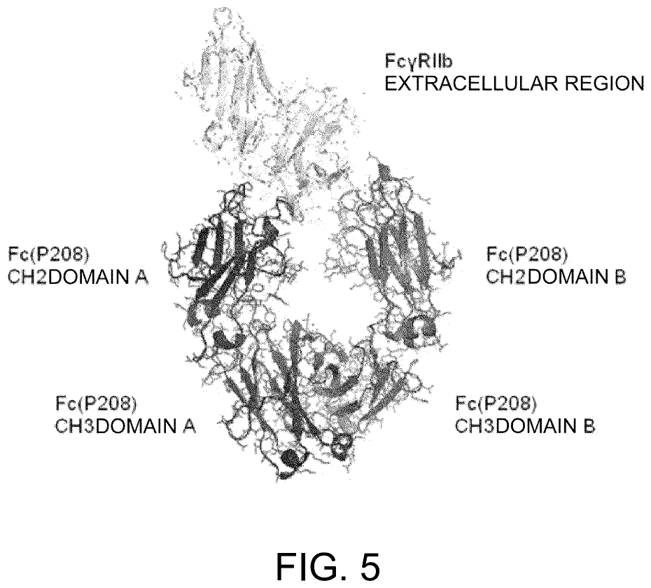

The three-dimensional structure of the Fc(P208)/FcγRIIb extracellular region complex was determined by X-ray structure analysis at 2.81 Å resolution. The structure obtained as a result of this analysis is shown in FIG. 5. The extracellular region of FcγRIIb is bound between two Fc CH2 domains, and this is similar to the three-dimensional structures of complexes formed between Fc(WT) which is an Fc of a native IgG and the respective extracellular region of FcγRIIIa (Proc. Natl. Acad. Sci. USA, 2011, 108, 12669-126674), FcγRIIIb (Nature, 2000, 400, 267-273; J. Biol. Chem. 2011, 276, 16469-16477), or FcγRIIa analyzed so far.

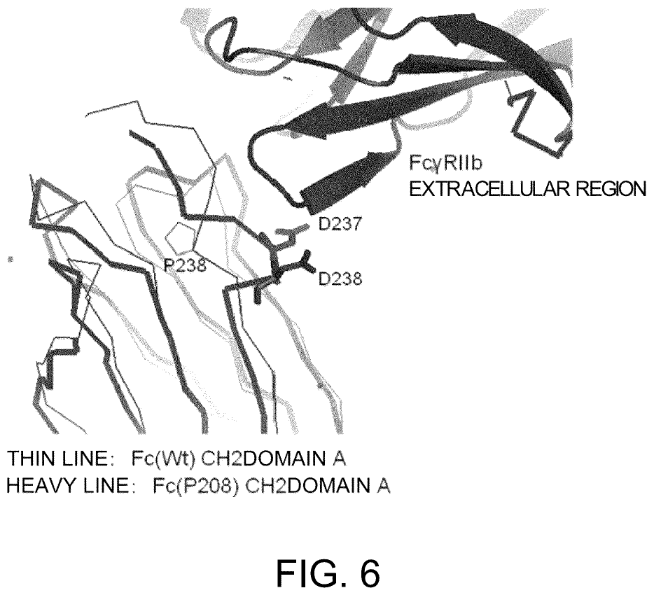

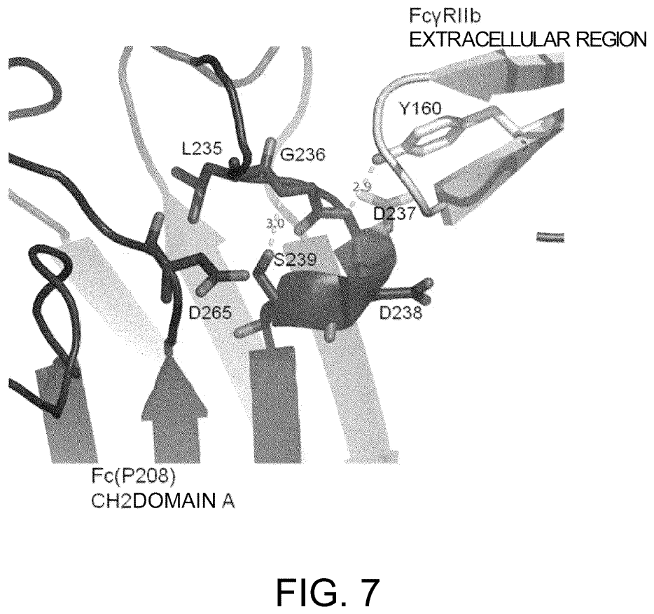

However, detailed analysis revealed that in the Fc(P208)/FcγRIIb extracellular region complex, the loop structure of positions 233 to 239 (EU numbering) continuing from the hinge region of Fc CH2 domain A was changed compared to that of the Fc(WT)/FcγRIIa type R extracellular region complex due to introduction of the G237D and P238D mutations (FIG. 6). As a result, formation of a strong hydrogen bond was observed between the main chain of Asp at position 237 (EU numbering) of Fc(P208) and the side chain of Tyr at position 160 of FcγRIIb (FIG. 7). As this Tyr160 is Phe in both the type H and type R of FcγRIIa, hydrogen bond formation is impossible. Therefore, this hydrogen bond was considered to have an important contribution to acquisition of selectivity which refers to enhancement of FcγRIIb-binding activity and decrease in FcγRIIa-binding activity.

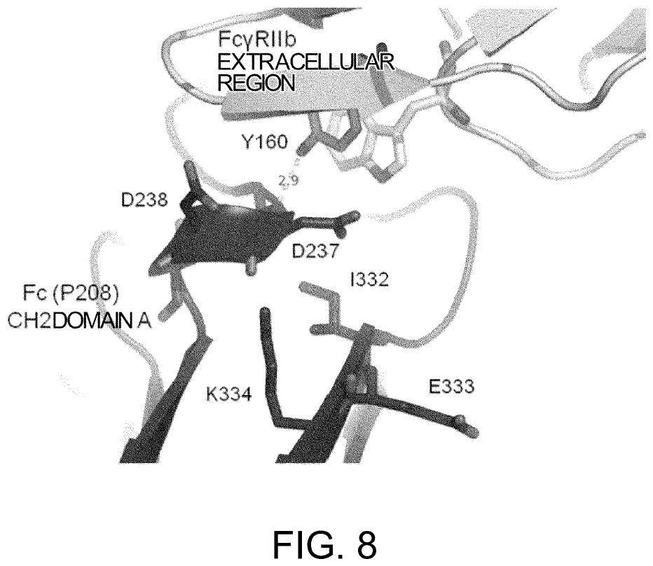

On the other hand, the side chain of Asp at position 237 (EU numbering) of Fc(P208) does not show remarkable interaction with FcγRIIb, and no interaction was observed with residues within Fc. Ile at position 332 (EU numbering), Glu at position 333 (EU numbering), and Lys at position 334 (EU numbering) in Fc were positioned in close proximity around this Asp at position 237 (EU numbering) (FIG. 8). If the loop structure can be stabilized by substituting these positions with hydrophilic resides to form interaction with the side chain of Asp at position 237 (EU numbering), entropic energy loss accompanying formation of hydrogen bond with Tyr at position 160 of FcγRIIb can be decreased, and this may lead to increase in the binding free energy, that is, enhancement of binding activity.

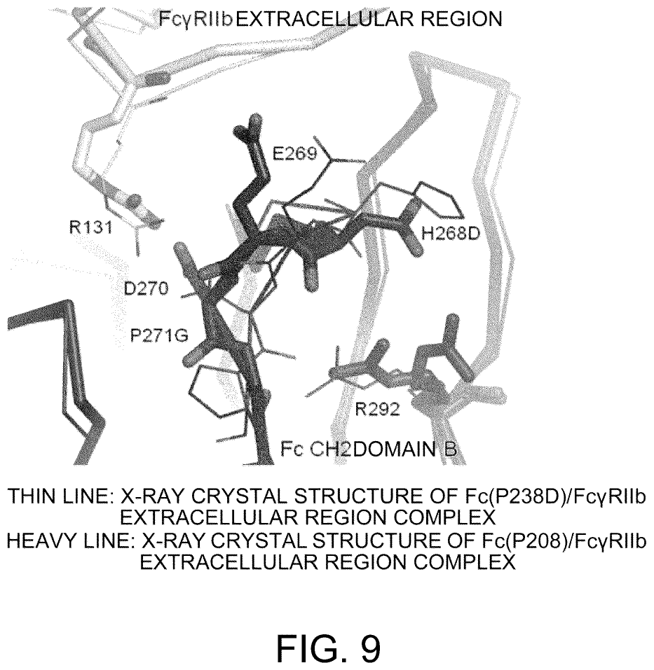

Comparison of the X-ray crystal structure of the complex formed between Fc(P238D) carrying the P238D alteration and the extracellular region of FcγRIIb, which is shown in Reference Example 7, and the X-ray crystal structure of the complex formed between Fc(P208) and the extracellular region of FcγRIIb, showed that compared to Fc(P238D), Fc(P208) contains five new mutations, and most of them were only changes at the side-chain level. However, by altering Pro at position 271 (EU numbering) to Gly in the CH2 domain B of Fc, change in location was observed at the main-chain-level, and structural changes had taken place at the upstream loop formed by positions 266-270 (EU numbering) (FIG. 9). As shown in Reference Example 8, in Fc(P238D), it has been suggested that the Pro portion at position 271 (EU numbering) may be stereochemically strained when Asp at position 270 (EU numbering) forms a strong electrostatic interaction with Arg at position 131 of FcγRIIb. Structural changes observed this time by introducing Gly at position 271 (EU numbering) may be the result of releasing structural strain accumulated at the Pro portion prior to alteration, and the amount of release of strain is considered to have led to improvement of binding free energy with FcγRIIb, that is, the enhancement of binding activity.

Furthermore, Arg at position 292 (EU numbering) was confirmed to undergo structural changes with taking two forms as a result of structural changes of the loop at positions 266-271 (EU numbering). In this case, Arg at position 292 (EU numbering) forms electrostatic interaction with Asp at position 268 (EU numbering) which is one of the other altered residues in Fc(P208) (FIG. 9), and this may contribute to the stabilization of this loop structure. The electrostatic interaction formed between Asp at position 270 (EU numbering) in this loop and Arg at position 131 of FcγRIIb greatly contributes to FcγRIIb-binding activity; therefore, introduction of the H268D alteration may have led to reduction of entropic energy loss accompanying binding and increase in binding free energy, that is, enhancement of binding activity by stabilizing the conformation of this loop structure to its FcγRIIb-bound form.

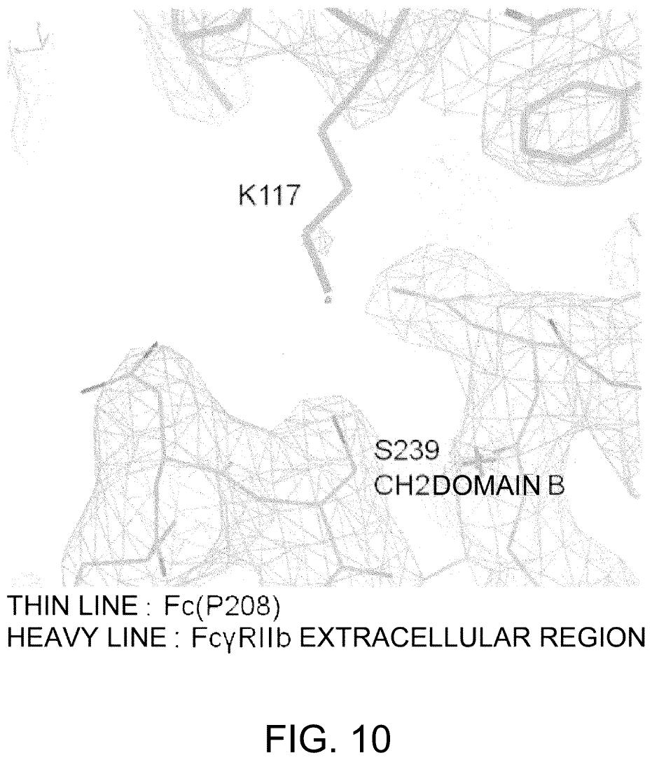

When further investigations for the possibility of alterations aimed at further improving the activity were carried out based on the structural analysis results, Ser at position 239 (EU numbering) was found as one of the candidate positions for introducing alterations. As shown in FIG. 10, this Ser at position 239 (EU numbering) of CH2 domain B is positioned in the direction where Lys at position 117 of FcγRIIb extends in a most structurally natural manner. However, as the electron density of Lys at position 117 of FcγRIIb was not detected in this analysis, this Lys residue does not form a steady structure. Thus, currently, involvement of this Lys residue in the interaction with Fc(P208) is considered to be limited; however, if this Ser at position 239 (EU numbering) of CH2 domain B is altered to negatively-charged Asp or Glu, electrostatic interaction with the positively-charged Lys at position 117 of FcγRIIb can be expected, and as a result, FcγRIIb-binding activity can be expected to become enhanced.

On the other hand, observing the structure of Ser at position 239 (EU numbering) in CH2 domain A, it is considered that the side chain of this amino acid forms hydrogen bonds with the main chain of Gly at position 236 (EU numbering) and stabilizes the loop structure from positions 233 to 239 which continues from the hinge region and includes Asp at position 237 (EU numbering) which forms a hydrogen bond with the FcγRIIb Tyr160 side chain (FIG. 7). Stabilizing this loop structure to the conformation taken during binding may lead to reduction of entropic energy loss accompanying binding, and as a result, enhancement of binding free energy, that is, enhancement of binding activity. On the other hand, if this Ser at position 239 (EU numbering) in CH2 domain A is altered to Asp or Glu, the hydrogen bond with the main chain of Gly at position 239 (EU numbering) may be lost, and may cause electrostatic repulsion between Asp at position 265 (EU numbering) which is present in close proximity, and thus may lead to large destabilization of the loop structure. This destabilized energy operates to decrease the binding free energy with FcγRIIb; therefore, this may lead to decrease in binding activity.

### [Expression and Purification of Fc(P208)]

Fc(P208) was prepared as follows. First, IL6R-P208 was produced by altering Glu at position 439 (EU numbering) of IL6R-BP208 (SEQ ID NO: 24) to Lys, which is the sequence of a native human IgG1. Then, Cys at position 220 (EU numbering) of IL6R-P208 was substituted with Ser. Then, genetic sequence of Fc(P208) from Glu at position 216 (EU numbering) to its C terminal was cloned by PCR. Using this cloned genetic sequence, production of expression vectors, and expression and purification of Fc(P208) were carried out according to the method of Reference Example 1. Cys at position 220 (EU numbering) forms a disulfide bond with Cys of the L chain in general IgG1. The L chain is not co-expressed when Fc alone is prepared, and therefore, this residue was substituted with Ser to avoid formation of unnecessary disulfide bonds.

### [Expression and Purification of the FcγRIIb Extracellular Region]

This was prepared according to the method of Reference Example 2.

### [Purification of the Fc(P208)/FcγRIIb Extracellular Region Complex]

To 1.5 mg of the FcγRIIb extracellular region sample obtained for crystallization, 0.15 mg of Endo F1 (Protein Science 1996, 5:2617-2622) expressed and purified from Escherichia coli as a glutathione S-transferase fusion protein was added. This was allowed to remain at room temperature for three days in 0.1 M Bis-Tris buffer at pH 6.5, and the N-linked oligosaccharide was cleaved, leaving N-acetylglucosamine directly bound to Asn. Next, this FcγRIIb extracellular region sample subjected to carbohydrate cleavage treatment was concentrated by ultrafiltration with 5000 MWCO, and purified by gel filtration chromatography (Superdex® 200 10/300 chromatography) using a column equilibrated in 20 mM HEPES at pH 7.5 containing 0.1 M NaCl. Furthermore, to the obtained carbohydrate-cleaved FcγRIIb extracellular region fraction, Fc(P208) was added so that the molar ratio of the FcγRIIb extracellular region would be present in slight excess, and after concentration by ultrafiltration with 10000 MWCO, a sample of the Fc(P208)/FcγRIIb extracellular region complex was obtained through purification by gel filtration chromatography (Superdex® 200 10/300 chromatography) using a column equilibrated in 25 mM HEPES at pH 7.5 containing 0.1 M NaCl.

### [Crystallization of the Fc(P208)/FcγRIIb Complex Extracellular Region Complex]

A sample of Fc(P208)/FcγRIIb extracellular region complex were concentrated to about 10 mg/ml using 10000 MWCO ultrafiltration filter, and crystallized using the hanging drop vapor diffusion method in combination with the seeding method. VDXm plate (Hampton Research) was used for crystallization. Using a reservoir solution containing 0.1 M Bis-Tris (pH 6.5), 19% (w/v) PEG3350, and 0.2 M potassium phosphate dibasic, crystallization drops were prepared at a mixing ratio of reservoir solution: crystallization sample=0.85 μl: 0.85 μl. Crystals of the complex obtained under the same condition were crushed with Seed Bead (Hampton Research) to prepare a seed crystal solution. 0.15 μl of a diluted solution produced from the seed crystal solution was added to the crystallization drops, which were sealed in the wells containing reservoirs, and allowed to stand at 20° C. This successfully yielded plate-like crystals.

[Measurement of X-Ray Diffraction Data from an Fc(P208)/FcγRIIb Extracellular Region Complex Crystal]

One of the obtained single crystals of the Fc(P208)/FcγRIIb extracellular region complex was soaked into a solution of 0.1 M Bis-Tris pH 6.5, 24% (w/v) PEG3350, 0.2 M potassium phosphate dibasic, 20% (v/v) ethylene glycol. The crystal was fished out of the solution using a pin with attached tiny nylon loop, and frozen in liquid nitrogen; and then X-ray diffraction data was measured by BL32XU at Spring-8. During the measurement, the crystal was constantly placed in a nitrogen stream at −178° C. to maintain in a frozen state, and a total of 300 X ray diffraction images were collected using MX-225HE CCD detector (RAYONIX) attached to a beam line with rotating the crystal 0.6° at a time. Determination of cell parameters, indexing of diffraction spots, and diffraction data processing from the obtained diffraction images were performed using the Xia2 program (J. Appl. Cryst. 2010, 43:186-190), XDS Package (Acta. Cryst. 2010, D66: 125-132) and Scala (Acta. Cryst. 2006, D62: 72-82); and finally, diffraction intensity data up to 2.81 Å resolution was obtained. The crystal belongs to the space group C2221, and has the following cell parameters; a=156.69 Å, b=260.17 Å, c=56.85 Å, α=90°, β=90°, γ=90°.

### [X Ray Structure Analysis of the Fc(P208)/FcγRIIb Extracellular Region Complex]

Crystal structure of the Fc(P208)/FcγRIIb extracellular region complex was determined by the molecular replacement method using the program Phaser (J. Appl. Cryst. 2007, 40:658-674). From the size of the obtained crystal lattice and the molecular weight of the Fc(P208)/FcγRIIb extracellular region complex, the number of complexes in the asymmetric unit was predicted to be one. From the structural coordinates of PDB code: 3SGJ which is the crystal structure of the Fc(WT)/FcγRIIIa extracellular region complex, the amino acid residue portions of the A chain positions 239-340 and the B chain positions 239-340 were taken out as separate coordinates, and they were used respectively as models for searching the Fc CH2 domains. The amino acid residue portions of the A chain positions 341-444 and the B chain positions 341-443 were taken out as a single set of coordinates from the same structural coordinates of PDB code: 3SGJ; and this was used as a model for searching the Fc CH3 domains. Finally, from the structural coordinates of PDB code: 2FCB which is a crystal structure of the FcγRIIb extracellular region, the amino acid residue portions of the A chain positions 6-178 was taken out and used as a model for searching the Fc(P208). The present inventors tried to determine the orientations and positions of each search model of Fc CH3 domains, FcγRIIb extracellular region, and Fc CH2 domain in the crystal lattices using rotation function and translation function, but failed to determine the position of one of the CH2 domains. Then, with reference to the crystal structure of the complex of Fc(WT)/FcγRIIIa extracellular region, the position of the last CH2 domain was determined from an electron density map that was calculated based on the phase determined from the remaining three parts. Thus, the present inventors obtained an initial model for the crystal structure of the Fc(P208)/FcγRIIb extracellular region complex. When rigid body refinement which moves the two Fc CH2 domains, the two Fc CH3 domains, and the FcγRIIb extracellular region was performed on the obtained initial model, the crystallographic reliability factor, R value became 42.6%, and the Free R value became 43.7% to diffraction intensity data from 25 Å to 3.0 Å at this point. Furthermore, structural refinement using the program REFMAC5 (Acta Cryst. 2011, D67, 355-367), and revision of the model to observe the electron density maps whose coefficient have 2Fo-Fc or Fo-Fc, which are calculated based on the experimentally determined structural factor Fo, the calculated structural factor Fc and the calculated phase using the model, was carried out by the Coot program (Acta Cryst. 2010, D66: 486-501), and model refinement was carried out by repeating these steps. Finally, as a result of incorporation of water molecules into the model based on the electron density maps which use 2Fo-Fc or Fo-Fc as the coefficient, and the following refinement, the crystallographic reliability factor, R values and the Free R value of the model containing 4786 non-hydrogen atoms became 24.5% and 28.2% to 27259 diffraction intensity data from 25 Å to 2.81 Å resolution, respectively.

### 3-2. X-Ray Structure Analysis of a Complex Formed Between Fc(P208) and the Extracellular Region of FcγRIIa Type R

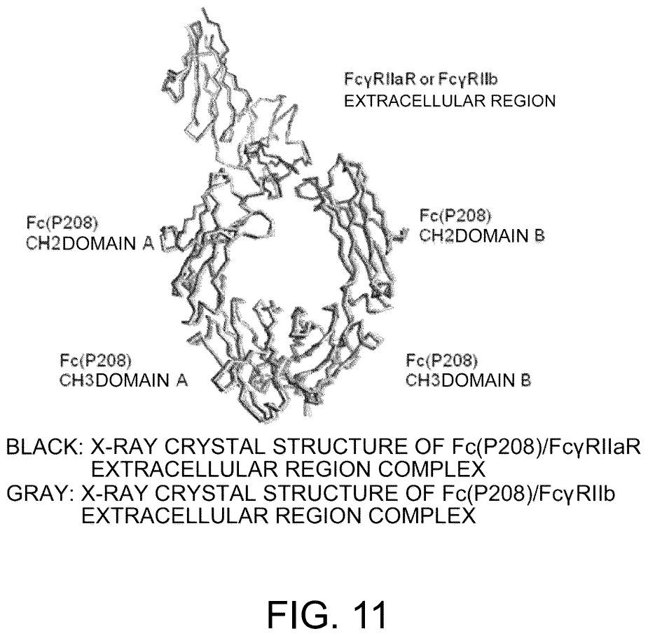

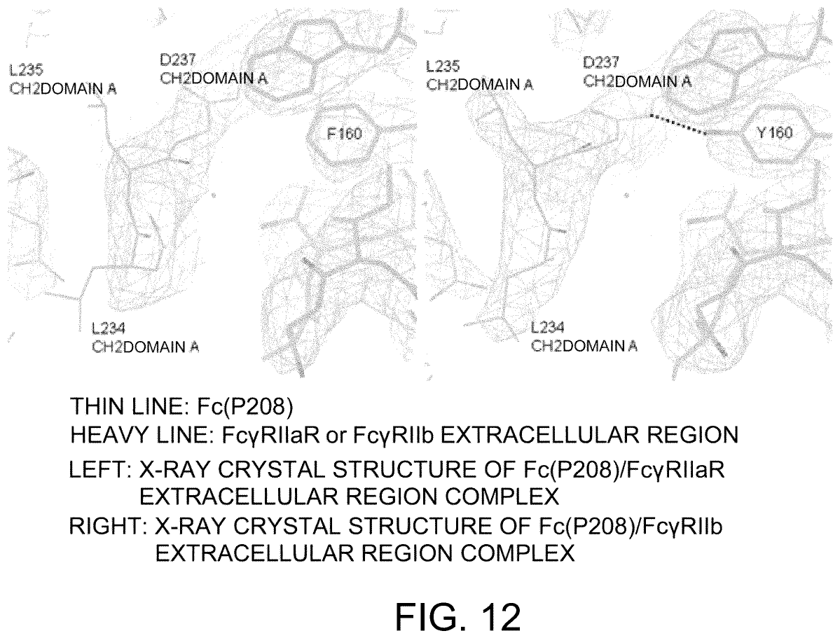

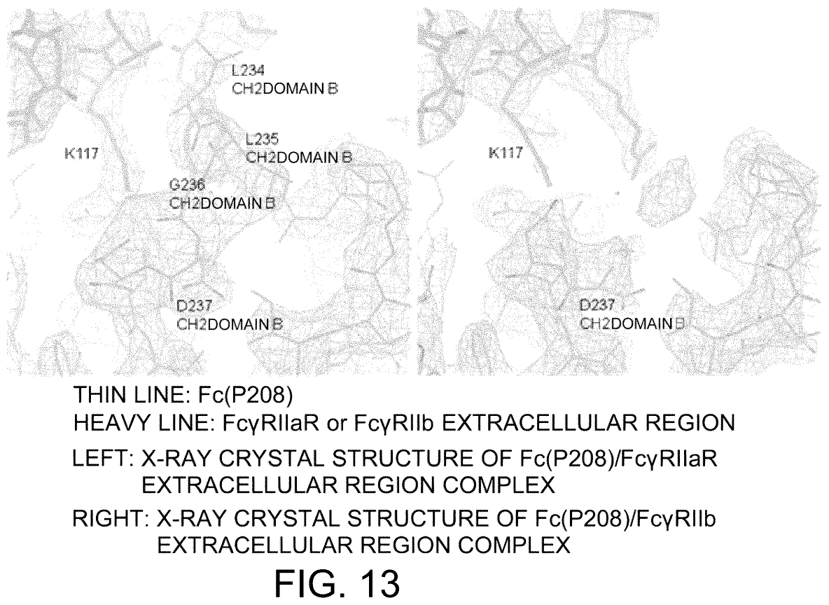

As a result of structural analysis, the crystal structure of the Fc(P208)/FcγRIIa type R extracellular region complex was determined at 2.87 Å resolution. The crystal structure of the Fc(P208)/FcγRIIa type R extracellular region complex was compared with the crystal structure of the Fc(P208)/FcγRIIb extracellular region complex shown in Example 3-1, and reflecting the very high amino acid homology of the two receptors, hardly any differences were observed for the overall structures (FIG. 11). However, when the structure was examined in detail at the electron density level, differences that may be used for improving selectivity were found. In FcγRIIa type R, the residue at position 160 was Phe instead of Tyr, and as shown in FIG. 12, this Phe cannot form a hydrogen bond with the main chain of amino acid residue at position 237 (EU numbering) of the Fc CH2 domain A, that was present in binding between FcgRIIb and Fc containing the P238D alteration. While this may be the major factor for improvement of selectivity for FcγRIIa type R by introduction of the P238D alteration, further comparison at the electron density level showed that in the complex formed with FcγRIIb, the electron density of the side chains of Leu at position 235 (EU numbering) and Leu at position 234 (EU numbering) in the Fc CH2 domain A can be confirmed, whereas the electron densities of these side chains were not clear in the complex formed with FcγRIIa type R, and the loop around position 237 (EU numbering) seems to be fluctuating as a result of decrease in interaction with FcgRIIa type R around this region. On the other hand, when structures of the same region are compared for CH2 domain B (FIG. 13), the electron density to Asp at position 237 (EU numbering) can be confirmed in the structure of the complex formed with FcγRIIb, whereas the electron density to about three residues upstream of Asp at position 237 (EU numbering) can be confirmed for the complex formed with FcγRIIa type R, and compared to binding with FcgRIIb, a wider region seems to be used for the interaction. The above suggested that in the region of positions 234 to 238 (EU numbering) of Fc(P208), the CH2 domain A side may largely contribute to binding with FcγRIIb, and the CH2 domain B side may largely contribute to binding with FcγRIIaR.

### [Expression and Purification of FcγRIIa Type R Extracellular Region]

This was prepared according to the method of Reference Example 2.

### [Purification of the Fc(P208)/FcγRIIa Type R Extracellular Region Complex]

To 1.5 mg of the purified FcγRIIa type R extracellular region sample, 0.15 mg of Endo F1 (Protein Science 1996, 5, 2617-2622) expressed and purified from Escherichia coli as a fusion protein with glutathione S-transferase and 20 μL of 5 U/mL Endo F2 (QA-bio) and 20 μL of 5 U/mL Endo F3 (QA-bio) were added. This was left to stand at room temperature for nine days in 0.1 M sodium acetate buffer (pH 4.5) condition, and then 0.07 mg of Endo F1 (Protein Science 1996, 5, 2617-2622) expressed and purified from Escherichia coli as a fusion protein with glutathione S-transferase and 7.5 μL of 5 U/mL Endo F2 (QA-bio) and 7.5 μL of 5 U/mL Endo F3 (QA-bio) were added. This was left to stand for three more days, and the N-linked oligosaccharide was cleaved, while leaving N-acetylglucosamine directly bound to Asn. Next, this FcγRIIa type R extracellular domain sample subjected to sugar chain cleavage treatment was concentrated using 10000 MWCO ultrafiltration filter, and purified by gel filtration chromatography (Superdex® 200 10/300 chromatography) using a column equilibrated with 25 mM HEPES (pH 7), 0.1 M NaCl. Furthermore, to the obtained sugar chain-cleaved FcγRIIa type R extracellular region fraction, Fc(P208) was added so that the molar ratio of FcγRIIa type R extracellular region would be present in slight excess, and after concentration using 10000 MWCO ultrafiltration filter, a sample of the Fc(P208)/FcγRIIa type R extracellular region complex was obtained through purification by gel filtration chromatography (Superdex® 200 10/300 chromatography) using a column equilibrated with 25 mM HEPES (pH 7), 0.1 M NaCl.

### [Crystallization of the Complex of Fc(P208)/FcγRIIaR Type Extracellular Region]

A sample of Fc(P208)/FcγRIIa R type extracellular region complex concentrated to about 10 mg/ml with a 10000 MWCO ultrafiltration filter was crystallized using the sitting drop vapor diffusion method. Using a reservoir solution of 0.1 M Bis-Tris (pH 7.5), 26% (w/v) PEG3350, 0.2 M ammonium sulfate, crystallization drops were prepared at a mixing ratio of reservoir solution: crystallization sample=0.8 μl: 1.0 μl. The drops were then tightly sealed and allowed to stand at 20° C. This succeeded in yielding plate-like crystals.

[X-Ray Diffraction Data Measurement from Fc(P208)/FcγRIIa Type R Extracellular Region Complex Crystal]

A single crystal of Fc(P208)/FcγRIIa type R extracellular region complex prepared was soaked into a solution of 0.1 M Bis-Tris (pH 7.5)), 27.5% (w/v) PEG3350, 0.2 M ammonium sulfate, 20% (v/v) glycerol. Then, the crystal was fished out of the solution using a pin with attached tiny nylon loop, and frozen in liquid nitrogen. X-ray diffraction data of the single crystal was measured at synchrotron radiation facility Photon Factory BL-17A in the High Energy Accelerator Research Organization. The crystal was constantly placed in a nitrogen stream at −178° C. to maintain in a frozen state during the measurement. A total of 225 X-ray diffraction images from the single crystal were collected using CCD detector Quantum 315r (ADSC) equipped to the beam line with rotating the single crystal at 0.6° at a time. Based on the obtained diffraction images, lattice constant determination, diffraction spot indexing, and diffraction data processing were performed using programs Xia2 (J. Appl. Cryst. (2010) 43, 186-190), XDS Package (Acta Cryst. (2010) D66, 125-132), and Scala (Acta Cryst. (2006) D62, 72-82). Finally, diffraction intensity data up to 2.87 Å resolution was obtained. The crystal belongs to the space group C2221 with lattice constant a=154.31 Å, b=257.61 Å, c=56.19 Å, α=90°, β=90°, and γ=90°.

### [X-Ray Crystal Structure Analysis of Fc(P208)/FcγRIIa Type R Extracellular Region Complex]

The structure was determined by a molecular replacement method using program Phaser (J. Appl. Cryst. (2007) 40, 658-674). The number of complexes in an asymmetrical unit was estimated to be one from the size of the obtained crystal lattice and the molecular weight of Fc(P208)/FcγRIIa type R extracellular region complex. Using, as a search model, the crystallographic structure of Fc(P208)/FcγRIIb extracellular region complex obtained as described in Example 3-1, the orientation and position in the crystal lattices were determined based on the rotation function and translation function. The crystallographic reliability factor R value for the data of diffracted intensity at 25 to 3.0 Å was 38.4% and Free R value was 38.0% after rigid body refinement of the obtained initial model which moves the two CH2 domains and two CH3 domains of the Fc, and the extracellular region of FcγRIIa type R. Then, structural model refinement was achieved by repeating structural refinement using program REFMAC5 (Acta Cryst. (2011) D67, 355-367) followed by revision of the model performed using program Coot (Acta Cryst. (2010) D66, 486-501) with reference to the electron density maps where the coefficients 2Fo-Fc and Fo-Fc were calculated using experimentally determined structural factor Fo, structural factor Fc calculated according to the model, and the phases calculated according to the model. Finally, as a result of incorporation of water molecules into the model based on the electron density maps which use 2Fo-Fc or Fo-Fc as the coefficient, and the following refinement, the crystallographic reliability factor, R values and the Free R value of the model containing 4758 non-hydrogen atoms became 26.3% and 29.8% to 24838 diffraction intensity data from 25 Å to 2.87 Å resolution, respectively.

### [Example 4] Fc Variants Whose Alteration Sites were Determined Based on Crystal Structures

As shown in Example 3, it was suggested that electrostatic interaction between Asp at position 268 (EU numbering) and Arg at position 292 (EU numbering) is formed as a result of structural changes nearby which accompany introduction of the P271G alteration into the CH2 domain B of the FcgRIIb-binding-enhanced variant Fc(P208) (FIG. 9). This interaction functions to stabilize the loop structure of positions 266 to 271 (EU numbering), and as a result it may have contributed to the enhancement of FcγRIIb binding. Accordingly, it was examined whether strengthening the electrostatic interaction with Arg at position 292 (EU numbering) to stabilize this loop structure by altering Asp at position 268 (EU numbering) to Glu leads to enhancement of interaction with FcgRIIb. Furthermore, as shown in FIG. 8, Tyr at position 160 (EU numbering) of FcgRIIb forms hydrogen bonds with the main chain of Asp at position 237 (EU numbering) of the Fc(P208) CH2 domain A, and plays an important role in binding with FcgRIIb. While the side chain portion of Asp at position 237 (EU numbering) does not form a particular interaction, Ile at position 332 (EU numbering), Glu at position 333 (EU numbering), and Lys at position 334 (EU numbering) are positioned nearby in the molecule. Examination of whether substituting these sites with hydrophilic residues to strengthen the interaction with Asp at position 237 (EU numbering) and stabilize the loop structure near this residue will enhance interaction with Tyr at position 160 of FcγRIIb were carried out.

Variants were produced by introducing H268E, I332T, I332S, I332E, I332K, E333K, E333R, E333S, E333T, K334S, K334T, and K334E individually into IL6R-BP230/IL6R-L (SEQ ID NO: 27/SEQ ID NO: 21) which was produced in Example 2. IL6R-L (SEQ ID NO: 21) was commonly used as the antibody L chain. These variants were used for antibody expression and purification according to the method of Reference Example 1, and binding to each FcgR (FcgRIa, FcgRIIa type H, FcgRIIa type R, FcgRIIb, and FcgRIIIa type V) was assessed using the method of Reference Example 2.

The KD of each variant to each FcgR is shown in Table 2. In the table, “alteration” refers to an alteration introduced into IL6R-BP3 (SEQ ID NO: 23). IL6R-B3/IL6R-L which is used as the template to produce IL6R-BP230 is indicated by an asterisk (*). “KD (IIb) of parent polypeptide/KD (IIb) of altered polypeptide” in the table shows the value obtained by dividing the KD value of IL6R-B3/IL6R-L for FcgRIIb by the KD value of each variant for FcgRIIb. Meanwhile, “KD (IIaR) of parent polypeptide/KD (IIaR) of altered polypeptide” shows the value obtained by dividing the KD value of IL6R-B3/IL6R-L for FcgR IIaR by the KD value of each variant for FcgR IIaR. “KD (IIaR)/KD (IIb)” shows the value obtained by dividing the KD of each variant for FcgRIIaR by the KD of the variant for FcgRIIb. The greater the value, the higher the selectivity to FcgRIIb is. In Table 2, values shown in bold italicized font are ones for which the binding of FcgR to IgG was concluded to be too weak to analyze correctly by kinetic analysis and thus was calculated using:

KD=C·Rₘₐₓ/(R_{eq}−RI)−C [Equation 2]

described in Reference Example 2.

TABLE 2

| | | | | | | | | KD(IIaR) OF | KD(IIb) OF |
|---|---|---|---|---|---|---|---|---|---|
| | | | | | | | | PARENT | PARENT |
| | | | | | | | | POLYPEPTIDE/ | POLYPEPTIDE/ |
| | ALTERATION | KD FOR | KD FOR | KD FOR | KD FOR | KD FOR | | KD(IIaR) OF | KD(IIb) OF |
| | ADDED TO | FcgRIa | FcgRIIaR | FcgRIIaH | FcgRIIb | FcgRIIIaV | KD(IIaR)/ | ALTERED | ALTERED |
| VARIANT NAME | IL6R-BP230 | (mol/L) | (mol/L) | (mol/L) | (mol/L) | (mol/L) | KD(IIb) | POLYPEPTIDE | POLYPEPTIDE |
| IL6R-G1d/IL6R-1L | | 3.2E−10 | 1.0E−06 | 6.7E−07 | 2.6E−06 | 3.5E−07 | 0.4 | 1.1 | 1.2 |
| IL6R-B3/IL6R-L | * | 4.2E−1.0 | 1.1E−06 | 7.7E−07 | 3.1E−06 | 3.3E−07 | 0.3 | 1.0 | 1.0 |
| IL6R-BP230/IL6R-L | | 1.4E−08 | 5.7E−07 | [symbol] | 2.1E−08 | [symbol] | 27.5 | 1.9 | 149.0 |
| IL6R-BP264/IL6R-L | H268E | 6.5E−09 | 4.8E−07 | [symbol] | 1.2E−08 | [symbol] | 40.6 | 2.3 | 265.0 |
| IL6R-BP384/IL6R-L | K334R | 3.0E−09 | 1.3E−06 | [symbol] | 7.1E−08 | [symbol] | 17.7 | 0.9 | 43.5 |
| IL6R-BP390/IL6R-L | I332S | 1.6E−09 | 4.9E−07 | [symbol] | 2.1E−08 | [symbol] | 22.9 | 2.2 | 144.9 |
| IL6R-BP391/IL6R-L | I332T | 9.6E−10 | 3.4E−07 | [symbol] | 1.3E−08 | [symbol] | 26.6 | 3.2 | 242.2 |
| IL6R-BP392/IL6R-L | I332K | 7.9E−09 | 7.3E−06 | [symbol] | 9.9E−07 | [symbol] | 7.3 | 0.2 | 3.1 |
| IL6R-BP393/IL6R-L | I332R | 1.1E−08 | 3.9E−06 | [symbol] | 2.7E−06 | [symbol] | 1.4 | 0.3 | 1.2 |
| IL6R-BP465/IL6R-L | E333K | 1.6E−08 | 6.1E−07 | [symbol] | 2.1E−08 | [symbol] | 29.8 | 1.8 | 151.2 |
| IL6R-BP466/IL6R-L | E333R | 1.5E−08 | 5.2E−07 | [symbol] | 1.7E−08 | [symbol] | 30.4 | 2.1 | 181.3 |
| IL6R-BP467/IL6R-L | K334S | 8.9E−10 | 1.1E−06 | [symbol] | 4.1E−08 | [symbol] | 25.8 | 1.0 | 75.4 |
| IL6R-BP468/IL6R-L | K334T | 9.7E−10 | 1.1E−06 | [symbol] | 4.0E−08 | [symbol] | 26.3 | 1.0 | 77.7 |
| IL6R-BP469/IL6R-L | E333S | 1.3E−08 | 6.0E−07 | [symbol] | 2.3E−08 | [symbol] | 26.4 | 1.8 | 136.6 |
| IL6R-BP470/IL6R-L | E333T | 1.5E−08 | 4.9E−07 | [symbol] | 1.6E−08 | [symbol] | 30.6 | 2.2 | 192.5 |

[symbol]

indicates data missing or illegible when filed

Both FcgRIIb binding and FcgRIIb selectivity of IL6R-BP264/IL6R-L, IL6R-BP465/IL6R-L, IL6R-BP466/IL6R-L, and IL6R-BP470, resulting from introducing H268E, E333K, E333R, and E333T, respectively, into IL6R-BP230/IL6R-L were increased as compared to those of IL6R-BP230/IL6R-L. The FcgRIIb selectivity of IL6R-BP391/IL6R-L introduced with the I332T was reduced while its FcgRIIb binding was increased as compared to IL6R-BP230/IL6R-L.

### [Example 5] Introduction of Comprehensive Alterations into the Area Around Position 271 (EU Numbering)

When the X-ray crystal structure of the complex formed between Fc(P238D) carrying the P238D alteration and the FcγRIIb extracellular region and the X-ray crystal structure of the complex formed between Fc(P208) and the FcγRIIb extracellular region were compared, the structure near position 271 (EU numbering) showed the greatest structural change (FIG. 9). As shown in Reference Example 8, in Fc(P238D), it was suggested that when Asp at position 270 (EU numbering) forms a strong electrostatic interaction with Arg at position 131 of FcγRIIb, the Pro at position 271 (EU numbering) portion may be stereochemically strained. In the structure of Fc(P208)/FcγRIIb, introduction of the P271G alteration causes positional changes at the main-chain level to remove this structural strain. As a result, the structure near position 271 (EU numbering) may have been largely changed. If alterations that further stabilize this changed structure can be efficiently introduced, entropic energy loss accompanying formation of electrostatic interaction with Arg at position 131 of FcγRIIb can be reduced, and this may lead to enhancement of binding activity. Accordingly, comprehensive alterations were introduced into the area around position 271 (EU numbering) to screen for alterations that show effects of enhancing binding to FcgRIIb or improving selectivity to FcgRIIb.

IL6R-BP267 (SEQ ID NO: 29) produced by introducing E233D, G237D, P238D, H268E, and P271G into IL6R-B3 (SEQ ID NO: 23) was used as the template for introducing comprehensive alterations. Amino acids at positions 264, 265, 266, 267, 269, and 272 (EU numbering)) in IL6R-BP267 were individually substituted with any of the 18 amino acids other than the original amino acids and Cys. IL6R-L (SEQ ID NO: 21) was commonly used as the antibody L chain. These variants were used for antibody expression and purification according to the method of Reference Example 1, and binding to each FcgR (FcgRIa, FcgRIIa type H, FcgRIIa type R, FcgRIIb, and FcgRIIIa type V) was assessed using the method of Reference Example 2. From among the obtained variants, those that enhanced FcgRIIb-binding or improved selectivity to FcgRIIb compared to those of the IL6R-BP267/IL6R-L prior to alteration introduction are summarized in Table 3.

TABLE 3

| | | | | | | | | KD(IIaR) OF | KD(IIb) OF |
|---|---|---|---|---|---|---|---|---|---|
| | | | | | | | | PARENT | PARENT |
| | | | | | | | | POLYPEPTIDE/ | POLYPEPTIDE/ |
| | ALTERATION | KD FOR | KD FOR | KD FOR | KD FOR | KD FOR | | KD(IIaR) OF | KD(IIb) OF |
| | ADDED TO | FcgRIa | FcgRIIaR | FcgRIIaH | FcgRIIb | FcgRIIIaV | KD(IIaR)/ | ALTERED | ALTERED |
| VARIANT NAME | IL6R-BP230 | (mol/L) | (mol/L) | (mol/L) | (mol/L) | (mol/L) | KD(IIb) | POLYPEPTIDE | POLYPEPTIDE |
| IL6R-BP/IL6R-L | * | 4.2E−10 | 1.1E−06 | 7.7E−07 | 3.1E−06 | 3.3E−07 | 0.3 | 1.0 | 1.0 |
| IL6R-BP267/IL6R-L | | 4.0E−09 | 1.7E−06 | [symbol] | 1.3E−07 | [symbol] | 13.0 | 0.7 | 24.3 |
| IL6R-BP348/IL6R-L | S267A | 5.5E−10 | 7.0E−07 | [symbol] | 4.6E−08 | [symbol] | 15.3 | 1.5 | 67.6 |
| IL6R-BP300/IL6R-L | V264I | 9.6E−09 | 6.9E−07 | [symbol] | 5.8E−08 | [symbol] | 11.9 | 1.6 | 53.3 |
| IL6R-BP367/IL6R-L | E269D | 3.1E−09 | 1.2E−06 | [symbol] | 1.0E−07 | [symbol] | 11.7 | 0.9 | 30.6 |
| IL6R-BP350/IL6R-L. | S267E | 8.9E−10 | 1.5E−07 | [symbol] | 1.0E−07 | [symbol] | 1.5 | 7.0 | 30.0 |
| IL6R-BP333/IL6R-L | V266F | 9.1E−09 | 1.5E−06 | [symbol] | 1.2E−07 | [symbol] | 12.5 | 0.7 | 26.4 |
| IL6R-BP352/IL6R-L | S267G | 1.8E−09 | 1.9E−06 | [symbol] | 1.2E−07 | [symbol] | 15.7 | 0.6 | 25.3 |
| IL6R-BP339/IL6R-L | V266M | 4.6E−09 | 1.4E−06 | [symbol] | 1.3E−07 | [symbol] | 11.3 | 0.8 | 24.5 |
| IL6R-BP520/IL6R-L | E272M | 3.9E−09 | 3.0E−06 | [symbol] | 1.7E−07 | [symbol] | 17.5 | 0.4 | 17.8 |
| IL6R-BP523/IL6R-L | E272Q | 3.7E−09 | 2.7E−06 | [symbol] | 1.7E−07 | [symbol] | 15.9 | 0.4 | 18.4 |
| IL6R-BP313/IL6R-L | D265E | 2.6E−08 | 1.3E−05 | [symbol] | 8.4E−07 | [symbol] | 15.6 | 0.1 | 3.7 |
| IL6R-BP513/IL6R-L | E2720 | 3.8E−09 | 1.7E−06 | [symbol] | 1.1E−07 | [symbol] | 15.4 | 0.6 | 28.1 |
| IL6R-BP521/IL6R-L | E272N | 3.6E−09 | 2.9E−06 | [symbol] | 1.9E−07 | [symbol] | 15.2 | 0.4 | 16.0 |
| IL6R-BP338/IL6R-L | V266L | 1.5E−08 | 2.2E−06 | [symbol] | 1.5E−07 | [symbol] | 15.0 | 0.5 | 21.3 |
| IL6R-BP517/IL6R-L | E272I | 3.2E−09 | 2.1E−06 | [symbol] | 1.4E−07 | [symbol] | 14.7 | 0.5 | 21.5 |
| IL6R-BP514/IL6R-L | E272F | 4.3E−09 | 3.0E−06 | [symbol] | 2.1E−07 | [symbol] | 14.0 | 0.4 | 14.6 |

[symbol]

indicates data missing or illegible when filed

The KD value of each variant to each FcgR is shown in Table 3. In the table, “alteration added to IL6R-BP267” refers to an alteration introduced into IL6R-BP267 (SEQ ID NO: 29), which was used as a template. IL6R-B3/IL6R-L which is used as the parent to produce IL6R-B3 is indicated by asterisk (*). In the table, “KD (IIb) of parent polypeptide/KD (IIb) of altered polypeptide” shows the value obtained by dividing the KD value of IL6R-B3/IL6R-L for FcgRIIb by the KD value of each variant for FcgRIIb. Meanwhile, “KD (IIaR) of parent polypeptide/KD (IIaR) of altered polypeptide” shows the value obtained by dividing the KD of IL6R-B3/IL6R-L for FcgRIIaR by the KD of each variant for FcgR IIaR. “KD (IIaR)/KD (IIb)” shows the value obtained by dividing the KD value of each variant for FcgRIIaR by the KD value of the variant for FcgRIIb. The greater the value is, the higher the selectivity to FcgRIIb is. In Table 3, the values shown in bold italicized font are ones for which the binding of FcgR to IgG was concluded to be too weak to analyze correctly by kinetic analysis and thus was calculated using:

KD=C·Rₘₐₓ/(R_{eq}−RI)−C [Equation 2]

described in Reference Example 2.

All the binding of variants shown in Table 3 to FcgRIa, FcgRIIaH, and FcgRIIIaV were comparable or reduced as compared to that of IL6R-B3/IL6R-L. Meanwhile, the FcgRIIb binding of variants resulting from adding the S267A, V264I, E269D, S267E, V266F, S267G, and V266M, respectively, to IL6R-BP267/IL6R-L was increased as compared to that of IL6R-BP267/IL6R-L prior to addition of alteration. Meanwhile, the KD (IIaR)/KD (IIb) values of variants resulting from adding the S267A, S267G, E272M, E272Q, D265E, E272D, E272N, V266L, E272I, and E272F alterations, respectively, to IL6R-BP267/IL6R-L were increased as compared to that of IL6R-BP267/IL6R-L prior to addition of alteration, demonstrating the effect to improve the FcgRIIb selectivity.

### [Example 6] Enhancement of the FcgRIIb Binding by Introducing Alterations into CH3 Region

An alteration substituting Leu for Pro at position 396 (EU numbering) has been reported to enhance the FcgRIIb binding (Cancer Res. (2007) 67, 8882-8890). Position 396 (EU numbering) is present at a position which is not directly involved in the interaction with FcgR. However, the amino acid can be assumed to have an effect on the interaction with FcgR by changing the antibody structure. Thus, the present inventors assessed whether the FcgRIIb binding is enhanced or its FcgRIIb selectivity is increased by comprehensive introduction of amino acid alterations at position 396 (EU numbering).

IL6R-BP423 (SEQ ID NO: 33) produced by introducing E233D, G237D, P238D, S267A, H268E, P271G, and A330R into IL6R-B3 (SEQ ID NO: 23) was used as the template. Variants were produced by substituting the amino acid at position 396 (EU numbering)) in IL6R-BP423 with any of the 18 amino acids other than the original amino acid and cysteine. IL6R-L (SEQ ID NO: 21) was commonly used as the antibody L chain. These variants were used for antibody expression and purification according to the method of Reference Example 1, and binding to each FcgR (FcgRIa, FcgRIIa type H, FcgRIIa type R, FcgRIIb, and FcgRIIIa type V) was assessed using the method of Reference Example 2. Binding of the obtained variants to each FcgR are summarized in Table 4.

TABLE 4

| | | | | | | | | KD(IIaR) OF | KD(IIb) OF |
|---|---|---|---|---|---|---|---|---|---|
| | | | | | | | | PARENT | PARENT |
| | | | | | | | | POLYPEPTIDE/ | POLYPEPTIDE/ |
| | ALTERATION | KD FOR | KD FOR | KD FOR | KD FOR | KD FOR | | KD(IIaR) OF | KD(IIb) OF |
| | ADDED TO | FcgRIa | FcgRIIaR | FcgRIIaH | FcgRIIb | FcgRIIIaV | KD(IIaR)/ | ALTERED | ALTERED |
| VARIANT NAME | IL6R-BP423 | (mol/L) | (mol/L) | (mol/L) | (mol/L) | (mol/L) | KD(IIb) | POLYPEPTIDE | POLYPEPTIDE |
| IL6R-G1d/IL6R-L | | 3.2E−10 | 1.0E−06 | 6.7E−07 | 2.6E−06 | 3.5E−07 | 0.4 | 1.1 | 1.2 |
| IL6R-B3/IL6R-L | * | 4.2E−10 | 1.1E−06 | 7.7E−07 | 3.1E−06 | 3.3E−07 | 0.3 | 1.0 | 1.0 |
| IL6R-BP423/IL6R-L | | 7.78-10 | 1.8E−07 | [symbol] | 5.1E−09 | [symbol] | 34.2 | 6.3 | 604 |
| IL6R-BP447/IL6R-L | P396A | 9.0E−10 | 1.6E−07 | [symbol] | 5.3E−09 | [symbol] | 29.7 | 7.0 | 584 |
| IL6R-BP448/IL6R-L | P396D | 7.5E−10 | 1.3E−07 | [symbol] | 4.1E−09 | [symbol] | 31.7 | 8.5 | 759 |
| IL6R-BP449/IL6R-L | P396E | 9.1E−10 | 1.4E−07 | [symbol] | 4.6E−09 | [symbol] | 29.8 | 8.0 | 667 |
| IL6R-BP450/IL6R-L | P396F | 8.4E−10 | 1.2E−07 | [symbol] | 4.1E−09 | [symbol] | 29.4 | 9.2 | 763 |
| IL6R-BP451/IL6R-L | P396G | 9.8E−10 | 1.8E−07 | [symbol] | 6.2E−09 | [symbol] | 29.2 | 6.1 | 499 |
| IL6R-BP452/IL6R-L | P396H | 7.5E−10 | 1.3E−07 | [symbol] | 5.1E−09 | [symbol] | 25.9 | 8.3 | 602 |
| IL6R-BP453/IL6R-L | P396I | 7.5E−10 | 1.2E−07 | [symbol] | 4.6E−09 | [symbol] | 25.5 | 9.4 | 675 |
| IL6R-BP454/IL6R-L | P396K | 8.2E−09 | 1.3E−07 | [symbol] | 4.8E−09 | [symbol] | 27.5 | 8.4 | 649 |
| IL6R-BP455/IL6R-L | P396L | 7.5E−10 | 1.3E−07 | [symbol] | 4.0E−09 | [symbol] | 31.8 | 8.6 | 767 |
| IL6R-BP456/IL6R-L | P396M | 6.0E−10 | 1.2E−07 | [symbol] | 3.5E−09 | [symbol] | 35.3 | 8.9 | 888 |
| IL6R-BP457/IL6R-L | P396N | 9.1E−10 | 1.5E−07 | [symbol] | 5.2E−09 | [symbol] | 28.9 | 7.3 | 591 |
| IL6R-BP458/IL6R-L | P396Q | 7.8E−10 | 1.4E−07 | [symbol] | 4.5E−09 | [symbol] | 31.1 | 7.9 | 687 |
| IL6R-BP459/IL6R-L | P396R | 1.1E−09 | 1.5E−07 | [symbol] | 5.1E−09 | [symbol] | 28.9 | 7.5 | 607 |
| IL6R-BP460/IL6R-L | P396S | 8.7E−10 | 1.6E−07 | [symbol] | 6.5E−09 | [symbol] | 25.2 | 6.7 | 478 |
| IL6R-BP461/IL6R-L | P396T | 1.3E−09 | 1.3E−07 | [symbol] | 5.1E−09 | [symbol] | 24.4 | 8.8 | 602 |
| IL6R-BP462/IL6R-L | P396V | 9.7E−10 | 1.3E−07 | [symbol] | 5.2E−09 | [symbol] | 25.0 | 8.5 | 593 |
| IL6R-BP463/IL6R-L | P396W | 1.3E−09 | 1.6E−07 | [symbol] | 5.6E−09 | [symbol] | 28.1 | 7.0 | 554 |
| IL6R-BP464/IL6R-L | P396Y | 1.1E−09 | 1.3E−07 | [symbol] | 4.0E−09 | [symbol] | 31.5 | 8.7 | 773 |

[symbol]

indicates data missing or illegible when filed

In the Table, “alteration added to IL6R-BP423” refers to an alteration introduced into IL6R-BP423. IL6R-B3/IL6R-L which was used as the template to produce IL6R-BP423 is indicated by asterisk (*). In the table, “KD (IIb) of parent polypeptide/KD (IIb) of altered polypeptide” shows the value obtained by dividing the KD value of IL6R-B3/IL6R-L for FcgRIIb by the KD value of each variant for FcgRIIb. Meanwhile, “KD (IIaR) of parent polypeptide/KD (IIaR) of altered polypeptide” shows the value obtained by dividing the KD value of IL6R-B3/IL6R-L for FcgR IIaR by the KD value of each variant for FcgR IIaR. “KD (IIaR)/KD (IIb)” shows the value obtained by dividing the KD of each variant for FcgRIIaR by the KD of the variant for FcgRIIb. The greater the value, the higher the selectivity to FcgRIIb is. In Table 4, the values shown in bold italicized font are ones for which the binding of FcgR to IgG was concluded to be too weak to analyze correctly by kinetic analysis and thus was calculated using:

KD=C·Rₘₐₓ/(R_{eq}−RI)−C [Equation 2]

described in Reference Example 2.

The result shown in Table 4 demonstrates that: the FcgRIIb-binding activity of IL6R-BP456/IL6R-L resulting from introducing P396M into IL6R-BP423/IL6R-L, IL6R-BP455/IL6R-L resulting from introducing P396L into IL6R-BP423/IL6R-L, IL6R-BP464/IL6R-L resulting from introducing P396Y into IL6R-BP423/IL6R-L, IL6R-BP450/IL6R-L resulting from introducing P396F into IL6R-BP423/IL6R-L, IL6R-BP448/IL6R-L resulting from introducing P396D into IL6R-BP423/IL6R-L, IL6R-BP458/IL6R-L resulting from introducing P396Q into IL6R-BP423/IL6R-L, IL6R-BP453/IL6R-L resulting from introducing P396I into IL6R-BP423/IL6R-L, IL6R-BP449/IL6R-L resulting from introducing P396E into IL6R-BP423/IL6R-L, IL6R-BP454/IL6R-L resulting from introducing P396K into IL6R-BP423/IL6R-L, and IL6R-BP459/IL6R-L resulting from introducing P396R into IL6R-BP423/IL6R-L was all increased as compared to that of IL6R-BP423/IL6R-L prior to introduction of the alterations. Meanwhile, the KD (IIaR)/KD (IIb) value of IL6R-BP456/IL6R-L resulting from introducing P396M into IL6R-BP423/IL6R-L was larger as compared to that of IL6R-BP423/IL6R-L prior to introduction of the alteration, demonstrating the improved FcgRIIb selectivity. As seen in Table 4, the affinity of the prepared variants to FcgRIa, FcgRIIaH, and FcgRIIIaV was all lower than that of IL6R-B3/IL6R-L, which was the parent polypeptide.

### [Example 7] Preparation of Variants with Enhanced FcgRIIb Binding Using Subclass Sequences

Subclass exists in human IgG and its FcgR binding profile varies. The present inventors assessed whether the difference in the affinity to each FcgR between IgG1 and IgG4 could be utilized to increase the FcgRIIb binding and/or improve the selectivity.

First, IgG1 and IgG4 were analyzed for their affinity to each FcgR. IL6R-G4d (SEQ ID NO: 30) containing G4d was constructed as the antibody H chain. G4d lacks the C-terminal Gly and Lys and contains a substitution of Pro for Ser at position 228 (EU numbering) in human IgG4. IL6R-L (SEQ ID NO: 21) was commonly used as the antibody L chain. IL6R-G1d/IL6R-L and IL6R-G4d/IL6R-L were expressed and purified according to the method described in Reference Example 1. These were assessed for their binding to each FcgR (FcgRIa, FcgRIIa type H, FcgRIIa type R, FcgRIIb, or FcgRIIIa type V) by the method described in Reference Example 2. The binding of the resulting variants to each FcgR is summarized in Table 5.

TABLE 5

| | KD FOR | KD FOR | KD FOR | KD FOR | KD FOR |
|---|---|---|---|---|---|
| VARIANT | FcgRIa | FcgRIIaR | FcgRIIaH | FcgRIIb | FcgRIIIaV |
| NAME | (mol/L) | (mol/L) | (mol/L) | (mol/L) | (mol/L) |
| IL6R-G1d/ | 1.2E−10 | 9.7E−07 | 6.5E−07 | 3.9E−06 | 4.2E−07 |
| IL6R-L | | | | | |
| IL6R-G4d/ | 6.6E−10 | 2.1E−06 | 3.4E−06 | 2.6E−06 | 3.4E−06 |
| IL6R-L | | | | | |

Compared to IL6R-G1d/IL6R-L, IL6R-G4d/IL6R-L was found to have 1.5-fold stronger binding to FcgRIIb and 2.2-fold weaker binding to FcgRIIaR. Furthermore, compared to IL6R-G1d/IL6R-L, IL6R-G4d/IL6R-L had weaker affinity to FcgRIa, FcgRIIaH, and FcgRIIIaV. The above-mentioned results revealed that compared to IL6R-G1d, IL6R-G4d has excellent selectivity and binding to FcgRIIb.

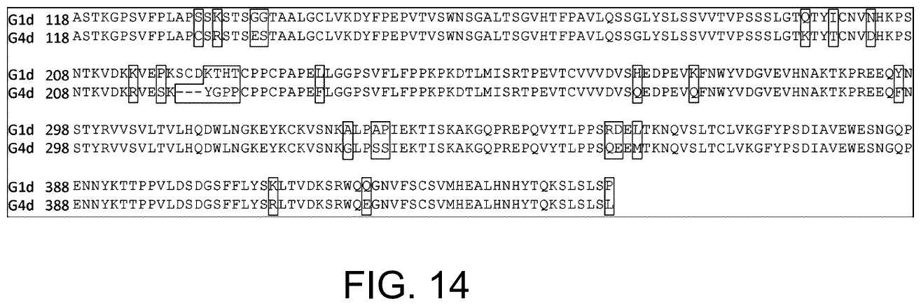

FIG. 14 shows the comparison of the sequences of G1d and G4d from CH1 to the C terminus (positions 118 to 445 (EU numbering)). The boxed amino acids in FIG. 14 show residues that are different between G1d and G4d. Several sites predicted to be involved in interaction with FcgR were selected from among these different amino acids, and whether further improvement of selectivity and binding is possible was examined by transferring the sequence of G4d having excellent selectivity and binding to FcgRIIb into FcgRIIb-enhanced variants.

Specifically, IL6R-BP473 was produced by introducing A327G into IL6R-BP230, IL6R-BP472 was produced by introducing A330S into IL6R-BP230, IL6R-BP471 was produced by introducing P331S into IL6R-BP230, IL6R-BP474 was produced by introducing A330S and P331S into IL6R-BP230, IL6R-BP475 was produced by introducing A327G and A330S into IL6R-BP230, IL6R-BP476 was produced by introducing A327G, A330S, and P331S into IL6R-BP230, and IL6R-BP477 was produced by introducing A327G and P331S into IL6R-BP230. Furthermore IL6R-BP478 (SEQ ID NO: 31) was produced by substituting Ala at position 118 to Thr at position 225 (EU numbering) in IL6R-BP230 with a G4d sequence (Ala at position 118 to Pro at position 222 (EU numbering)). IL6R-L (SEQ ID NO: 21) was commonly used as the antibody L chain. These variants were used for antibody expression and purification according to the method of Reference Example 1, and binding to each FcgR (FcgRIa, FcgRIIa type H, FcgRIIa type R, FcgRIIb, and FcgRIIIa type V) was assessed using the method of Reference Example 2.

The KD value of each variant to each FcgR is shown in Table 6. “KD (IIb) of parent polypeptide/KD (IIb) of altered polypeptide” in the table shows the value obtained by dividing the KD value of IL6R-B3/IL6R-L for FcgRIIb by the KD value of each variant for FcgRIIb. “Alteration(s) added to IL6R-BP230” refers to an alteration introduced into IL6R-BP230. IL6R-B3/IL6R-L used as the template to produce IL6R-BP230 is indicated by *1. Meanwhile, IL6R-BP478 (SEQ ID NO: 31), in which the segment from Ala at position 118 up to Thr at position 225 (EU numbering) in IL6R-BP230 is substituted with the sequence of G4d (Ala at position 118 up to Pro at position 222 (EU numbering)), is indicated by *2. “KD (IIaR) of parent polypeptide/KD (IIaR) of altered polypeptide” shows the value obtained by dividing the KD value of IL6R-B3/IL6R-L for FcgR IIaR by the KD value of the variant for FcgR IIaR. “KD (IIaR)/KD (IIb)” shows the value obtained by dividing the KD of each variant for FcgRIIaR by the KD of the variant for FcgRIIb. The greater the value, the higher the selectivity to FcgRIIb is. In Table 6, the values shown in bold italicized font are ones for which the binding of FcgR to IgG was concluded to be too weak to analyze correctly by kinetic analysis and thus was calculated using:

KD=C·Rₘₐₓ/(R_{eq}−RI)−C [Equation 2]

described in Reference Example 2.

TABLE 6

| | | | | | | | | KD(IIaR) OF | KD(IIb) OF |
|---|---|---|---|---|---|---|---|---|---|
| | | | | | | | | PARENT | PARENT |
| | | | | | | | | POLYPEPTIDE/ | POLYPEPTIDE/ |
| | ALTERATION(S) | KD FOR | KD FOR | KD FOR | KD FOR | KD FOR | | KD(IIaR) OF | KD(IIb) OF |
| | ADDED TO | FcgRIa | FcgRIIaR | FcgRIIaH | FcgRIIb | FcgRIIIaV | KD(IIaR)/ | ALTERED | ALTERED |
| VARIANT NAME | IL6R-BP230 | (mol/L) | (mol/L) | (mol/L) | (mol/L) | (mol/L) | KD(IIb) | POLYPEPTIDE | POLYPEPTIDE |
| IL6R-G1d/IL6R-L | | 3.2E−10 | 1.0E−06 | 6.7E−07 | 2.6E−06 | 3.5E−07 | 0.4 | 1.1 | 1.2 |
| IL6R-B3/IL6R-L | *1 | 4.2E−10 | 1.1E−06 | 7.7E−07 | 3.1E−06 | 3.3E−07 | 0.3 | 1.0 | 1.0 |
| IL6R-BP230/IL6R-L | | 1.4E−08 | 5.7E−07 | [symbol] | 2.1E−08 | [symbol] | 27.5 | 1.9 | 149.0 |
| IL6R-BP471/IL6R-L | P331S | 7.3E−09 | 8.0E−07 | [symbol] | 3.5E−08 | [symbol] | 22.7 | 1.4 | 88.1 |
| IL6R-BP472/IL6R-L | A330S | 5.2E−09 | 3.3E−06 | [symbol] | 1.5E−07 | [symbol] | 21.5 | 0.3 | 20.3 |
| IL6R-BP473/IL6R-L | A327G | 6.2E−09 | 3.8E−07 | +Z899 | 1.8E−08 | [symbol] | 21.1 | 2.9 | 172.2 |
| IL6R-BP474/IL6R-L | A330S/P331S | 4.1E−09 | 3.0E−06 | [symbol] | 1.8E−07 | [symbol] | 16.6 | 0.4 | 16.9 |
| IL6R-BP475/IL6R-L | A327G/A330S | 4.9E−09 | 1.0E−06 | [symbol] | 1.1E−07 | [symbol] | 9.7 | 1.1 | 29.2 |
| IL6R-BP476/IL6R-L | A327G/A330S/ | 5.9E−09 | 1.3E−06 | [symbol] | 1.3E−07 | [symbol] | 9.7 | 0.9 | 23.7 |
| | P331S | | | | | | | | |
| IL6R-BP477/IL6R-L | A327G/P331S | 9.2E−09 | 5.1E−07 | [symbol] | 3.7E−08 | [symbol] | 14.0 | 2.2 | 84.9 |
| IL6R-BP478/IL6R-L | *2 | 7.7E−09 | 5.4E−07 | [symbol] | 1.9E−08 | [symbol] | 28.0 | 2.0 | 160.6 |

[symbol]

indicates data missing or illegible when filed

Of the variants shown in Table 6, IL6R-BP473/IL6R-L introduced with A327G showed FcgRIIb binding increased by 1.2 times compared to that of IL6R-BP230/IL6R-L. Compared to IL6R-BP230/IL6R-L, IL6R-BP478/IL6R-L produced by substituting Ala at position 118 to Thr at position 225 (EU numbering) in IL6R-BP230 with a G4d sequence (Ala at position 118 to Pro at position 222 (EU numbering)), showed 1.1-fold increase in both FcgRIIb-binding and FcgRIIaR-binding. Compared to IL6R-B3/IL6R-L which is the parent peptide, all variants had lower affinity to FcgRIa, FcgRIIaH, and FcgRIIIaV.

Furthermore, as shown in FIG. 14, the other sites where the amino acids are different between G1d and G4d include positions 268, 274, 296, 355, 356, 358, 409, 419, and 445 (EU numbering). Therefore, by substituting these sites with IgG4-derived amino acids, selectivity and binding to FcgRIIb may be enhanced.

In the examination carried out so far, transferring A327G, which is in the human IgG4 sequence, to the variant IL6R-BP230/IL6R-L was shown to enhance FcγRIIb-binding activity. A further examination was performed for portions that do not match between the IgG4 and IgG1 sequences.

Specifically, variants were produced by introducing the following alterations into IL6R-BP230 as the antibody H chain: K274Q was introduced to produce IL6R-BP541; Y296F was introduced to produce IL6R-BP542; H268Q was introduced to produce IL6R-BP543; R355Q was introduced to produce IL6R-BP544; D356E was introduced to produce IL6R-BP545; L358M was introduced to produce IL6R-BP546; K409R was introduced to produce IL6R-BP547; and Q419E was introduced to produce IL6R-BP548. Meanwhile, IL6R-L was used as the common antibody L chain. Antibodies that contain the above heavy chain variant and the light chain IL6R-L were purified according to the methods described in Reference Example 1. The purified antibodies were assessed for their binding to each FcγR (FcγRIa, FcγRIIaH, FcγRIIaR, FcγRIIb, or FcγRIIIaV) by the method of Reference Example 2.

The KD of each variant for each FcγR is shown in Table 7. In this Table, “parent polypeptide KD(IIb)/altered polypeptide KD(IIb)” refers to a value obtained by dividing the KD value of IL6R-B3/IL6R-L for FcγRIIb by the KD value of each variant for FcγRIIb. In the Table, “alterations added to IL6R-BP230” indicate alterations introduced into IL6R-BP230. Meanwhile, IL6R-B3/IL6R-L used as a template when producing IL6R-BP230 was indicated as *1. “Parent polypeptide KD(IIaR)/altered polypeptide KD(IIaR)” refers to a value obtained by dividing the KD value of IL6R-B3/IL6R-L for FcγRIIaR by the KD value of each variant for FcγRIIaR. “KD(IIaR)/KD(IIb)” is a value obtained by dividing the KD of each variant for FcγRIIaR by the KD of this variant for FcγRIIb. The larger this value is, the higher the selectivity to FcγRIIb is. The Table 7 values shown in bold italicized font were calculated using the following equation

KD=C·Rₘₐₓ/(R_{eq}−RI)−C [Equation 2]

shown in Reference Example 2 since the binding of FcγR to IgG was determined to be too weak to analyze correctly by kinetic analysis.

TABLE 7

| | | | | | | | | KD(IIaR) OF | KD(IIb) OF |
|---|---|---|---|---|---|---|---|---|---|
| | | | | | | | | PARENT | PARENT |
| | | KD | KD | KD | KD | KD | | POLYPEPTIDE/ | POLYPEPTIDE/ |
| | ALTERATION(S) | AGAINST | AGAINST | AGAINST | AGAINST | AGAINST | | KD(IIaR) OF | KD(IIb) OF |
| VARIANT | ADDED TO | FcgRIa | FcgRIIaR | FcgRIIaH | FcgRIIb | FcgRIIIaV | KD(IIaR)/ | ALTERED | ALTERED |
| NAME | IL6R-BP230 | (mol/L) | (mol/L) | (mol/L) | (mol/L) | (mol/L) | KD(IIb) | POLYPEPTIDE | POLYPEPTIDE |
| IL6R-G1d/ | | 3.2E−10 | 1.0E−06 | 6.7E−07 | 2.6E−06 | 3.5E−07 | 0.4 | 1.1 | 1.2 |
| IL6R-L | | | | | | | | | |
| IL6R-B3/ | *1 | 4.2E−10 | 1.1E−06 | 7.7E−07 | 3.1E−06 | 3.3E−07 | 0.3 | 1.0 | 1.0 |
| IL6R-L | | | | | | | | | |
| IL6R-B230/ | | 1.0E−08 | 4.9E−07 | [symbol] | 1.8E−08 | [symbol] | 28.0 | 2.2 | 175.6 |
| IL6R-L | | | | | | | | | |
| IL6R-BP541/ | K274Q | 1.1E−08 | 4.5E−07 | [symbol] | 1.6E−08 | [symbol] | 27.8 | 2.4 | 189.6 |
| IL6R-L | | | | | | | | | |
| IL6R-BP542/ | Y296F | 1.3E−08 | 4.9E−07 | [symbol] | 2.0E−08 | [symbol] | 24.4 | 2.2 | 153.7 |
| IL6R-L | | | | | | | | | |
| IL6R-BP543/ | H268Q | 2.3E−08 | 5.6E−07 | [symbol] | 2.0E−08 | [symbol] | 27.3 | 1.9 | 151.5 |
| IL6R-L | | | | | | | | | |
| IL6R-BP544/ | R355Q | 9.8E−09 | 4.8E−07 | [symbol] | 1.7E−08 | [symbol] | 28.8 | 2.2 | 183.9 |
| IL6R-L | | | | | | | | | |
| IL6R-BP545/ | D356E | 9.9E−09 | 5.7E−07 | [symbol] | 1.7E−08 | [symbol] | 32.7 | 1.9 | 178.6 |
| IL6R-L | | | | | | | | | |
| IL6R-BP546/ | L358M | 9.0E−09 | 5.0E−07 | [symbol] | 1.5E−08 | [symbol] | 32.8 | 2.2 | 204.6 |
| IL6R-L | | | | | | | | | |
| IL6R-BP547/ | K409R | 1.2E−08 | 4.9E−07 | [symbol] | 1.9E−08 | [symbol] | 25.5 | 2.2 | 162.6 |
| IL6R-L | | | | | | | | | |
| IL6R-BP548/ | Q419E | 1.2E−08 | 5.0E−07 | [symbol] | 1.9E−08 | [symbol] | 26.2 | 2.2 | 161.8 |
| IL6R-L | | | | | | | | | |

[symbol]

indicates data missing or illegible when filed

As shown in Table 7, compared to the IL6R-BP230/IL6R-L prior to alteration, IL6R-BP541/IL6R-L produced by introducing K274Q into IL6R-BP230/IL6R-L, IL6R-BP544/IL6R-L produced by introducing R355Q into IL6R-BP230/IL6R-L, IL6R-BP545/IL6R-L produced by introducing D356E into IL6R-BP230/IL6R-L, and IL6R-BP546/IL6R-L produced by introducing L358M into IL6R-BP230/IL6R-L, had enhanced FcγRIIb-binding. Among them, IL6R-BP544/IL6R-L produced by introducing R355Q into IL6R-BP230/IL6R-L, IL6R-BP545/IL6R-L produced by introducing D356E into IL6R-BP230/IL6R-L, and IL6R-BP546/IL6R-L produced by introducing L358M into IL6R-BP230/IL6R-L show increased KD(IIaR)/KD(IIb) values when compared to that of the IL6R-BP230/IL6R-L prior to alteration, showing that these alterations enhance selectivity to FcγRIIb as well.

### [Example 8] Examination of Combining Alterations that Bring about Improvement of Selectivity and Enhancement of Binding to FcgRIIb

The combinations of alterations that were found in the examinations carried out so far to improve selectivity or binding activity to FcγRIIb were examined to achieve further optimization.

Combination of alterations that achieves improvement of selectivity and/or enhancement of binding to FcγRIIb in the examinations carried out so far was introduced into IL6R-B3. As a comparative control, IL6R-BP253 was produced by introducing the S267E and L328F alterations, which are known to enhance binding to FcγRIIb (Seung et al., Mol. Immunol. (2008) 45, 3926-3933), into IL6R-B3. IL6R-L was used for the antibody L chain. An antibody containing the above-mentioned heavy-chain variant and the light chain of IL6R-L, which was expressed according to the method of Reference Example 1, was purified. Binding of the purified antibody to each FcγR (FcγRIa, FcγRIIaH, FcγRIIaR, FcγRIIb, and FcγRIIIaV) was assessed using the method of Reference Example 2.

The KD of each variant for each FcγR is shown in Table 8. “Alteration” in the Table refers to alterations introduced into IL6R-B3. Meanwhile, IL6R-B3/IL6R-L used as a template for producing each of the variants is indicated by an asterisk (*). “Parent polypeptide KD(IIb)/altered polypeptide KD(IIb)” refers to a value obtained by dividing the KD value of IL6R-B3/IL6R-L for FcγRIIb by the KD value of each variant for FcγRIIb. Furthermore, “parent polypeptide KD(IIaR)/altered polypeptide KD(IIaR)” refers to a value obtained by dividing the KD value of IL6R-B3/IL6R-L for FcγR IIaR by the KD of the respective variant for FcγRIIaR. “KD(IIaR)/KD(IIb)” shows a value obtained by dividing the KD of each variant for FcγRIIaR by the KD of the respective variant for FcγRIIb. The larger this value is, the higher the selectivity to FcγRIIb compared to FcγRIIaR is. “KD(IIaH)/KD(IIb)” shows a value obtained by dividing the KD of each variant for FcγRIIaH by the KD of the respective variant for FcγRIIb. The larger this value is, the higher the selectivity to FcγRIIb compared to FcγRIIaH is. The values shown in bold italicized font in Table 8 were calculated using the following equation

KD=C·Rₘₐₓ/(R_{eq}−RI)−C [Equation 2]

shown in Reference Example 2 since the binding of FcγR to IgG was determined to be too weak to correctly analyze by kinetic analysis.

TABLE 8

| VARIANT | | KD (mol/L) | | | | | KD(IIaR)/ | KD(IIaH)/ | KD(IIaR) OF THE PARENT POLYPEPTIDE/ | KD(IIb) OF THE PARENT POLYPEPTIDE/ |
|---|---|---|---|---|---|---|---|---|---|---|
| NAME | ALTERATION | FcγRIa | FcγRIIaR | FcγRIIaH | FcγRIIb | FcγRIIIaV | KD(IIb) | KD(IIb) | KD(IIaR) OF THE ALTERED POLYPEPTIDE | KD(IIb) OF THE ALTERED POLYPEPTIDE |
| G1d | G1d | 3.2E−10 | 1.0E−06 | 6.7E−07 | 2.6E−06 | 3.5E−07 | 0.4 | 0.3 | 1.1 | 1.2 |
| B3 | B3 | 4.2E−10 | 1.1E−06 | 7.7E−07 | 3.1E−06 | 3.3E−07 | 0.3 | 0.2 | 1.0 | 1.0 |
| BP253 | S267E/L328F | 6.7E−11 | 2.1E−09 | 1.2E−06 | 1.1E−08 | 3.6E−06 | 0.2 | 107.1 | 528.8 | 276.8 |
| BP262 | G237D/P238D/H268E/P271G | 1.0E−08 | 2.0E−06 | [symbol] | 1.2E−07 | [symbol] | 17.0 | 375.0 | 0.5 | 25.8 |
| BP264 | E233D/G237D/P238D/H268E/ | 7.4E−09 | 3.5E−07 | [symbol] | 1.2E−08 | [symbol] | 28.3 | 227.6 | 3.2 | 252.0 |
| | P271G/Y296D/A330R | | | | | | | | | |
| BP265 | G237D/P238D/H268E/P271G/ | 2.3E−08 | 6.3E−07 | [symbol] | 1.5E−08 | [symbol] | 41.2 | 789.5 | 1.8 | 203.9 |
| | Y296D/A330R | | | | | | | | | |
| BP266 | E233D/G237D/P238D/H268E/ | 1.4E−08 | 3.2E−07 | [symbol] | 1.8E−08 | [symbol] | 18.0 | 621.5 | 3.4 | 175.1 |
| | P271G/A330R | | | | | | | | | |
| BP268 | E233D/G237D/P238D/H268E/ | 4.5E−09 | 1.8E−06 | [symbol] | 9.2E−08 | [symbol] | 19.6 | 228.3 | 0.6 | 33.7 |
| | P271G/Y296D | | | | | | | | | |
| BP269 | G237D/P238D/H268E/P271G/ | 1.4E−08 | 2.2E−06 | [symbol] | 1.1E−07 | [symbol] | 19.6 | 637.2 | 0.5 | 27.4 |
| | Y296D | | | | | | | | | |
| BP423 | E233D/G237D/P238D/S267A/ | 7.7E−10 | 1.8E−07 | [symbol] | 5.1E−09 | [symbol] | 34.2 | 390.6 | 6.3 | 605.5 |
| | H268E/P271G/A330R | | | | | | | | | |
| BP425 | E233D/G237D/P238D/V266L/ | 4.1E−09 | 2.2E−07 | [symbol] | 9.1E−09 | [symbol] | 23.6 | 1644.7 | 5.1 | 339.9 |
| | S267A/H268E/P271G/A330R | | | | | | | | | |
| BP426 | E233D/G237D/P238D/S267A/ | 1.0E−09 | 1.6E−07 | [symbol] | 5.9E−09 | [symbol] | 27.6 | 8361.8 | 6.8 | 529.0 |
| | H268E/E269D/P271G/A330R | | | | | | | | | |
| BP428 | E233D/G237D/P238D/S267G/ | 4.9E−09 | 3.9E−07 | [symbol] | 1.4E−08 | [symbol] | 28.0 | 3000.0 | 2.8 | 221.4 |
| | H268E/P271G/A330R | | | | | | | | | |
| BP429 | E233D/G237D/P238D/V264I/ | 6.2E−09 | 1.7E−07 | [symbol] | 5.4E−09 | [symbol] | 31.5 | 648.1 | 6.5 | 574.1 |
| | S267G/H268E/P271G/A330R | | | | | | | | | |
| BP430 | E233D/G237D/P238D/V266L/ | 1.7E−08 | 2.2E−07 | [symbol] | 1.2E−08 | [symbol] | 18.5 | 909.1 | 4.9 | 256.2 |
| | S267G/H268E/P271G/A330R | | | | | | | | | |
| BP431 | E233D/G237D/P238D/S267G/ | 3.6E−09 | 4.1E−07 | [symbol] | 1.2E−08 | [symbol] | 34.6 | 649.6 | 2.7 | 265.0 |
| | H268E/E269D/P271G/A330R | | | | | | | | | |
| BP433 | E233D/G237D/P238D/H268D/ | 7.5E−10 | 6.8E−07 | [symbol] | 3.4E−08 | [symbol] | 20.0 | 216.0 | 1.6 | 91.7 |
| | P271G/Y296D/A330K/I332T | | | | | | | | | |
| BP434 | E233D/G237D/P238D/H268D/ | 5.5E−10 | 3.4E−07 | [symbol] | 1.2E−08 | [symbol] | 27.2 | 333.3 | 3.3 | 252.0 |
| | P271G/Y296D/K326D/A330R/ | | | | | | | | | |
| | I332T | | | | | | | | | |
| BP435 | E233D/G237D/P238D/H268D/ | 1.0E−09 | 4.2E−07 | [symbol] | 1.6E−08 | [symbol] | 27.1 | 217.9 | 2.6 | 198.7 |
| | P271G/Y296D/K326A/A330R/ | | | | | | | | | |
| | I332T | | | | | | | | | |
| BP436 | E233D/G237D/P238D/S267A/ | 2.6E−10 | 2.2E−07 | 2.1E−06 | 5.1E−09 | [symbol] | 43.8 | 411.0 | 4.9 | 606.7 |
| | H268E/P271G/Y296D/A330R/ | | | | | | | | | |
| | I332T | | | | | | | | | |
| BP437 | G237D/P238D/S267A/H268E/ | 7.5E−10 | 2.2E−07 | [symbol] | 5.9E−09 | [symbol] | 37.7 | 236.5 | 4.9 | 523.6 |
| | P271G/Y296D/A330R/I332T | | | | | | | | | |
| BP438 | E233D/G237D/P238D/S267A/ | 2.1E−10 | 1.8E−07 | 1.6E−06 | 5.5E−09 | [symbol] | 32.7 | 293.6 | 6.2 | 568.8 |
| | H268E/P271G/A330R/I332T | | | | | | | | | |
| BP439 | E233D/G237D/P238D/V264I/ | 8.7E−09 | 1.3E−07 | [symbol] | 6.1E−09 | [symbol] | 20.9 | 460.5 | 8.7 | 509.9 |
| | V266L/S267A/H268E/P271G/ | | | | | | | | | |
| | A330R | | | | | | | | | |
| BP440 | E233D/G237D/P238D/V264I/ | 8.7E−09 | 1.3E−07 | [symbol] | 5.2E−09 | [symbol] | 24.0 | 307.1 | 8.8 | 595.0 |
| | H268E/P271G/A330R | | | | | | | | | |
| BP441 | E233D/G237D/P238D/V266L/ | 1.7E−08 | 3.6E−07 | [symbol] | 1.5E−08 | [symbol] | 24.0 | 582.8 | 3.0 | 205.3 |
| | H268E/P271G/A330R | | | | | | | | | |
| BP442 | E233D/G237D/P238D/H268E/ | 4.5E−09 | 3.8E−07 | [symbol] | 1.2E−08 | [symbol] | 30.6 | 379.0 | 2.9 | 250.0 |
| | E269D/P271G/A330R | | | | | | | | | |
| BP443 | E233D/G237D/P238D/V266L/ | 1.8E−08 | 5.1E−07 | [symbol] | 2.3E−08 | [symbol] | 21.7 | 406.0 | 2.2 | 132.5 |
| | H268E/E269D/P271G/A330R | | | | | | | | | |
| BP445 | E233D/G237D/P238D/V264I/ | 2.0E−09 | 8.0E−08 | [symbol] | 2.6E−09 | [symbol] | 31.0 | 581.4 | 13.8 | 1201.6 |
| | S267A/H268E/P271G/A330R | | | | | | | | | |
| BP479 | E233D/G237D/P2380D/V264I/ | 5.3E−09 | 9.0E−07 | [symbol] | 5.6E−08 | [symbol] | 16.1 | 268.3 | 1.2 | 55.5 |
| | V266L/S267A/H268E/P271G | | | | | | | | | |
| BP480 | E233D/G237D/P238D/V266L/ | 1.3E−08 | 6.3E−06 | [symbol] | 2.0E−07 | [symbol] | 32.1 | 107.7 | 0.2 | 15.9 |
| | H268E/E269D/P271G | | | | | | | | | |
| BP481 | E233D/G237D/P238D/V264I/ | 1.0E−09 | 4.0E−07 | [symbol] | 1.9E−08 | [symbol] | 20.5 | 350.5 | 2.8 | 159.8 |
| | S267A/H268E/P271G | | | | | | | | | |
| BP483 | E233D/6237D/P238D/V266L/ | 1.3E−09 | 1.3E−06 | [symbol] | 7.8E−08 | [symbol] | 16.8 | 230.8 | 0.8 | 39.7 |
| | S267A/H268E/P271G | | | | | | | | | |
| BP484 | E233D/G237D/P238D/S267A/ | 8.2E−10 | 7.8E−07 | [symbol] | 4.6E−08 | [symbol] | 17.1 | 240.7 | 1.4 | 67.8 |
| | H268E/E269D/P271G | | | | | | | | | |
| BP487 | E233D/G237D/P238D/V264I/ | 1.2E−09 | 3.9E−08 | 8.4E−07 | 1.2E−09 | [symbol] | 33.8 | 730.4 | 28.3 | 2695.7 |
| | S267A/H268E/P271G/A330R/ | | | | | | | | | |
| | P396M | | | | | | | | | |
| BP488 | E233D/G237D/P238D/V264I/ | 2.2E−09 | 7.4E−08 | 1.6E−06 | 1.9E−09 | [symbol] | 40.1 | 864.9 | 14.8 | 1675.7 |
| | S267A/H268E/P271G/Y296D/ | | | | | | | | | |
| | A330R | | | | | | | | | |
| BP489 | E233D/G237D/P238D/V264I/ | 1.3E−09 | 4.3E−08 | 8.7E−07 | 1.0E−09 | [symbol] | 42.8 | 870.0 | 25.7 | 3100.0 |
| | S267A/H268E/P271G/Y296D/ | | | | | | | | | |
| | A330R/P396M | | | | | | | | | |
| BP490 | G237D/P238D/V264I/S267A/ | 4.5E−09 | 1.1E−07 | 2.4E−06 | 2.4E−09 | [symbol] | 46.7 | 1000.0 | 9.8 | 1291.7 |
| | H268E/P271G/A330R | | | | | | | | | |
| BP491 | G237D/P238D/V264I/S267A/ | 5.3E−09 | 1.2E−07 | 2.2E−06 | 3.0E−09 | [symbol] | 38.8 | 723.7 | 9.3 | 1019.7 |
| | H268E/P271G/Y296D/A330R | | | | | | | | | |
| BP492 | P238D/V264I/S267A/H268E/ | 7.9E−10 | 9.2E−07 | [symbol] | 2.4E−08 | [symbol] | 38.8 | 678.0 | 1.2 | 131.4 |
| | P271G | | | | | | | | | |
| BP493 | P238D/V264I/S267A/H268E/ | 8.2E−10 | 1.1E−06 | [symbol] | 2.1E−08 | [symbol] | 52.1 | 900.5 | 1.0 | 146.9 |
| | P271G/Y296D | | | | | | | | | |
| BP494 | G237D/P238D/S267A/H268E/ | 3.9E−09 | 2.5E−07 | [symbol] | 6.6E−09 | [symbol] | 38.6 | 820.7 | 4.3 | 471.1 |
| | P271G/Y296D/A330R | | | | | | | | | |
| BP495 | G237D/P238D/S267G/H268E/ | 8.3E−09 | 4.9E−07 | [symbol] | 9.7E−09 | [symbol] | 50.9 | 1243.5 | 2.2 | 321.2 |
| | P271G/Y296D/A330R | | | | | | | | | |
| BP496 | E233D/G237D/P238D/V264I/ | 1.2E−09 | 4.7E−07 | 3.7E−06 | 1.8E−08 | [symbol] | 25.5 | 201.1 | 2.3 | 168.5 |
| | S267A/H268E/P271G/Y296D | | | | | | | | | |
| BP497 | E233D/G237D/P238D/V264I/ | 2.1E−09 | 8.5E−08 | 9.6E−07 | 4.1E−09 | [symbol] | 21.0 | 236.5 | 12.9 | 763.5 |
| | S267A/H268E/P271G/A327G/ | | | | | | | | | |
| | A330R | | | | | | | | | |
| BP498 | E233D/G237D/P238D/V264I/ | 1.3E−09 | 5.1E−08 | 9.3E−07 | 1.7E−09 | [symbol] | 30.8 | 563.6 | 21.7 | 1878.8 |
| | S267A/H268E/P271G/A330R/ | | | | | | | | | |
| | P396L | | | | | | | | | |
| BP499 | E233D/G237D/P238D/V264I/ | 1.2E−09 | 4.9E−08 | 1.0E−06 | 1.5E−09 | [symbol] | 33.8 | 684.9 | 22.3 | 2123.3 |
| | S267A/H268E/P271G/Y296D/ | | | | | | | | | |
| | A330R/P396L | | | | | | | | | |
| BP500 | G237D/P238D/V264I/S267A/ | 2.3E−09 | 7.2E−07 | [symbol] | 2.4E−08 | [symbol] | 29.9 | 1033.1 | 1.5 | 128.1 |
| | H268E/P271G/Y296D | | | | | | | | | |
| BP501 | G237D/P238D/V264I/S267A/ | 2.1E−09 | 6.3E−07 | [symbol] | 2.5E−08 | [symbol] | 25.1 | 555.6 | 1.7 | 123.0 |
| | H268E/P271G | | | | | | | | | |
| BP502 | E233D/G237D/P238D/V264I/ | 2.1E−09 | 1.1E−07 | 1.3E−06 | 3.7E−09 | [symbol] | 29.5 | 352.3 | 10.1 | 840.1 |
| | S267A/H268E/P271G/Y296D/ | | | | | | | | | |
| | A327G/A330R | | | | | | | | | |
| BP503 | E233D/G237D/P2380D/V264I/ | 1.2E−09 | 5.7E−08 | 8.6E−07 | 1.7E−09 | [symbol] | 33.2 | 502.9 | 19.4 | 1812.9 |
| | S267A/H268E/P271G/Y296D/ | | | | | | | | | |
| | A327G/A330R/P396M | | | | | | | | | |
| BP504 | E233D/G237D/P238D/V264I/ | 1.4E−09 | 4.5E−07 | [symbol] | 2.4E−08 | [symbol] | 18.5 | 658.4 | 2.4 | 127.6 |
| | S267A/H268E/P271G/E272P | | | | | | | | | |
| BP505 | E233D/G237D/P238D/V264I/ | 1.1E−09 | 4.3E−07 | [symbol] | 2.1E−08 | [symbol] | 20.0 | 514.0 | 2.6 | 144.9 |
| | S267A/H268E/P271G/E272D | | | | | | | | | |
| BP506 | E233D/G237D/P238D/V264I/ | 3.1E−09 | 1.2E−07 | 2.5E−06 | 3.4E−09 | [symbol] | 35.1 | 731.0 | 9.2 | 906.4 |
| | S267A/H268E/P271G/E272P/ | | | | | | | | | |
| | Y296D/A330R | | | | | | | | | |
| BP507 | E233D/G237D/P238D/V264I/ | 2.6E−09 | 1.0E−07 | 1.8E−06 | 2.9E−09 | [symbol] | 34.2 | 618.6 | 11.1 | 1065.3 |
| | S267A/H268E/P271G/E272P/ | | | | | | | | | |
| | A330R | | | | | | | | | |
| BP508 | E233D/G237D/P238D/V264I/ | 1.4E−09 | 5.4E−07 | [symbol] | 2.1E−08 | [symbol] | 26.0 | 961.5 | 2.0 | 149.0 |
| | S267A/H268E/P271G/E272P/ | | | | | | | | | |
| | Y296D | | | | | | | | | |
| BP509 | E233D/G237D/P238D/V264I/ | 1.1E−09 | 5.2E−07 | [symbol] | 1.8E−08 | [symbol] | 29.2 | 443.8 | 2.1 | 174.2 |
| | S267A/H268E/P271G/E272D/ | | | | | | | | | |
| | Y296D | | | | | | | | | |
| BP510 | G237D/P238D/V264I/S267A/ | 6.0E−09 | 1.7E−07 | 4.0E−06 | 3.8E−09 | [symbol] | 43.5 | 1041.7 | 6.6 | 807.3 |
| | H268E/P271G/E272P/A330R | | | | | | | | | |
| BP511 | G237D/P238D/V264I/S267A/ | 6.0E−09 | 1.8E−07 | 4.3E−06 | 3.5E−09 | [symbol] | 50.6 | 1235.6 | 6.3 | 890.8 |
| | H268E/P271G/E272P/Y296D/ | | | | | | | | | |
| | A330R | | | | | | | | | |
| BP531 | E233D/G237D/P238D/V264I/ | 9.4E−09 | 1.2E−07 | 3.5E−06 | 3.8E−09 | [symbol] | 33.1 | 933.3 | 8.9 | 826.7 |
| | S267G/H268E/P271G/Y296D/ | | | | | | | | | |
| | A330R/P396M | | | | | | | | | |
| BP532 | E233D/G237D/P238D/V264I/ | 1.2E−08 | 9.4E−08 | 1.9E−06 | 3.2E−09 | [symbol] | 29.3 | 593.8 | 11.7 | 968.8 |
| | H268E/P271G/Y296D/A330R/ | | | | | | | | | |
| | P396M | | | | | | | | | |
| BP533 | E233D/G237D/P238D/V264I/ | 7.7E−09 | 1.2E−07 | 2.6E−06 | 4.1E−09 | [symbol] | 29.3 | 634.1 | 9.2 | 756.1 |
| | S267G/H268E/P271G/Y296D/ | | | | | | | | | |
| | A330R/P396L | | | | | | | | | |
| BP534 | E233D/G237D/P238D/V264I/ | 9.3E−09 | 9.1E−08 | 1.8E−06 | 3.0E−09 | [symbol] | 30.7 | 606.1 | 12.1 | 1043.8 |
| | H268E/P271G/Y296D/A330R/ | | | | | | | | | |
| | P396L | | | | | | | | | |
| BP535 | E233D/G237D/P238D/V264I/ | 1.1E−08 | 9.2E−08 | 3.2E−06 | 4.0E−09 | [symbol] | 23.2 | 806.0 | 11.9 | 780.9 |
| | S267G/H268E/P271G/Y296D/ | | | | | | | | | |
| | A327G/A330R/P396M | | | | | | | | | |
| BP536 | E233D/G237D/P238D/V264I/ | 8.9E−09 | 7.9E−08 | 1.3E−06 | 3.0E−09 | [symbol] | 26.6 | 437.7 | 13.9 | 1043.8 |
| | H268E/P271G/Y296D/A327G/ | | | | | | | | | |
| | A330R/P396M | | | | | | | | | |
| BP537 | G237D/P238D/V264I/S267G/ | 2.9E−08 | 2.7E−07 | 3.1E−06 | 6.9E−09 | [symbol] | 39.1 | 447.3 | 4.1 | 447.3 |
| | H268E/P271G/A330R | | | | | | | | | |
| BP538 | G237D/P238D/V264I/H268E/ | 5.5E−08 | 2.0E−07 | 3.0E−06 | 5.3E−09 | [symbol] | 38.6 | 568.2 | 5.4 | 587.1 |
| | P271G/A330R | | | | | | | | | |
| BP539 | G237D/P238D/V264I/S267G/ | 6.4E−08 | 3.3E−07 | 5.6E−06 | 8.4E−09 | [symbol] | 39.0 | 666.7 | 3.4 | 369.0 |
| | H268E/P271G/E272P/Y296D/ | | | | | | | | | |
| | A330R | | | | | | | | | |
| BP540 | G237D/P238D/V264I/H268E/ | 9.6E−08 | 2.1E−07 | 4.6E−06 | 5.7E−09 | [symbol] | 36.6 | 802.8 | 5.2 | 541.0 |
| | P271G/E272P/Y296D/A330R | | | | | | | | | |
| BP549 | G237D/P238D/S267G/H268E/ | 1.8E−08 | 5.7E−07 | [symbol] | 1.6E−08 | [symbol] | 35.9 | 696.2 | 1.9 | 196.2 |
| | P271G/A330R | | | | | | | | | |
| BP550 | G237D/P238D/V264I/S267G/ | 2.5E−08 | 3.4E−07 | 5.0E−06 | 7.6E−09 | [symbol] | 44.2 | 655.3 | 3.3 | 406.3 |
| | H268E/P271G/E272D/Y296D/ | | | | | | | | | |
| | A330R | | | | | | | | | |
| BP551 | G237D/P238D/V264I/H268E/ | 3.2E−08 | 2.5E−07 | 2.8E−06 | 6.4E−09 | [symbol] | 38.1 | 435.5 | 4.5 | 482.1 |
| | P271G/E272D/Y296D/A330R | | | | | | | | | |
| BP552 | E233D/G237D/P238D/V264I/ | 3.2E−09 | 9.7E−08 | 1.9E−06 | 2.6E−09 | [symbol] | 37.3 | 733.6 | 11.4 | 1196.9 |
| | S267A/H268E/P271G/E272D/ | | | | | | | | | |
| | Y296D/A330R | | | | | | | | | |
| BP553 | E233D/G237D/P238D/V264I/ | 3.4E−09 | 8.6E−08 | 1.4E−06 | 3.1E−09 | [symbol] | 27.8 | 453.1 | 12.8 | 1003.2 |
| | S267A/H268E/P271G/E272D/ | | | | | | | | | |
| | A330R | | | | | | | | | |
| BP554 | G237D/P238D/V264I/S267A/ | 8.0E−09 | 1.5E−07 | 2.3E−06 | 4.4E−09 | [symbol] | 32.7 | 518.0 | 7.6 | 698.2 |
| | H268E/P271G/E272D/A330R | | | | | | | | | |
| BP555 | G237D/P238D/V264I/S267A/ | 9.4E−09 | 1.6E−07 | 3.2E−06 | 4.1E−09 | [symbol] | 39.7 | 778.6 | 6.7 | 754.3 |
| | H268E/P271G/E272D/Y296D/ | | | | | | | | | |
| | A330R | | | | | | | | | |
| BP556 | G237D/P238D/V264I/S267G/ | 4.3E−08 | 3.0E−07 | 5.8E−06 | 8.4E−09 | [symbol] | 35.4 | 692.1 | 3.7 | 369.9 |
| | H268E/P271G/Y296D/A330R | | | | | | | | | |
| BP557 | G237D/P238D/S267G/H268D/ | 1.3E−08 | 8.5E−07 | [symbol] | 2.0E−08 | [symbol] | 42.0 | 746.3 | 1.3 | 154.2 |
| | P271G/Y296D/A330R | | | | | | | | | |
| BP558 | G237D/P238D/V264I/S267G/ | 1.3E−08 | 3.3E−07 | 4.9E−06 | 9.0E−09 | [symbol] | 36.4 | 543.2 | 3.4 | 343.7 |
| | H268E/P271G/E272D/A330R | | | | | | | | | |
| BP559 | P238D/V264I/S267A/H268E/ | 1.1E−09 | 1.6E−06 | [symbol] | 2.8E−08 | [symbol] | 58.4 | 711.7 | 0.7 | 110.3 |
| | P271G/E272D/Y296D | | | | | | | | | |
| BP560 | P238D/S267G/H268E/P271G/ | 5.6E−09 | 4.2E−06 | [symbol] | 1.8E−07 | [symbol] | 22.8 | 168.5 | 0.3 | 16.8 |
| | Y296D/A330R | | | | | | | | | |
| BP561 | E233D/G237D/P238D/H268D/ | 9.4E−09 | 5.1E−07 | 5.3E−06 | 1.8E−08 | [symbol] | 28.0 | 291.2 | 2.2 | 170.3 |
| | P271G/E272D/Y296D/A330R | | | | | | | | | |
| BP562 | G237D/P238D/H268D/P271G/ | 2.5E−08 | 6.8E−07 | [symbol] | 2.4E−08 | [symbol] | 29.0 | 466.1 | 1.6 | 131.4 |
| | E272D/Y296D/A330R | | | | | | | | | |
| BP563 | E233D/G237D/P238D/H268E/ | 1.2E−08 | 4.6E−07 | 8.3E−06 | 1.6E−08 | [symbol] | 29.1 | 525.3 | 2.4 | 196.2 |
| | P271G/E272D/Y296D/A330R | | | | | | | | | |
| BP564 | G237D/P238D/H268E/P271G/ | 3.1E−08 | 5.8E−07 | [symbol] | 2.2E−08 | [symbol] | 26.2 | 454.5 | 1.9 | 140.9 |
| | E272D/Y296D/A330R | | | | | | | | | |
| BP565 | E233D/G237D/P238D/S267A/ | 2.4E−09 | 2.3E−07 | 4.7E−06 | 5.5E−09 | [symbol] | 41.5 | 856.1 | 4.8 | 564.7 |
| | H268E/P271G/Y296D/A330R | | | | | | | | | |
| BP567 | E233D/P238D/V264I/S267A/ | 2.1E−10 | 8.9E−07 | [symbol] | 1.4E−08 | [symbol] | 64.4 | 1231.9 | 1.2 | 224.6 |
| | H268E/P271G/Y296D | | | | | | | | | |
| BP568 | E233D/P238D/V264I/S267A/ | 1.9E−10 | 6.8E−07 | [symbol] | 1.5E−08 | [symbol] | 46.1 | 748.3 | 1.6 | 210.9 |
| | H268E/P271G | | | | | | | | | |

[symbol]

indicates data missing or illegible when filed

Among the variants described in Table 8, IL6R-BP253/IL6R-L produced by adding known alterations that enhance FcγRIIb binding showed 277-fold and 529-fold enhanced binding activities to FcγRIIb and FcγRIIaR, respectively, compared to those of the IL6R-B3/IL6R-L prior to alteration. Furthermore, FcγRIa-binding activity of IL6R-BP253/IL6R-L was also enhanced compared to that of IL6R-B3/IL6R-L. On the other hand, binding of IL6R-BP253/IL6R-L to FcγRIIaH and FcγRIIIaV was decreased compared to those of IL6R-B3/IL6R-L. Among the other variants, binding to FcγRIa was slightly enhanced for IL6R-BP436/IL6R-L, IL6R-BP438/IL6R-L, IL6R-BP567/IL6R-L, and IL6R-BP568/IL6R-L, compared to that of the IL6R-B3/IL6R-L prior to alteration but FcγRIa binding was decreased in all of the other variants. Furthermore, binding to FcγRIIaH and FcγRIIIaV were decreased in all variants when compared to those of IL6R-B3/IL6R-L.

Comparison of variants produced in this examination with the existing variant IL6R-BP253/IL6R-L having enhanced FcγRIIb binding showed that the value of KD(IIaH)/KD(IIb) is 107.7 for IL6R-BP480/IL6R-L which showed the lowest value and is 8362 for IL6R-BP426/IL6R-L which showed the highest value, and the values for all variants were higher than 107.1 for IL6R-BP253/IL6R-L. Furthermore, the value of KD(IIaR)/KD(IIb) is 16.1 for IL6R-BP479/IL6R-L which showed the lowest value and is 64.4 for IL6R-BP567/IL6R-L which showed the highest value, and the values for all variants were higher than 0.2 for IL6R-BP253/IL6R-L. From these results, all of the variants shown in Table 8 have been shown to be variants with improved selectivity to FcγRIIb as compared to the existing variant into which alteration(s) to enhance FcγRIIb binding is introduced. In particular, IL6R-BP559/IL6R-L, IL6R-BP493/IL6R-L, IL6R-BP557/IL6R-L, IL6R-BP492/IL6R-L, IL6R-BP500/IL6R-L, and IL6R-BP567/IL6R-L all have FcγRIIaR binding maintained at not more than 1.5 times that of IL6R-B3/IL6R-L, and at the same time FcγRIIb-binding activity enhanced by 100 times or more; therefore, these variants were expected to show effects yielded by enhanced binding to FcγRIIb while avoiding side effects caused by enhanced binding to FcγRIIaR.

In addition, regarding IL6R-BP489/IL6R-L, IL6R-BP487/IL6R-L, IL6R-BP499/IL6R-L, IL6R-BP498/IL6R-L, IL6R-BP503/IL6R-L, IL6R-BP488/IL6R-L, IL6R-BP490/IL6R-L, IL6R-BP445/IL6R-L, IL6R-BP552/IL6R-L, IL6R-BP507/IL6R-L, IL6R-BP536/IL6R-L, IL6R-BP534/IL6R-L, IL6R-BP491/IL6R-L, IL6R-BP553/IL6R-L, IL6R-BP532/IL6R-L, IL6R-BP506/IL6R-L, IL6R-BP511/IL6R-L, IL6R-BP502/IL6R-L, IL6R-BP531/IL6R-L, IL6R-BP510/IL6R-L, IL6R-BP535/IL6R-L, IL6R-BP497/IL6R-L, IL6R-BP533/IL6R-L, IL6R-BP555/IL6R-L, IL6R-BP554/IL6R-L, IL6R-BP436/IL6R-L, IL6R-BP423/IL6R-L, IL6R-BP440/IL6R-L, IL6R-BP538/IL6R-L, IL6R-BP429/IL6R-L, IL6R-BP438/IL6R-L, IL6R-BP565/IL6R-L, IL6R-BP540/IL6R-L, IL6R-BP426/IL6R-L, IL6R-BP437/IL6R-L, IL6R-BP439/IL6R-L, IL6R-BP551/IL6R-L, IL6R-BP494/IL6R-L, IL6R-BP537/IL6R-L, IL6R-BP550/IL6R-L, IL6R-BP556/IL6R-L, IL6R-BP539/IL6R-L, IL6R-BP558/IL6R-L, IL6R-BP425/IL6R-L, and IL6R-BP495/IL6R-L, their FcγRIIb binding was higher than that of IL6R-BP253/IL6R-L added with the existing alteration that enhances the FcγRIIb binding. Further, the enhancement of the FcγRIIb binding ranges from 321 times (lowest) to 3100 times (highest), compared to the binding of IL6R-B3/IL6R-L (which is defined to be 1), from IL6R-BP495/IL6R-L to IL6R-BP489/IL6R-L, respectively. Therefore, one may say that these variants are superior to existing technology with regard to both selectivity and binding to FcγRIIb.

Here, variants related to IL6R-BP567/IL6R-L which is considered to be the best in terms of selectivity for FcγRIIb were studied from the perspective of immunogenicity. The Y296D alteration has been introduced into IL6R-BP567/IL6R-L which showed the highest selectivity and into IL6R-BP493/IL6R-L which showed FcγRIIaR binding that is completely equivalent to that of the native form and showed 147-fold enhanced binding to FcγRIIb. Y296 has been reported to be included in a Tregitope sequence (De Groot et al., Blood (2008) 112, 3303-3311), and introducing alterations into this site may lead to loss of immunosuppressive functions that a native IgG1 normally have. Therefore, from the perspective of immunogenicity, variants that do not include the Y296D alteration are more preferred. IL6R-BP568/IL6R-L and IL6R-BP492/IL6R-L were produced by removing the Y296D alteration from IL6R-BP567/IL6R-L and IL6R-BP493/IL6R-L, respectively. Considering the selectivity and binding activity to FcγRIIb, removing the Y296D alteration from IL6R-BP492/IL6R-L and IL6R-BP568/IL6R-L decreased both selectivity and binding activity compared to when Y296D was included. However, compared to the native form, binding to FcγRIIaR is 1.6-fold and binding to FcγRIIb is 211-fold for IL6R-BP568/IL6R-L, and binding to FcγRIIaR is 1.2-fold and binding to FcγRIIb is 131-fold for IL6R-BP492/IL6R-L, and therefore high selectivity and binding activity were still maintained. These results allow one to say that IL6R-BP568/IL6R-L and IL6R-BP492/IL6R-L are excellent variants not only in terms of selectivity and binding activity to FcγRIIb but also in terms of immunogenicity.

### [Example 9] Enhancement of Binding to FcgRIIb by a Heterodimerized Antibody

9-1. Examination of Introducing P238D into Only One of the Chains

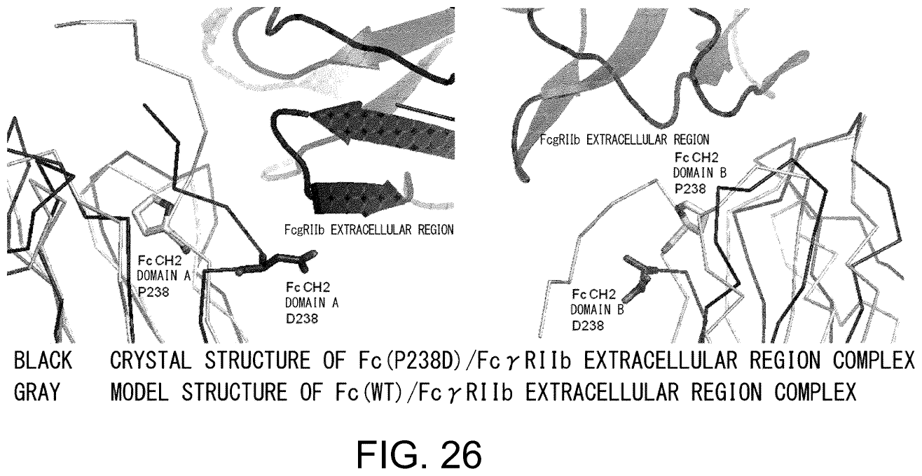

As shown in FIG. 26 in Reference Example 7, the reason why Fc(P238D) acquired high binding to FcgRIIb is by introducing the P238D alteration, the region that had formed a hydrophobic core with the surrounding residues in the case of Pro could no longer exist in the hydrophobic core upon change to Asp and directed to the solvent side, and resulting in the great change in the loop structure of domain A. However, whether it is necessary to introduce the P238D alteration into both chains or whether it is acceptable to introduce P238D into one of the chains and introduce other alterations into the other chain still has to be verified. Accordingly, heterodimerized antibodies with different alterations introduced into each of the antibody H chains were used for this verification.

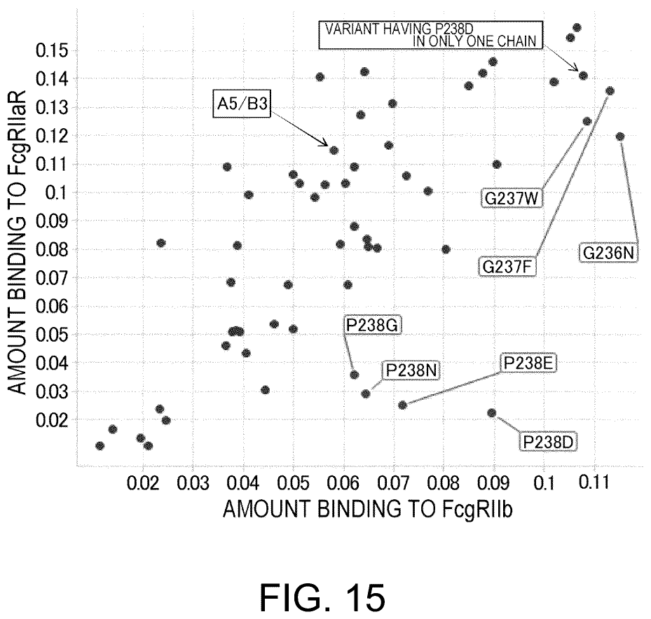

The variable region (SEQ ID NO: 15) of a glypican 3 antibody comprising the CDRs of GpH7 which is an anti-glypican 3 antibody with improved plasma kinetics disclosed in WO 2009/041062 was used as the antibody H chain. GpH7-A5 (SEQ ID NO: 35) produced by introducing the D356K and H435R alterations into GpH7-G1d (SEQ ID NO: 34) in which Gly and Lys are removed from the C terminus of IgG1 carrying GpH7 as the variable region, and GpH7-B3 (SEQ ID NO: 17) produced by introducing the K439E alteration into GpH7-G1d were used. The D356K and K439E alterations introduced into the respective H chains were introduced to efficiently form the heterodimers for each H chain when producing heterodimerized antibodies comprising two H chains (WO2006/106905). H435R is an alteration that inhibits binding to Protein A, and was introduced to efficiently separate the dimeric heteromer comprising two H chains each introduced with different alterations from the dimeric homomer comprising two H chains each introduced with the same alterations. Variants in which the amino acids at positions 236, 237, and 238 (EU numbering) in GpH7-B3 (SEQ ID NO: 17) produced in Reference Example 3 were substituted with any of the 18 amino acids other than the original amino acid and Cys were used as one of the H chains. GpH7-AP001 produced by introducing P238D into GpH7-A5 (SEQ ID NO: 35) was used as the other chain. GpL16-k0 (SEQ ID NO: 16) of glypican 3 antibody with improved plasma kinetics disclosed in WO 2009/041062 was commonly used as the antibody L chain. These variants were expressed and purified according to the method of Reference Example 1, and binding to each of the FcgRIIa type R and FcgRIIb was assessed using the method of Reference Example 2. The amount binding to FcgR of each variant is shown in FIG. 15.

The G237W, G237F, G236N, P238G, P238N, P238E, and P238D alterations shown in FIG. 15 refer to alterations introduced into GpH7-B3. A5/B3 refers to GpH7-A5/GpH7-B3/GpL16-k0 which has no alterations introduced into both chains, and a variant containing P238D in only one of the chains refers to GpH7-A5/GpH7-BF648/GpL16-k0. Their results are shown in Table 9.

TABLE 9

| | | | BINDING | BINDING | |
|---|---|---|---|---|---|
| | | | AMOUNT OF | AMOUNT OF | |
| | | | ALTERED | ALTERED | |
| | | | POLYPEPTIDE | POLYPEPTIDE | |
| | | | TO FcgRIIaR | TO FcgRIIb | AMOUNT |
| | | | WHEN THAT OF | WHEN THAT OF | BINDING |
| | | | GpH7-AP001/ | GpH7-AP001/ | TO FcgRIIb/ |
| | ALTERATION | ALTERATION | GpH7-BF-648 | GpH7-BF648 | AMOUNT |
| | INTRODUCED | INTRODUCED | IS DEFINED | IS DEFINED | BINDING |
| VARIANT NAME | INTO GpH7-A5 | INTO GpH7-B3 | AS 100 | AS 100 | TO FcgRIIaR |
| GpH7-G1d/GpL16-k0 | * | * | 515 | 65 | 0.5 |
| GpH7-A5/GpH7-B3/GpL16-k0 | | | 570 | 77 | 0.5 |
| GpH7-A5/GpH7-BF648/GpL16-k0 | | P238D | 677 | 131 | 0.8 |
| GpH7-AP001/GpH7-BF648/GpL16-k0 | P238D | P238D | 100 | 100 | 4.0 |
| GpH7-AP001/GpH7-BP032/GpL16-k0 | P238D | G236N | 538 | 129 | 1.0 |
| GpH7-AP001/GpH7-BP044/GpL16-k0 | P238D | G237F | 609 | 127 | 0.8 |
| GpH7-AP001/GpH7-BP057/GpL16-k0 | P238D | G237W | 561 | 121 | 0.9 |
| GpH7-AP001/GpH7-BP061/GpL16-k0 | P238D | P238E | 111 | 80 | 2.9 |
| GpH7-AP001/GpH7-BP063/GpL16-k0 | P238D | P238G | 161 | 69 | 1.7 |
| GpH7-AP001/GpH7-BP069/GpL16-k0 | P238D | P238N | 131 | 72 | 2.2 |

In Table 9, “amount binding to FcgRIIb/amount binding to FcgRIIaR” are values obtained by dividing the amount binding to FcgRIIb of each variant by the amount binding to FcgRIIaR of each variant, and shows that the higher the value is, the higher selectivity to FcgRIIb is. Furthermore, while the phrases “alteration introduced into GpH7-A5” and “alteration introduced into GpH7-B3” indicate alterations introduced into GpH7-A5 and GpH7-B3, respectively; and GpH7-G1d which was used as the template when producing GpH7-A5 and GpH7-B3 is indicated by an asterisk (*). From the results of Table 9, GpH7-AP001/GpH7-BF648/GpL16-k0 having the P238D alteration in both chains had the highest selectivity to FcgRIIb. GpH7-AP001/GpH7-BP061/GpL16-k0, GpH7-AP001/GpH7-BP069/GpL16-k0, and GpH7-AP001/GpH7-BP063/GpL16-k0 which have P238E, P238N, and P238G in the other chain show the values of “amount binding to FcgRIIb/amount binding to FcgRIIaR” that are 2.9, 2.2, and 1.7, respectively, and even when compared to GpH7-AP001/GpH7-BF648/GpL16-k0 which has the P238D alteration in both chains, high selectivity to FcgRIIb was maintained. Furthermore, since GpH7-AP001/GpH7-BF648/GpL16-k0 having the P238D alteration in both chains also maintained 69% or greater affinity for FcgRIIb, one can say that if the P238D alteration is present in one of the chains, the other chain may have the P238E, P238N, or P238G alteration. In addition, focusing on FcgRIIb-binding, compared to GpH7-AP001/GpH7-BF648/GpL16-k0 having P238D in both chains, GpH7-A5/GpH7-BF648/GpL16-k0 which has P238D only in one of the chains and does not have alteration in the other chain showed stronger binding to FcgRIIb; and GpH7-AP001/GpH7-BP032/GpL16-k0, GpH7-AP001/GpH7-BP044/GpL16-k0, and GpH7-AP001/GpH7-BP057/GpL16-k0 which have P238D in one of their chains and have G236N, G237F, and G237W, respectively, in the other chain were found to bind more strongly to FcgRIIb.

### 9-2. Verification of Alterations Based on the Structural Information of Fc(P208)/FcgRIIb

As shown in FIG. 10, in the crystal structure of Fc(P208)/FcgRIIb, electron density of Lys at position 117 of FcgRIIb was not observed, and this residue was considered not to be largely involved in binding with Fc(P208); but by substituting Asp or Glu for Ser at position 239 (EU numbering) of the CH2 domain B, which is positioned nearby, an electrostatic interaction may be formed with this Lys at position 117 of FcgRIIb. On the other hand, as shown in FIG. 7, in CH2 domain A, Ser at position 239 (EU numbering) forms a hydrogen bond with Gly at position 236 (EU numbering), and stabilization of the loop structure from positions 233 to 239 (EU numbering) may contribute to strengthen the binding with Tyr at position 160 (EU numbering), and substitutions in this portion is predicted to cause decrease in binding activity accompanying destabilization of the loop structure in CH2 domain A, and these effects were predicted to cancel each other in homologous alterations. Accordingly, in this examination, the S239D alteration or the S239E alteration was introduced only into one of the chains by heterodimerization, and effects of enhancement of binding to FcgRIIb were examined.

As one of the antibody H chains, IL6R-BP256 and IL6R-BP257 were produced by introducing S239D and S239E, respectively, into IL6R-BP208 (SEQ ID NO: 24). Similarly, S239D was introduced into IL6R-BP230 (SEQ ID NO: 27) to produce IL6R-BP259, and S239E was introduced into IL6R-BP230 to produce IL6R-BP260. IL6R-AP002 was produced by introducing the same alterations as those included in CH2 of IL6R-BP208, which are E233D, G237D, P238D, H268D, P271G, and A330R, into IL6R-A5 (SEQ ID NO: 69), and IL6R-AP009 was produced by introducing the same alterations as those included in CH2 of IL6R-BP230, which are E233D, G237D, P238D, H268D, P271G, Y296D, and A330R, into IL6R-A5, and they were used as the other antibody H chain. As a comparison, IL6R-BP253 (SEQ ID NO: 32) was produced by introducing S267E and L328F, which is a known FcgRIIb enhancement technique (Non-patent Document 28), into IL6R-B3. IL6R-L (SEQ ID NO: 21) was commonly used as the antibody L chain. Antibodies were expressed using these variants and then purified according to the method of Reference Example 1, and binding to each FcgR (FcgRIa, FcgRIIa type H, FcgRIIa type R, FcgRIIb, and FcgRIIIa type V) was assessed using the method of Reference Example 2.

The KD values of each variant for each FcgR are shown in Table 10. “Parent polypeptide KD(IIb)/altered polypeptide KD(IIb)” refers to a value obtained by dividing the KD value of IL6R-B3/IL6R-L for FcgRIIb by the KD value of each variant for FcgRIIb. Furthermore, “parent polypeptide KD(IIaR)/altered polypeptide KD(IIaR)” refers to a value obtained by dividing the KD value of IL6R-B3/IL6R-L for FcgRIIaR by the KD value of each variant for FcgRIIaR. “KD(IIaR)/KD(IIb)” is a value obtained by dividing the KD of each variant for FcgRIIaR by the KD of each variant for FcgRIIb. The larger this value is, the higher the selectivity to FcgRIIb is. The Table 10 values shown in bold italicized font were_calculated using the following equation

KD=C·Rₘₐₓ/(R_{eq}−RI)−C [Equation 2]

shown in Reference Example 2 since the binding of FcgR to IgG was too weak to accurately analyze by kinetic analysis.

TABLE 10

| | | | | | | | KD(IIaR) OF | KD(IIb) OF |
|---|---|---|---|---|---|---|---|---|
| | | | | | | | PARENT | PARENT |
| | | | | | | | POLYPEPTIDE/ | POLYPEPTIDE/ |
| | KD FOR | KD FOR | KD FOR | KD FOR | KD FOR | | KD(IIaR) OF | KD(IIb) OF |
| | FcgRIa | FcgRIIaR | FcgRIIaH | FcgRIIb | FcgRIIIaV | KD(IIaR)/ | ALTERED | ALTERED |
| VARIANT NAME | (mol/L) | (mol/L) | (mol/L) | (mol/L) | (mol/L) | KD(IIb) | POLYPEPTIDE | POLYPEPTIDE |
| IL6R-G1d/IL6R-L | 3.2E−10 | 1.0E−06 | 6.7E−07 | 2.6E−06 | 3.5E−07 | 0.4 | 1.1 | 1 |
| IL6R-B3/IL6R-L | 4.2E−10 | 1.1E−06 | 7.7E−07 | 3.1E−06 | 3.3E−07 | 0.3 | 1.0 | 1 |
| IL6R-BP253/IL6R-L | 5.0E−11 | 2.3E−09 | 8.6E−07 | 8.9E−09 | 4.0E−07 | 0.3 | 480.0 | 349 |
| IL6R-BP208/IL6R-L | 1.9E−08 | 8.5E−07 | 8.3E−06 | 3.2E−08 | 5.3E−05 | 26.3 | 1.3 | 95 |
| IL6R-BP256/IL6R-L | 2.0E−09 | 7.3E−07 | 2.1E−05 | 8.7E−08 | 1.1E−05 | 8.4 | 1.5 | 36 |
| IL6R-BP257/IL6R-L | 3.1E−10 | 1.3E−06 | 3.9E−05 | 4.3E−07 | 1.9E−05 | 3.0 | 0.9 | 7 |
| IL6R-AP002/IL6R-BP256/IL6R-L | 3.6E−09 | 1.4E−07 | 4.2E−06 | 4.1E−09 | 1.7E−05 | 34.3 | 7.7 | 752 |
| IL6R-AP002/IL6R-BP257/IL6R-L | 1.9E−09 | 1.3E−07 | 4.9E−06 | 4.7E−09 | 1.4E−05 | 27.7 | 8.3 | 657 |
| IL6R-BP230/IL6R-L | 1.4E−08 | 5.7E−07 | 9.6E−06 | 2.1E−08 | 6.7E−05 | 27.5 | 1.9 | 149 |
| IL6R-BP259/IL6R-L | 3.1E−09 | 8.6E−07 | 1.8E−05 | 1.1E−07 | 1.1E−05 | 8.0 | 1.3 | 29 |
| IL6R-BP260/IL6R-L | 3.1E−10 | 1.2E−06 | 3.9E−06 | 3.8E−07 | 2.1E−05 | 3.2 | 0.9 | 8 |
| IL6R-AP009/IL6R-BP259/IL6R-L | 3.5E−09 | 1.9E−07 | 6.6E−06 | 3.9E−09 | 2.2E−05 | 47.7 | 5.8 | 788 |
| IL6R-AP009/IL6R-BP260/IL6R-L | 1.7E−09 | 1.4E−07 | 5.4E−06 | 4.2E−09 | 1.6E−05 | 34.2 | 7.6 | 739 |

As shown in Table 10, compared to IL6R-BP208/IL6R-L, both IL6R-AP002/IL6R-BP256/IL6R-L and IL6R-AP002/IL6R-BP257/IL6R-L produced by introducing S239D and S239E, respectively, into one of the chains of IL6R-BP208/IL6R-L showed enhanced binding to FcgRIIb. Furthermore, the value of KD(IIaR)/KD(IIb) was greater than that of IL6R-BP256/IL6R-L, and selectivity to FcgRIIb was also improved. On the other hand, compared to IL6R-BP208/IL6R-L, both selectivity and binding to FcgRIIb of IL6R-BP256/IL6R-L produced by introducing S239D into both chains of IL6R-BP208/IL6R-L and IL6R-BP257/IL6R-L produced by introducing S239E into both chains of IL6R-BP208/IL6R-L had significantly decreased. This way, when S239D or S239E was introduced into only one of the chains, effects of enhancing binding to FcgRIIb was observed, whereas when S239D or S239E was introduced into both chains, binding to FcgRIIb significantly decreased. The main reason why this happened may be, as described previously, the destabilization of loop structure in CH2 domain A. Similar results were observed when S239D and S239E were introduced using IL6R-BP230/IL6R-L as the template. IL6R-AP009/IL6R-BP259/IL6R-L and IL6R-AP009/IL6R-BP260/IL6R-L produced by introducing S239D and S239E, respectively, into one of the chains of IL6R-BP230/IL6R-L showed both higher selectivity and binding to FcgRIIb than those of IL6R-BP230/IL6R-L. On the other hand, IL6R-BP259/IL6R-L and IL6R-BP260/IL6R-L produced by introducing S239D and S239E respectively into both chains showed greatly decreased selectivity and binding to FcgRIIb as compared to those of IL6R-BP230/IL6R-L. Furthermore, variants produced by introducing S239D or S239E into one of the chains of IL6R-BP208/IL6R-L and IL6R-BP230/IL6R-L all showed greater selectivity as well as binding to FcgRIIb as compared to those of IL6R-BP253/IL6R-L utilizing a known FcgRIIb-enhancement technique.

### 9-3. Verification of Alterations Based on Structural Information on Fc(P208)/FcgRIIaR

Comparison of the crystal structures of Fc(P208) with FcgRIIb and with FcgRIIaR in Example 3 showed that there is a difference in electron density around position 237 (EU numbering) where a hydrogen bond is formed with Tyr at position 160 of FcgRIIb, and the CH2 domain A side was suggested to have a larger contribution to binding with FcgRIIb, and the CH2 domain B side was suggested to have a larger contribution to binding with FcgRIIaR (FIGS. 12 and 13). For example, from how the electron density looks, in the binding with the FcgRIIa type R, Leu at position 234 and Leu at position 235 (EU numbering) in CH2 domain B are considered to be involved in binding with the receptor, whereas these residues may only have little involvement in the binding to FcgRIIb. Then, by substituting these two residues with residues other than hydrophobic residues, interaction with FcgRIIa type R may be reduced by a greater degree. However, at the CH2 domain A side, the residues of Leu at position 234 and Leu at position 235 (EU numbering) are considered to contribute to stabilization of the loop structure around position 237 (EU numbering), and in particular, it is highly likely that they are more greatly involved in binding with FcgRIIb. Therefore, substituting these residues with residues other than hydrophobic residues may decrease interaction of CH2 domain A with FcgRIIb. In particular, Leu at position 235 (EU numbering) forms a favorable hydrophobic interaction in CH2 domain A of a complex structure formed with FcgRIIb, and since it is considered to have a large contribution to stabilization of the loop structure around position 237 (EU numbering), this residue was examined by substituting a residue in only one of the chains with a non-hydrophobic residue. If the hydrophobic interaction at the CH2 domain A in particular can further be strengthened to further stabilize the loop structure around position 237 (EU numbering) by substituting Leu at position 235 (EU numbering) with hydrophobic amino acids other than Leu in both chains, that may lead to reduction of entropic energy loss accompanying hydrogen bond formation with Tyr at position 160 of FcgRIIb and may cause enhancement of selectivity and binding to FcgRIIb; therefore, these were examined as well.

As the antibody H chain, IL6R-BP264 (SEQ ID NO: 28) was produced by introducing E233D, G237D, P238D, H268E, P271G, Y296D, and A330R into IL6R-B3 (SEQ ID NO: 23) was used as the template. Variants in which Leu at position 234 (EU numbering) of IL6R-BP264 individually was substituted with Asn, Ser, Asp, Gln, Glu, Thr, Arg, His, Gly, Lys, and Tyr were produced. Variants in which the amino acid at position 235 (EU numbering) in IL6R-BP264 was substituted with any of the 18 amino acids other than the original amino acid and Cys were also produced. For the other antibody H chain, IL6R-AP029 (SEQ ID NO: 42) was produced by introducing E233D, G237D, P238D, H268E, P271G, Y296D, and A330R into IL6R-A5 (SEQ ID NO: 69). IL6R-L (SEQ ID NO: 21) was commonly used as the antibody L chain. Variants produced by introducing L234N, L234S, L234D, L234Q, L234E, L234T, L234R, L234H, L234G, L234K, and L234Y into IL6R-BP264 and variants produced by introducing L235W, L235M, L235P, L235F, L235A, L235V, and L235I, respectively, into IL6R-BP264 were prepared as homologous antibodies containing the same alteration in both chains and variants produced by introducing L235N, L235S, L235D, L235Q, L235E, L235T, L235R, L235H, L235G, L235K, and L235Y, respectively, were combined with IL6R-AP029 to prepare heterodimeric antibodies, and those were then subjected to examination.

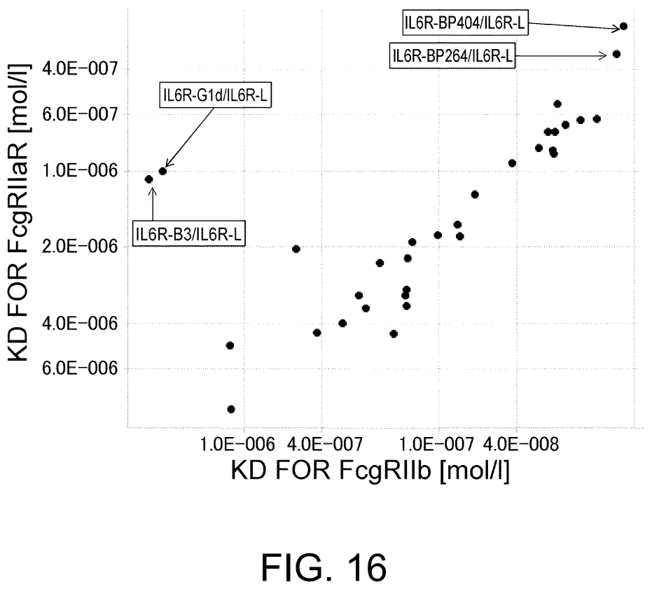

These variants were used for antibody expression and purification according to the method of Reference Example 1, and binding to each FcgR (FcgRIa, FcgRIIa type H, FcgRIIa type R, FcgRIIb, and FcgRIIIa type V) was assessed using the method of Reference Example 2. FIG. 16 shows a graph in which KD values of each variant for FcgRIIb are shown on the horizontal axis and the KD values of each variant for FcgRIIaR are shown on the vertical axis.

As shown in FIG. 16, IL6R-BP404/IL6R-L produced by introducing L234Y into both chains of IL6R-BP264/IL6R-L showed slightly enhanced binding to FcgRIIb as compared to that of the IL6R-BP264/IL6R-L prior to alteration.

From among these variants, IL6R-BP404/IL6R-L with enhanced FcgRIIb binding and variants with improved selectivity to FcgRIIb are summarized in Table 11. In this Table, “parent polypeptide KD(IIb)/altered polypeptide KD(IIb)” refers to a value obtained by dividing the KD value of IL6R-B3/IL6R-L for FcgRIIb by the KD value of each variant for FcgRIIb. “Parent polypeptide KD(IIaR)/altered polypeptide KD(IIaR)” refers to a value obtained by dividing the KD value of IL6R-B3/IL6R-L for FcgR IIaR by the KD value of each variant for FcgRIIaR. “KD(IIaR)/KD(IIb)” is a value obtained by dividing the KD of each variant for FcgRIIaR by the KD of each variant for FcgRIIb. The larger this value is, the higher the selectivity to FcgRIIb is. The Table 11 values shown in bold italicized font were calculated using the following equation

KD=C·Rₘₐₓ/(R_{eq}−RI)−C [Equation 2]

shown in Reference Example 2 since the binding of FcgR to IgG was too weak to accurately analyze by kinetic analysis.

TABLE 11

| | | | | | | | KD(IIaR) OF | KD(IIb) OF |
|---|---|---|---|---|---|---|---|---|
| | | | | | | | PARENT | PARENT |
| | | | | | | | POLYPEPTIDE/ | POLYPEPTIDE/ |
| | KD FOR | KD FOR | KD FOR | KD FOR | KD FOR | | KD(IIaR) OF | KD(IIb) OF |
| | FcgRIa | FcgRIIaR | FcgRIIaH | FcgRIIb | FcgRIIIaV | KD(IIaR)/ | ALTERED | ALTERED |
| VARIANT NAME | (mol/L) | (mol/L) | (mol/L) | (mol/L) | (mol/L) | KD(IIb) | POLYPEPTIDE | POLYPEPTIDE |
| IL6R-G1d/IL6R-L | 3.2E−10 | 1.0E−06 | 6.7E−07 | 2.6E−06 | 3.5E−07 | 0.4 | 1.1 | 1.2 |
| IL6R-B3/IL6R-L | 4.2E−10 | 1.1E−06 | 7.7E−07 | 3.1E−06 | 3.3E−07 | 0.3 | 1.0 | 1.0 |
| IL6R-BP264/IL6R-L | 7.4E−09 | 3.5E−07 | 2.8E−06 | 1.2E−08 | 2.6E−05 | 28.3 | 3.2 | 252.0 |
| IL6R-BP404/IL6R-L | 1.0E−08 | 3.6E−07 | 3.4E−06 | 1.1E−08 | 3.3E−05 | 32.1 | 3.1 | 276.8 |
| IL6R-BP408/IL6R-L | 1.4E−07 | 3.4E−05 | 7.7E−05 | 7.2E−07 | 5.0E−05 | 47.0 | 0.03 | 4.3 |
| IL6R-BP419/IL6R-L | 4.0E−08 | 8.3E−07 | 3.8E−05 | 2.6E−08 | 3.8E−05 | 32.0 | 1.3 | 118.8 |
| IL6R-AP029/IL6R-BP407/IL6R-L | 1.3E−08 | 6.3E−07 | 8.9E−06 | 1.9E−08 | 2.3E−05 | 33.5 | 1.7 | 164.0 |
| IL6R-AP029/IL6R-BP408/IL6R-L | 2.0E−08 | 8.6E−07 | 8.2E−06 | 2.6E−08 | 3.1E−05 | 33.6 | 1.3 | 121.1 |
| IL6R-AP029/IL6R-BP409/IL6R-L | 1.9E−08 | 6.3E−07 | 9.4E−06 | L5E−08 | 2.8E−05 | 40.8 | 1.8 | 201.3 |
| IL6R-AP029/IL6R-BP410/IL6R-L | 1.7E−08 | 6.6E−07 | 5.1E−06 | 2.2E−08 | 2.1E−05 | 29.7 | 1.7 | 139.0 |

As shown in Table 11, IL6R-BP404/IL6R-L produced by introducing L234Y into both chains of IL6R-BP264/IL6R-L showed 1.1-fold increase in FcgRIIb binding as compared to that of the IL6R-BP264/IL6R-L prior to introduction of alterations. IL6R-BP408/IL6R-L produced by introducing L235Q into both chains of IL6R-BP264/IL6R-L, IL6R-BP419/IL6R-L produced by introducing L235F into both chains of IL6R-BP264/IL6R-L, IL6R-AP029/IL6R-BP407/IL6R-L produced by introducing L235D into one of the chains of IL6R-BP264/IL6R-L, IL6R-AP029/IL6R-BP408/IL6R-L produced by introducing L235Q into one of the chains of IL6R-BP264/IL6R-L, IL6R-AP029/IL6R-BP409/IL6R-L produced by introducing L235E into one of the chains of IL6R-BP264/IL6R-L, and IL6R-AP029/IL6R-BP410/IL6R-L produced by introducing L235T into one of the chains of IL6R-BP264/IL6R-L all showed KD(IIaR)/KD(IIb) values that were larger compared to that of the IL6R-BP264/IL6R-L prior to introduction of alteration, and they were variants with improved selectivity to FcgRIIb.

### [Example 10] Assessment of Immunogenicity of Fc Variants with Enhanced FcgRIIb Binding Using an in Silico Immunogenicity Prediction Tool

When using the Fc variants described in this Example as therapeutic antibodies, it is preferred that production of anti-drug antibodies that weaken their pharmacological effect is not induced. Since antibodies with high immunogenicity tend to induce production of anti-drug antibodies, immunogenicity of therapeutic antibodies is preferably as low as possible. As efforts to avoid increase in immunogenicity of the variants as much as possible, one can use in silico immunogenicity prediction tools that predict T-cell epitopes such as Epibase™ and EpiMatrix prediction tools. Epibase™ Light (Lonza) is an in silico immunogenicity prediction tool to calculate the binding ability of 9-mer peptide to MHC class II which contains major DRB1 alleles using the FASTER algorithm (Expert Opin Biol Ther. 2007 March; 7(3): 405-18). This tool can identify T-cell epitopes with strong binding (strong epitopes) and medium binding (medium epitopes) to MHC class II.

DRB1 allotype population frequency is reflected in the calculation, and for this, Caucasian population frequency shown in Table 12 below can be used.

TABLE 12

| | |
|---|---|
| DRB1*1501 | 24.5% |
| DRB1*0301 | 23.7% |
| DRB1*0701 | 23.3% |
| DRB1*0401 | 16.2% |
| DRB1*0101 | 15.0% |
| DRB1*1101 | 11.6% |
| DRB1*1301 | 10.9% |
| DRB1*1302 | 8.2% |
| DRB1*0404 | 5.9% |
| DRB1*1104 | 5.8% |
| DRB1*1601 | 5.0% |
| DRB1*1401/1454 | 4.9% |
| DRB1*0801 | 4.9% |
| DRB1*0102 | 3.8% |
| DRB1*1201/1206/1210 | 3.3% |
| DRB1*0407 | 2.7% |
| DRB1*0901 | 2.3% |
| DRB1*1303 | 2.0% |
| DRB1*1001 | 1.9% |
| DRB1*0405 | 1.5% |
| DRB1*0403 | 1.0% |
| DRB1*1102 | 0.7% |
| DRB1*0802 | 0.7% |
| DRB1*1502 | 0.5% |
| DRB1*0804 | 0.4% |
| DRB1*1404 | 0.4% |
| DRB1*0803 | 0.3% |
| DRB1*0406 | 0.2% |
| DRB1*1402 | 0.2% |
| DRB1*1602 | 0.2% |
| DRB1*1202 | 0.1% |
| DRB1*0304 | 0.1% OR LESS |
| DRB1*1405 | 0.1% OR LESS |
| DRB1*0410 | 0.1% OR LESS |
| DRB1*1503 | 0.1% OR LESS |
| DRB1*1106 | 0.1% OR LESS |
| DRB1*1504 | 0.1% OR LESS |
| DRB1*1304 | 0.1% OR LESS |
| DRB1*1110 | 0.1% OR LESS |
| DRB1*1406 | 0.1% OR LESS |
| DRB1*0411 | 0.1% OR LESS |
| DRB1*0302 | 0.1% OR LESS |
| DRB1*1312 | 0.1% OR LESS |

This tool was used to compare the total number of T-cell epitopes with strong binding and medium binding that are included in the sequences (the sequence from position 118 to the C terminus (EU numbering)) of various Fc variants reported so far and Fc variants with selectively enhanced binding to FcgRIIb described in the Example. Specifically, the following antibodies were produced as comparison controls for evaluating pre-existing techniques: Fc(DLE) (SEQ ID NO: 78) which is an antibody Fc region introduced with the S239D, A330L, and I332E alterations, which has been previously reported to enhance FcgRIIIa-binding (Proc Natl Acad Sci USA. 2006, 103:4005-10); Fc(YTE) (SEQ ID NO: 79) which is an antibody Fc region introduced with the M252Y, S254T, and T256E alterations, which has been previously reported to enhance FcRn-binding (J Biol Chem. 2006, 281:23514-24); Fc(EF) (SEQ ID NO: 80) which is an antibody Fc region introduced with the S267E and L328F alterations, which has been reported to enhance FcgRIIb-binding (Mol Immunol. 2008, 45:3926-33); and Fc(P208) (SEQ ID NO: 81) which is an Fc region of an antibody introduced with the E233D, G237D, P238D, H268D, P271G, and A330R alterations, which has been reported to enhance FcgRIIb-binding and is described in WO2012/115241. Furthermore, Fc(P587) (SEQ ID NO: 70), which is an antibody Fc region introduced with the E233D, P238D, S264I, S267A, H268E, and P271G alterations in a similar manner to the BP568 variant, which enhances FcgRIIb-binding and is described in the Examples, and Fc(P588) (SEQ ID NO: 71), which is an antibody Fc region introduced with the P238D, S264I, S267A, H268E, and P271G alterations in a similar manner to BP492, were produced. The total number of strong and medium binding epitopes in these Fc variants was compared using the Epibase™ prediction tool. The results are shown in Table 13.

TABLE 13

| | NUMBER OF |
|---|---|
| | T-cell epitope |
| Fc (DLE) | 2 |
| Fc (YTE) | 5 |
| Fc (EF) | 4 |
| Fc (P208) | 5 |
| Fc (P587) | 2 |
| Fc (P588) | 2 |

These results indicate that among the existing Fc variants, Fc(P587) and Fc(P588) which are Fc regions of variants described in the Examples have small number of T-cell epitopes and low immunogenicity risk. When using the variants as pharmaceuticals, this property indicates that the possibility of inducing anti-drug antibodies is lowered and that the variants have excellent properties.

### [Example 11] Assessment of Blood Kinetics of Fc Variants with Enhanced Human FcgRIIb Binding Using Human FcgRIIb Transgenic Mice

### (11-1) Outline of the Examination

As indicated in WO2013/047752, compared to a native human IgG, the plasma concentration of a target soluble antigen can be reduced significantly in a living organism by administering an antigen-binding molecule having human-FcRn-binding activity under an acidic pH range condition and comprising an antigen-binding domain whose antigen binding activity of the antigen-binding molecule changes depending on an ion concentration condition, and an FcgR-binding domain with higher FcgR-binding activity than the FcgR-binding domain of a native human IgG Fc region, wherein the sugar chain linked to position 297 (EU numbering) is a fucose-containing sugar chain. It has also been reported that when an antigen-binding molecule that has enhanced binding activity particularly to FcgRIIb among the FcgRs is administered in vivo, elimination of soluble antigens in plasma is accelerated, and the concentration of soluble antigens in plasma can be reduced effectively. In this Example, an Fc variant with enhanced binding to human FcgRIIb was administered to genetically-modified transgenic mice introduced with human FcgRIIb to test whether the elimination rate of the target soluble antigens can be accelerated by the Fc variant with actually enhanced binding to human FcgRIIb described herein.

(11-2) Preparation of Antibodies with Enhanced Binding to FcgRIIb

The following antibodies were used as the Fc variants with enhanced human FcgRIIb-binding:
IL6R-P587 was produced by introducing the E233D, P238D, S264I, S267A, H268E, and P271G alterations in a similar manner to BP568 into IL6R-G1d (SEQ ID NO: 19) consisting of a constant region of G1d which has the C-terminal Gly and Lys removed from human IgG1, and a variable region of an antibody against human interleukin-6 receptor (human IL-6R) disclosed in WO2009/125825. Fv4-P587 comprising IL-6R-P587 as the antibody H chain and IL6R-L2 (SEQ ID NO: 74) which is the L chain of an anti-human IL-6R antibody disclosed in WO2009/125825 as the antibody L chain was prepared according to the method of Reference Example 1. As a comparison control, Fv4-IgG1 comprising IL6R-G1d (SEQ ID NO: 19) and IL6R-L2 (SEQ ID NO: 74) as the antibody H chain and L chain, respectively, was prepared similarly according to the method of Reference Example 1. As described in WO2009/125825, Fv4-G1d and Fv4-P587 prepared herein comprises an antigen-binding domain whose antigen-binding activity of the antigen-binding molecule changes depending on the condition of proton ion concentration, that is, the antigen-binding activity of the antigen-binding domain binds to a human IL-6R (antigen) under acidic pH conditions weaker than under neutral pH conditions.

### (11-3) Production of Human FcgRIIb Transgenic Mice

Human FcgRIIb transgenic mice were produced by the following method.

Transgenic mice were produced by introducing the human FcgRIIb gene into C57BL/6 (B6) mice. Production of transgenic mice was carried out in accordance with the procedure described in “Nagy et al., (Manipulating the mouse embryo, CSHL press. (2003) 399-506)” and in “Ueda et al. (Latest Technology for Gene Targeting”, Yodosha. (2000) 190-207)”. More specifically, transgenic mice were produced by microinjecting into pronuclear fertilized eggs of B6 mice a bacterial artificial chromosome into which the genomic region of the human FcgRIIb gene (GeneBank #NW_004077999: 18,307,411-18,381,603) was cloned. Mice transferred with the human FcgRIIb gene were selected from the obtained mice by Southern blotting using a probe that specifically hybridizes with the human FcgRIIb gene and by performing PCR. Blood and liver were collected from the human FcgRIIb transgenic mice, and expression of the human FcgRIIb gene was confirmed by Reverse Transcription Polymerase Chain Reaction (RT-PCR) using primers that specifically amplify the human FcgRIIb gene. As a result, expression of the human FcgRIIb gene was detected. Furthermore, mouse peripheral blood mononuclear cells (PBMC) were isolated from the blood of the human FcgRIIb transgenic mice, and the expression of human FcgRIIb in PBMC was confirmed by fluorescence activated cell sorting (FACS) analyses. As a result, expression of human FcgRIIb was detected. The above confirmed that human FcgRIIb transgenic mice which express human FcgRIIb were established.

### (11-4) In Vivo Test of Simultaneous Administration of Antigens and Antibodies Using the Human FcgRIIb Transgenic Mice

Using the human FcgRIIb transgenic mice produced in (11-3), soluble human IL-6R which is the antigen and the anti-human IL-6R antibody prepared in (11-2) were administered simultaneously, and the plasma concentrations of soluble human IL-6R and anti-human IL-6R antibody after the administration were evaluated.

A mixed solution of soluble human IL-6R and anti-human IL-6R antibody (5 μg/mL and 0.1 mg/mL, respectively) was administered in a single dose at 10 mL/kg to the tail vein. In this case, since anti-human IL-6R antibody is present sufficiently in excess with respect to soluble human IL-6R, almost all of the soluble human IL-6R is considered to be bound to the antibody. Blood was collected five minutes, 1 hour, 4 hours, 7 hours, 1 day, 3 days, 7 days, 14 days, 21 days, and 28 days after administration. The collected blood was immediately centrifuged at 4° C. and 15,000 rpm for 15 minutes to obtain the plasma. The separated plasma was stored in a freezer set at −20° C. or lower until the time of measurement. The above-described Fv4-P587 and Fv4-IgG1 were used for the anti-human IL-6R antibody.

### (11-5) Measurement of Plasma Anti-Human IL-6R Antibody Concentration by ELISA

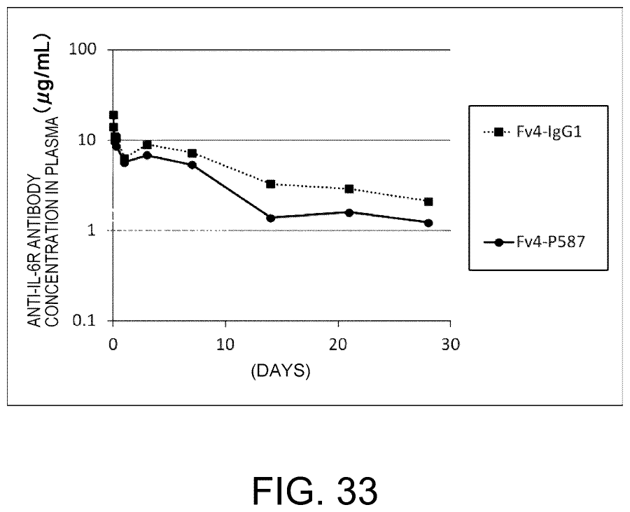

Concentration of anti-human IL-6R antibody in mouse plasma was measured by ELISA. First, anti-human IgG (γ-chain specific) antibody F(ab′) 2 fragment (Sigma) was aliquoted into a Nunc-Immuno™ MaxiSorp™ plate (Nalge Nunc International), followed by allowing to stand overnight at 4° C. to prepare an anti-human IgG-immobilized plate. Calibration curve samples of plasma concentration at 0.8, 0.4, 0.2, 0.1, 0.05, 0.025 and 0.0125 μg/mL and mouse plasma assay samples diluted to 100-fold or more were prepared. Mixtures obtained by adding 200 μL of 20 ng/mL soluble human IL-6R to 100 μl of the calibration curve samples or plasma assay samples were then stirred for 1 hour at room temperature. Subsequently, the anti-human IgG-immobilized plate in which the mixtures had been dispensed was further stirred for one hour at room temperature. Then, a biotinylated anti-human IL-6R antibody (R&D) was reacted with the samples at room temperature for one hour and Streptavidin-PolyHRP80 (Stereospecific Detection Technologies) was reacted with the samples at room temperature for one hour. The chromogenic reaction was carried out using TMB One Component HRP Microwell Substrate (BioFX Laboratories) as substrate. After the reaction was stopped by adding 1N sulfuric acid (Showa Chemical), absorbance at 450 nm was measured with a microplate reader. Antibody concentrations in the mouse plasma were calculated from absorbance values of the calibration curve using the SOFTmax™ PRO analysis software (Molecular Devices). The time course of plasma antibody concentration in human FcgRIIb transgenic mice after intravenous administration measured by this method is shown in FIG. 33.

### (11-5) Measurement of Plasma Human IL-6R Concentration by Electrochemiluminescence

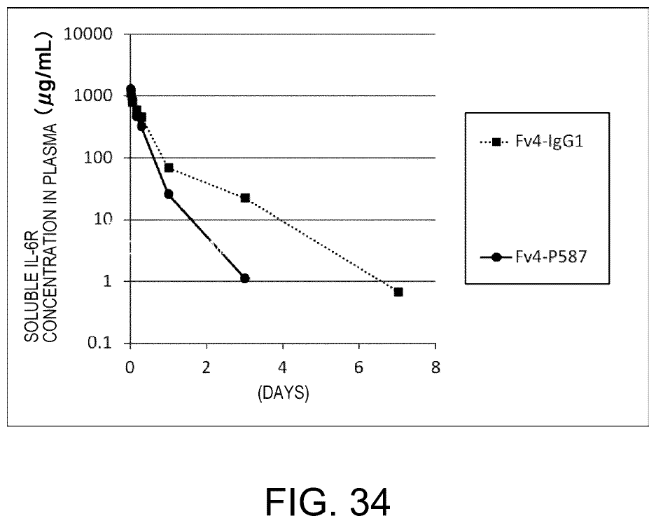

The human IL-6R concentration in mouse plasma was measured by electrochemiluminescence. Calibration curve samples of human IL-6R were prepared at plasma concentrations of 12.5, 6.25, 3.13, 1.56, 0.781, 0.391, and 0.195 ng/mL, and mouse plasma assay samples were prepared by diluting 50 fold or more. Monoclonal Anti-human IL-6R Antibody (R&D) which has been ruthenium-labeled using SULFO-TAG NHS Ester (Meso Scale Discovery), Biotinylated Anti-human IL-6 R Antibody (R&D), and tocilizumab solution were mixed in and allowed to react overnight at 37° C. Then, the mixed solution was aliquoted into the Streptavidin Gold Multi-ARRAY Plate (Meso Scale Discovery) subjected to blocking using a TBS-Tween solution containing 0.5% BSA (w/v) overnight at 5° C. After allowing to react for two more hours at room temperature, the plate was washed. Immediately after the Read Buffer T (×2) (Meso Scale Discovery) was aliquoted into the plate, and measurements were carried out using the SECTOR® Imager 2400 (Meso Scale Discovery). The hSIL-6R concentrations were calculated based on the response from the calibration curve using the analytical software SOFTmax™ PRO (Molecular Devices). Time course of the soluble human IL-6R concentration in plasma of human FcgRIIb transgenic mice after intravenous administration, which was measured by this method, is shown in FIG. 34.

### (11-6) Effects of Enhancing Human FcgRIIb Binding

The in vivo test results were compared for Fv4-P587 whose human FcgRIIb-binding has been enhanced and Fv4-IgG1. As shown in FIG. 33, the plasma retention of both antibodies were nearly equal; however, as shown in FIG. 34, elimination of human IL-6R was confirmed to be accelerated when human IL-6R was administered simultaneously with Fv4-P587 having enhanced human FcgRIIb-binding as compared to when human IL-6R was administered simultaneously with Fv4-IgG1. More specifically, an antibody that binds to human IL-6R in a pH-dependent manner was found to be able to decrease the concentration of soluble human IL-6R by enhancing its binding ability to human FcgRIIb.

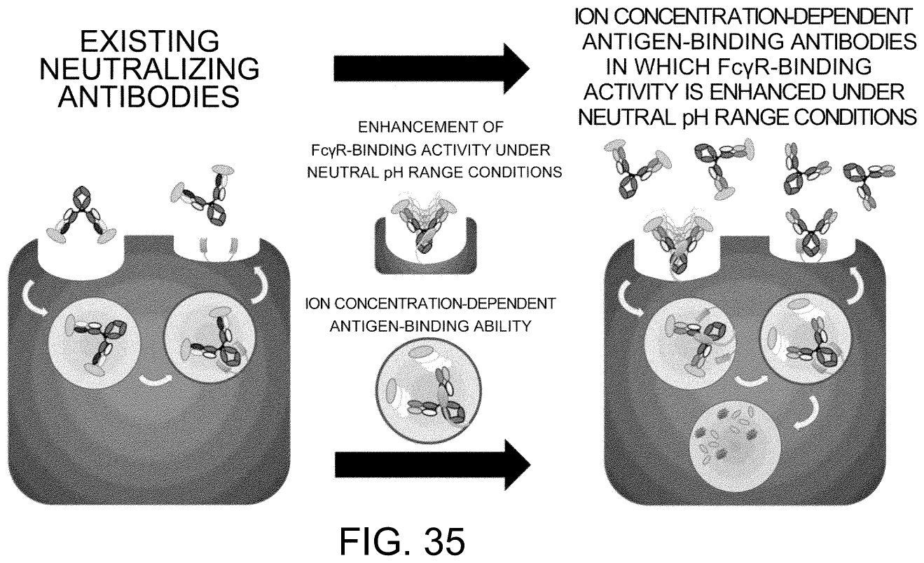

Without being bound by a particular theory, one may consider from this result that according to the mechanism indicated in FIG. 35, the soluble antigens in plasma that bind to this antibody disappear as a result of being incorporated into FcγRIIb-expressing cells via human FcγRIIb.

Soluble human IL-6R bound to an antibody that binds soluble human IL-6R is recycled into plasma by FcRn along with the antibody. In contrast, Fv4-IgG1 which is an antibody that binds to soluble human IL-6R in a pH-dependent manner dissociates the antibody-bound soluble human IL-6R under acidic conditions in the endosome. Since the dissociated soluble human IL-6R is degraded by the lysosome, elimination of soluble human IL-6R can be significantly accelerated, and Fv4-IgG1 which is an antibody that binds to soluble human IL-6R in a pH-dependent manner binds to FcRn in the endosome and is then recycled into plasma. This recycled antibody can bind again to soluble human IL-6R; therefore, antigen (soluble human IL-6R)-binding and recycling into plasma by FcRn are repeated. It is considered that as a result, single antibody molecule can bind to the soluble human IL-6R several times repeatedly. Furthermore, it is considered that by enhancing the FcgRIIb-binding activity of Fv4-IgG1 which shows pH-dependent antigen binding, a complex formed between an antibody that binds to the soluble human IL-6R and a soluble human IL-6R is quickly incorporated into cells via FcgRIIb to enable decrease in the soluble human IL-6R concentration more efficiently (FIG. 35).

### [Example 12] Evaluation of Blood Kinetics of Fc Variants with Enhanced Binding to Human FcgRIIb Using Human FcgRIIb and Human FcRn Transgenic Mice

### (12-1) Outline of the Examination

As indicated in WO2013/047752, use of an antigen-binding molecule having higher FcγR-binding activity than that of the native IgG Fc region and whose human FcRn-binding activity under an acidic pH range condition is enhanced confirmed that plasma retention properties are improved compared to antigen-binding molecules whose human FcRn-binding activity under an acidic pH range condition is not enhanced. On the other hand, there have been reports that an antigen-binding molecule having higher FcγR-binding activity than that of the native human IgG Fc region and whose human FcRn-binding activity under an acidic pH range condition is enhanced showed decreased plasma concentration of target antigen as compared to an antigen-binding molecule having higher FcγR-binding activity than that of the native human IgG Fc region and whose human FcRn-binding activity under an acidic pH range condition is not enhanced. Accordingly, whether the antigen-binding molecules carrying the Fc region variants with enhanced human FcgRIIb binding described in the Examples have similar properties was investigated.

(12-2) Production of an Antigen-Binding Molecule Having Higher FcγR-Binding Activity than that of a Native Human IgG Fc Region and Whose Human FcRn-Binding Activity Under an Acidic pH Range Condition is Enhanced

In addition to Fv4-IgG1 and Fv4-P587 described in Example 11-2, Fv4-P587-LS was prepared according to the method of Reference Example 1, where Fv4-P587-LS contains IL6R-L2 as the antibody L chain and IL6R-P587-LS (SEQ ID NO: 73) as the antibody H chain. IL6R-P587-LS was produced by introducing into IL6R-P587, the H chain of Fv4-P587, alterations consisting of substitution of Met at position 428 with Leu and substitution of Asn at position 434 with Ser, according to EU numbering, which are substitutions that have been previously reported to improve blood kinetics of antibodies (Nat. Biotechnol. 2010. 28; 157-159).

(12-3) Analysis of Interaction with Human FcRn

Analysis of interaction between a prepared antibody and the human FcRn was carried out using a Biacore™ T200 surface plasmon resonance system. An appropriate amount of protein L (BioVision) was immobilized onto Sensor Chip CM4 (GE Healthcare) by the amine coupling method and the antibodies of interest were captured onto it. Next, a diluted FcRn and a running buffer (used as a control solution) were injected to allow interaction of the antibodies captured onto this sensor chip with human FcRn. 50 mmol/L sodium phosphate, 150 mmol/L NaCl, and 0.05% (w/v) Tween20 (pH 6.0) was used as the running buffer, and this running buffer was also used to dilute FcRn. 10 mmol/L glycine-HCl (pH 1.5) was used for chip regeneration. All measurements were performed at 25° C. Kinetic parameters such as association rate constants ka (1/Ms) and dissociation rate constants kd (1/s) were determined from the sensorgram obtained from the measurements, and the KD(M) of each antibody for human FcRn were determined from the values of these constants. The Biacore™ T200 Evaluation Software (GE Healthcare) was used to calculate each parameter. KD values of the antibodies prepared this time for human FcRn as measured by this method are shown in Table 14. As shown in Table 14, compared to Fv4-P587, Fv4-P587-LS was confirmed to have enhanced FcRn binding under acidic conditions.

TABLE 14

| | KD FOR |
|---|---|
| | HUMAN FcRn AT |
| | pH 6.0 (μmol/L) |
| IgG1 | 1.4 |
| P587 | 1.5 |
| P587-LS | 0.12 |

### (12-4) Production of Human FcgRIIb and Human FcRn Transgenic Mice

Human FcgRIIb and human FcRn transgenic mice, and mouse FcRn knockout mice were produced by the following method.

First, mouse FcRn knockout mice were produced. Production of knockout mice was carried out according to the procedure described in “Nagy et al., (Manipulating the mouse embryo, CSHL press. (2003) 399-506)”. More specifically, a targeting vector to destroy the mouse FcRn gene is prepared and introduced into ES cells (derived from C57BL/6 mice) to destroy mouse FcRn gene by homologous recombination. RNA was extracted from the liver of the established mouse FcRn knockout mice, and by using cDNA synthesized from this RNA as a template, RT-PCR was carried out using primers that specifically amplify mouse FcRn. As a result, the mouse FcRn gene was not detected from mouse FcRn knockout mice. Next, transgenic mice were produced by introducing the human FcgRIIb and human FcRn genes into the mouse FcRn knockout mice. Transgenic mice were produced according to the procedures described in “Nagy et al., (Manipulating the mouse embryo, CSHL press. (2003) 399-506)” and in “Ueda et al. (Latest Technology for Gene Targeting, Yodosha. (2000) 190-207)”. More specifically, the mice were produced by microinjecting into pronuclear fertilized eggs of mouse FcRn knockout mice a bacterial artificial chromosome into which the genomic regions of the human FcRn gene (GeneBank #NC_000019.9:50,000,108-50,039,865) and the human FcgRIIb gene (GeneBank #NW_004077999: 18,307,411-18,381,603) were cloned. Mice introduced with the human FcRn gene and the human FcgRIIb gene and made to be homozygous for the mouse FcRn knockout allele were selected from the obtained mice by Southern blotting using a probe that specifically hybridizes with each gene and by PCR. Blood was collected from the human FcgRIIb and human FcRn transgenic mice and mouse FcRn knockout mice, and expression of the human FcRn gene and the human FcgRIIb gene were confirmed by RT-PCR using primers that specifically amplify the human FcRn gene and the human FcgRIIb gene. As a result, expressions of the human FcRn gene and the human FcgRIIb gene were detected. The above confirmed that human FcgRIIb and human FcRn transgenic mice and mouse FcRn knockout mice which express human FcRn and human FcgRIIb and do not express mouse FcRn were established.

### (12-5) Improvement of Pharmacokinetics by Enhancing Human FcRn-Binding Activity Under an Acidic pH Range Condition

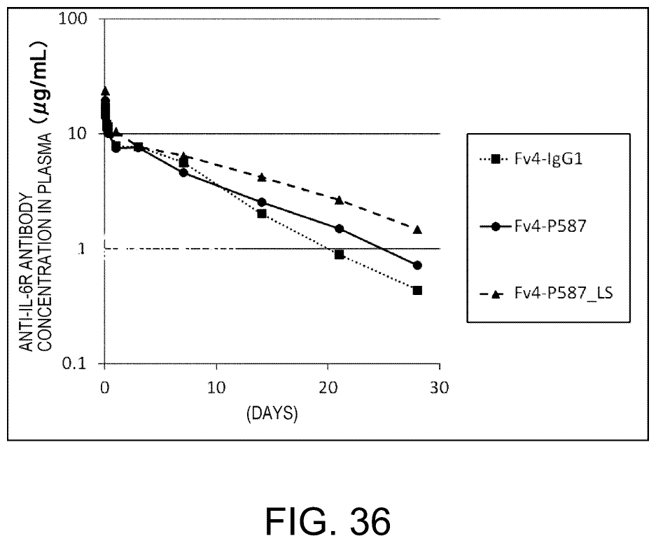

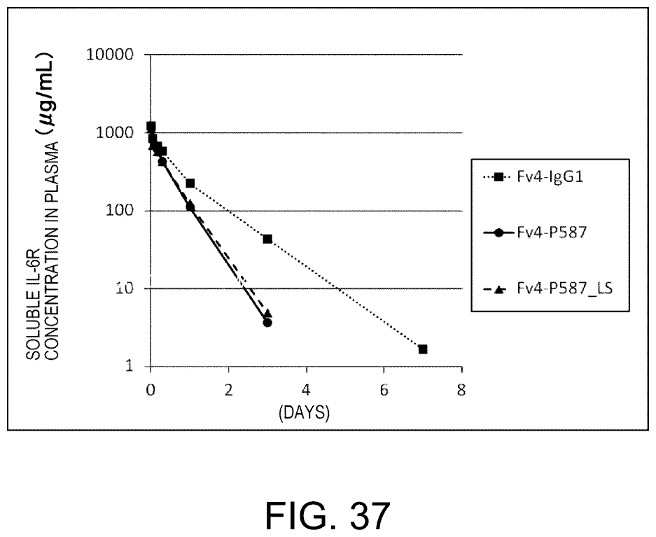

In vivo examination was carried out in a manner similar to the method of Example 11 by administering Fv4-IgG1, Fv4-P587, and Fv4-P587-LS individually to human FcgRIIb and human FcRn transgenic mice, and plasma concentrations of soluble IL-6R and anti-human IL-6R antibody were measured for the mouse groups. The results of measuring the plasma concentrations of anti-human IL-6R antibody and soluble IL-6R are shown in FIG. 36 and FIG. 37, respectively.

In the group of mice administered with Fv4-P587-LS in which the binding activity of Fv4-P587 to human FcRn under an acidic pH range condition has been enhanced, plasma retention of antibodies was found to be improved compared to that in the group of mice administered with Fv4-P587. In addition, Fv4-P587-LS showed improved plasma retention than Fv4-IgG1. On the other hand, the plasma concentration of soluble IL-6R in the group of Fv4-P587-LS-administered mice was equivalent to that in the group of Fv4-P587-administered mice. In the group of Fv4-P587-LS- or Fv4-P587-administered mice, plasma concentration of soluble IL-6R was decreased compared to in the group of Fv4-IgG1-administered mice.

Accordingly, administration of antibody in which the human FcRn-binding activity of an antigen-binding molecule under an acidic pH range condition has been enhanced, wherein human FcγRIIb-binding activity of the antigen-binding molecule is higher than that of a native human IgG Fc region, showed that plasma retention of the administered antigen molecule can be improved in a living organism receiving the administration. Furthermore, even if the plasma retention is improved in a living organism administered with the antigen-binding molecule, the antigen-eliminating effect of the living organism was shown not to decrease, but rather can be maintained.

Alterations to enhance the human FcRn-binding activity under an acidic pH range condition are not particularly limited; and include: the method for substituting Leu for Met at position 428 and Ser for Asn at position 434 (EU numbering) in an IgG antibody (Nat. Biotechnol, (2010) 28, 157-159); the method for substituting Ala for Asn at position 434 (Drug. Metab. Dispos. (2010) 38 (4), 600-605); the method for substituting Tyr for Met at position 252, Thr for Ser at position 254, and Glu for Thr at position 256 (J. Biol. Chem. (2006) 281, 23514-23524); the method for substituting Gln for Thr at position 250 and Leu for Met at position 428 (J. Immunol. (2006) 176 (1) 346-356); the method for substituting His for Asn at position 434 (Clin. Pharm. & Ther. (2011) 89 (2) 283-290.); and alterations disclosed in WO2010/106180, WO2010/045193, WO2009/058492, WO2008/022152, WO2006/050166, WO2006/053301, WO2006/031370, WO2005/123780, WO2005/047327, WO2005/037867, WO2004/035752, WO2002/060919, and such.

### [Reference Example 1] Construction of Antibody Expression Vectors; and Expression and Purification of Antibodies

Synthesis of full-length genes encoding the nucleotide sequences of the H chain and L chain of the antibody variable regions was carried out by production methods known to those skilled in the art using Assemble PCR and such. Introduction of amino acid substitutions was carried out by methods known to those skilled in the art using PCR or such. The obtained plasmid fragment was inserted into an animal cell expression vector, and the H-chain expression vector and L-chain expression vector were produced. The nucleotide sequence of the obtained expression vector was determined by methods known to those skilled in the art. The produced plasmids were introduced transiently into the HEK293H cell line derived from human embryonic kidney cancer cells (Invitrogen) or into FreeStyle293™ cells (Invitrogen) for antibody expression. The obtained culture supernatant was collected, and then passed through a 0.22 μm MILLEX(R)-GV filter (Millipore), or through a 0.45 μm MILLEX(R [symbol] GV filter (Millipore) to obtain the culture supernatant. Antibodies were purified from the obtained culture supernatant by methods known to those skilled in the art using a Protein A Sepharose® 4 Fast Flow gel filtration medium (GE Healthcare) or a Protein G Sepharose® 4 Fast Flow gel filtration medium (GE Healthcare). For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4:2411-2423).

### [Reference Example 2] Method for Preparing FcγR and Method for Analyzing the Interaction Between an Altered Antibody and FcγR

Extracellular domains of FcγRs were prepared by the following method. First, a gene of the extracellular domain of FcγR was synthesized by a method well known to those skilled in the art. At that time, the sequence of each FcγR was produced based on the information registered at NCBI. Specifically, FcγRI was produced based on the sequence of NCBI Accession No. NM_000566.3, FcγRIIa was produced based on the sequence of NCBI Accession No. NM_001136219.1, FcγRIIb was produced based on the sequence of NCBI Accession No. NM_004001.3, FcγRIIIa was produced based on the sequence of NCBI Accession No. NM_001127593.1, and FcγRIIIb was produced based on the sequence of NCBI Accession No. NM_000570.3, and a His tag was attached to the C terminus. Furthermore, polymorphism is known for FcγRIIa, FcγRIIIa, and FcγRIIIb, and the polymorphic sites were produced by referring to J. Exp. Med., 1990, 172:19-25 for FcγRIIa; J. Clin. Invest., 1997, 100 (5): 1059-1070 for FcγRIIIa; and J. Clin. Invest., 1989, 84, 1688-1691 for FcγRIIIb.

The obtained gene fragments were inserted into an animal cell expression vector, and expression vectors were produced. The produced expression vectors were introduced transiently into human embryonic kidney cancer cell line-derived FreeStyle293™ cells (Invitrogen) to express the proteins of interest. Regarding FcγRIIb used for crystallographic analysis, the protein of interest was expressed in the presence of Kifunensine at a final concentration of 10 μg/mL, so that the sugar chain added to FcγRIIb will be the high-mannose type. Cells were cultured, and after collection of the obtained culture supernatant, this was passed through a 0.22 μm filter to obtain the culture supernatant. In principle, the obtained culture supernatants were purified in the following four steps. The steps carried out were, cation exchange column chromatography (SP Sepharose® 4 FF gel filtration medium) in step 1, affinity column chromatography (HisTrap™ HP affinity column) for His tag in step 2, gel filtration column chromatography (Superdex® 200 gel filtration column) in step 3, and aseptic chromatography in step 4. However, for FcγRI, anion exchange column chromatography using Q Sepharose® 4 FF chromatography medium was performed as step 1. The purified proteins were subjected to absorbance measurements at 280 nm using a spectrophotometer; and from the obtained values, the concentrations of the purified proteins were calculated using the absorption coefficient calculated using methods such as PACE (Protein Science 1995; 4:2411-2423).

Analysis of interaction between each altered antibody and the Fcγ receptor prepared as mentioned above was carried out using a Biacore™ T100 surface plasmon resonance system (GE Healthcare), a Biacore™ T200 surface plasmon resonance system (GE Healthcare), a Biacore™ A100 surface plasmon resonance system, and Biacore™ 4000 surface plasmon resonance system. HBS-EP+ (0.01 M HEPES pH 7.4, 0.15 M NaCl, 3 mM ethylene diamine tetraacetic acid (EDTA), 0.05% polysorbate 20) (GE Healthcare) was used as the running buffer, and the measurement temperature was set to 25° C. Chips produced by immobilizing the antigen peptide, Protein A (Thermo Scientific), Protein A/G (Thermo Scientific), and Protein L (ACTIGEN or Bio Vision) by the amine coupling method to a Series S sensor Chip CM5 (GE Healthcare) or Series S sensor Chip CM4 (GE Healthcare), or alternatively, chips produced by allowing preliminarily biotinylated antigen peptides to interact with and immobilize onto a Series S Sensor Chip SA (certified) (GE Healthcare) were used.

After capturing of antibodies of interest onto these sensor chips, an Fcγ receptor diluted with the running buffer was allowed to interact, the amount bound to an antibody was measured, and the antibodies were compared. However, since the amount of Fcγ receptor bound depends on the amount of the captured antibodies, the amount of Fcγ receptor bound was divided by the amount of each antibody captured to obtain corrected values, and these values were compared. Furthermore, antibodies captured onto the chips were washed by reaction with 10 mM glycine-HCl, pH 1.5, and the chips were regenerated and used repeatedly.

Kinetic analyses for calculating the KD values of each altered antibody for FcγR were performed according to the following method. First, antibodies of interest were captured onto the above-mentioned sensor chips, and an Fcγ receptor diluted with the running buffer was allowed to interact. The Biacore™ Evaluation Software was used to globally fit the measured results to the obtained sensorgram using the 1:1 Langmuir binding model, and the association rate constant ka (L/mol/s) and the dissociation rate constant kd (1/s) were calculated; and from those values the dissociation constants KD(mol/L) were calculated.

When the interaction between each of the altered antibodies and FcγR was weak, and correct analysis was determined to be impossible by the above-mentioned kinetic analysis, the KD for such interactions were calculated using the following 1:1 binding model equation described in the Biacore™ T100 Software Handbook BR1006-48 Edition AE.

The behavior of interacting molecules according to the 1:1 binding model on a Biacore™ surface plasmon resonance system can be described by Equation 1 shown below.

R_{eq}=C·Rₘₐₓ/(KD+C)+RI [Equation 1]
R_{eq}: a plot of steady-state binding levels against analyte concentration
C: concentration
RI: bulk refractive index contribution in the sample
Rₘₐₓ: analyte binding capacity of the surface

When this equation is rearranged, KD can be expressed as Equation 2 shown below.

KD=C·Rₘₐₓ/(R_{eq}−RI)−C [Equation 2]

KD can be calculated by substituting the values of Rₘₐₓ, RI, and C into this equation. The values of RI and C can be determined from the sensorgram of the measurement results and measurement conditions. Rₘₐₓ was calculated according to the following method. As a target of comparison, for antibodies that had sufficiently strong interactions as evaluated simultaneously in the same round of measurement, the Rₘₐₓ value was obtained through global fitting using the 1:1 Langmuir binding model, and then it was divided by the amount of the comparison antibody captured onto the sensor chip, and multiplied by the captured amount of an altered antibody to be evaluated.

### [Reference Example 3] Comprehensive Analysis of the Binding of Fc Variants to FcγR

Mutations were introduced into IgG1 antibodies to generate antibodies that have decreased Fc-mediated binding towards activating FcγR, specifically both allotypes of FcγRIIa, types H and R, as well as enhanced FcγRIIb binding relative to IgG1; and binding to each FcγR was analyzed comprehensively.

The variable region (SEQ ID NO: 15) of a glypican 3 antibody comprising the CDR of GpH7 which is an anti-glypican 3 antibody with improved plasma kinetics disclosed in WO 2009/041062 was used as the common antibody H chain. Similarly, for the common antibody L chain, GpL16-k0 (SEQ ID NO: 16) of the glypican 3 antibody with improved plasma kinetics disclosed in WO 2009/041062 was used. Furthermore, B3 in which a K439E mutation has been introduced into G1d produced by removing the C terminal Gly and Lys of IgG1 was used as the antibody H chain constant region. This H chain is referred to as GpH7-B3 (SEQ ID NO: 17), and the L chain is referred to as GpL16-k0 (SEQ ID NO: 16).

With respect to GpH7-B3, the amino acids that are considered to be involved in FcγR binding and the surrounding amino acids (positions 234 to 239, 265 to 271, 295, 296, 298, 300, and 324 to 337, according to EU numbering) were substituted respectively with 18 types of amino acids excluding the original amino acids and Cys. These Fc variants are referred to as B3 variants. B3 variants were expressed and purified using the method of Reference Example 1, and the binding to each FcγR (FcγRIa, FcγRIIa type H, FcγRIIa type R, FcγRIIb, and FcγRIIIa) was comprehensively evaluated using the method of Reference Example 2.

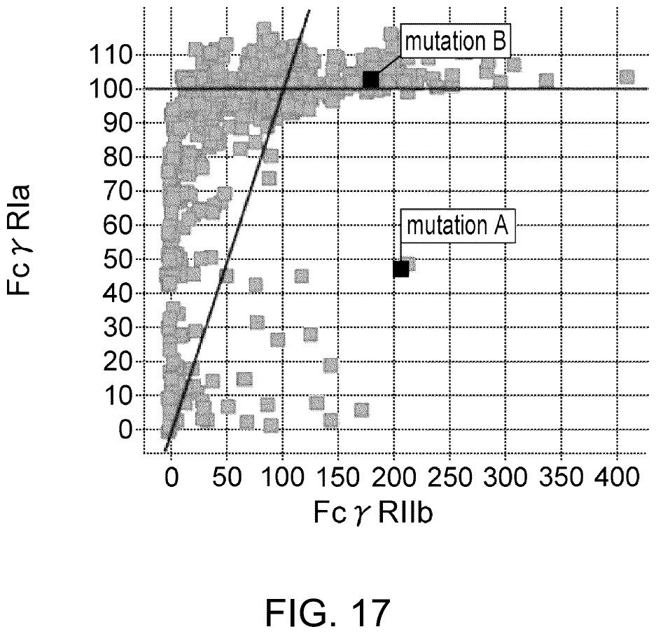

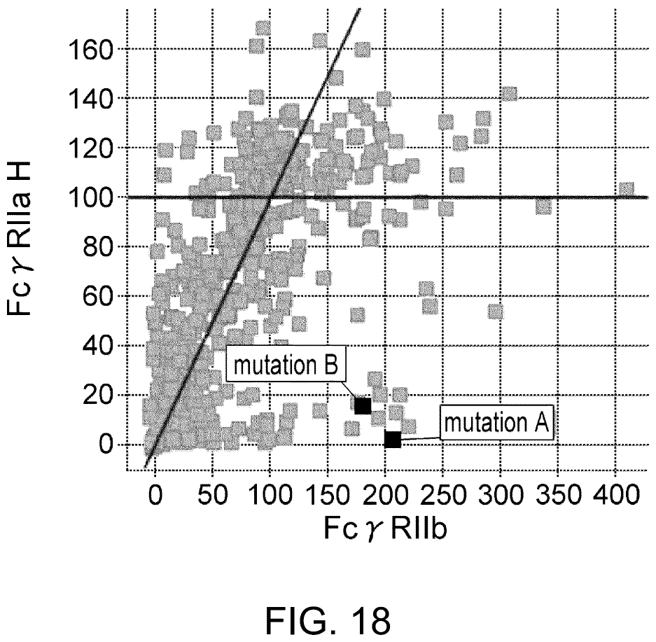

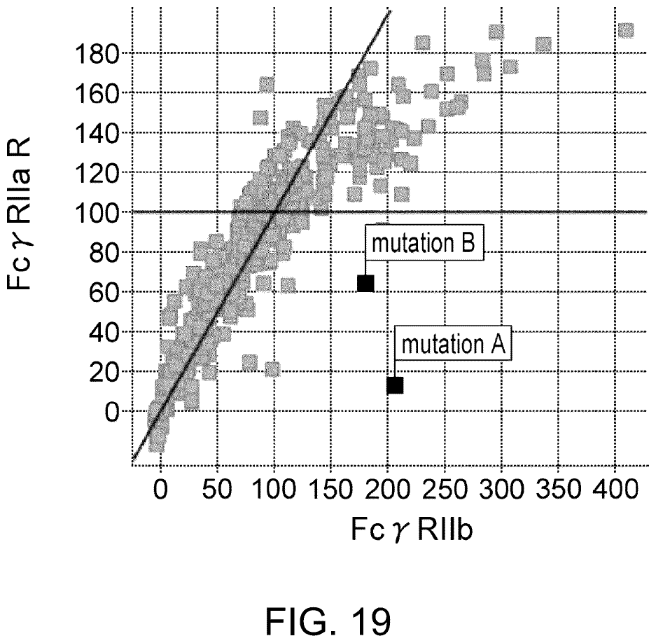

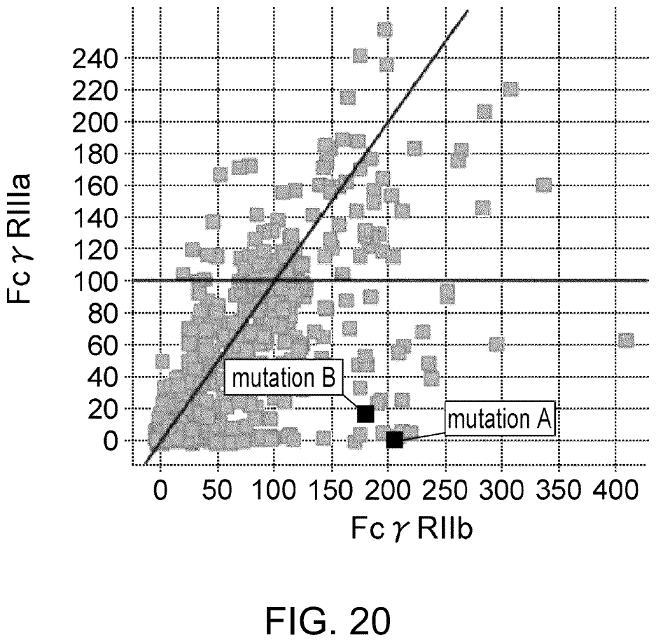

Figures were produced based on the results of interaction analysis with each FcγR by the method below. The value of the amount of FcγR binding of each B3 variant-derived antibody was divided by the value of the amount of FcγR binding of the antibody used for comparison which does not have mutations introduced into B3 (an antibody having the sequence of a naturally-occurring human IgG1 at positions 234 to 239, 265 to 271, 295, 296, 298, 300, and 324 to 337, according to EU numbering). The value obtained by multiplying this value by 100 was used as an indicator of the relative FcγR-binding activity of each variant. The horizontal axis shows the value of the relative FcγRIIb-binding activity of each variant, and the vertical axis shows the value of the respective relative binding activity of each variant towards activating FcγRs: FcγRIa, FcγRIIa type H, FcγRIIa type R, and FcγRIIIa (FIGS. 17, 18, 19, and 20).

As shown by labels in FIGS. 17-20, the results show that of all alterations, when only mutations called mutation A (alteration produced by substituting Pro at position 238 (EU numbering) with Asp) and mutation B (alteration produced by substituting Leu at position 328 (EU numbering) with Glu) were introduced, there were remarkable enhancement of binding to FcγRIIb and remarkable suppression of binding to both types of FcγRIIa compared with before the introduction.

### [Reference Example 4] SPR Analysis of Variants that Selectively Bind to FcγRIIb

With regard to the alteration identified in Example 1 where Pro at position 238 (EU numbering) is substituted with Asp, the binding to each FcγR was analyzed in detail.

The variable region of IL6R-H (SEQ ID NO: 18), which is the variable region of the antibody against the human interleukin 6 receptor disclosed in WO 2009/125825, was used as the antibody H chain variable region, and IL6R-G1d (SEQ ID NO: 19) which comprises G1d with deletion of C-terminal Gly and Lys of human IgG1 was used as the antibody H chain constant region in the IgG1 H chain. Pro at position 238 (EU numbering) in IL6R-G1d was substituted with Asp to produce IL6R-G1d-v1 (SEQ ID NO: 20). Next, Leu at position 328 (EU numbering) in IL6R-G1d was substituted with Glu to produce IL6R-G1d-v2. Furthermore, for comparison, Ser at position 267 (EU numbering) was substituted with Glu, and Leu at position 328 (EU numbering) was substituted with Phe in IL6R-G1d to produce IL6R-G1d-v3 as described in Non-patent Document 28. IL6R-L (SEQ ID NO: 21), which is the L chain of tocilizumab, was utilized as a mutual antibody L chain; and together with each H chain, the antibodies were expressed and purified according to the method of Reference Example 1. The obtained antibodies which comprise an amino acid sequence derived from IL6R-G1d, IL6R-G1d-v1, IL6R-G1d-v2, or IL6R-G1d-v3 as the antibody H chain are referred to as IgG1, IgG1-v1, IgG1-v2, and IgG1-v3, respectively.

Next, kinetic analysis of interactions between these antibodies and FcγR was carried out using a Biacore™ T100 surface plasmon resonance system (GE Healthcare). HBS-EP+ (GE Healthcare) was used as the running buffer, and the measurement temperature was set to 25° C. A chip produced by immobilizing Protein A onto a Series S Sensor Chip CM5 (GE Healthcare) by the amine-coupling method was used. An antibody of interest was captured onto this chip to interact with each FcγR that had been diluted with the running buffer, and binding to the antibody was measured. After the measurement, the antibody captured on the chip was washed off by allowing reaction with 10 mM glycine-HCl, pH 1.5, and the chip was regenerated and used repeatedly. The sensorgrams obtained as measurement results were analyzed by the 1:1 Langmuir binding model using the Biacore™ Evaluation Software to calculate the binding rate constant ka (L/mol/s) and dissociation rate constant kd (1/s), and the dissociation constant KD (mol/L) was calculated from these values.

This time, since the binding of IgG1-v1 and IgG1-v2 to FcγRIIa type H and to FcγRIIIa was weak, kinetic parameters such as KD could not be calculated from the above-mentioned analytical method. Regarding such interactions, KD values were calculated using the following 1:1 binding model described in the Biacore™ T100 Software Handbook BR1006-48 Edition AE.

The behavior of interacting molecules according to the 1:1 binding model on a Biacore™ surface plasmon resonance system can be described by Equation 1 shown below.

R_{eq}=C·Rₘₐₓ/(KD+C)+RI [Equation 1]
R_{eq}: a plot of steady-state binding levels against analyte concentration
C: concentration
RI: bulk refractive index contribution in the sample
Rₘₐₓ: analyte binding capacity of the surface

When this equation is rearranged, KD can be expressed as Equation 2 shown below.

KD=C·Rₘₐₓ/(R_{eq}−RI)−C [Equation 2]

KD can be calculated by substituting the values of Rₘₐₓ, RI, and C into this equation. From the current measurement conditions, RI=0, C=2 μmol/L can be used. Furthermore, the Rₘₐₓ value obtained when globally fitting the sensorgram obtained as a result of analyzing the interaction of each FcγR with IgG1 using the 1:1 Langmuir binding model was divided by the amount of IgG1 captured, this was multiplied by the amount of IgG1-v1 and IgG1-v2 captured, and the resulting value was used as Rₘₐₓ. This calculation is based on the hypothesis that the limit quantity of each FcγR that can be bound by IgG1 remains unchanged for all variants produced by introducing mutations into IgG1, and the Rₘₐₓ at the time of measurement is proportional to the amount of antibody bound on the chip at the time of measurement. R_{eq} was defined as the amount of binding of each FcγR to each variant on the sensor chip observed at the time of measurement.

Under these measurement conditions, the amount of binding (R_{eq}) of IgG1-v1 and IgG1-v2 to FcγRIIa type H was approximately 2.5 RU and 10 RU, respectively, and the amount of binding (R_{eq}) of IgG1-v1 and IgG1-v2 to FcγRIIIa was approximately 2.5 RU and 5 RU, respectively. The amount of IgG1, IgG1-v1, and IgG1-v2 captured in the analysis of interactions with H-type FcγRIIa was 452 RU, 469.2 RU, and 444.2 RU, respectively, and the amount of IgG1, IgG1-v1, and IgG1-v2 captured in the analysis of interactions with FcγRIIIa was 454.5 RU, 470.8 RU, and 447.1 RU, respectively. The Rₘₐₓ values obtained from global fitting of sensorgrams obtained as a result of analyzing the interaction of IgG1 with H-type FcγRIIa and FcγRIIIa using the 1:1 Langmuir binding model were 69.8 RU and 63.8 RU, respectively. When these values were used, the calculated Rₘₐₓ values of IgG1-v1 and IgG1-v2 to FcγRIIa type H were 72.5 RU and 68.6 RU, respectively, and the calculated Rₘₐₓ values of IgG1-v1 and IgG1-v2 to FcγRIIIa were 66.0 RU and 62.7 RU, respectively. These values were substituted into Equation 2 to calculate the KD of IgG1-v1 and IgG1-v2 for FcγRIIa type H and FcγRIIIa.

KD=C·Rₘₐₓ/(R_{eq}−RI)−C [Equation 2]

The KD values of IgG1, IgG1-v1, IgG1-v2, and IgG1-v3 for each FcγR (the KD values of each antibody for each FcγR) are shown in Table 15, and the relative KD values of IgG1-v1, IgG1-v2, and IgG1-v3 obtained by taking the KD values of IgG1 for each FcγR and dividing them by the KD values of IgG1-v1, IgG1-v2, and IgG1-v3 for each FcγR (the relative KD values of each antibody for each FcγR) are shown in Table 16.

TABLE 15

| | IgG1 | IgG1-v1 | IgG1-v2 | IgG1-v3 |
|---|---|---|---|---|
| FcγRIa | 3.4E−10 | 7.3E−09 | 4.6E−10 | 1.9E−10 |
| FcγRIIa R | 1.2E−06 | 1.2E−05 | 2.9E−06 | 2.3E−09 |
| FcγRIIa H | 7.7E−07 | 5.6E−05* | 1.2E−05* | 1.5E−06 |
| FcγRIIb | 5.3E−06 | 1.1E−06 | 2.3E−06 | 1.3E−08 |
| FcγRIIIa | 3.1E−06 | 5.1E−05* | 2.3E−05* | 8.8E−06 |

(mol/L)

In Table 15 shown above, “*” means that the KD value was calculated using Equation 2 because binding of FcγR to IgG was not sufficiently observed.

KD=C·Rₘₐₓ/(R_{eq}−RI)−C [Equation 2]

TABLE 16

| | IgG1-v1 | IgG1-v2 | IgG1-v3 |
|---|---|---|---|
| FcγRIa | 0.047 | 0.74 | 1.8 |
| FcγRIIa R | 0.10 | 0.41 | 522 |
| FcγRIIa H | 0.014 | 0.064 | 0.51 |
| FcγRIIb | 4.8 | 2.3 | 408 |
| FcγRIIIa | 0.061 | 0.14 | 0.35 |

(THE VALUE OBTAINED BY DIVIDING THE KD VALUE OF IgG1 FOR EACH FcγR BY THE KD VALUE OF EACH ANTIBODY IgG1 FOR EACH FcγR)

According to Table 16, when compared with that of IgG1, the binding activity of IgG1-v1 was decreased to 0.047-fold for FcγRIa, decreased to 0.10-fold for FcγRIIa type R, decreased to 0.014-fold for FcγRIIa type H, decreased to 0.061-fold for FcγRIIIa, and increased to 4.8-fold for FcγRIIb.

Furthermore, according to Table 16, when compared with that of IgG1, the binding activity of IgG1-v2 was decreased to 0.74-fold for FcγRIa, decreased to 0.41-fold for FcγRIIa type R, decreased to 0.064-fold for FcγRIIa type H, decreased to 0.14-fold for FcγRIIIa, and increased to 2.3-fold for FcγRIIb.

More specifically, these results demonstrated that IgG1-v1 having an alteration of substituting Pro at position 238 (EU numbering) with Asp and IgG1-v2 having an alteration of substituting Leu at position 328 (EU numbering) with Glu have the properties of weakening the binding to all activating FcγRs including both allotypes of FcγRIIa, while enhancing the binding to FcγRIIb which is an inhibitory FcγR.

Next, selectivity of the obtained variant to FcγRIIb was evaluated by using the ratio of FcγRIIb-binding activity to the binding activity towards type R or type H of FcγRIIa as the indicator. Specifically, I/A(R) or I/A(H), which is a value obtained by dividing the KD value for FcγRIIa type R or type H by the KD value for FcγRIIb, was used as an indicator for the selectivity of FcγRIIb with respect to each FcγRIIa. This indicator has a greater value when the KD value for FcγRIIb becomes smaller or when the KD value for FcγRIIa becomes larger. That is, a variant that shows a larger value shows an increased binding activity for FcγRIIb relative to FcγRIIa. These indicators are summarized in Table 17 for each variant.

TABLE 17

| | IgG1 | IgG1-v1 | IgG1-v2 | IgG1-v3 |
|---|---|---|---|---|
| I/A (R) | 0.23 | 11 | 1.3 | 0.18 |
| I/A (H) | 0.15 | 51 | 5.2 | 115 |

According to the results of Table 17, in comparison with IgG1, IgG1-v3 which was produced by applying the existing technology showed a greater I/A(H) value than that of IgG1 and a greater selectivity for FcγRIIb, but a smaller I/A(R) value than that of IgG1 and an improved selectivity for FcγRIIb. On the other hand, IgG1-v1 and IgG1-v2 found in the Examples have larger I/A(R) and I/A(H) values than those of IgG1, and improved selectivity for FcγRIIb over both allotypes of FcγRIIa.

So far, alterations having such properties have not been reported, and they are in fact very rare as shown in FIGS. 17, 18, 19, and 20. Alterations produced by substituting Pro at position 238 (EU numbering) with Asp or substituting Leu at position 328 (EU numbering) with Glu are very useful for the development of therapeutic agents for immunological inflammatory diseases and such

Furthermore, Table 16 shows that IgG1-v3 described in Non-patent Document 28 certainly shows a 408-fold enhanced binding to FcγRIIb, while the binding to FcγRIIa type His decreased to 0.51 fold, and the binding to FcγRIIa type R is enhanced to 522 fold. According to these results, since IgG1-v1 and IgG1-v2 suppress their binding to both FcγRIIa types R and H, and enhance their binding to FcγRIIb, they are considered to be variants that bind with a greater FcγRIIb selectivity compared with IgG1-v3. Specifically, alterations produced by substituting Pro at position 238 (EU numbering) with Asp or substituting Leu at position 328 (EU numbering) with Glu are very useful for the development of therapeutic agents for immunological inflammatory diseases and such.

### [Reference Example 5] Effects of Combining FcγRIIb-Selective Binding Alterations with Other Fc Region Amino Acid Substitutions

Further enhancement of the selectivity for FcγRIIb was attempted based on the variant which has improved selectivity for FcγRIIb and has a substitution of Pro at position 238 (EU numbering) with Asp found in Reference Examples 3 and 4.

First, into IL6R-G1d_v1 (SEQ ID NO: 20) produced by introducing into IL6R-G1d the alteration produced by substituting Pro at position 238 (EU numbering) with Asp, the substitution of Leu at position 328 (EU numbering) with Glu as described in Reference Example 4 which enhances selectivity for FcγRIIb was introduced to produce the IL6R-G1d-v4 variant. This was combined with IL6R-L (SEQ ID NO: 21) and prepared according to the method of Reference Example 1. The obtained antibody having the amino acid sequence derived from IL6R-G1d-v4 as the antibody H chain has been named IgG1-v4. The binding activities of IgG1, IgG1-v1, IgG1-v2, and IgG1-v4 to FcγRIIb were evaluated according to the method of Reference Example 2, and those results are shown in Table 18.

TABLE 18

| | | | Relative KD |
|---|---|---|---|
| | | KD for | for FcγRIIb |
| | | FcγRIIb | (KD of IgG1/KD |
| Variant | Alteration | (mol/L) | of each variant) |
| IgG1 | — | 5.30E−06 | 1 |
| IgG1-v1 | Substitution of Pro at position | 1.10E−06 | 4.8 |
| | 238 (EU numbering) with Asp | | |
| IgG1-v2 | Substitution of Leu at position | 2.30E−06 | 2.3 |
| | 328 (EU numbering) with Glu | | |
| IgG1-v4 | Substitution of Pro at position | 1.10E−05 | 0.47 |
| | 238 (EU numbering) with Asp | | |
| | and substitution of Leu at | | |
| | position 328 (EU numbering) | | |
| | with Glu | | |

From the results of Table 18, since L328E improves the FcγRIIb-binding activity by 2.3 fold compared with IgG1, combining it with P238D which similarly improves the FcγRIIb-binding activity by 4.8 fold compared with IgG1 was anticipated to further increase the degree of improvement of FcγRIIb-binding activity; however, in reality, the FcγRIIb-binding activity of the variant containing a combination of these alterations was decreased to 0.47 fold compared with that of IgG1. This result is an effect that could not have been predicted from the respective alterations.

Similarly, into IL6R-G1d-v1 (SEQ ID NO: 20) produced by introducing into IL6R-G1d the alteration produced by substituting Pro at position 238 (EU numbering) with Asp, the substitutions of Ser at position 267 (EU numbering) with Glu and of Leu at position 328 (EU numbering) with Phe as described in Reference Example 4 which improve FcγRIIb-binding activity were introduced, and the IL6R-G1d-v5 variant was prepared according to the method of Reference Example 1. The obtained antibody having the amino acid sequence derived from IL6R-G1d-v5 as the antibody H chain has been named IgG1-v5. The FcγRIIb-binding activities of IgG1, IgG1-v1, IgG1-v3, and IgG1-v5 were evaluated according to the method of Reference Example 2, and those results are shown in Table 19.

S267E/L328F which had an enhancing effect on FcγRIIb in Reference Example 4 was introduced into the P238D variant and its FcγRIIb-binding activities before and after introducing this alteration were evaluated. The results are shown in Table 19.

TABLE 19

| | | | Relative KD |
|---|---|---|---|
| | | KD for | for FcγRIIb |
| | | FcγRIIb | (KD of IgG1/KD |
| Variant | Alteration | (mol/L) | of each variant) |
| IgG1 | — | 5.30E−06 | 1 |
| IgG1-v1 | Substitution of Pro at position | 1.10E−06 | 4.8 |
| | 238 (EU numbering) with Asp | | |
| IgG1-v3 | Substitution of Ser at position | 1.30E−08 | 408 |
| | 267 (EU numbering) with Glu | | |
| | and substitution of Leu at | | |
| | position 328 (EU numbering) | | |
| | with Phe | | |
| IgG1-v5 | Substitution of Pro at position | 4.50E−07 | 12 |
| | 238 (EU numbering) with Asp, | | |
| | substitution of Ser at | | |
| | position 267 (EU numbering) | | |
| | with Glu, and substitution | | |
| | of Leu at position 328 (EU | | |
| | numbering) with Phe | | |

From the results of Table 19, since S267E/L328F improves the FcγRIIb-binding activity by 408 fold compared with IgG1, combining it with P238D which similarly improves the FcγRIIb-binding activity by 4.8 fold as compared with IgG1 was anticipated to further increase the degree of improvement of FcγRIIb-binding activity; however, in reality, in a similar manner to the former example, the FcγRIIb-binding activity of the variant containing a combination of these alterations was improved only 12 fold or so as compared with that of IgG1. This result is also an effect that could not have been predicted from the effects of the respective alterations.

These results showed that while the substitution of Pro at position 238 (EU numbering) with Asp alone improves FcγRIIb-binding activity, the effect is not exhibited when it is combined with other alterations that improve the FcγRIIb-binding activity. A reason for this may be that the structure at the interacting interface between Fc and FcγR is changed by introducing the substitution of Pro at position 238 (EU numbering) with Asp and the effects of alterations observed in the naturally-occurring antibody are no longer reflected in the results. Accordingly, it was considered to be extremely difficult to create an Fc with excellent selectivity for FcγRIIb using an Fc comprising substitution of Pro at position 238 (EU numbering) with Asp as a template, since the information on effects of alterations obtained with naturally-occurring antibodies could not be applied.

### [Reference Example 6] Comprehensive Analysis of FcγRIIb Binding of Variants Introduced with an Alteration at the Hinge Portion in Addition to the P238D Alteration

As shown in Reference Example 5, in an Fc produced by substituting Pro at position 238 (EU numbering) with Asp in a naturally-occurring human IgG1, an anticipated combinatorial effect could not be obtained even by combining it with another alteration predicted to further increase FcγRIIb binding. Therefore, based on the Fc variant produced by substituting Pro at position 238 (EU numbering) with Asp, examination was carried out by comprehensively introducing alterations into the Fc to find variants that further enhance FcγRIIb binding. For the antibody H chains, IL6R-F11 (SEQ ID NO: 22) was produced by introducing an alteration of substituting Met at position 252 (EU numbering) with Tyr and an alteration of substituting Asn at position 434 (EU numbering) with Tyr into IL6R-G1d (SEQ ID NO: 19), and IL6R-F652 was prepared by introducing an additional alteration of substituting Pro at position 238 (EU numbering) with Asp. Expression plasmids containing an antibody H chain sequence were prepared for each of the antibody H chain sequences produced by substituting the region near the residue at position 238 (EU numbering) (positions 234 to 237, and 239 (EU numbering)) in IL6R-F652 each with 18 amino acids excluding the original amino acids and cysteine. IL6R-L (SEQ ID NO: 21) was utilized as a common antibody L chain for all of the antibodies. These variants were expressed, purified, and expressed by the method of Reference Example 1. These Fc variants are called PD variants. Interactions of each PD variant with FcγRIIa type R and FcγRIIb were comprehensively evaluated by the method of Reference Example 2.

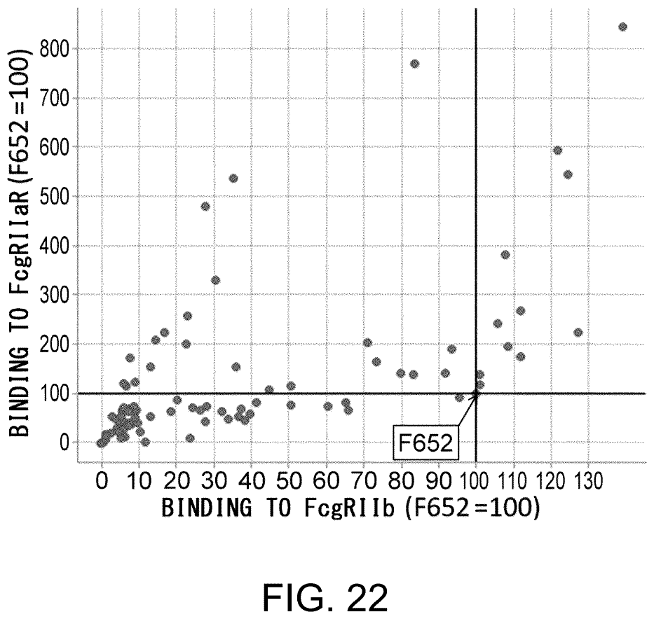

With regard to the results of analyzing the interaction with the respective FcγRs, a figure was produced according to the following method. The value obtained by dividing the value for the amount of binding of each PD variant to each FcγR by the value for the amount of FcγR binding of the pre-altered antibody which is used as the control (IL6R-F652/IL6R-L, which has an alteration of substituting Pro at position 238 (EU numbering) with Asp) and then multiplying the result by 100, was used as the relative binding activity value of each PD variant to each FcγR. The horizontal axis shows relative values of the FcγRIIb-binding activity of each PD variant, and the vertical axis shows relative values of the FcγRIIa type R-binding activity values of each PD variant (FIG. 22).

As a result, eleven types of alterations were found to have the effects of enhancing FcγRIIb binding and maintaining or enhancing FcγRIIa type R-binding in comparison with the antibody before introducing alterations. The activities of these eleven variants to bind FcγRIIb and FcγRIIa R are summarized in Table 20. In the table, “alteration” refers to the alteration introduced into IL6R-F11 (SEQ ID NO: 22).

TABLE 20

| | | RELATIVE | RELATIVE |
|---|---|---|---|
| | | BINDING | BINDING |
| | | ACTIVITY | ACTIVITY |
| VARIANT NAME | ALTERATION | TO FcγRIIb | TO FcγRIIaR |
| IL6R-F652/IL6R-L | P238D | 100 | 100 |
| IL6R-PD042/IL6R-L | P238D/L234W | 106 | 240 |
| IL6R-PD043/IL6R-L | P238D/L234Y | 112 | 175 |
| IL6R-PD079/IL6R-L | P238D/G237A | 101 | 138 |
| IL6R-PD080/IL6R-L | P238D/G237D | 127 | 222 |
| IL6R-PD081/IL6R-L | P238D/G237E | 101 | 117 |
| IL6R-PD082/IL6R-L | P238D/G237F | 108 | 380 |
| IL6R-PD086/IL6R-L | P238D/G237L | 112 | 268 |
| IL6R-PD087/IL6R-L | P238D/G237M | 109 | 196 |
| IL6R-PD094/IL6R-L | P238D/G237W | 122 | 593 |
| IL6R-PD095/IL6R-L | P238D/G237Y | 124 | 543 |
| IL6R-PD097/IL6R-L | P238D/S239D | 139 | 844 |

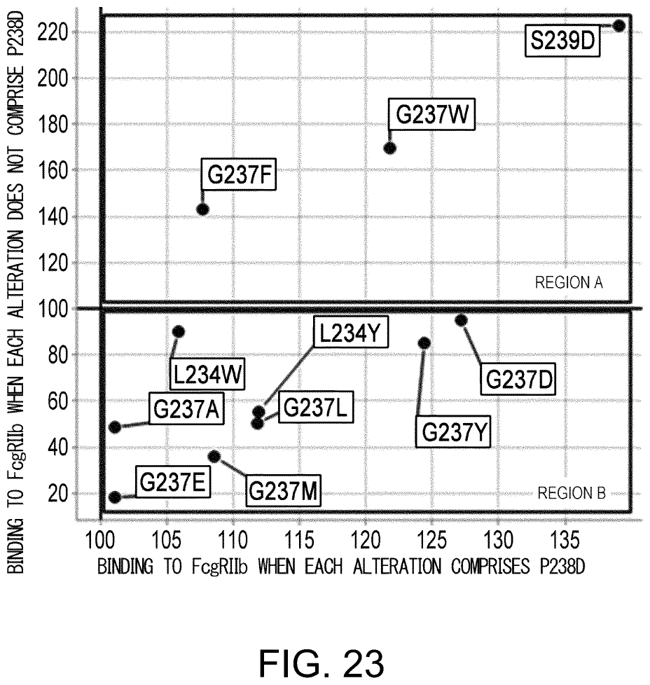

FIG. 23 shows relative values for the FcγRIIb-binding activity obtained by additionally introducing these eleven alterations into a variant carrying the P238D alteration, and relative values for the FcγRIIb-binding activity obtained by introducing these alterations into an Fc that does not contain the P238D alteration in Reference Example 3. These eleven alterations enhanced the amount of FcγRIIb binding compared with before introduction when they were further introduced into the P238D variant, but on the contrary, the effect of lowering FcγRIIb binding was observed for eight of those alterations except G237F, G237W, and S239D, when they were introduced into the variant that does not contain P238D (GpH7-B3/GpL16-k0) used in Reference Example 3.

Reference Example 5 and these results showed that from the effects of introducing alterations into a naturally-occurring IgG1, it is difficult to predict the effects of introducing the same alterations into the variant containing an Fc with the P238D alteration. In other words, it would not have been possible to discover these eight alterations identified this time without this investigation.

The results of measuring KD values of the variants indicated in Table 20 for FcγRIa, FcγRIIaR, FcγRIIaH, FcγRIIb, and FcγRIIIaV by the method of Reference Example 2 are summarized in Table 21. In the table, “alteration” refers to the alteration introduced into IL6R-F11 (SEQ ID NO: 22). The template used for producing IL6R-F11, IL6R-G1d/IL6R-L, is indicated with an asterisk (*). Furthermore, “KD(IIaR)/KD(IIb)” and “KD(IIaH)/KD(IIb)” in the table respectively show the value obtained by dividing the KD value of each variant for FcγRIIaR by the KD value of each variant for FcγRIIb, and the value obtained by dividing the KD value of each variant for FcγRIIaH by the KD value of each variant for FcγRIIb. KD(IIb) of the parent polypeptide/KD(IIb) of the altered polypeptide refers to a value obtained by dividing the KD value of the parent polypeptide for FcγRIIb by the KD value of each variant for FcγRIIb. In addition, Table 21 shows KD values for the stronger of the FcγRIIaR- and FcγRIIaH-binding activities of each variant/KD values for the stronger of the FcγRIIaR- and FcγRIIaH-binding activities of the parent polypeptide. Here, parent polypeptide refers to a variant which has IL6R-F11 (SEQ ID NO: 22) as the H chain. It was determined that due to weak binding of FcγR to IgG, it was impossible to accurately analyze by kinetic analysis, and thus the values shown in bold italicized font in Table 21 were calculated by using Equation 2 of Reference Example 2.

KD=C·Rₘₐₓ/(R_{eq}−RI)−C [Equation 2]

Table 21 shows that all variants improved their affinity for FcγRIIb in comparison with IL6R-F11, and the range of improvement was 1.9 fold to 5.0 fold. The ratio of KD value of each variant for FcγRIIaR/KD value of each variant for FcγRIIb, and the ratio of KD value of each variant for FcγRIIaH/KD value of each variant for FcγRIIb represent an FcγRIIb-binding activity relative to the FcγRIIaR-binding activity and FcγRIIaH-binding activity, respectively. That is, these values show the degree of binding selectivity of each variant for FcγRIIb, and a larger value indicates a higher binding selectivity for FcγRIIb. For the parent polypeptide IL6R-F11/IL6R-L, the ratio of KD value for FcγRIIaR/KD value for FcγRIIb and the ratio of KD value for FcγRIIaH/KD value for FcγRIIb are both 0.7, and accordingly all variants in Table 21 showed improvement of binding selectivity for FcγRIIb in comparison with the parent polypeptide. When the KD value for the stronger of the FcγRIIaR- and FcγRIIaH-binding activities of a variant/KD value for the stronger of the FcγRIIaR- and FcγRIIaH-binding activities of the parent polypeptide is 1 or more, this means that the stronger of the FcγRIIaR- and FcγRIIaH-binding activities of a variant has equivalent or reduced binding compared with the binding by the stronger of the FcγRIIaR- and FcγRIIaH-binding activities of the parent polypeptide. Since this value was 0.7 to 5.0 for the variants obtained this time, one may say that binding by the stronger of the FcγRIIaR- and FcγRIIaH-binding activities of the variants obtained this time was nearly the same or decreased in comparison with the parent polypeptide. These results showed that compared with the parent polypeptide, the variants obtained this time have enhanced binding activity to FcγRIIb while having maintained or decreased binding activities to FcγRIIa type R and type H, and thus have improved selectivity for FcγRIIb. Furthermore, compared with IL6R-F11, all variants had lower affinity to FcγRIa and FcγRIIIaV.

TABLE 21

| | | KD (mol/L) | | | | | |
|---|---|---|---|---|---|---|---|
| VARIANT NAME | ALTERATION | FcγRIa | FcgRIIaR | FcgRIIaH | FcgRIIb | FcgRIIIaV | KD(IIaR)/KD(IIb) |
| IL6R-G1d/IL6R-L | * | 3.2E−10 | 1.0E−06 | 6.7E−07 | 2.6E−06 | 3.5E−07 | 0.4 |
| IL6R-F11/IL6R-L | | 9.0E−10 | 5.0E−06 | 5.0E−06 | 6.8E−06 | 1.5E−06 | 0.7 |
| IL6R-PD042/IL6R-L | L234W/P238D | 6.3E−08 | 1.6E−05 | 1.9E−05 | 2.0E−06 | 3.7E−05 | 8.1 |
| IL6R-PD043/IL6R-L | L234Y/P238D | 7.5E−08 | 2.6E−05 | 2.3E−05 | 1.6E−06 | 4.5E−05 | 15.9 |
| IL6R-PD079/IL6R-L | G237A/P238D | 1.4E−07 | 3.2E−05 | 2.1E−05 | 3.0E−06 | 3.7E−05 | 10.5 |
| IL6R-PD080/IL6R-L | G237D/P238D | 1.4E−07 | 2.1E−05 | 2.5E−05 | 2.0E−06 | 4.3E−05 | 10.7 |
| IL6R-PD081/IL6R-L | G237E/P238D | 3.4E−07 | 3.8E−05 | 2.5E−05 | 3.6E−06 | 4.1E−05 | 10.6 |
| IL6R-PD082/IL6R-L | G237F/P238D | 5.2E−08 | 1.4E−05 | 1.6E−05 | 3.4E−06 | 4.3E−05 | 4.1 |
| IL6R-PD086/IL6R-L | G237L/P238D | 1.2E−07 | 1.8E−05 | 1.8E−05 | 2.6E−06 | 4.1E−05 | 6.9 |
| IL6R-PD087/IL6R-L | G237M/P238D | 5.2E−08 | 2.2E−05 | 2.0E−05 | 2.9E−06 | 3.7E−05 | 7.7 |
| IL6R-PD094/IL6R-L | G237W/P238D | 3.6E−08 | 7.2E−06 | 1.2E−05 | 2.3E−06 | 3.8E−05 | 3.1 |
| IL6R-PD095/IL6R-L | G237Y/P238D | 9.3E−08 | 7.9E−06 | 1.5E−05 | 2.3E−06 | 4.2E−05 | 3.4 |
| IL6R-PD097/IL6R-L | P238D/S239D | 4.9E−09 | 3.5E−06 | 1.9E−05 | 1.4E−06 | 1.7E−05 | 2.6 |

| | | KD(IIb) OF PARENT | KD VALUE FOR THE STRONGER OF THE BINDING |
|---|---|---|---|
| | | POLYPEPTIDE/KD(IIb) | ACTIVITIES OF A VARIANT TO FcγRIIaR AND FcγRIIaH/KD |
| | | OF ALTERED | VALUE FOR THE STRONGER OF THE BINDING ACTIVITIES |
| VARIANT NAME | KD(IIaH)/KD(IIb) | POLYPEPTIDE | OF THE PARENT POLYPEPTIDE TO FcγRIIaR AND FcγRIIaH |
| IL6R-G1d/IL6R-L | 0.3 | 2.6 | 0.1 |
| IL6R-F11/IL6R-L | 0.7 | 1.0 | 1.0 |
| IL6R-PD042/IL6R-L | 9.5 | 3.4 | 3.2 |
| IL6R-PD043/IL6R-L | 14.4 | 4.2 | 4.6 |
| IL6R-PD079/IL6R-L | 7.0 | 2.3 | 4.2 |
| IL6R-PD080/IL6R-L | 12.8 | 3.5 | 4.2 |
| IL6R-PD081/IL6R-L | 7.0 | 1.9 | 5.0 |
| IL6R-PD082/IL6R-L | 4.7 | 2.0 | 2.8 |
| IL6R-PD086/IL6R-L | 7.1 | 2.7 | 3.5 |
| IL6R-PD087/IL6R-L | 7.0 | 2.4 | 4.0 |
| IL6R-PD094/IL6R-L | 5.2 | 2.9 | 1.4 |
| IL6R-PD095/IL6R-L | 6.4 | 2.9 | 1.6 |
| IL6R-PD097/IL6R-L | 14.0 | 5.0 | 0.7 |

### [Reference Example 7] X-Ray Structure Analysis of a Complex Formed Between an Fc Containing P238D and an Extracellular Region of FcγRIIb

As indicated earlier in Reference Example 5, even though an alteration that improves FcγRIIb-binding activity or selectivity for FcγRIIb is introduced into an Fc containing P238D, the FcγRIIb-binding activity was found to decrease, and the reason for this may be that the structure of the interacting interface between Fc and FcγRIIb is changed due to introduction of P238D. Therefore, to pursue the reason for this phenomena, the three-dimensional structure of the complex formed between an IgG1 Fc containing the P238D mutation (hereinafter, Fc(P238D)) and the extracellular region of FcγRIIb was elucidated by X-ray crystal structure analysis, and the three-dimensional structure and binding mode were compared to those of the complex formed between the Fc of a naturally-occurring IgG1 (hereinafter, Fc(WT)) and the extracellular region of FcγRIIb. Many reports have been made on the three-dimensional structure of a complex formed between an Fc and an FcγR extracellular region; and the three-dimensional structures of the Fc(WT)/FcγRIIIb extracellular region complex (Nature, 2000, 400:267-273; J. Biol. Chem. 2011, 276:16469-16477), the Fc(WT)/FcγRIIIa extracellular region complex (Proc. Natl. Acad. Sci. USA, 2011, 108:12669-126674), and the Fc(WT)/FcγRIIa extracellular region complex (J. Imunol. 2011, 187:3208-3217) have been analyzed. While the three-dimensional structure of the Fc(WT)/FcγRIIb extracellular region complex has not been analyzed, the three-dimensional structure of a complex formed with Fc(WT) is known for FcγRIIa, and the extracellular regions of FcγRIIa and FcγRIIb match 93% in amino acid sequence and have very high homology. Thus, the three-dimensional structure of the Fc(WT)/FcγRIIb extracellular region complex was predicted by modeling using the crystal structure of the Fc(WT)/FcγRIIa extracellular region complex.

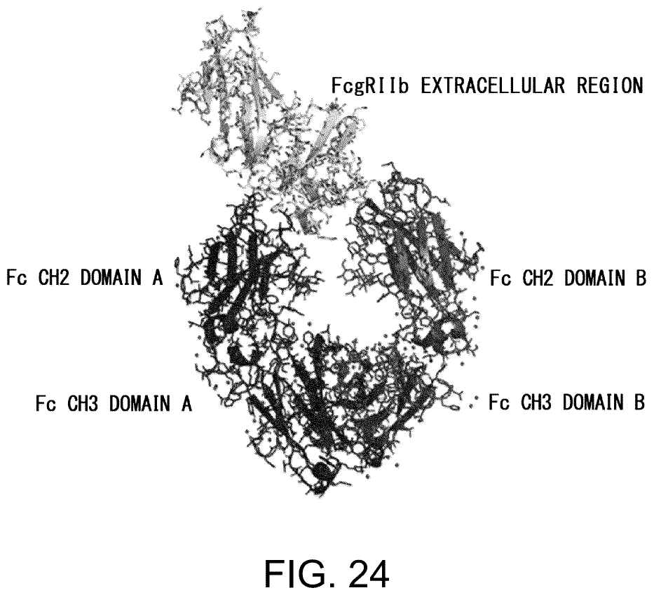

The three-dimensional structure of the Fc(P238D)/FcγRIIb extracellular region complex was determined by X-ray crystal structure analysis at 2.6 Å resolution. The structure obtained as a result of this analysis is shown in FIG. 24. The FcγRIIb extracellular region is bound between two Fc CH2 domains, and this is similar to the three-dimensional structures of complexes formed between Fc(WT) and the respective extracellular region of FcγRIIIa, FcγRIIIb, or FcγRIIa analyzed so far.

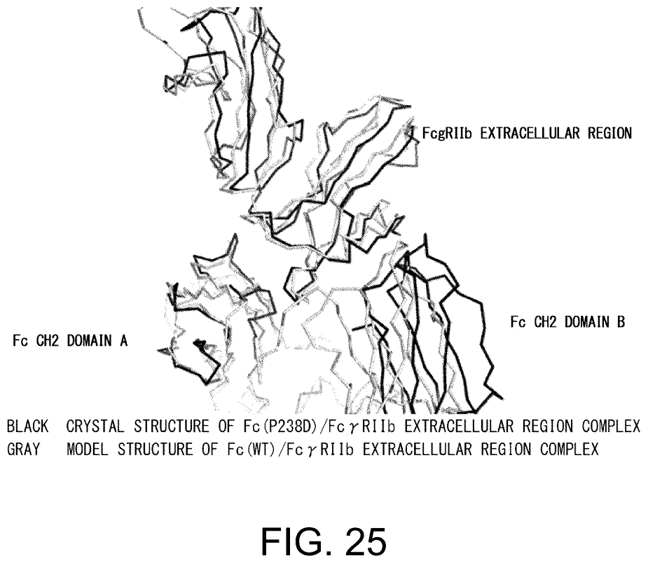

Next, for detailed comparison, the crystal structure of the Fc(P238D)/FcγRIIb extracellular region complex and the model structure of the Fc(WT)/FcγRIIb extracellular region complex were superimposed by least square fitting based on Cα atom pair distances with respect to the FcγRIIb extracellular region and the Fc CH2 domain A (FIG. 25). In that case, the degree of overlap between Fc CH2 domains B was not satisfactory, and conformational differences were found in this portion. Furthermore, using the crystal structure of the Fc(P238D)/FcγRIIb extracellular region complex and the model structure of the Fc(WT)/FcγRIIb extracellular region complex, pairs of atoms that have a distance of 3.7 Å or less between the FcγRIIb extracellular region and Fc CH2 domain B were extracted and compared in order to observe the differences in interatomic interactions between FcγRIIb and Fc CH2 domain B in Fc(WT) and Fc(P238D). As shown in Table 22, the interatomic interactions between Fc CH2 domain B and FcγRIIb in Fc(P238D) and Fc(WT) do not match.

TABLE 22

| | | | Fc(P238D) CH2 DOMAIN B | | | | Fc(WT) CH2 DOMAIN B | | | |
|---|---|---|---|---|---|---|---|---|---|---|
| | | | INTERACTION PARTNER | | | | INTERACTION PARTNER | | | |
| FcgRIIb ATOM | | | (DISTANCE BETWEEN ATOMS, A) | | | | (DISTANCE BETWEEN ATOMS, A) | | | |
| Val | 116 | CG2 | | | | | Asp | 265 | OD2 | (3.47) |
| | | | | | | | Gly | 237 | O | (3.65) |
| Ser | 126 | OG | Ser | 298 | N | (3.31) | | | | |
| | | | Ser | 298 | CB | (3.32) | | | | |
| | | | Tyr | 296 | O | (3.05) | | | | |
| Lys | 128 | CA | Ser | 298 | OG | (3.50) | | | | |
| Phe | 129 | CB | Ser | 298 | O | (3.36) | | | | |
| Phe | 129 | CD2 | | | | | Asn | 297 | CB | (3.50) |
| | | | | | | | Asn | 297 | CG | (3.43) |
| Lys | 128 | C | Ser | 298 | OG | (3.47) | | | | |
| Phe | 129 | N | Ser | 298 | OG | (3.30) | | | | |
| Phe | 129 | O | Ser | 267 | OG | (3.54) | | | | |
| Arg | 131 | CB | | | | | Val | 266 | O | (3.02) |
| Arg | 131 | CG | | | | | Val | 266 | O | (3.22) |
| Arg | 131 | CD | | | | | Val | 266 | CG1 | (3.45) |
| | | | | | | | Val | 266 | C | (3.55) |
| | | | | | | | Val | 266 | O | (3.10) |
| Arg | 131 | NE | Ala | 327 | O | (3.60) | Val | 266 | C | (3.66) |
| | | | | | | | Val | 266 | O | (3.01) |
| | | | | | | | Val | 266 | N | (3.49) |
| Arg | 131 | CZ | Asp | 270 | CG | (3.64) | Val | 266 | N | (3.13) |
| | | | Asp | 270 | OD2 | (3.22) | | | | |
| | | | Asp | 270 | OD1 | (3.27) | | | | |
| | | | Ala | 327 | CB | (3.63) | | | | |
| Arg | 131 | NH1 | Asp | 270 | CG | (3.19) | Val | 266 | CG1 | (3.47) |
| | | | Asp | 270 | OD2 | (2.83) | Val | 266 | N | (3.43) |
| | | | Asp | 270 | OD1 | (2.99) | Thr | 299 | OG1 | (3.66) |
| | | | Ser | 267 | CB | (3.56) | Ser | 298 | O | (3.11) |

Furthermore, the X-ray crystal structure of the Fc(P238D)/FcγRIIb extracellular region complex and the model structure of the Fc(WT)/FcγRIIb extracellular region complex were superimposed by least square fitting based on Cα atom pair distances with respect to the only Fc CH2 domain A or the only Fc CH2 domain B, and the detailed structures near P238D were compared. The location of the amino acid residue at position 238 (EU numbering), which is the mutation introduction position, is changed between Fc(P238D) and Fc(WT), one can see that along with this change, the nearby loop structure continuing from the hinge region is changed between Fc(P238D) and Fc(WT) (FIG. 26). Originally in Fc(WT), Pro at position 238 (EU numbering) is present on the inner side of the protein, and forms a hydrophobic core with the surrounding residues. However, when this residue is changed to a charged and very hydrophilic Asp, the presence in the same hydrophobic core would cause energetical disadvantage in terms of desolvation. Therefore, in Fc(P238D), to cancel this energetically disadvantageous situation, the amino acid residue at position 238 (EU numbering) changes its orientation to face the solvent side, and this may have caused this change in the nearby loop structure. Furthermore, since this loop continues from the hinge region crosslinked by an S—S bond, its structural change will not be limited to a local change, and will affect the relative positioning of the FcCH2 domain A and domain B. As a result, the interatomic interactions between FcγRIIb and Fc CH2 domain B have been changed. Therefore, predicted effects could not be observed when alterations that improve selectivity and binding activity towards FcγRIIb in a naturally-occurring IgG were combined with an Fc containing the P238D alteration.

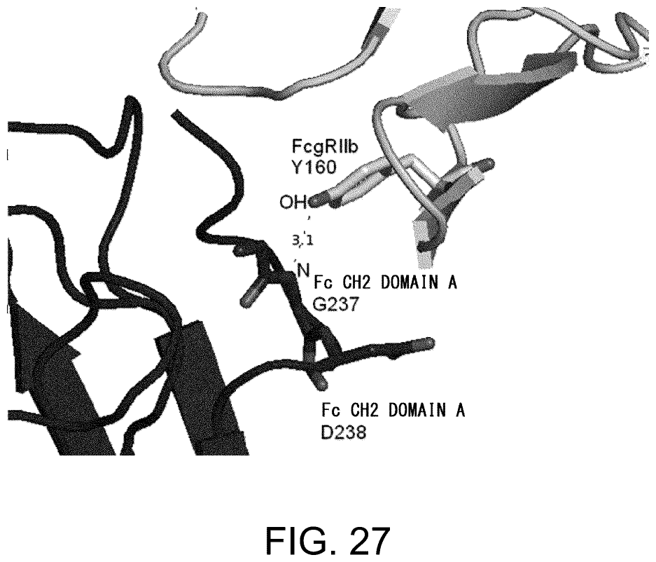

Furthermore, as a result of structural changes due to introduction of P238D in Fc CH2 domain A, a hydrogen bond has been found between the main chain of Gly at adjacent position 237 (EU numbering) and Tyr at position 160 in FcγRIIb (FIG. 27). The residue in FcγRIIa that corresponds to this Tyr 160 is Phe; and when the binding is to FcγRIIa, this hydrogen bond is not formed. The amino acid at position 160 is one of the few differences between FcγRIIa and FcγRIIb at the interface of interaction with Fc, the presence of this hydrogen bond which is specific to FcγRIIb is presumed to have led to improvement of FcγRIIb-binding activity and decrease of FcγRIIa-binding activity in Fc(P238D), and improvement of its selectivity. Furthermore, in Fc CH2 domain B, an electrostatic interaction is observed between Asp at position 270 (EU numbering) and Arg at position 131 in FcγRIIb (FIG. 28). In FcγRIIa type H, which is one of the allotypes of FcγRIIa, the corresponding residue is His, and therefore cannot form this electrostatic interaction. This can explain why the Fc(P238D)-binding activity is lowered in FcγRIIa type H compared with FcγRIIa type R. Observations based on such results of X-ray crystal structure analysis showed that the change of the loop structure beside P238D due to P238D introduction and the accompanying change in the relative domain positioning causes formation of new interactions not found in the naturally-occurring IgG, and this led to a selective binding profile of P238D variants for FcγRIIb.

### [Expression and Purification of Fc(P238D)]

An Fc containing the P238D alteration was prepared as follows. First, Cys at position 220 (EU numbering) of hIL6R-IgG1-v1 (SEQ ID NO: 20) was substituted with Ser. Then, genetic sequence of Fc(P238D) from Glu at position 216 (EU numbering) to its C terminus was cloned by PCR. Using this cloned genetic sequence, production of expression vectors, and expression and purification of Fc(P238D) were carried out according to the method of Reference Example 1. Cys at position 220 (EU numbering) forms a disulfide bond with Cys of the L chain in general IgG1. The L chain is not co-expressed when Fc alone is prepared, and therefore, this residue was substituted with Ser to avoid formation of unnecessary disulfide bonds.

### [Expression and Purification of the FcγRIIb Extracellular Region]

This was prepared according to the method of Reference Example 2.

### [Purification of the Fc(P238D)/FcγRIIb Extracellular Region Complex]

To 2 mg of the FcγRIIb extracellular region sample obtained for crystallization, 0.29 mg of Endo F1 (Protein Science 1996, 5:2617-2622) expressed and purified from Escherichia coli as a glutathione S-transferase fusion protein was added. This was allowed to remain at room temperature for three days in 0.1 M Bis-Tris buffer at pH 6.5, and the N-linked oligosaccharide was cleaved, leaving N-acetylglucosamine directly bound to Asn. Next, this FcγRIIb extracellular domain sample subjected to carbohydrate cleavage treatment was concentrated by ultrafiltration with 5000 MWCO, and purified by gel filtration chromatography (Superdex® 200 10/300 chromatography) using a column equilibrated in 20 mM HEPS at pH 7.5 containing 0.05 M NaCl. Furthermore, to the obtained carbohydrate-cleaved FcγRIIb extracellular region fraction, Fc(P238D) was added so that the molar ratio of the FcγRIIb extracellular region would be present in slight excess, and after concentration by ultrafiltration with 10000 MWCO, a sample of the Fc(P238D)/FcγRIIb extracellular region complex was obtained through purification by gel filtration chromatography (Superdex® 200 10/300 chromatography) using a column equilibrated in 20 mM HEPS at pH 7.5 containing 0.05 M NaCl.

### [Crystallization of the Fc(P238D)/FcγRIIb Extracellular Region Complex]

A sample of the Fc(P238D)/FcγRIIb extracellular region complex was concentrated to approximately 10 mg/mL by ultrafiltration with 10000 MWCO, and crystallization was carried out by the sitting drop vapor diffusion method. A Hydra® II Plus One pipetting robot (MATRIX) was used for crystallization; and for a reservoir solution containing 100 mM Bis-Tris pH 6.5, 17% PEG3350, 0.2 M ammonium acetate, and 2.7% (w/v) D-Galactose, a crystallization drop was produced by mixing at a ratio of reservoir solution: crystallization sample=0.2 μL: 0.2 μL, and after sealing, this was allowed to remain at 20° C., and thin plate-like crystals were successfully obtained.

[Measurement of X-Ray Diffraction Data from an Fc(P238D)/FcγRIIb Extracellular Region Complex Crystal]

One of the obtained single crystals of the Fc(P238D)/FcγRIIb extracellular region complex was soaked into a solution of 100 mM Bis-Tris pH 6.5, 20% PEG3350, ammonium acetate, 2.7% (w/v) D-Galactose, 22.5% (v/v) ethylene glycol. The crystal was fished out of the solution using a pin with attached tiny nylon loop, and frozen in liquid nitrogen; and then X-ray diffraction data was measured at synchrotron radiation facility Photon Factory BL-1A in High Energy Accelerator Research Organization. During the measurement, the crystal was constantly placed in a nitrogen stream at −178° C. to maintain in a frozen state, and a total of 225 X ray diffraction images were collected using Quantum 270 CCD detector (ADSC) attached to a beam line with rotating the crystal 0.8° at a time. Determination of cell parameters, indexing of diffraction spots, and diffraction data processing from the obtained diffraction images were performed using the Xia2 program (CCP4 Software Suite), XDS Package (Walfgang Kabsch) and Scala (CCP4 Software Suite); and finally, diffraction intensity data up to 2.46 Å resolution was obtained. The crystal belongs to the space group P21, and has the following cell parameters; a=48.85 Å, b=76.01 Å, c=115.09 Å, α=90°, β=100.70°, γ=90°.

### [X Ray Structure Analysis of the Fc(P238D)/FcγRIIb Extracellular Region Complex]

Crystal structure of the Fc(P238D)/FcγRIIb extracellular region complex was determined by the molecular replacement method using the program Phaser (CCP4 Software Suite). From the size of the obtained crystal lattice and the molecular weight of the Fc(P238D)/FcγRIIb extracellular region complex, the number of complexes in the asymmetric unit was predicted to be one. From the structural coordinates of PDB code: 3SGJ which is the crystal structure of the Fc(WT)/FcγRIIIa extracellular region complex, the amino acid residue portions of the A chain positions 239-340 and the B chain positions 239-340 were taken out as separate coordinates, and they were used respectively as models for searching the Fc CH2 domains. The amino acid residue portions of the A chain positions 341-444 and the B chain positions 341-443 were taken out as a single set of coordinates from the same structural coordinates of PDB code: 3SGJ; and this was used as a model for searching the Fc CH3 domains. Finally, from the structural coordinates of PDB code: 2FCB which is a crystal structure of the FcγRIIb extracellular region, the amino acid residue portions of the A chain positions 6-178 was taken out and used as a model for searching the FcγRIIb extracellular region. The orientation and position of each search model in the crystal lattice were determined in the order of Fc CH3 domain, FcγRIIb extracellular region, and Fc CH2 domain, based on the rotation function and translation function to obtain the initial model for the crystal structure of the Fc(P238D)/FcγRIIb extracellular region complex. When rigid body refinement which moves the two Fc CH2 domains, the two Fc CH3 domains, and the FcγRIIb extracellular region was performed on the obtained initial model, the crystallographic reliability factor, R value became 40.4%, and the Free R value became 41.9% to diffraction intensity data from 25 Å to 3.0 Å at this point. Furthermore, structural refinement using the program Refmac5 (CCP4 Software Suite), and revision of the model to observe the electron density maps whose coefficient have 2Fo-Fc or Fo-Fc, which are calculated based on the experimentally determined structural factor Fo, the calculated structural factor Fc and the calculated phase using the model, was carried out by the Coot program (Paul Emsley), and model refinement was carried out by repeating these steps. Finally, as a result of incorporation of water molecules into the model based on the electron density maps which use 2Fo-Fc or Fo-Fc as the coefficient, and the following refinement, the crystallographic reliability factor, R values and the Free R value of the model containing 4846 non-hydrogen atoms became 23.7% and 27.6% to 24291 diffraction intensity data from 25 Å to 2.6 Å resolution, respectively.

### [Production of a Model Structure of the Fc(WT)/FcγRIIb Extracellular Region Complex]

Based on the structural coordinates of PDB code: 3RY6 which is a crystal structure of the Fc(WT)/FcγRIIa extracellular region complex, the Build Mutants function of the Discovery Studio 3.1 program (Accelrys) was used to introduce mutations to match the amino acid sequence of FcγRIIb into FcγRIIa in this structural coordinates. In that case, the Optimization Level was set to High, Cut Radius was set to 4.5, five models were generated, and the one with the best energy score among them was employed as the model structure for the Fc(WT)/FcγRIIb extracellular region complex.

### [Reference Example 8] Analysis of FcγR Binding of Fc Variants Whose Alteration Sites were Determined Based on Crystal Structures

Based on the results of X-ray structure analysis on the complex formed between Fc(P238D) and the FcγRIIb extracellular region obtained in Reference Example 7, comprehensive alterations were introduced into sites on the Fc variant having substitution of Pro at position 238 (EU numbering) with Asp that were predicted to affect interaction with FcγRIIb, (residues of positions 233, 240, 241, 263, 265, 266, 267, 268, 271, 273, 295, 296, 298, 300, 323, 325, 326, 327, 328, 330, 332, and 334 (EU numbering)) and variants with a combination of alterations that enhance FcγRIIb binding were examined.

IL6R-B3 (SEQ ID NO: 23) was produced by introducing into IL6R-G1d (SEQ ID NO: 19) produced in Reference Example 4, the alteration produced by substituting Lys at position 439 (EU numbering) with Glu. Next, IL6R-BF648 was produced by introducing into IL6R-B3, the alteration produced by substituting Pro at position 238 (EU numbering) with Asp. IL6R-L (SEQ ID NO: 21) was utilized as the common antibody L chain for all of the antibodies. These antibody variants were expressed and purified according to the method of Reference Example 1, and binding to each of the FcγRs (FcγRIa, FcγRIIa type H, FcγRIIa type R, FcγRIIb, and FcγRIIIa type V) was comprehensively evaluated by the method of Reference Example 2.

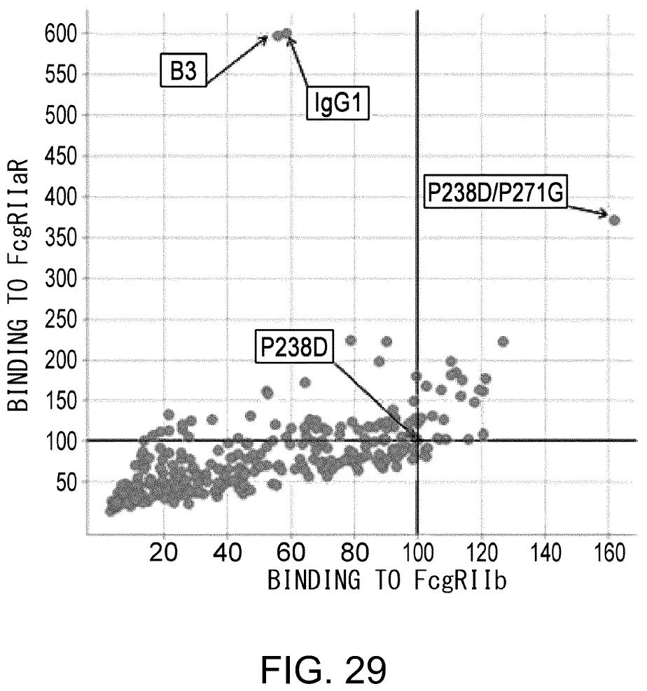

A figure was produced according to the following method for the results of analyzing the interactions with the respective FcγRs. The value for the amount of binding of each variant to each FcγR was divided by the value for the amount of binding of the pre-altered control antibody (IL6R-BF648/IL6R-L with Pro at position 238 (EU numbering) substituted with Asp) to each FcγR, and the obtained was then multiplied by 100 and used as the relative binding activity value of each variant to each FcγR. The horizontal axis shows the relative binding activity value of each variant to FcγRIIb, and the vertical axis shows the relative binding activity value of each variant to FcγRIIa type R (FIG. 29).

As shown in FIG. 29, the results show that of all the alterations, 24 types of alterations were found to have an effect of maintaining or enhancing FcγRIIb binding in comparison with the pre-altered antibody. The binding of these variants to each of the FcγRs are shown in Table 23. In the table, “alteration” refers to the alteration introduced into IL6R-B3 (SEQ ID NO: 23; IL6R-2B999 in Table 23). The template used for producing IL6R-B3, IL6R-G1d/IL6R-L, is indicated with an asterisk (*).

TABLE 23

| | | RELATIVE | RELATIVE | RELATIVE | RELATIVE | RELATIVE |
|---|---|---|---|---|---|---|
| | | BINDING | BINDING | BINDING | BINDING | BINDING |
| | | ACTIVITY | ACTIIVTY | ACTIVITY | ACTIVITY | ACTIVITY |
| VARIANT NAME | ALTERNATION | TO FcgRIa | TO FcgRIIaR | TO FcgRIIaH | TO FcgRIIb | TO FcgRIIIaV |
| IL6R-G1d/IL6R-L | * | 140 | 650 | 1670 | 62 | 3348 |
| IL6R-2B999/IL6R-L | | 145 | 625 | 1601 | 58 | 3264 |
| IL6R-BF648/IL6R-L | P238D | 100 | 100 | 100 | 100 | 100 |
| IL6R-2B002/IL6R-L | P238D/E233D | 118 | 103 | 147 | 116 | 147 |
| IL6R-BP100/IL6R-L | P238D/S267A | 121 | 197 | 128 | 110 | 138 |
| IL6R-BP102/IL6R-L | P238D/S267Q | 104 | 165 | 66 | 106 | 86 |
| IL6R-BP103/IL6R-L | P238D/S267V | 56 | 163 | 69 | 107 | 77 |
| IL6R-BP106/IL6R-L | P238D/H268D | 127 | 150 | 110 | 116 | 127 |
| IL6R-BP107/IL6R-L | P238D/H268E | 123 | 147 | 114 | 118 | 129 |
| IL6R-BP110/IL6R-L | P238D/H268N | 105 | 128 | 127 | 101 | 127 |
| IL6R-BP112/IL6R-L | P238D/P271G | 119 | 340 | 113 | 157 | 102 |
| IL6R-2B128/IL6R-L | P238D/Y296D | 95 | 87 | 37 | 103 | 96 |
| IL6R-2B169/IL6R-L | P238D/V323I | 73 | 92 | 83 | 104 | 94 |
| IL6R-2B171/IL6R-L | P238D/V323L | 116 | 117 | 115 | 113 | 122 |
| IL6R-2B172/IL6R-L | P238D/V323M | 140 | 244 | 179 | 132 | 144 |
| IL6R-BP136/IL6R-L | P238D/K326A | 117 | 159 | 103 | 119 | 102 |
| IL6R-BP117/IL6R-L | P238D/K326D | 124 | 166 | 96 | 118 | 105 |
| IL6R-BP120/IL6R-L | P238D/K326E | 125 | 175 | 92 | 114 | 103 |
| IL6R-BP126/IL6R-L | P238D/K326L | 113 | 167 | 132 | 103 | 146 |
| IL6R-BP119/IL6R-L | P238D/K326M | 117 | 181 | 133 | 110 | 145 |
| IL6R-BP142/IL6R-L | P238D/K326N | 98 | 103 | 97 | 106 | 102 |
| IL6R-BP121/IL6R-L | P238D/K326Q | 118 | 155 | 135 | 113 | 157 |
| IL6R-BP118/IL6R-L | P238D/K326S | 101 | 132 | 128 | 104 | 144 |
| IL6R-BP116/IL6R-L | P238D/K326T | 110 | 126 | 110 | 108 | 114 |
| IL6R-BP911/IL6R-L | P238D/A330K | 52 | 101 | 108 | 119 | 120 |
| IL6R-BP078/IL6R-L | P238D/A330M | 106 | 101 | 89 | 105 | 91 |
| IL6R-BP912/IL6R-L | P238D/A330R | 60 | 81 | 93 | 103 | 97 |

The results of measuring KD values of the variants shown in Table 23 for FcγRIa, FcγRIIaR, FcγRIIaH, FcγRIIb, and FcγRIIIa type V by the method of Reference Example 2 are summarized in Table 24. In the table, “alteration” refers to the alteration introduced into IL6R-B3 (SEQ ID NO: 23). The template used for producing IL6R-B3, IL6R-G1d/IL6R-L, is indicated with an asterisk (*). Furthermore, “KD(IIaR)/KD(IIb)” and “KD(IIaH)/KD(IIb)” in the table respectively represent the value obtained by dividing the KD value of each variant for FcγRIIaR by the KD value of each variant for FcγRIIb, and the value obtained by dividing the KD value of each variant for FcγRIIaH by the KD value of each variant for FcγRIIb. “KD(IIb) of the parent polypeptide/KD(IIb) of the altered polypeptide” refers to the value obtained by dividing the KD value of the parent polypeptide for FcγRIIb by the KD value of each variant for FcγRIIb. In addition, the “KD value for the stronger of the FcγRIIaR- and FcγRIIaH-binding activities of each variant/KD value for the stronger of the FcγRIIaR- and FcγRIIaH-binding activities of the parent polypeptide” are shown in Table 24. Here, parent polypeptide refers to the variant which has IL6R-B3 (SEQ ID NO: 23) as the H chain. It was determined that due to weak binding of FcγR to IgG, it was impossible to accurately analyze by kinetic analysis, and thus the values shown in bold italicized font in Table 24 were calculated by using Equation 2 of Reference Example 2.

KD=C·Rₘₐₓ/(R_{eq}−RI)−C [Equation 2]

Table 24 shows that in comparison with IL6R-B3 (IL6R-2B999 in Table 24), all variants showed improvement of affinity for FcγRIIb, and the range of improvement was 2.1 fold to 9.7 fold. The ratio of KD value of each variant for FcγRIIaR/KD value of each variant for FcγRIIb, and the ratio of KD value of each variant for FcγRIIaH/KD value of each variant for FcγRIIb represent an FcγRIIb-binding activity relative to the FcγRIIaR-binding activity and FcγRIIaH-binding activity, respectively. That is, these values show the degree of binding selectivity of each variant for FcγRIIb, and a greater value indicates a higher binding selectivity for FcγRIIb. Since the ratio of KD value for FcγRIIaR/KD value for FcγRIIb, and the ratio of KD value for FcγRIIaH/KD value for FcγRIIb in the parent polypeptide IL6R-B3/IL6R-L were 0.3 and 0.2, respectively, all variants in Table 24 showed improvement of binding selectivity for FcγRIIb in comparison with the parent polypeptide. When the KD value for the stronger of the FcγRIIaR- and FcγRIIaH-binding activities of a variant/KD value for the stronger of the FcγRIIaR- and FcγRIIaH-binding activities of the parent polypeptide is 1 or more, this means that the stronger of the FcγRIIaR- and FcγRIIaH-binding activities of a variant has equivalent or decreased binding compared with the binding by the stronger of the FcγRIIaR- and FcγRIIaH-binding activities of the parent polypeptide. Since this value was 4.6 to 34.0 for the variants obtained this time, one may say that in comparison with the parent polypeptide, the variants obtained this time had reduced binding by the stronger of the FcγRIIaR- and FcγRIIaH-binding activities. These results showed that compared with the parent polypeptide, the variants obtained this time have maintained or decreased FcγRIIa type R- and type H-binding activities, enhanced FcγRIIb-binding activity, and improved selectivity for FcγRIIb. Furthermore, compared with IL6R-B3, all variants had lower affinity to FcγRIa and FcγRIIIa V.

TABLE 24

| | | | | | | | | | | KD VALUE FOR |
|---|---|---|---|---|---|---|---|---|---|---|
| | | | | | | | | | | THE STRONGER OF |
| | | | | | | | | | | THE FcγRIIaR- AND |
| | | | | | | | | | | FcγRIIaH-BINDING |
| | | | | | | | | | KD(IIb) OF | ACTIVITIES OF |
| | | | | | | | | | THE | THE VARIANT/KD |
| | | | | | | | | | PARENT | VALUE FOR THE |
| | | | | | | | | | POLY | STRONGER OF |
| | | | | | | | | | PEPTIDE/ | THE FcγRIIaR- AND |
| | | | | | | | | | KD(IIb) OF | FcγRIIaH-BINDING |
| | | | | | | | | | ALTERED | ACTIVITIES OF |
| VARIANT | ALTER- | KD (mol/L) | | | | | KD(IIaR)/ | KD(IIaH)/ | POLY- | THE PARENT |
| NAME | ATION | FcγRIa | FcgRIIaR | FcgRIIH | FcgRIIb | FcgRIIIaV | KD(IIb) | KD(IIb) | PEPTIDE | POLYPEPTIDE |
| IL6R-G1d/ | * | 3.2E−10 | 1.0E−06 | 6.7E−07 | 2.6E−06 | 3.5E−07 | 0.4 | 0.3 | 1.2 | 0.9 |
| IL6R-L | | | | | | | | | | |
| IL6R-B3/ | | 4.2E−10 | 1.1E−06 | 7.7E−07 | 3.1E−06 | 3.3E−07 | 0.3 | 0.2 | 1.0 | 1.0 |
| IL6R-L | | | | | | | | | | |
| IL6R-BF648/ | P238D | 1.1E−08 | 1.5E−05 | 4.0E−05 | 1.2E−06 | 7.1E−05 | 13.0 | 33.9 | 2.6 | 19.9 |
| IL6R-L | | | | | | | | | | |
| IL6R-2B002/ | P238D/ | 6.4E−09 | 1.9E−05 | 8.6E−05 | 9.3E−07 | 5.3E−05 | 20.4 | 92.3 | 3.3 | 24.7 |
| IL6R-L | E233D | | | | | | | | | |
| IL6R-BP100/ | P238D/ | 1.1E−09 | 7.8E−06 | 4.6E−05 | 1.1E−06 | 5.9E−05 | 7.3 | 42.6 | 2.9 | 10.2 |
| IL6R-L | S267A | | | | | | | | | |
| IL6R-BP102/ | P238D/ | 8.2E−09 | 8.4E−06 | 6.1E−05 | 9.0E−07 | 8.2E−05 | 9.4 | 67.6 | 3.4 | 11.0 |
| IL6R-L | S267Q | | | | | | | | | |
| IL6R-BP103/ | P238D/ | 3.5E−08 | 1.1E−05 | 8.8E−05 | 1.2E−06 | 1.1E−05 | 9.0 | 71.5 | 2.5 | 14.4 |
| IL6R-L | S267V | | | | | | | | | |
| IL6R-BP106/ | P238D/ | 4.0E−09 | 1.1E−05 | 3.6E−05 | 9.3E−07 | 5.5E−05 | 11.6 | 38.7 | 3.3 | 14.0 |
| IL6R-L | H268D | | | | | | | | | |
| IL6R-BP107/ | P238D/ | 1.5E−09 | 1.2E−05 | 5.2E−05 | 9.3E−07 | 6.3E−05 | 12.7 | 56.1 | 3.3 | 15.3 |
| IL6R-L | H268E | | | | | | | | | |
| IL6R-BP110/ | P238D/ | 7.3E−09 | 1.7E−05 | 4.7E−05 | 1.5E−06 | 6.4E−05 | 11.7 | 31.5 | 2.1 | 22.6 |
| IL6R-L | H268N | | | | | | | | | |
| IL6R-BP112/ | P238D/ | 6.5E−09 | 3.5E−06 | 3.5E−05 | 3.2E−07 | 6.9E−05 | 11.0 | 109.4 | 9.7 | 4.6 |
| IL6R-L | P271G | | | | | | | | | |
| IL6R-2B128/ | P238D/ | 1.3E−08 | 2.6E−05 | 3.4E−05 | 1.4E−06 | 7.2E−05 | 17.7 | 23.6 | 2.1 | 33.1 |
| IL6R-L | Y296D | | | | | | | | | |
| IL6R-2B169/ | P238D/ | 2.5E−08 | 1.9E−05 | 4.8E−05 | 1.2E−06 | 7.5E−05 | 15.8 | 40.7 | 2.6 | 24.3 |
| IL6R-L | V323I | | | | | | | | | |
| IL6R-2B171/ | P238D/ | 9.1E−09 | 1.6E−05 | 3.4E−05 | 1.1E−06 | 5.7E−05 | 15.0 | 31.8 | 2.9 | 20.8 |
| IL6R-L | V323L | | | | | | | | | |
| IL6R-2B172/ | P238D/ | 3.0E−09 | 6.1E−06 | 2.1E−05 | 7.7E−07 | 4.8E−05 | 8.0 | 27.3 | 4.0 | 8.0 |
| IL6R-L | V323M | | | | | | | | | |
| IL6R-BP136/ | P238D/ | 6.6E−09 | 9.1E−06 | 3.8E−05 | 8.0E−07 | 6.9E−05 | 11.4 | 47.6 | 3.9 | 11.8 |
| IL6R-L | K326A | | | | | | | | | |
| IL6R-BP117/ | P238D/ | 4.1E−09 | 9.2E−06 | 4.1E−05 | 8.0E−07 | 6.7E−05 | 11.6 | 51.4 | 3.9 | 12.0 |
| IL6R-L | K326D | | | | | | | | | |
| IL6R-BP120/ | P238D/ | 6.6E−09 | 9.6E−06 | 6.5E−05 | 1.0E−06 | 7.9E−05 | 9.3 | 63.1 | 3.0 | 12.5 |
| IL6R-L | K326E | | | | | | | | | |
| IL6R-BP126/ | P238D/ | 7.4E−09 | 1.1E−05 | 4.5E−05 | 1.4E−06 | 5.6E−05 | 7.8 | 31.7 | 2.2 | 14.4 |
| IL6R-L | K326L | | | | | | | | | |
| IL6R-BP119/ | P238D/ | 7.0E−09 | 9.9E−06 | 4.5E−05 | 1.1E−06 | 5.6E−05 | 8.7 | 39.5 | 2.7 | 12.8 |
| IL6R-L | K326M | | | | | | | | | |
| IL6R-BP142/ | P238D/ | 5.3E−09 | 1.8E−05 | 9.3E−05 | 1.2E−06 | 1.1E−04 | 15.5 | 79.5 | 2.6 | 23.5 |
| IL6R-L | K326N | | | | | | | | | |
| IL6R-BP121/ | P238D/ | 1.1E−08 | 1.3E−05 | 4.4E−05 | 1.1E−06 | 5.2E−05 | 11.7 | 40.4 | 2.8 | 16.6 |
| IL6R-L | K326Q | | | | | | | | | |
| IL6R-BP118/ | P238D/ | 1.2E−08 | 1.5E−05 | 4.6E−05 | 1.2E−06 | 5.6E−05 | 13.2 | 40.0 | 2.7 | 19.7 |
| IL6R-L | K326S | | | | | | | | | |
| IL6R-BP116/ | P238D/ | 2.6E−09 | 1.5E−05 | 5.4E−05 | 1.1E−06 | 7.2E−05 | 13.3 | 48.2 | 2.8 | 19.4 |
| IL6R-L | K326T | | | | | | | | | |
| IL6R-BP911/ | P238D/ | 4.9E−08 | 1.6E−05 | 3.7E−05 | 8.9E−07 | 5.8E−05 | 18.5 | 41.7 | 3.5 | 21.3 |
| IL6R-L | A330K | | | | | | | | | |
| IL6R-BP078/ | P238D/ | 8.2E−09 | 1.5E−05 | 4.5E−05 | 1.1E−06 | 7.8E−05 | 13.4 | 41.3 | 2.8 | 19.0 |
| IL6R-L | A330M | | | | | | | | | |
| IL6R-BP912/ | P238D/ | 3.8E−08 | 2.6E−05 | 3.8E−05 | 1.5E−06 | 7.8E−05 | 17.8 | 25.9 | 2.1 | 34.0 |
| IL6R-L | A330R | | | | | | | | | |

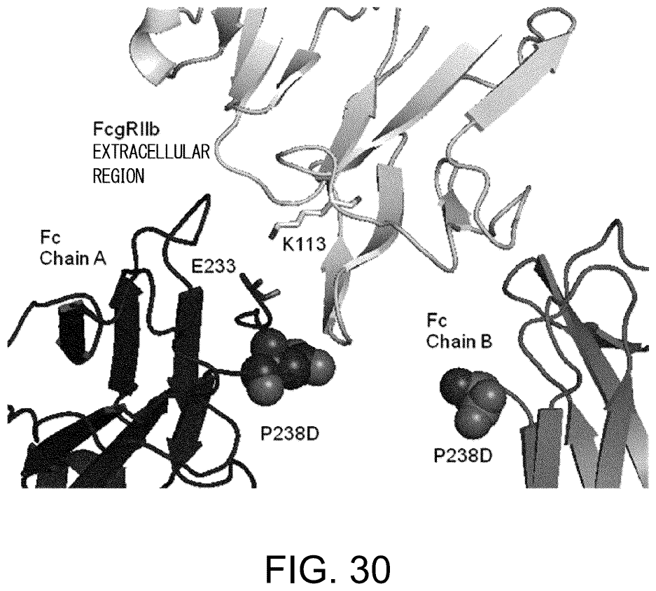

With regard to the promising variants among the obtained combination variants, the factors leading to their effects were studied using the crystal structure. FIG. 30 shows the crystal structure of the Fc(P238D)/FcγRIIb extracellular region complex. In this figure, the H chain positioned on the left side is Fc Chain A, and the H chain positioned on the right side is Fc Chain B. Here, one can see that the site at position 233 (EU numbering) in Fc Chain A is located near Lys at position 113 (EU numbering) of FcγRIIb. However, in this crystal structure, the E233 side chain is in a condition of considerably high mobility, and its electron density is not well observed. Therefore, the alteration produced by substituting Glu at position 233 (EU numbering) with Asp leads to decrease in the degree of freedom of the side chain since the side chain becomes one carbon shorter. As a result, the entropy loss when forming an interaction with Lys at position 113 (EU numbering) of FcγRIIb may be decreased, and consequently this is speculated to contribute to improvement of binding free energy.

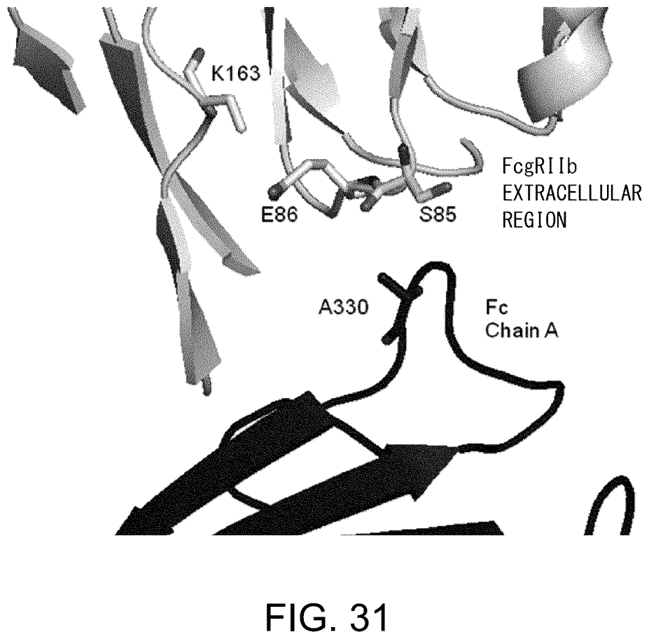

Similarly, FIG. 31 shows the environment near the site at position 330 (EU numbering) in the structure of the Fc(P238D)/FcγRIIb extracellular region complex. This figure shows that the environment around the site at position 330 (EU numbering) of Fc Chain A of Fc(P238D) is a hydrophilic environment composed of Ser at position 85, Glu at position 86, Lys at position 163, and such (EU numbering) of FcγRIIb. Therefore, the alteration produced by substituting Ala at position 330 (EU numbering) with Lys or Arg is speculated to contribute to strengthening the interaction with Ser at position 85 (EU numbering) or Glu at position 86 (EU numbering) in FcγRIIb.

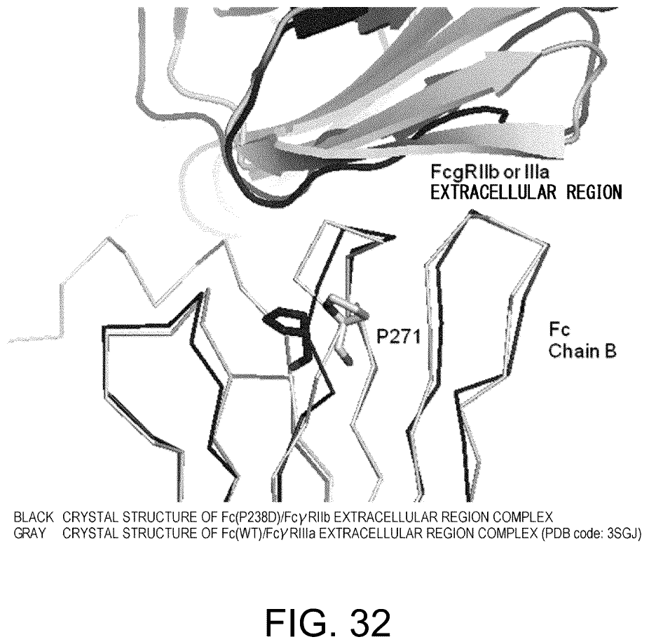

FIG. 32 depicts the structures of Pro at position 271 (EU numbering) of Fc Chain B after superimposing the crystal structures of the Fc(P238D)/FcγRIIb extracellular region complex and the Fc(WT)/FcγRIIIa extracellular region complex by least square fitting based on Cα atom pair distances with respect to Fc Chain B. These two structures match well, but have different three-dimensional structures of Pro at position 271 (EU numbering). When the weak electron density around this area in the crystal structure of the Fc(P238D)/FcγRIIb extracellular region complex is also taken into consideration, it is suggested that there is possibility that Pro at position 271 (EU numbering) in Fc(P238D)/FcγRIIb causes a large strain on the structure, thus disturbing the loop structure to attain an optimal structure. Therefore, one may consider that the alteration produced by substituting Pro at position 271 (EU numbering) with Gly gives flexibility to this loop structure and contributes to enhancement of binding by reducing the energetic barrier when allowing to form an optimum structure upon interaction with FcγRIIb.

### [Reference Example 9] Examination of the Combinatorial Effect of Alterations that Enhance FcγRIIb Binding when Combined with P238D

Of the alterations obtained in Reference Examples 6 and 8, those that enhanced FcγRIIb binding or maintained FcγRIIb binding and showed effects of suppressing binding to other FcγRs were combined with each other, and their effects were examined.

Particularly good alterations were selected from Tables 19 and 22, and they were combined and introduced into the antibody H chain IL6R-BF648 in a similar manner to the method of Reference Example 8. IL6R-L was utilized as the common antibody L chain for all of the antibodies, the antibodies were expressed and purified according to the method of Reference Example 1, and binding to each of the FcγRs (FcγRIa, FcγRIIa type H, FcγRIIa type R, FcγRIIb, and FcγRIIIa type V) was comprehensively evaluated by the method of Reference Example 2.

Relative binding activities were calculated for the results of analyzing interactions with the respective FcγRs according to the following method. The value for the amount of binding of each variant to each FcγR was divided by the value for the amount of binding of the pre-altered control antibody (IL6R-BF648/IL6R-L with substitution of Pro at position 238 (EU numbering) with Asp) to each FcγR, and multiplied by 100; and then the value was used as the relative binding activity value of each variant to each FcγR. The horizontal axis shows the relative binding activity value of each variant to FcγRIIb, and the vertical axis shows the relative binding activity value of each variant to FcγRIIa type R (Table 25).

In the table, “alteration” refers to the alteration introduced into IL6R-B3 (SEQ ID NO: 23). The template used for producing IL6R-B3, IL6R-G1d/IL6R-L, is indicated with an asterisk (*).

TABLE 25

| | | RELATIVE | RELATIVE | RELATIVE | RELATIVE | RELATIVE |
|---|---|---|---|---|---|---|
| | | BINDING | BINDING | BINDING | BINDING | BINDING |
| | | ACTIVITY | ACTIIVTY | ACTIVITY | ACTIVITY | ACTIVITY |
| | | TO | TO | TO | TO | TO |
| VARIANT NAME | ALTERATION | FcgRIa | FcgRIIaR | FcgRIIaH | FcgRIIb | FcgRIIIaV |
| IL6R-G1d/IL6R-L | * | 140 | 650 | 1670 | 62 | 3348 |
| IL6R-B3/IL5R-L | | 145 | 625 | 1601 | 58 | 3264 |
| IL6R-BF648/IL6R-L | P238D | 100 | 100 | 100 | 100 | 100 |
| IL6R-2B253/IL6R-L | E233D/P238D/V323M | 155 | 288 | 207 | 156 | 126 |
| IL6R-2B261/IL6R-L | E233D/P238D/Y296D | 100 | 94 | 91 | 115 | 87 |
| IL6R-BP082/IL6R-L | E233D/P238D/A330K | 74 | 126 | 106 | 136 | 87 |
| IL6R-BP083/IL6R-L | P238D/Y296D/A330K | 50 | 87 | 91 | 122 | 107 |
| IL6R-BP084/IL6R-L | P238D/V323M/A330K | 109 | 203 | 162 | 141 | 106 |
| IL6R-BP085/IL6R-L | G237D/P238D/A330K | 19 | 279 | 158 | 152 | 104 |
| IL6R-BP086/IL6R-L | P238D/K326A/A330K | 72 | 155 | 116 | 137 | 123 |
| IL6R-BP087/IL6R-L | L234Y/P238D/A330K | 33 | 163 | 179 | 137 | 158 |
| IL6R-BP088/IL6R-L | G237D/P238D/K326A/A330K | 25 | 377 | 166 | 161 | 122 |
| IL6R-BP089/IL6R-L | L234Y/P238D/K326A/A330K | 43 | 222 | 186 | 147 | 136 |
| IL6R-BP129/IL6R-L | E233D/P238D/Y296D/A330K | 68 | 111 | 98 | 138 | 95 |
| IL6R-BP130/IL6R-L | E233D/P238D/V323M/A330K | 104 | 272 | 224 | 160 | 115 |
| IL6R-BP131/IL6R-L | E233D/G237D/P238D/A330K | 33 | 364 | 253 | 160 | 118 |
| IL6R-BP132/IL6R-L | E233D/P238D/K326A/A330K | 91 | 191 | 130 | 150 | 120 |
| IL6R-BP133/IL6R-L | E233D/L234Y/P238D/A330K | 41 | 174 | 151 | 137 | 114 |
| IL6R-BP143/IL6R-L | L234Y/P238D/K326A | 86 | 238 | 143 | 133 | 114 |
| IL6R-BP144/IL6R-L | G237D/P238D/K326A | 64 | 204 | 108 | 121 | 128 |
| IL6R-BP145/IL6R-L | L234Y/G237D/P238D | 41 | 350 | 224 | 152 | 153 |
| IL6R-BP146/IL6R-L | L234Y/G237D/P238D/K326A | 50 | 445 | 203 | 156 | 180 |
| IL6R-BP147/IL6R-L | L234Y/G237D/P238D/K326A/A330K | 24 | 650 | 582 | 177 | 209 |
| IL6R-BP148/IL6R-L | E233D/L234Y/G237D/P238D/K326A/A330K | 33 | 603 | 462 | 176 | 227 |
| IL6R-BP149/IL6R-L | E233D/L234Y/G237D/P238D/Y296D/K326A/A330K | 29 | 539 | 401 | 173 | 186 |
| IL6R-BP150/IL6R-L | L234Y/G237D/P238D/K326A/A330R | 30 | 757 | 770 | 183 | 204 |
| IL6R-BP151/IL6R-L | E233D/L234Y/G237D/P238D/K326A/A330R | 39 | 705 | 621 | 180 | 221 |
| IL6R-BP152/IL6R-L | E233D/L234Y/G237D/P238D/Y296D/K326A/A330R | 34 | 638 | 548 | 178 | 146 |
| IL6R-BP176/IL6R-L | E233D/P238D/K326D/A330K | 102 | 201 | 128 | 147 | 131 |
| IL6R-BP177/IL6R-L | E233D/L234Y/G237D/P238D/P271G/K326D/A330K | 57 | 691 | 409 | 177 | 186 |
| IL6R-BP178/IL6R-L | E233D/G237D/P238D/P271G/A330K | 51 | 653 | 259 | 179 | 110 |
| IL6R-BP179/IL6R-L | G237D/P238D/P271G/K326A/A330K | 39 | 570 | 226 | 177 | 125 |
| IL6R-BP180/IL6R-L | G237D/P238D/P271G/A330K | 29 | 602 | 203 | 179 | 100 |
| IL6R-BP181/IL6R-L | E233D/P238D/P271G/K326A/A330K | 108 | 362 | 150 | 170 | 122 |
| IL6R-BP182/IL6R-L | E233D/P238D/P271G/Y296D/A330K | 95 | 413 | 139 | 173 | 120 |
| IL6R-BP183/IL6R-L | E233D/L234Y/P238D/P271G/K326A/A330K | 83 | 423 | 191 | 164 | 113 |
| IL6R-BP184/IL6R-L | E233D/P238D/P271G/A330K | 96 | 436 | 131 | 171 | 106 |
| IL6R-BP185/IL6R-L | E233D/L234Y/G237D/P238D/P271G/K326A/A330K | 47 | 670 | 446 | 179 | 191 |
| IL6R-BP186/IL6R-L | E233D/L234Y/G237D/P238D/P271G/ | 43 | 614 | 368 | 175 | 143 |
| | Y296D/K326A/A330K | | | | | |
| IL6R-BP187/IL6R-L | L234Y/P238D/P271G/K326A/A330K | 68 | 387 | 205 | 157 | 124 |
| IL6R-BP188/IL6R-L | E233D/G237D/P238D/H268D/P271G/A330K | 74 | 636 | 234 | 179 | 121 |
| IL6R-BP189/IL6R-L | G237D/P238D/H268D/P271G/K326A/A330K | 56 | 557 | 183 | 177 | 141 |
| IL6R-BP190/IL6R-L | G237D/P238D/H268D/P271G/A330K | 50 | 615 | 224 | 181 | 155 |
| IL6R-BP191/IL6R-L | E233D/P238D/H268D/P271G/K326A/A330K | 125 | 382 | 145 | 170 | 142 |
| IL6R-BP192/IL6R-L | E233D/P238D/H268D/P271G/Y296D/A330K | 109 | 406 | 122 | 172 | 118 |
| IL6R-BP193/IL6R-L | E233D/P238D/H268D/P271G/A330K | 113 | 449 | 154 | 173 | 135 |
| IL6R-BP194/IL6R-L | E233D/L234Y/G237D/P238D/H268D/ | 69 | 672 | 395 | 178 | 249 |
| | P271G/K326A/A330K | | | | | |
| IL6R-BP195/IL6R-L | E233D/L234Y/G237D/P238D/H268D/P271G/ | 68 | 651 | 344 | 181 | 221 |
| | Y296D/K326A/A330K | | | | | |
| IL6R-BP196/IL6R-L | L234Y/P238D/H268D/P271G/K326A/A330K | 89 | 402 | 195 | 157 | 137 |
| IL6R-BP197/IL6R-L | E233D/L234Y/G237D/P238D/H268D/ | 71 | 642 | 294 | 179 | 206 |
| | P271G/Y296D/K326A/A330K | | | | | |
| IL6R-BP198/IL6R-L | E233D/L234Y/P238D/H268D/P271G/K326A/A330K | 104 | 449 | 188 | 164 | 157 |
| IL6R-BP199/IL6R-L | E233D/P238D/K326A/A330R | 112 | 172 | 116 | 144 | 103 |
| IL6R-BP200/IL6R-L | E233D/L234Y/G237D/P238D/P271G/K326A/A330R | 60 | 754 | 517 | 188 | 164 |
| IL6R-BP201/IL6R-L | E233D/G237D/P238D/P271G/A330R | 57 | 696 | 359 | 186 | 121 |
| IL6R-BP202/IL6R-L | G237D/P238D/P271G/K326A/A330R | 43 | 615 | 285 | 185 | 108 |
| IL6R-BP203/IL6R-L | G237D/P238D/P271G/A330R | 35 | 637 | 255 | 185 | 88 |
| IL6R-BP204/IL6R-L | E233D/P238D/P271G/K326A/A330R | 110 | 301 | 137 | 165 | 121 |
| IL6R-BP205/IL6R-L | E233D/P238D/P271G/Y296D/A330R | 97 | 335 | 108 | 167 | 93 |
| IL6R-BP206/IL6R-L | E233D/P238D/P271G/A330R | 101 | 362 | 123 | 168 | 92 |
| IL6R-BP207/IL6R-L | E233D/P238D/A330R | 74 | 103 | 103 | 124 | 97 |
| IL6R-BP208/IL6R-L | E233D/G237D/P238D/H268D/P271G/A330R | 81 | 690 | 310 | 188 | 118 |
| IL6R-BP209/IL6R-L | G237D/P238D/H268D/P271G/K326A/A330R | 68 | 625 | 267 | 186 | 153 |
| IL6R-BP210/IL6R-L | G237D/P238D/H268D/P271G/A330R | 57 | 661 | 279 | 187 | 135 |
| IL6R-BP211/IL6R-L | E233D/P238D/H268D/P271G/K326A/A330R | 128 | 312 | 111 | 165 | 87 |
| IL6R-BP212/IL6R-L | E233D/P238D/H268D/P271G/Y296D/A330R | 117 | 363 | 135 | 173 | 122 |
| IL6R-BP213/IL6R-L | E233D/P238D/H268D/P271G/A330R | 118 | 382 | 123 | 169 | 100 |
| IL6R-BP214/IL6R-L | E233D/L234Y/G237D/P238D/Y296D/K326A/A330K | 36 | 498 | 285 | 174 | 165 |

The results of measuring KD values of the variants shown in Table 25 for FcγRIa, FcγRIIaR, FcγRIIaH, FcγRIIb, and FcγRIIIa type V by the method of Reference Example 2 are summarized in Table 26. In the table, “alteration” refers to the alteration introduced into IL6R-B3 (SEQ ID NO: 23). The template used for producing IL6R-B3, IL6R-G1d/IL6R-L, is indicated with an asterisk (*). Furthermore, “KD(IIaR)/KD(IIb)” and “KD(IIaH)/KD(IIb)” in the table respectively represent the value obtained by dividing the KD value of each variant for FcγRIIaR by the KD value of each variant for FcγRIIb, and the value obtained by dividing the KD value of each variant for FcγRIIaH by the KD value of each variant for FcγRIIb. “KD(IIb) of the parent polypeptide/KD(IIb) of the altered polypeptide” refers to the value obtained by dividing the KD value of the parent polypeptide for FcγRIIb by the KD value of each variant for FcγRIIb. In addition, the “KD value for the stronger of the FcγRIIaR- and FcγRIIaH-binding activities of each variant/KD value for the stronger of the FcγRIIaR- and FcγRIIaH-binding activities of the parent polypeptide” are shown in Table 26. Here, parent polypeptide refers to the variant which has IL6R-B3 (SEQ ID NO: 23) as the H chain. It was determined that due to weak binding of FcγR to IgG, it was impossible to accurately analyze by kinetic analysis, and thus the values shown in bold italicized font in Table 26 were calculated by using Equation 2 of Reference Example 2.

KD=C·Rₘₐₓ/(R_{eq}−RI)−C [Equation 2]

Table 26 shows that in comparison with IL6R-B3, all variants showed improvement of affinity for FcγRIIb, and the range of improvement was 3.0 fold to 99.0 fold. The ratio of KD value of each variant for FcγRIIaR/KD value of each variant for FcγRIIb, and the ratio of KD value of each variant for FcγRIIaH/KD value of each variant for FcγRIIb represent an FcγRIIb-binding activity relative to the FcγRIIaR-binding activity and FcγRIIaH-binding activity, respectively. That is, those values show the degree of binding selectivity of each variant for FcγRIIb, and a greater value indicates a higher binding selectivity for FcγRIIb. Since the ratio of KD value for FcγRIIaR/KD value for FcγRIIb, and the ratio of KD value for FcγRIIaH/KD value for FcγRIIb of the parent polypeptide IL6R-B3/IL6R-L were 0.3 and 0.2, respectively, all variants in Table 26 showed improvement of binding selectivity for FcγRIIb in comparison with the parent polypeptide. When the KD value for the stronger of the FcγRIIaR- and FcγRIIaH-binding activities of a variant/KD value for the stronger of the FcγRIIaR- and FcγRIIaH-binding activities of the parent polypeptide is 1 or more, this means that the stronger of the FcγRIIaR- and FcγRIIaH-binding activities of a variant has equivalent or decreased binding compared with the binding by the stronger of the FcγRIIaR- and FcγRIIaH-binding activities of the parent polypeptide. Since this value was 0.7 to 29.9 for the variants obtained this time, one may say that binding by the stronger of the FcγRIIaR- and FcγRIIaH-binding activities of the variants obtained this time was nearly equivalent or decreased compared with that of the parent polypeptide. These results showed that compared with the parent polypeptide, the variants obtained this time have maintained or decreased FcγRIIa type R- and type H-binding activities, enhanced FcγRIIb-binding activity, and improved selectivity for FcγRIIb. Furthermore, compared with IL6R-B3, all variants had lower affinity for FcγRIa and FcγRIIIa V.

TABLE 26

| | | | | | | | | | | KD VALUE FOR |
|---|---|---|---|---|---|---|---|---|---|---|
| | | | | | | | | | | THE STRONGER OF |
| | | | | | | | | | | THE FcγRIIaR- AND |
| | | | | | | | | | | FcγRIIaH-BINDING |
| | | | | | | | | | KD(IIb) OF | ACTIVITIES OF |
| | | | | | | | | | THE | THE VARIANT/KD |
| | | | | | | | | | PARENT | VALUE FOR THE |
| | | | | | | | | | POLY | STRONGER OF |
| | | | | | | | | | PEPTIDE/ | THE FcγRIIaR- AND |
| | | | | | | | | | KD(IIb) OF | FcγRIIaH-BINDING |
| | | KD (mol/L) | | | | | | | ALTERED | ACTIVITIES OF |
| VARIENT | | | | | | POLY- | KD(IIaR)/ | KD(IIaH)/ | POLY- | THE PARENT |
| NAME | ALTERATION | FcγRIa | FcγRIIaR | FcγRIIaH | PEPTIDE | PEPTIDE | KD(IIb) | KD(IIb) | PEPTIDE | POLYPEPTIDE |
| IL6R-G1d/ | * | 3.2E−10 | 1.0E−06 | 6.7E−07 | 2.6E−06 | 3.5E−07 | 0.4 | 0.3 | 1.2 | 0.9 |
| IL6R-L | | | | | | | | | | |
| IL6R-B3/ | | 4.2E−10 | 1.1E−06 | 7.7E−07 | 3.1E−06 | 3.3E−07 | 0.3 | 0.2 | 1.0 | 1.0 |
| IL6R-L | | | | | | | | | | |
| IL6R- | P238D | 1.1E−08 | 1.5E−05 | 4.0E−05 | 1.2E−06 | 7.1E−05 | 13.0 | 33.9 | 2.6 | 19.9 |
| BF648/ | | | | | | | | | | |
| IL6R-L | | | | | | | | | | |
| IL6R- | E233D/P238D/ | 1.4E−09 | 5.0E−06 | 1.3E−05 | 4.3E−07 | 5.0E−05 | 11.5 | 30.1 | 7.2 | 6.5 |
| 2B253/ | V323M | | | | | | | | | |
| IL6R-L | | | | | | | | | | |
| IL6R- | E233D/P238D/ | 9.0E−09 | 2.2E−05 | 3.3E−05 | 1.0E−06 | 7.3E−05 | 21.8 | 32.4 | 3.0 | 28.8 |
| 2B261/ | Y296D | | | | | | | | | |
| IL6R-L | | | | | | | | | | |
| IL6R- | E233D/P238D/ | 1.8E−08 | 1.2E−05 | 3.7E−05 | 5.4E−07 | 8.1E−05 | 22.8 | 69.0 | 5.8 | 15.8 |
| BP082/ | A330K | | | | | | | | | |
| IL6R-L | | | | | | | | | | |
| IL6R- | P238D/Y296D/ | 3.8E−08 | 2.3E−05 | 4.4E−05 | 7.9E−07 | 6.6E−05 | 29.0 | 55.5 | 3.9 | 29.9 |
| BP083/ | A330K | | | | | | | | | |
| IL6R-L | | | | | | | | | | |
| IL6R- | P238D/V323M/ | 7.0E−09 | 7.2E−06 | 2.4E−05 | 5.0E−07 | 6.7E−05 | 14.3 | 47.6 | 6.1 | 9.4 |
| BP084/ | A330K | | | | | | | | | |
| IL6R-L | | | | | | | | | | |
| IL6R- | G237D/P238D/ | 2.9E−07 | 4.2E−06 | 2.4E−05 | 3.2E−07 | 6.8E−05 | 13.1 | 74.5 | 9.6 | 5.5 |
| BP085/ | A330K | | | | | | | | | |
| IL6R-L | | | | | | | | | | |
| IL6R- | P238D/K326A/ | 2.7E−08 | 9.7E−06 | 3.4E−05 | 5.7E−07 | 5.7E−05 | 17.1 | 59.9 | 5.4 | 12.6 |
| BP086/ | A330K | | | | | | | | | |
| IL6R-L | | | | | | | | | | |
| IL6R- | L234Y/P238D/ | 3.8E−08 | 9.7E−06 | 2.1E−05 | 6.1E−07 | 4.4E−05 | 16.0 | 34.7 | 5.1 | 12.6 |
| BP087/ | A330K | | | | | | | | | |
| IL6R-L | | | | | | | | | | |
| IL6R- | G237D/P238D/ | 3.9E−07 | 2.9E−06 | 2.3E−05 | 2.2E−07 | 5.7E−05 | 13.3 | 106.5 | 14.3 | 3.7 |
| BP088/ | K326A/A330K | | | | | | | | | |
| IL6R-L | | | | | | | | | | |
| IL6R- | L234Y/P238D/ | 6.3E−08 | 6.4E−06 | 2.0E−05 | 3.9E−07 | 5.1E−05 | 16.6 | 51.9 | 8.0 | 8.3 |
| BP089/ | K326A/A330K | | | | | | | | | |
| IL6R-L | | | | | | | | | | |
| IL6R- | E233D/P238D/ | 2.5E−08 | 1.5E−05 | 4.0E−05 | 5.2E−07 | 7.5E−05 | 29.3 | 77.5 | 6.0 | 19.6 |
| BP129/ | Y296D/A330K | | | | | | | | | |
| IL6R-L | | | | | | | | | | |
| IL6R- | E233D/P238D/ | 1.8E−09 | 5.3E−06 | 2.6E−05 | 3.0E−07 | 7.1E−05 | 17.5 | 85.5 | 10.2 | 6.9 |
| BP130/ | V323M/A330K | | | | | | | | | |
| IL6R-L | | | | | | | | | | |
| IL6R- | E233D/G237D/ | 1.2E−07 | 3.1E−06 | 1.4E−05 | 2.5E−07 | 5.9E−05 | 12.5 | 56.9 | 12.6 | 4.0 |
| BP131/ | P238D/A330K | | | | | | | | | |
| IL6R-L | | | | | | | | | | |
| IL6R- | E233D/P238D/ | 1.5E−08 | 8.0E−06 | 3.0E−05 | 3.7E−07 | 5.8E−05 | 21.5 | 81.1 | 8.4 | 10.3 |
| BP132/ | K326A/A330K | | | | | | | | | |
| IL6R-L | | | | | | | | | | |
| IL6R- | E233D/L234Y/ | 1.3E−07 | 8.6E−06 | 2.6E−05 | 5.6E−07 | 6.2E−05 | 15.5 | 46.8 | 5.6 | 11.2 |
| BP133/ | P238D/A330K | | | | | | | | | |
| IL6R-L | | | | | | | | | | |
| IL6R- | L234Y/P238D/ | 1.6E−08 | 5.7E−06 | 2.7E−05 | 5.7E−07 | 6.2E−05 | 10.0 | 47.1 | 5.4 | 7.5 |
| BP143/ | K326A | | | | | | | | | |
| IL6R-L | | | | | | | | | | |
| IL6R- | G237D/P238D/ | 3.7E−08 | 6.9E−06 | 3.6E−05 | 7.9E−07 | 5.5E−05 | 8.7 | 45.8 | 3.9 | 8.9 |
| BP144/ | K326A | | | | | | | | | |
| IL6R-L | | | | | | | | | | |
| IL6R- | L234Y/G237D/ | 1.2E−07 | 3.4E−06 | 1.7E−05 | 3.4E−07 | 4.5E−05 | 9.9 | 49.9 | 9.1 | 4.4 |
| BP145/ | P238D | | | | | | | | | |
| IL6R-L | | | | | | | | | | |
| IL6R- | L234Y/G237D/ | 7.4E−08 | 2.1E−06 | 1.8E−05 | 2.3E−07 | 3.8E−05 | 9.3 | 80.0 | 13.7 | 2.7 |
| BP146/ | P238D/K326A | | | | | | | | | |
| IL6R-L | | | | | | | | | | |
| IL6R- | L234Y/G237D/ | 1.4E−07 | 8.9E−07 | 5.1E−06 | 6.6E−08 | 3.3E−05 | 13.6 | 77.7 | 47.1 | 1.2 |
| BP147/ | P238D/K326A/ | | | | | | | | | |
| IL6R-L | A330K | | | | | | | | | |
| IL6R- | E233D/L234Y/ | 8.9E−08 | 1.1E−06 | 7.0E−06 | 7.5E−08 | 3.0E−05 | 14.5 | 93.8 | 41.4 | 1.4 |
| BP148/ | G237D/P238D/ | | | | | | | | | |
| IL6R-L | K326A/A330K | | | | | | | | | |
| IL6R- | E233D/L234Y/ | 1.2E−07 | 1.4E−06 | 8.4E−06 | 9.3E−08 | 3.7E−05 | 15.0 | 89.9 | 33.1 | 1.8 |
| BP149/ | G237D/P238D/ | | | | | | | | | |
| IL6R-L | Y296D/K326A/ | | | | | | | | | |
| | A330K | | | | | | | | | |
| IL6R- | L234Y/G237D/ | 3.2E−07 | 5.5E−07 | 3.4E−06 | 3.1E−08 | 3.4E−05 | 17.7 | 109.0 | 99.0 | 0.7 |
| BP150/ | P238D/K326A/ | | | | | | | | | |
| IL6R-L | A330R | | | | | | | | | |
| IL6R- | E233D/L234Y/ | 8.4E−08 | 6.7E−07 | 4.7E−06 | 4.0E−08 | 3.1E−05 | 16.9 | 117.8 | 77.4 | 0.9 |
| BP151/ | G237D/P238D/ | | | | | | | | | |
| IL6R-L | K326A/A330R | | | | | | | | | |
| IL6R- | E233D/L234Y/ | 7.3E−08 | 8.1E−07 | 5.6E−06 | 4.1E−08 | 4.8E−05 | 19.5 | 135.9 | 75.0 | 1.0 |
| BP152/ | G237D/P238D/ | | | | | | | | | |
| IL6R-L | V296D/K326A/ | | | | | | | | | |
| | A330R | | | | | | | | | |
| IL6R- | E233D/P238D/ | 7.3E−09 | 6.9E−06 | 3.0E−05 | 3.6E−07 | 5.4E−05 | 19.1 | 83.1 | 8.6 | 8.9 |
| BP176/ | K326D/A330K | | | | | | | | | |
| IL6R-L | | | | | | | | | | |
| IL6R- | E233D/L234Y/ | 3.3E−08 | 7.1E−07 | 8.2E−06 | 5.2E−08 | 3.7E−05 | 13.8 | 159.2 | 60.0 | 0.9 |
| BP177/ | G237D/P238D/ | | | | | | | | | |
| IL6R-L | P271G/K326D/ | | | | | | | | | |
| | A330K | | | | | | | | | |
| IL6R- | E233D/G237D/ | 4.3E−08 | 9.3E−07 | 1.4E−05 | 5.1E−08 | 6.4E−05 | 18.1 | 272.4 | 60.1 | 1.2 |
| BP178/ | P238D/P271G/ | | | | | | | | | |
| IL6R-L | A330K | | | | | | | | | |
| IL6R- | G237D/P238D/ | 6.4E−08 | 1.4E−06 | 1.6E−05 | 8.4E−08 | 5.6E−05 | 16.7 | 190.9 | 36.9 | 1.8 |
| BP179/ | P271G/K326A/ | | | | | | | | | |
| IL6R-L | A330K | | | | | | | | | |
| IL6R- | G237D/P238D/ | 9.8E−08 | 1.2E−06 | 1.8E−05 | 6.2E−08 | 7.0E−05 | 18.6 | 290.8 | 49.9 | 1.5 |
| BP180/ | P271G/A330K | | | | | | | | | |
| IL6R-L | | | | | | | | | | |
| IL6R- | E233D/P238D/ | 7.5E−09 | 3.2E−06 | 2.6E−05 | 1.6E−07 | 5.7E−05 | 20.3 | 162.5 | 19.3 | 4.2 |
| BP181/ | P271G/K326A/ | | | | | | | | | |
| IL6R-L | A330K | | | | | | | | | |
| IL6R- | E233D/P238D/ | 1.0E−08 | 2.6E−06 | 2.8E−05 | 1.1E−07 | 5.8E−05 | 23.5 | 256.9 | 28.3 | 3.3 |
| BP182/ | P271G/Y296D/ | | | | | | | | | |
| IL6R-L | A330K | | | | | | | | | |
| IL6R- | E233D/L234Y/ | 1.7E−08 | 2.6E−06 | 1.5E−05 | 2.4E−07 | 5.6E−05 | 10.7 | 62.5 | 12.9 | 3.3 |
| BP183/ | P238D/P271G/ | | | | | | | | | |
| IL6R-L | K326A/A330K | | | | | | | | | |
| IL6R- | E233D/P238D/ | 1.1E−08 | 2.3E−06 | 3.0E−05 | 1.3E−07 | 6.6E−05 | 18.2 | 238.1 | 24.5 | 3.0 |
| BP184/ | P271G/A330K | | | | | | | | | |
| IL6R-L | | | | | | | | | | |
| IL6R- | E233D/L234Y/ | 6.3E−08 | 8.8E−07 | 7.3E−06 | 6.9E−08 | 3.6E−05 | 12.6 | 105.2 | 44.5 | 1.1 |
| BP185/ | G237D/P238D/ | | | | | | | | | |
| IL6R-L | P271G/K326A/ | | | | | | | | | |
| | A330K | | | | | | | | | |
| IL6R- | E233D/L234Y/ | 4.5E−08 | 9.6E−07 | 9.3E−06 | 6.1E−08 | 4.9E−06 | 15.8 | 152.5 | 50.7 | 1.3 |
| BP186/ | G237D/P238D/ | | | | | | | | | |
| IL6R-L | P271G/Y296D/ | | | | | | | | | |
| | K326A/A330K | | | | | | | | | |
| IL6R- | L234Y/P238D/ | 2.5E−08 | 2.8E−06 | 1.8E−05 | 2.9E−07 | 5.6E−05 | 9.7 | 62.3 | 10.7 | 3.6 |
| BP187/ | P271G/K326A/ | | | | | | | | | |
| IL6R-L | A330K | | | | | | | | | |
| IL6R- | E233D/G237D/ | 2.1E−08 | 1.0E−06 | 1.6E−05 | 4.6E−08 | 5.8E−05 | 21.9 | 350.1 | 67.6 | 1.3 |
| BP188/ | P238D/H268D/ | | | | | | | | | |
| IL6R-L | P271G/A330K | | | | | | | | | |
| IL6R- | G237D/P238D/ | 4.2E−08 | 1.4E−06 | 2.1E−05 | 7.4E−08 | 4.9E−05 | 18.5 | 283.8 | 41.8 | 1.8 |
| BP189/ | H268D/P271G/ | | | | | | | | | |
| IL6R-L | K326A/A330K | | | | | | | | | |
| IL6R- | G237D/P238D/ | 6.3E−08 | 1.1E−06 | 1.7E−05 | 5.8E−08 | 4.5E−05 | 19.3 | 292.6 | 53.2 | 1.5 |
| BP190/ | H268D/P271G/ | | | | | | | | | |
| IL6R-L | A330K | | | | | | | | | |
| IL6R- | E233D/P238D/ | 4.0E−09 | 3.0E−06 | 2.7E−05 | 1.5E−07 | 4.9E−05 | 20.3 | 184.9 | 21.2 | 3.8 |
| BP191/ | H268D/P271G/ | | | | | | | | | |
| IL6R-L | K326A/A330K | | | | | | | | | |
| IL6R- | E233D/P238D/ | 6.6E−09 | 2.6E−06 | 3.2E−05 | 1.1E−07 | 5.9E−05 | 23.1 | 283.2 | 27.3 | 3.4 |
| BP192/ | H268D/P271G/ | | | | | | | | | |
| IL6R-L | Y296D/A330K | | | | | | | | | |
| IL6R- | E233D/P238D/ | 6.3E−09 | 2.2E−06 | 2.5E−05 | 1.2E−07 | 5.2E−05 | 18.3 | 206.6 | 25.5 | 2.9 |
| BP193/ | H268D/P271G/ | | | | | | | | | |
| IL6R-L | A330K | | | | | | | | | |
| IL6R- | E233D/L234Y/ | 2.4E−08 | 8.2E−07 | 8.5E−06 | 5.2E−08 | 2.7E−05 | 15.8 | 163.5 | 59.4 | 1.1 |
| BP194/ | G237D/P238D/ | | | | | | | | | |
| IL6R-L | H268D/P271G/ | | | | | | | | | |
| | K326A/A330K | | | | | | | | | |
| IL6R- | E233D/L234Y/ | 2.3E−08 | 9.1E−07 | 1.0E−05 | 5.0E−08 | 3.1E−05 | 18.2 | 200.8 | 62.0 | 1.2 |
| BP195/ | G237D/P238D/ | | | | | | | | | |
| IL6R-L | H268D/P271G/ | | | | | | | | | |
| | Y296D/K326A/ | | | | | | | | | |
| | A330K | | | | | | | | | |
| IL6R- | L234Y/P238D/ | 1.4E−08 | 3.0E−06 | 1.9E−05 | 2.2E−07 | 5.1E−05 | 13.4 | 85.2 | 13.9 | 3.9 |
| BP196/ | H268D/P271G/ | | | | | | | | | |
| IL6R-L | K326A/A330K | | | | | | | | | |
| IL6R- | E233D/L234Y/ | 1.9E−08 | 9.8E−07 | 1.2E−05 | 5.8E−08 | 3.3E−05 | 17.1 | 208.7 | 53.7 | 1.3 |
| BP197/ | G237D/P238D/ | | | | | | | | | |
| IL6R-L | H268D/P271G/ | | | | | | | | | |
| | Y296D/K326D/ | | | | | | | | | |
| | A330K | | | | | | | | | |
| IL6R- | E233D/L234Y/ | 1.1E−08 | 2.2E−06 | 2.0E−05 | 2.0E−07 | 4.4E−05 | 11.0 | 101.5 | 15.7 | 2.8 |
| BP198/ | P238D/H268D/ | | | | | | | | | |
| IL6R-L | P271G/K326A/ | | | | | | | | | |
| | A330K | | | | | | | | | |
| IL6R- | E233D/P238D/ | 6.4E−09 | 8.6E−06 | 2.6E−05 | 4.9E−07 | 6.1E−05 | 17.5 | 53.0 | 6.3 | 11.1 |
| BP199/ | K326D/A330R | | | | | | | | | |
| IL6R-L | | | | | | | | | | |
| IL6R- | E233D/L234Y/ | 3.3E−08 | 6.3E−07 | 4.2E−06 | 3.4E−08 | 3.8E−05 | 18.6 | 123.9 | 91.2 | 0.8 |
| BP200/ | G237D/P238D/ | | | | | | | | | |
| IL6R-L | P271G/K326D/ | | | | | | | | | |
| | A330R | | | | | | | | | |
| IL6R- | E233D/G237D/ | 5.1E−08 | 8.4E−07 | 6.9E−06 | 4.0E−08 | 5.2E−05 | 21.0 | 172.1 | 77.1 | 1.1 |
| BP201/ | P238D/P271G/ | | | | | | | | | |
| IL6R-L | A330R | | | | | | | | | |
| IL6R- | G237D/P238D/ | 9.5E−08 | 1.2E−06 | 9.2E−06 | 6.4E−08 | 5.9E−05 | 19.2 | 144.0 | 48.4 | 1.6 |
| BP202/ | P271G/K326A/ | | | | | | | | | |
| IL6R-L | A330R | | | | | | | | | |
| IL6R- | G237D/P238D/ | 1.8E−07 | 9.9E−07 | 1.1E−05 | 4.9E−08 | 7.2E−05 | 20.5 | 226.8 | 63.7 | 1.3 |
| BP203/ | P271G/A330R | | | | | | | | | |
| IL6R-L | | | | | | | | | | |
| IL6R- | E233D/P238D/ | 7.6E−09 | 4.5E−06 | 2.1E−05 | 2.5E−07 | 5.2E−05 | 17.6 | 82.7 | 12.2 | 5.8 |
| BP204/ | P271G/K326A/ | | | | | | | | | |
| IL6R-L | A330R | | | | | | | | | |
| IL6R- | E233D/P238D/ | 7.7E−09 | 3.5E−06 | 2.8E−05 | 1.6E−07 | 6.8E−05 | 21.8 | 176.1 | 19.4 | 4.5 |
| BP205/ | P271G/Y296D/ | | | | | | | | | |
| IL6R-L | A330R | | | | | | | | | |
| IL6R- | E233D/P238D/ | 8.2E−09 | 3.1E−06 | 2.4E−05 | 2.0E−07 | 6.9E−05 | 16.1 | 123.1 | 15.8 | 4.1 |
| BP206/ | P271G/A330R | | | | | | | | | |
| IL6R-L | | | | | | | | | | |
| IL6R- | E233D/P238D/ | 2.2E−08 | 1.9E−05 | 2.9E−05 | 8.4E−07 | 6.5E−05 | 23.0 | 34.5 | 3.7 | 25.1 |
| BP207/ | A330R | | | | | | | | | |
| IL6R-L | | | | | | | | | | |
| IL6R- | E233D/G237D/ | 1.9E−08 | 8.5E−07 | 8.3E−06 | 3.2E−08 | 5.3E−05 | 26.3 | 256.2 | 95.4 | 1.1 |
| BP208/ | P238D/H268D/ | | | | | | | | | |
| IL6R-L | P271G/A330R | | | | | | | | | |
| IL6R- | G237D/P238D/ | 3.9E−08 | 1.2E−06 | 1.0E−05 | 5.1E−08 | 4.1E−05 | 22.7 | 195.3 | 60.4 | 1.5 |
| BP209/ | H268D/P271G/ | | | | | | | | | |
| IL6R-L | K326A/A330R | | | | | | | | | |
| IL6R- | G237D/P238D/ | 6.5E−08 | 1.0E−06 | 9.5E−06 | 3.9E−08 | 4.6E−05 | 25.4 | 241.1 | 78.4 | 1.3 |
| BP210/ | H268D/P271G/ | | | | | | | | | |
| IL6R-L | A330R | | | | | | | | | |
| IL6R- | E233D/P238D/ | 4.2E−09 | 4.1E−06 | 2.7E−05 | 2.2E−07 | 7.3E−05 | 18.5 | 120.5 | 13.8 | 5.4 |
| BP211/ | H268D/P271G/ | | | | | | | | | |
| IL6R-L | K326A/A330R | | | | | | | | | |
| IL6R- | E233D/P238D/ | 5.2E−09 | 3.5E−06 | 2.2E−05 | 1.7E−07 | 5.2E−05 | 21.1 | 133.3 | 18.7 | 4.5 |
| BP212/ | H268D/P271G/ | | | | | | | | | |
| IL6R-L | Y296D/A330R | | | | | | | | | |
| IL6R- | E233D/P238D/ | 4.1E−09 | 3.1E−06 | 2.4E−05 | 1.8E−07 | 6.3E−05 | 17.7 | 136.4 | 17.6 | 4.0 |
| BP213/ | H268D/P271G/ | | | | | | | | | |
| IL6R-L | A330R | | | | | | | | | |
| IL6R- | E233D/L234Y/ | 5.9E−08 | 1.7E−06 | 9.2E−06 | 1.2E−07 | 3.8E−05 | 14.5 | 78.0 | 26.2 | 2.2 |
| BP214/ | G237D/P238D/ | | | | | | | | | |
| IL6R-L | Y296D/K326D/ | | | | | | | | | |
| | A330K | | | | | | | | | |

The variable region and constant region in the sequence of the respective SEQ ID NOS are summarized in the following Table. In the table, “B3” refers to “2B999 (B3)”, “omlizH” refers to “omalizumab_VH”, and “omlizL” refers to “omalizumab_VL”.

TABLE 27

| SEQ | VARIABLE | CONSTANT |
|---|---|---|
| ID NO | REGION | REGION |
| 15 | GpH7 | |
| 16 | GpL16 | k0 |
| 17 | GpH7 | B3 |
| 18 | IL6R | |
| 19 | IL6R | G1d |
| 20 | IL6R | IgG1-v1 |
| 21 | IL6R-L | k0 |
| 22 | IL6R | F11 |
| 23 | IL6R | B3 |
| 24 | IL6R | BP208 |
| 25 | omlizH | G1d |
| 26 | omlizL | CK |
| 27 | IL6R | BP230 |
| 28 | IL6R | BP264 |
| 29 | IL6R | BP267 |
| 30 | IL6R | G4d |
| 31 | IL6R | BP478 |
| 32 | IL6R | BP253 |
| 33 | IL6R | BP423 |
| 34 | GpH7 | G1d |
| 35 | GpH7 | A5 |
| 36 | IL6R | BP404 |
| 37 | IL6R | BP408 |
| 38 | IL6R | BP419 |
| 39 | IL6R | BP407 |
| 40 | IL6R | BP409 |
| 41 | IL6R | BP410 |
| 42 | IL6R | AP029 |
| 43 | | BP230 |
| 44 | | BP231 |
| 45 | | BP265 |
| 46 | | BP391 |
| 47 | | BP429 |
| 48 | | BP436 |
| 49 | | BP437 |
| 50 | | BP445 |
| 51 | | BP473 |
| 52 | | BP478 |
| 53 | | BP481 |
| 54 | | BP487 |
| 55 | | BP488 |
| 56 | | BP489 |
| 57 | | BP490 |
| 58 | | BP491 |
| 59 | | BP492 |
| 60 | | BP493 |
| 61 | | BP494 |
| 62 | | BP495 |
| 63 | | BP498 |
| 64 | | BP499 |
| 65 | | BP503 |
| 66 | | BP509 |
| 67 | | BP510 |
| 68 | | BP511 |
| 69 | IL6R | A5 |
| 70 | | Fc(P587) |
| 71 | | Fc(P588) |
| 72 | IL6R | P587 |
| 73 | IL6R | P587-LS |
| 74 | IL6R-L2 | k0 |
| 75 | | BP557 |
| 76 | | BP559 |
| 77 | | BP567 |
| 78 | | Fc(DLE) |
| 79 | | Fc(YTE) |
| 80 | | Fc(EF) |
| 81 | | Fc(P208) |

### INDUSTRIAL APPLICABILITY

An Fc region variant with enhanced FcγRIIb-binding activity, and enhanced binding selectivity to FcγRIIb compared to FcγRIIa (type R), as compared to those of a polypeptide comprising an Fc region to which amino acid alteration(s) have not been introduced; and a polypeptide which comprises the Fc region variant. Use of the polypeptide enables transmission of an inhibitory signal of inflammatory immune response mediated by phosphorylation of ITIM of FcγRIIb. Furthermore, by conferring an antibody Fc with the property of selective FcγRIIb binding, anti-drug antibody production may be suppressed through FcγRIIb-mediated immunosuppressive actions.

1. An Fc region variant in which amino acid at position 238 according to EU numbering and at least one amino acid selected from those at positions 233, 234, 235, 237, 264, 265, 266, 267, 268, 269, 271, 272, 274, 296, 326, 327, 330, 331, 332, 333, 334, 355, 356, 358, 396, 409, and 419 according to EU numbering are altered to other amino acids, wherein binding activity of the variant to Fcγ receptors [KD value for FcγRIIb of a polypeptide comprising an Fc region to which an amino acid alteration(s) has not been introduced]/[KD value for FcγRIIb of a polypeptide comprising the Fc region variant] has a value which is 15.0 or greater.

2. The Fc region variant of claim 1, wherein the amino acids at positions 238, 268, and 271 according to EU numbering are altered to other amino acids, and in addition, at least one amino acid selected from amino acids at positions 233, 237, 264, 267, 272, 296, 327, 330, 332, and 396 according to EU numbering are altered to other amino acids.

3. An Fc region variant whose amino acid at position 238 according to EU numbering is Asp, and which comprises at least one amino acid selected from the amino acid group consisting of Asp at amino acid position 233, Tyr at amino acid position 234, Phe at amino acid position 235, Asp at amino acid position 237, Ile at amino acid position 264, Glu at amino acid position 265, Phe, Leu, or Met at amino acid position 266, Ala, Glu, Gly, or Gln at amino acid position 267, Asp, Gln, or Glu at amino acid position 268, Asp at amino acid position 269, Gly at amino acid position 271, Asp, Phe, Ile, Met, Asn, Pro, or Gln at amino acid position 272, Gln at amino acid position 274, Asp or Phe at amino acid position 296, Ala or Asp at amino acid position 326, Gly at amino acid position 327, Lys, Arg, or Ser at amino acid position 330, Ser at amino acid position 331, Lys, Arg, Ser, or Thr at amino acid position 332, Lys, Arg, Ser, or Thr at amino acid position 333, Arg, Ser, or Thr at amino acid position 334, Ala or Gln at amino acid position 355, Glu at amino acid position 356, Met at amino acid position 358, Ala, Asp, Glu, Phe, Gly, His, Ile, Lys, Leu, Met, Asn, Gln, Arg, Ser, Thr, Val, Trp, or Tyr at amino acid position 396, Arg at amino acid position 409, and Glu at amino acid position 419, wherein binding activity of the variant to Fcγ receptors [KD value for FcγRIIb of a polypeptide comprising an Fc region to which an amino acid alteration(s) has not been introduced]/[KD value for FcγRIIb of a polypeptide comprising the Fc region variant] has a value which is 15.0 or greater.

4. The Fc region variant of claim 3, wherein the amino acid at position 238 is Asp, the amino acid at position 268 is Asp or Glu, and the amino acid at position 271 is Gly, according to EU numbering, and wherein the Fc region variant further comprises at least one amino acid selected from the amino acid group consisting of Asp at amino acid position 233, Asp at amino acid position 237, Ile at amino acid position 264, Ala or Gly at amino acid position 267, Asp or Pro at amino acid position 272, Asp at amino acid position 296, Gly at amino acid position 327, Arg at amino acid position 330, Thr at amino acid position 332, and Leu or Met at amino acid position 396.

5. The Fc region variant of claim 1, wherein the value for [KD value for FcγRIIb of a polypeptide comprising an Fc region to which an amino acid alteration(s) has not been introduced]/[KD value for FcγRIIb of a polypeptide comprising an Fc region variant] is 50.0 or more.

6. The Fc region variant of claim 1, wherein the value for [KD value for FcγRIIb of a polypeptide comprising an Fc region to which an amino acid alteration(s) has not been introduced]/[KD value for FcγRIIb of a polypeptide comprising an Fc region variant] is 100.0 or more.

7. The Fc region variant of claim 1, wherein the value for [KD value for FcγRIIa (type R) of a polypeptide comprising an Fc region variant]/[KD value for FcγRIIb of a polypeptide comprising an Fc region variant] is 10.0 or greater.

8. The Fc region variant of claim 1, wherein the value for [KD value for FcγRIIa (type R) of a polypeptide comprising an Fc region variant]/[KD value for FcγRIIb of a polypeptide comprising an Fc region variant] is 20.0 or greater.

9. The Fc region variant of claim 1, wherein the Fc region variant comprises any one of the following set of amino acid alterations of (a) to (x):
(a) amino acid alterations at positions 238, 233, 237, 268, 271, 296, and 330 (EU numbering) of an Fc region;
(b) amino acid alterations at positions 238, 237, 268, 271, 296, and 330 (EU numbering) of an Fc region;
(c) amino acid alterations at positions 238, 233, 237, 268, 271, 296, 330, and 332 (EU numbering) of an Fc region;
(d) amino acid alterations at positions 238, 233, 237, 264, 267, 268, 271, and 330 (EU numbering) of an Fc region;
(e) amino acid alterations at positions 238, 233, 237, 267, 268, 271, 296, 330, and 332 (EU numbering) of an Fc region;
(f) amino acid alterations at positions 238, 237, 267, 268, 271, 296, 330, and 332 (EU numbering) of an Fc region;
(g) amino acid alterations at positions 238, 233, 237, 268, 271, 296, 327, and 330 (EU numbering) of an Fc region;
(h) amino acid alterations at positions 238, 233, 237, 264, 267, 268, and 271 (EU numbering) of an Fc region;
(i) amino acid alterations at positions 238, 233, 237, 264, 267, 268, 271, 296, and 330 (EU numbering) of an Fc region;
(j) amino acid alterations at positions 238, 233, 237, 264, 267, 268, 271, 296, 330, and 396 (EU numbering) of an Fc region;
(k) amino acid alterations at positions 238, 237, 264, 267, 268, 271, and 330 (EU numbering) of an Fc region;
(l) amino acid alterations at positions 238, 237, 264, 267, 268, 271, 296, and 330 (EU numbering) of an Fc region;
(m) amino acid alterations at positions 238, 264, 267, 268, and 271 (EU numbering) of an Fc region;
(n) amino acid alterations at positions 238, 264, 267, 268, 271, and 296 (EU numbering) of an Fc region;
(o) amino acid alterations at positions 238, 237, 267, 268, 271, 296, and 330 (EU numbering) of an Fc region;
(p) amino acid alterations at positions 238, 233, 237, 264, 267, 268, 271, 330, and 396 (EU numbering) of the Fc region;
(q) amino acid alterations at positions 238, 233, 237, 264, 267, 268, 271, 296, 327, 330, and 396 (EU numbering) of an Fc region;
(r) amino acid alterations at positions 238, 233, 237, 264, 267, 268, 271, 272, and 296 (EU numbering) of an Fc region;
(s) amino acid alterations at positions 238, 237, 264, 267, 268, 271, 272, and 330 (EU numbering) of an Fc region;
(t) amino acid alterations at positions 238, 237, 264, 267, 268, 271, 272, 296, and 330 (EU numbering) of an Fc region;
(u) amino acid alterations at positions 238, 233, 264, 267, 268, and 271 (EU numbering) of an Fc region;
(v) amino acid alterations at positions 238, 237, 267, 268, 271, 296, and 330 (EU numbering) of an Fc region;
(w) amino acid alterations at positions 238, 264, 267, 268, 271, 272, and 296 (EU numbering) of an Fc region; and
(x) amino acid alterations at positions 238, 233, 264, 267, 268, 271, and 296 (EU numbering) of an Fc region.

10. The Fc region variant of claim 1, wherein the Fc region variant comprises any one of the following amino acid sequences of (a) to (x):
(a) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, and the amino acid at position 330 is Arg, according to EU numbering, in an Fc region;
(b) an amino acid sequence in which the amino acid at position 238 is Asp, the amino acid at position 237 is Asp, the amino acid at position 268 is Asp or Glu, the amino acid at position 271 is Gly, the amino acid at position 296 is Asp, and the amino acid at position 330 is Arg, according to EU numbering, in an Fc region;
(c) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 330 is Arg, and the amino acid at position 332 is Thr, according to EU numbering, in an Fc region;
(d) an amino acid sequence in which the amino acid at position 238 is Asp, the amino acid at position 233 is Asp, the amino acid at position 237 is Asp, the amino acid at position 264 is Ile, the amino acid at position 267 is Gly or Ala, the amino acid at position 268 is Glu, the amino acid at position 271 is Gly, and the amino acid at position 330 is Arg, according to EU numbering, in an Fc region;
(e) an amino acid sequence in which the amino acid at position 238 is Asp, the amino acid at position 233 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(f) an amino acid sequence in which the amino acid at position 238 is Asp, the amino acid at position 237 is Asp, the amino acid at position 267 is Ala, the amino acid at position 268 is Glu, the amino acid at position 271 is Gly, the amino acid at position 296 is Asp, the amino acid at position 330 is Arg, and the amino acid at position 332 is Thr, according to EU numbering, in an Fc region;
(g) an amino acid sequence in which the amino acid at position 238 is Asp, the amino acid at position 233 is Asp, the amino acid at position 237 is Asp, the amino acid at position 268 is Asp, the amino acid at position 271 is Gly, the amino acid at position 296 is Asp, the amino acid at position 327 is Gly, and the amino acid at position 330 is Arg, according to EU numbering, in an Fc region;
(h) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, and the amino acid at position 271 is Gly, according to EU numbering, in an Fc region;
(i) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(j) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 330 is Arg, and the amino acid at position 396 is Met or Leu, according to EU numbering, in an Fc region;
(k) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, and the amino acid at position 330 is Arg, according to EU numbering, in an Fc region;
(l) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, and the amino acid at position 330 is Arg, according to EU numbering, in an Fc region;
(m) an amino acid sequence in which the amino acid at position 238 is Asp, the amino acid at position 264 is Ile, the amino acid at position 267 is Ala, the amino acid at position 268 is Glu, and the amino acid at position 271 is Gly, according to EU numbering, in an Fc region;
(n) an amino acid sequence in which the amino acid at position 238 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region;
(o) an amino acid sequence in which the amino acid at position 238 is Asp, the amino acid at position 237 is Asp, the amino acid at position 267 is Ala or Gly, the amino acid at position 268 is Glu, the amino acid at position 271 is Gly, the amino acid at position 296 is Asp, and the amino acid at position 330 is Arg, according to EU numbering, in an Fc region;
(p) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 330 is Arg, and the amino acid at position 396 is Met or Leu, according to EU numbering, in an Fc region;
(q) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 296 is Asp, the amino acid at position 327 is Gly, the amino acid at position 330 is Arg, and the amino acid at position 396 is Met, according to EU numbering, in an Fc region;
(r) an amino acid sequence in which the amino acid at position 238 is Asp, the amino acid at position 233 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region;
(s) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, and the amino acid at position 330 is Arg, according to EU numbering, in an Fc region;
(t) an amino acid sequence in which the amino acid at position 238 is Asp, the amino acid at position 237 is Asp, the amino acid at position 264 is Ile, the amino acid at position 267 is Ala, the amino acid at position 268 is Glu, the amino acid at position 271 is Gly, the amino acid at position 272 is Pro, the amino acid at position 296 is Asp, and the amino acid at position 330 is Arg, according to EU numbering, in an Fc region;
(u) an amino acid sequence in which the amino acid at position 238 is Asp, the amino acid at position 233 is Asp, the amino acid at position 264 is Ile, the amino acid at position 267 is Ala, the amino acid at position 268 is Glu, and the amino acid at position 271 is Gly, according to EU numbering, in an Fc region;
(v) an amino acid sequence in which the amino acid at position 238 is Asp, the amino acid at position 237 is Asp, the amino acid at position 267 is Gly, the amino acid at position 268 is Asp, the amino acid at position 271 is Gly, the amino acid at position 296 is Asp, and the amino acid at position 330 is Arg, according to EU numbering, in an Fc region;
(w) an amino acid sequence in which the amino acid at position 238 is Asp, the amino acid at position 264 is Ile, the amino acid at position 267 is Ala, the amino acid at position 268 is Glu, the amino acid at position 271 is Gly, the amino acid at position 272 is Asp, and the amino acid at position 296 is Asp, according to EU numbering, in an Fc region; and
(x) an amino acid sequence in which the amino acid at position 238 is Asp, the amino acid at position 233 is Asp, the amino acid at position 264 is Ile, the amino acid at position 267 is Ala, the amino acid at position 268 is Glu, the amino acid at position 271 is Gly, and the amino acid at position 296 is Asp, according to EU numbering, in an Fc region.

11. An Fc region variant consisting of any one amino acid sequence selected from among SEQ ID NOs: 43 to 68, SEQ ID NO: 70, SEQ ID NO: 71, and SEQ ID NOs: 75 to 77.

12. A polypeptide comprising at least two Fc region variants of claim 1 wherein the two Fc region variants are associated.

13. The polypeptide of claim 12, wherein the amino acid sequences of the two associated Fc region variants in the polypeptide are the same.

14. The polypeptide of claim 12, wherein the amino acid sequences of the two associated Fc region variants in the polypeptide are different.

15. The polypeptide of claim 14, wherein the amino acid sequences of the two associated Fc region variants have different amino acid(s) at at least one amino acid position selected from positions 235, 236, 237, 238, and 239 according to EU numbering in the Fc region variant.

16. The polypeptide of claim 15, wherein one of the amino acid sequences of the two associated Fc region variants is an amino acid sequence comprising at least one amino acid selected from Asp, Gln, Glu, or Thr at amino acid position 235, Asn at amino acid position 236, Phe or Trp at amino acid position 237, Glu, Gly, or Asn at amino acid position 238, and Asp or Glu at amino acid position 239 according to EU numbering.

17. The polypeptide of claim 12, wherein the polypeptide comprising the Fc region variant is an IgG antibody.

18. The polypeptide of claim 12, wherein the polypeptide comprising the Fc region variant is an Fc fusion protein molecule.

19. A pharmaceutical composition comprising the polypeptide of claim 12.


SEQUENCE LISTING (81 sequences)

SEQ ID NO: 1 - DNA, 1125 bp, Homo sapiens
  source 1..1125: mol_type = genomic DNA
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 2 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 3 - DNA, 951 bp, Homo sapiens
  source 1..951: mol_type = genomic DNA
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatgggggtcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 4 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 5 - DNA, 876 bp, Homo sapiens
  source 1..876: mol_type = genomic DNA
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 6 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 7 - DNA, 765 bp, Homo sapiens
  source 1..765: mol_type = genomic DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 8 - protein, 254 aa, Homo sapiens [sequence omitted]

SEQ ID NO: 9 - DNA, 702 bp, Homo sapiens
  source 1..702: mol_type = genomic DNA
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttga

SEQ ID NO: 10 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 11 - protein, 330 aa, synthetic construct [sequence omitted]

SEQ ID NO: 12 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 115 aa, synthetic construct
QVQLVQSGAEVKKPGASVTVSCKASGYTFTDYEMHWIRQPPGEGLEWIGAIDPKTGDTAYSESFQDRVTLTADKSTSTAYMELSSLTSEDTAVYYCTRFYSYTYWGQGTLVTVSS

SEQ ID NO: 16 - protein, 219 aa, synthetic construct
DIVMTQSPLSLPVTPGEPASISCQASESLVHSNRNTYLHWYLQKPGQSPQLLIYKVSNRFSGVPDRFSGSGSGTDFTLKISRVEAEDVGVYYCSQNTHVPPTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 443 aa, synthetic construct
QVQLVQSGAEVKKPGASVTVSCKASGYTFTDYEMHWIRQPPGEGLEWIGAIDPKTGDTAYSESFQDRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 18 - protein, 119 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 19 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 20 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 214 aa, synthetic construct [sequence omitted]

SEQ ID NO: 22 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSP

SEQ ID NO: 23 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 24 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 25 - protein, 449 aa, synthetic construct
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 26 - protein, 218 aa, synthetic construct
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 28 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 29 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 30 - protein, 444 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 31 - protein, 444 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPDLLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 32 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 33 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVAEEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 34 - protein, 443 aa, synthetic construct
QVQLVQSGAEVKKPGASVTVSCKASGYTFTDYEMHWIRQPPGEGLEWIGAIDPKTGDTAYSESFQDRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 35 - protein, 443 aa, synthetic construct
QVQLVQSGAEVKKPGASVTVSCKASGYTFTDYEMHWIRQPPGEGLEWIGAIDPKTGDTAYSESFQDRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNRYTQKSLSLSP

SEQ ID NO: 36 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDYLGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 37 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLQGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 38 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLFGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 39 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLDGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 40 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLEGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 41 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLTGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 42 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNRYTQKSLSLSP

SEQ ID NO: 43 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 44 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 45 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVSEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 46 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKALPRPTEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 47 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVGEEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 48 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVAEEDGEVKFNWYVDGVEVHNAKTKPREEQDNSTYRVVSVLTVLHQDWLNGKEYKCKVSNKALPRPTEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 49 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVAEEDGEVKFNWYVDGVEVHNAKTKPREEQDNSTYRVVSVLTVLHQDWLNGKEYKCKVSNKALPRPTEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 50 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 51 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKGLPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 52 - protein, 325 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPDLLGDDSVFLFPPKPKDTLMISRTPEVTCVVVDVSDEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 53 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 54 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPMVLDSDGSFFLYSKLTVDKSRWQQGNVFSCSVMHEALHNHYTQESLSLSP

SEQ ID NO: 55 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 56 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPMVLDSDGSFFLYSKLTVDKSRWQQGNVFSCSVMHEALHNHYTQESLSLSP

SEQ ID NO: 57 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVIDVAEEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 58 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVIDVAEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 59 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVIDVAEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 60 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVIDVAEEDGEVKFNWYVDGVEVHNAKTKPREEQDNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 61 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVAEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 62 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVGEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 63 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQESLSLSP

SEQ ID NO: 64 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQESLSLSP

SEQ ID NO: 65 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGEVKFNWYVDGVEVHNAKTKPREEQDNSTYRVVSVLTVLHQDWLNGKEYKCKVSNKGLPRPIEKTISKAKGQPREPQVYTLPPSRDELTKNQVSLTCLVKGFYPSDIAVEWESNGQPENNYKTTPMVLDSDGSFFLYSKLTVDKSRWQQGNVFSCSVMHEALHNHYTQESLSLSP

SEQ ID NO: 66 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVIDVAEEDGDVKFNWYVDGVEVHNAKTKPREEQDNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 67 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVIDVAEEDGPVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 68 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVIDVAEEDGPVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 69 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNRYTQKSLSLSP

SEQ ID NO: 70 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGGDSVFLFPPKPKDTLMISRTPEVTCVVIDVAEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 71 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVIDVAEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 72 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGGDSVFLFPPKPKDTLMISRTPEVTCVVIDVAEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 73 - protein, 447 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGGDSVFLFPPKPKDTLMISRTPEVTCVVIDVAEEDGEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSP

SEQ ID NO: 74 - protein, 214 aa, synthetic construct
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDDSVFLFPPKPKDTLMISRTPEVTCVVVDVGDEDGEVKFNWYVDGVEVHNAKTKPREEQDNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 76 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVIDVAEEDGDVKFNWYVDGVEVHNAKTKPREEQDNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 77 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGGDSVFLFPPKPKDTLMISRTPEVTCVVIDVAEEDGEVKFNWYVDGVEVHNAKTKPREEQDNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 78 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 79 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 80 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP